SEQUENCE LISTING (1 sequence)

SEQ ID NO: 1 - DNA, 21 bp, synthetic construct
  source 1..21: mol_type = other DNA
tgccatcatcacacagctcct

(19) United States
(12) Patent Application Publication
(10) Pub. No.: US 2026/0200939 A1
(43) Pub. Date: Jul. 16, 2026

(54) MOLECULAR GLUE DEGRADER COMPOUNDS AND USES THEREOF

(71) Applicants: Novartis AG, Basel (CH); THE REGENTS OF THE UNIVERSITY OF CALIFORNIA, Oakland, CA (US)

(72) Inventors: Jeffrey M. McKenna, Carlisle, MA (US); Daniel K. Nomura, Walnut Creek, CA (US); Ethan Shigeru Toriki, Berkeley, CA (US); James William Papatzimas, Berkeley, CA (US); Dustin Leard Dovala, San Leandro, CA (US); Matthew James Hesse, Oakland, CA (US); Kaila Yukika Nishikawa, Belmont, CA (US)

(21) Appl. No.: 19/122,981

(22) PCT Filed: Oct. 20, 2023
(86) PCT No.: PCT/US2023/035642

§ 371 Date: Apr. 21, 2025

Related U.S. Application Data
(60) Provisional application No. 63/418,053, filed on Oct. 21, 2022.

(51) Int. Cl. C07D 495/14; A61K 31/426; A61K 31/4965; A61K 31/50; A61K 31/519; C07C 235/78; C07D 233/88; C07D 237/20; C07D 241/04; C07D 471/04; C07D 487/04

(52) U.S. Cl. CPC C07D 495/14; A61K 31/426; A61K 31/4965; A61K 31/50; A61K 31/519; C07C 235/78; C07D 233/88; C07D 237/20; C07D 241/04; C07D 471/04; C07D 487/04

(57) ABSTRACT

Described herein are molecular glue degrader compounds that bind to both a target protein and a RING E3 Ubiquitin Ligase, as well as related compositions and methods of use, e.g., for degradation of the target protein and/or the treatment of a disease, disorder, or condition.

### CLAIM OF PRIORITY

The present application is a U.S. National Phase Application under 35 U.S.C. § 371 of International Application No. PCT/US2023/035642, filed Oct. 20, 2023, which claims priority to U.S. Provisional Application No. 63/418,053, filed Oct. 21, 2022; the contents of which are incorporated herein by reference in their entirety.

### STATEMENT REGARDING FEDERALLY-SPONSORED RESEARCH OR DEVELOPMENT

This invention was made with government support under Grant Number CA240981 awarded by the National Institutes of Health and under Grant Number 2127788 awarded by the National Science Foundation. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 3, 2026, is entitled “N2113-7000USFSR_ST26.xml”, and is 1,989 bytes in size.

### FIELD OF THE DISCLOSURE

Described herein are molecular glue degrader compounds that bind to both a target protein and a RING E3 Ubiquitin Ligase, as well as related compositions and methods of use, e.g., for degradation of the target protein and/or the treatment of a disease, disorder, or condition.

### BACKGROUND

Proteasome-mediated degradation of unneeded or damaged proteins plays an important role in maintaining regular cellular functions, such as cell survival, proliferation and growth. In particular, the Ubiquitin-Proteasome Pathway (UPP) is central to multiple cellular processes, and if defective or imbalanced, leads to pathogenesis of a variety of diseases. Targeted protein degradation (TPD) has arisen as a powerful therapeutic modality for eliminating classically undruggable disease-causing proteins through ubiquitination and proteasome-mediated degradation. Two major approaches for TPD include heteromolecular glue Proteolysis Targeting Chimeras (PROTACs) or molecular glue degraders, each which result in the ubiquitination and degradation of the target protein in a proteasomal dependent manner. While much is understood about PROTAC structure and mechanism, the mechanism of action of certain molecular glue degrader compounds is less clear. As such, there is a need for a deeper understanding of the chemical design principles for converting protein-targeting ligands into molecular glue degraders and related methods of use thereof.

### SUMMARY

The present disclosure features molecular glue degrader compounds, as well as pharmaceutically acceptable salts, hydrates, solvates, prodrugs, stereoisomers, or tautomers thereof, capable of inducing degradation of a target protein. In one aspect, the present disclosure features a molecular glue degrader compound comprising: (i) a Target Ligand capable of binding to a target protein; and (ii) a RING E3 Ligase Binder capable of binding to a RING E3 ubiquitin ligase. In an embodiment, the molecular glue degrader compound further comprises a bridge domain linking the Target Ligand and the RING E3 Ligase Binder. In another embodiment, the molecular glue degrader compound does not comprise a bridge domain linking the Target Ligand and the RING E3 Ligase Binder, i.e., the Target Ligand and the RING E3 Ligase Binder are directly bound to one another. In an embodiment, the molecular glue degrader compounds have the structure of Formula (I):

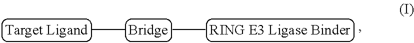

or a pharmaceutically acceptable salt, hydrate, solvate, prodrug, stereoisomer, or tautomer thereof, wherein (i) the Target Ligand is capable of binding to a target protein; (ii) Bridge is absent or a linker; and (iii) the RING E3 Ligase Binder is capable of binding to a RING E3 ubiquitin ligase (e.g., RNF126 or BCA2). In an embodiment, the RING E3 Ligase Binder binds to RNF126 E3 ubiquitin ligase, BCA2 E3 ubiquitin ligase, LRSAM1 E3 ubiquitin ligase, RNF40 E3 ubiquitin ligase, MID2 E3 ubiquitin ligase, RNF219 E3 ubiquitin ligase, or RNF14 E3 ubiquitin ligase. In an embodiment, the RING E3 Ligase Binder binds to RNF126 E3 ubiquitin ligase. In an embodiment, the RING E3 Ligase Binder binds to a cysteine residue within a RING E3 ubiquitin ligase. In an embodiment, the RING E3 Ligase Binder covalently binds to a cysteine residue within a RING E3 ubiquitin ligase. In an embodiment, the RING E3 Ligase Binder binds to a non-catalytic cysteine residue within RNF126 (e.g., C32). In an embodiment, the RING E3 Ligase Binder binds to BCA2 E3 ubiquitin ligase. In an embodiment, the RING E3 Ligase Binder binds to LRSAM1 E3 ubiquitin ligase. In an embodiment, the RING E3 Ligase Binder binds to MID2 E3 ubiquitin ligase. In an embodiment, the RING E3 Ligase Binder binds to RNF40 E3 ubiquitin ligase. In an embodiment, the RING E3 Ligase Binder binds to RNF219 E3 ubiquitin ligase. In an embodiment, the RING E3 Ligase Binder binds to RNF14 E3 ubiquitin ligase.

In an embodiment, the RING E3 Ligase Binder comprises an electrophilic moiety (e.g., C₂₋₁₀ alkenylene moiety). In an embodiment, the RING E3 Ligase Binder comprises a comprises a cinnamamide moiety or a fumarate moiety.

In an embodiment, the target protein selected from a tyrosine kinase, a serine/threonine kinase, a bromodomain-containing protein, an epigenetic protein, and a misfolded protein. In an embodiment, the target protein is selected from AR, BCL-2/BCL, BCL-XL, BCR-ABL, BRD2, BRD3, BRD4, BRD9, BRDT, BTK, BUB1, BUB1B, c-ABL, CDK1, CDK2, CDK3, CDK4, CDK5, CDK6, CDK7, CDK8, CDK9, CDK10, CDK11B, CDK12, CDK13, CDK14, CDK15, CDK16, CDK17, CDK18, CDK19, CDK20, CHEK1, CKS1B, CKS2, CSNK1A1, CSNK1E, CTNNB1, DSTYK, EEF2K, ER, ETNK1, FASTKD5, HRAS, ITPKB, KRAS, LRKK2, MAPKAPK2 (MK2), MARK2, MAP3K2, MELK, MYC, MYCN, NEK6, NRAS, PANK2, PANK3, PDE5, PHKA1, PHKA2, PKN2, PLK1, PTK6, RIOK2, SKP2, SMARCA2, SMARCA4, STAT1, STAT2, STAT3, STAT4, STAT5A, STAT5B, STAT6, TK1, TTK, and UCK2, or a mutant, isoform, or fragment thereof. For example, the target protein may be AR or a mutant or isoform thereof, e.g., AR H874Y, AR F876L, AR T877A, AR W741L/C, AR-V1, AR-V2, AR-V3, AR-V4, AR-V5, AR-V6, AR-V7, AR-V9, or ARv567es. For example, the target protein may be KRAS or a mutant or isoform thereof, e.g., KRAS4A, KRAS4B, KRAS G12A, KRAS G12C, KRAS G12D, KRAS G12S, KRAS G12V, or KRASG13D. In another example, the target protein may be AR or a mutant or isoform thereof, e.g., AR H874Y, AR F876L, AR T877A, or AR W741L/C.

In an embodiment, the molecular glue degrader compounds have the structure of Formula (I-a):

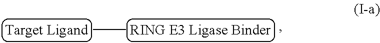

or a pharmaceutically acceptable salt, hydrate, solvate, prodrug, stereoisomer, or tautomer thereof, wherein (i) the Target Ligand is capable of binding to a target protein; and (iii) the RING E3 Ligase Binder is capable of binding to a RING E3 ubiquitin ligase (e.g., RNF126 or BCA2). In an embodiment, the RING E3 Ligase Binder binds to RNF126 E3 ubiquitin ligase, BCA2 E3 ubiquitin ligase, LRSAM1 E3 ubiquitin ligase, RNF40 E3 ubiquitin ligase, MID2 E3 ubiquitin ligase, RNF219 E3 ubiquitin ligase, or RNF14 E3 ubiquitin ligase. In an embodiment, the RING E3 Ligase Binder binds to RNF126 E3 ubiquitin ligase. In an embodiment, the RING E3 Ligase Binder binds to a cysteine residue within a RING E3 ubiquitin ligase. In an embodiment, the RING E3 Ligase Binder covalently binds to a cysteine residue within a RING E3 ubiquitin ligase. In an embodiment, the RING E3 Ligase Binder binds to a non-catalytic cysteine residue within RNF126 (e.g., C32). In an embodiment, the RING E3 Ligase Binder binds to BCA2 E3 ubiquitin ligase. In an embodiment, the RING E3 Ligase Binder binds to LRSAM1 E3 ubiquitin ligase In an embodiment, the RING E3 Ligase Binder binds to MID2 E3 ubiquitin ligase In an embodiment, the RING E3 Ligase Binder binds to RNF40 E3 ubiquitin ligase. In an embodiment, the RING E3 Ligase Binder binds to RNF219 E3 ubiquitin ligase. In an embodiment, the RING E3 Ligase Binder binds to RNF14 E3 ubiquitin ligase.

In an embodiment, the molecular glue degrader compound has a structure of Formula (II):

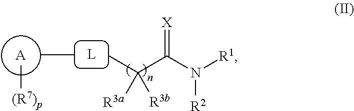

or a pharmaceutically acceptable salt, hydrate, solvate, stereoisomer, or tautomer thereof, wherein X is O or S; Ring A is selected from the group consisting of an aryl, a heteroaryl, a cycloalkyl, and a heterocycloalkyl; L is absent or a linker; R¹ is

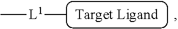

wherein L¹ is absent or a linker and the Target Ligand comprises a moiety capable of binding to a target protein; or —NR¹ is a Target Ligand; R² is selected from the group consisting of hydrogen and C₁₋₆ alkyl; or R¹ and R² together with the atoms to which they are attached form a 5- to 10-membered heteroaryl or a 3- to 12-membered heterocycloalkyl, wherein the heteroaryl or heterocycloalkyl are each substituted with one R⁸, wherein R⁸ is

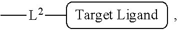

wherein L² is a linker, and the heteroaryl or heterocycloalkyl are each substituted with 0-4 occurrences of R⁹; each R⁹ is independently selected from the group consisting of —OH, halogen, C₁₋₆ alkyl, C₁₋₆ alkoxyl, C₁₋₆ haloalkyl, alkynyl, —CN, C₁₋₆ haloalkoxyl, —O-aryl, and heteroaryl; or two R⁹ on the same carbon atom together with the same carbon atom to which they are attached form a C₃₋₈ cycloalkyl or an oxo; or two R⁹ on adjacent carbon atoms together with the adjacent carbon atoms to which they are attached form a C₃₋₈ cycloalkyl; or two R⁹ on non-adjacent carbon atoms together with the non-adjacent carbon atoms to which they are attached form a bridging ring; R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; R⁴ and R⁵ are each independently selected from the group consisting of hydrogen, —OH, halogen, —CN, C₁₋₆ alkyl, and C₁₋₆ haloalkyl; R^{4′} and R^{5′} are each independently selected from the group consisting of hydrogen, —OH, halogen, —CN, C₁₋₆ alkyl, and C₁₋₆ haloalkyl; R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or R^{6a} and R^{6b}, together with the atoms to which they are attached, form an oxo group; R^{6c} is independently selected from the group consisting of hydrogen, C₁₋₆ alkyl, C₁₋₆ hydroxyalkyl, C₁₋₆ haloalkyl, and C₃₋₈ cycloalkyl; R⁷ are each independently selected from the group consisting of —OH, halogen, C₁₋₆ alkyl, C₁₋₆ alkoxyl, C₁₋₆ haloalkyl, alkynyl, —CN, C₁₋₆ haloalkoxyl, —O-aryl, and heteroaryl; or two R⁷ on the same carbon atom together with the atoms which they are attached, form an oxo group; or two R⁷ on adjacent atoms together with the atoms to which they are attached form an optionally substituted 5- to 10-membered aryl or a 3- to 12-membered heterocycloalkyl; n is 0, 1, or 2; and p is 0, 1, 2, 3, or 4.

In an embodiment, the molecular glue degrader compound has a structure of Formula (II-a):

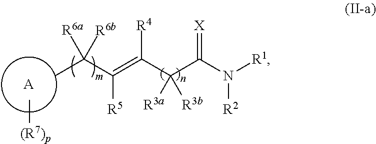

or a pharmaceutically acceptable salt, hydrate, solvate, stereoisomer, or tautomer thereof, wherein X is O or S; Ring A is selected from the group consisting of a 6- to 10-membered aryl, a 5- to 10-membered heteroaryl, a 3- to 12-membered cycloalkyl, and a 3- to 12-membered heterocycloalkyl; R¹ is

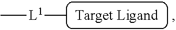

wherein L¹ is absent or a linker and the Target Ligand comprises a moiety capable of binding to a target protein; or —NR¹ is a Target Ligand; R² is selected from the group consisting of hydrogen and C₁₋₆ alkyl; or R¹ and R² together with the atoms to which they are attached form a 5- to 10-membered heteroaryl or a 3- to 12-membered heterocycloalkyl, wherein the heteroaryl or heterocycloalkyl are each substituted with one R⁸, wherein R⁸ is

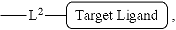

wherein L² is a linker, and the heteroaryl or heterocycloalkyl are each substituted with 0-4 occurrences of R⁹; each R⁹ is independently selected from the group consisting of —OH, halogen, C₁₋₆ alkyl, C₁₋₆ alkoxyl, C₁₋₆ haloalkyl, and C₁₋₆ haloalkoxyl; or two R⁹ on the same carbon atom together with the same carbon atom to which they are attached form a C₃₋₈ cycloalkyl or an oxo; or two R⁹ on adjacent carbon atoms together with the adjacent carbon atoms to which they are attached form a C₃₋₈ cycloalkyl; or two R⁹ on non-adjacent carbon atoms together with the non-adjacent carbon atoms to which they are attached form a bridging ring; R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; R⁴ and R⁵ are each independently selected from the group consisting of hydrogen, —OH, halogen, —CN, C₁₋₆ alkyl, and C₁₋₆ haloalkyl; R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or R^{6a} and R^{6b}, together with the atoms to which they are attached, form an oxo group; R⁷ are each independently selected from the group consisting of —OH, halogen, C₁₋₆ alkyl, C₁₋₆ alkoxyl, C₁₋₆ haloalkyl, and C₁₋₆ haloalkoxyl; m is 0, 1, or 2; n is 0, 1, or 2; and p is 0, 1, 2, 3, or 4.

In an embodiment, X is O. In an embodiment, R² is hydrogen. In an embodiment, each of R^{3a} and R^{3b} is independently hydrogen or C₁₋₆ alkyl. In an embodiment, each of R^{6a} and R^{6b} is independently hydrogen or C₁₋₆ alkyl. In an embodiment, R⁴ and R⁵ are each independently selected from the group consisting of hydrogen, halogen, —CN, C₁₋₆ alkyl, and C₁₋₆ haloalkyl. In an embodiment, each of R⁴ and R⁵ is independently hydrogen or C₁₋₆ alkyl. In an embodiment, m is 1 or 2. In an embodiment, n is 1 or 2. In an embodiment, Ring A is 6- to 10-membered aryl (e.g., phenyl). In an embodiment, p is 0, 1, 2, or 3. In an embodiment, L1 is absent. In an embodiment, L1 is a linker (e.g., a linker described herein). In an embodiment, L2 is absent. In an embodiment, L2 is a linker (e.g., a linker described herein). In an embodiment, the Target Ligand is a kinase inhibitor, bromodomain inhibitor, or phosphodiesterase inhibitor. In an embodiment, the Target Ligand is capable of binding to CDK4, CDK6, BRD4, PDE5, BCR-ABL, c-ABL, AR, AR-V7, BTK, LRRK2, or SMARCA2. In an embodiment, the Target Ligand is selected from ribociclib, dasatinib, palbociclib, sildenafil, HG-10-102-01, JQ1, VPC-14228, ibrutinib, or a derivative thereof.

In another aspect, the present disclosure features a method of treating a target disease, disorder, or condition (e.g., a protein-mediated disorder, disease, or condition) in a patient comprising administering to the patient any of the compounds described herein, or a pharmaceutically acceptable salt, hydrate, solvate, prodrug, stereoisomer, or tautomer thereof. In an embodiment, the disorder is selected from a respiratory disorder, a proliferative disorder, an autoimmune disorder, an autoinflammatory disorder, an inflammatory disorder, a neurological disorder, and an infectious disease or disorder. In an embodiment, the disorder is a proliferative disorder. In an embodiment, the proliferative disorder is cancer.

Another embodiment is a method of treating cancer in a subject in need thereof, the method comprising administering to the subject a therapeutically effective amount of a compound of described herein, or a pharmaceutically acceptable salt thereof.

Another embodiment is a pharmaceutical composition comprising any of the compounds described herein, or a pharmaceutically acceptable salt, hydrate, solvate, prodrug, stereoisomer, or tautomer thereof, and a pharmaceutically acceptable carrier.

Another embodiment is a method of treating a respiratory disorder, a proliferative disorder, an autoimmune disorder, an autoinflammatory disorder, an inflammatory disorder, a neurological disorder, and an infectious disease or disorder in a subject in need thereof, the method comprising administering to the subject a therapeutically effective amount of a compound described herein, or a pharmaceutically acceptable salt thereof. In an embodiment, the disorder is a proliferative disorder. In an embodiment, the proliferative disorder is cancer.

Another embodiment is the use of a compound described herein, or a pharmaceutically acceptable salt, hydrate, solvate, prodrug, stereoisomer, or tautomer thereof in the preparation of a medicament for treating a respiratory disorder, a proliferative disorder, an autoimmune disorder, an autoinflammatory disorder, an inflammatory disorder, a neurological disorder, and an infectious disease or disorder in a subject in need thereof. Exemplary embodiments of the present disclosure are described in further detail herein, including in the Drawings, Description, Examples, and Claims.

### BRIEF DESCRIPTION OF DRAWINGS

FIGS. 1A-1D are immunoblots depicting the results of screening studies for CDK4/6 reduction with exemplary Ribociclib analogs (Compounds 100-107, 170) in C33A cervical cancer cells. In FIG. 1D, the lanes are labeled 1-10, wherein: 1: Ribociclib, 2: Compound 100, 3: Compound 101, 4: Compound 102, 5: Compound 103, 6: Compound 104, 7: Compound 105, 8: Compound 170, 9: Compound 106, 10: Compound 107.

FIGS. 2A-2B are immunoblots demonstrating that the reduction of CDK4 by exemplary compounds occurs via proteosome-mediated degradation, as pre-treatment of C33A cells with proteasome inhibitor bortezomib attenuated the CDK4 degradation.

FIGS. 3A-3B are immunoblots showing the importance of a covalent handle in an exemplary compound (Compound 106) for degradation of CDK4.

FIGS. 4A-4F show that an exemplary compound (Compound 106) covalently binds to cysteine 32 (C32) on the RING-family E3 ubiquitin ligase RNF126. FIG. 4A depicts the results of isotopic tandem orthogonal activity-based protein profiling (isoTOP-ABPP) in which C33A cells were treated in situ with vehicle or Compound 106, then the resulting cell lysates were subsequently labeled with an alkyne-functionalized iodoacetamide probe to identify cysteines that were highly engaged by Compound 106 across the proteome. FIG. 4B confirms the interaction of Compound 106 with RNF126 using gel-based activity-based protein profiling (ABPP) in a dose responsive manner. FIG. 4C depicts the mass spectrometry analysis of Compound 106-labeled RNF126 tryptic digests, in which the mass adduct of Compound 106 on pure RNF126 protein is observed. FIGS. 4D-4F are immunoblots showing that RNF126 knockdown completely attenuated Compound 106-mediated CDK4 degradation in C33A cells, demonstrating that RNF126 is at least in-part responsible for the degradation of CDK4.

FIGS. 5A-5I demonstrate structure-activity studies of the covalent binding handle for inducing CDK4 degradation. FIGS. 5A-5B are gels showing the CDK4 degradation activity of closely related analogs bound to Ribociclib. FIGS. 5C-5D are a gels showing the CDK4 degradation activity of the fumarate analog, Compound 113, in a dose-responsive manner. FIG. 5E depicts the mass spectrometry analysis of Compound 113-labeled RNF126 tryptic digests, confirming that Compound 113 reacts with C32 of RNF126. A non-covalent derivative of Compound 113 exhibits binding to RNF126 (FIG. 5F), but does not induce CDK4 degradation in C33A cells (FIG. 5G). The fumarate handle bound to Palbociclib (Compound 136) maintained binding to RNF126 and was capable to degrading CDK4 (FIGS. 5H-5I).

FIGS. 6A-6K depict studies directed towards identifying the minimal covalent handle required for RNF126 interactions. FIGS. 6A-6J are gel-based ABPP studies, depicting RNF126 binding interactions of compounds comprising iterative additions to the Ribociclib scaffold. FIG. 6K shows the results of a quantitative proteomics study in which HEK293T cells were treated with Compound 122 derivatized with an alkyne or vehicle and subsequently appended an azide-functionalized biotin enrichment handle through copper-catalyzed “click-chemistry” followed by avidin-enrichment of probe modified peptides to assess probe enriched proteins. These studies confirmed that RNF126 is the most significantly enriched E3 ligase by the Compound 122 alkyne probe.

FIGS. 7A-7D show potent binding of Compound 126, comprising a dasatinib analog, to RNF126 (FIG. 7A) and degradation of both BCR-ABL and c-ABL kinase in a dose-responsive manner (FIGS. 7B-7D).

FIGS. 8A-8D show potent binding of Compound 127, comprising an analog of the phosphodiesterase 5 (PDE5) inhibitor Sildenafil, to RNF126 (FIG. 8A) and degradation of PDE5 in HEK293T cells in a dose-responsive and proteasome-dependent manner (FIGS. 8B-8D).

FIGS. 9A-9C show potent binding of Compound 128, comprising a SMARCA2-bromodomain ligand 1 compound, to RNF126 (FIG. 9A) and degradation of SMARCA2 in MV-4-11 leukemia cancer cells cell in a dose-responsive manner (FIGS. 9B-9C).

FIGS. 10A-10C show potent binding of Compound 129, comprising an analog of the LRRK2 inhibitor HG-10-102-01, to RNF126 (FIG. 10A) and loss of LRRK2 in A549 lung cancer cells in a dose-responsive manner (FIGS. 10B-10C).

FIGS. 11A-11H show potent binding of Compound 130, comprising an analog of the BET family bromodomain inhibitor JQ1, to RNF126 (FIG. 11A) and degradation of both the long and short isoforms of BRD4 in HEK293T cells in a dose-responsive, time-dependent, and proteasome-dependent manner (FIGS. 11B-11G). Quantitative proteomic profiling of Compound 130 in HEK293T cells also demonstrated relatively selective degradation of BRD4 with other targets (FIG. 11H).

FIGS. 12A-12C show potent binding of Compound 131, comprising the BTK inhibitor Ibrutinib, to RNF126 (FIG. 12A) and degradation of BTK in MINO lymphoma cancer cells (FIGS. 12B-12C).

FIGS. 13A-13G show potent binding of Compounds 132 and 133, comprising analogs of the androgen receptor (AR) mutant AR-V7 inhibitor VPC-14228, to RNF126 (FIG. 13A) and degradation of AR and AR-V7 in LNCaP and 22RV1 prostate cancer cells (FIGS. 13B-13G).

FIGS. 14A-14BB are gel-based binding studies of Compounds 138-163 with RNF126.

FIGS. 15A-15E are gel-based binding studies of exemplary compounds with either BRD4 (FIGS. 15A, 15C-D) or CDK4 (FIG. 15B).

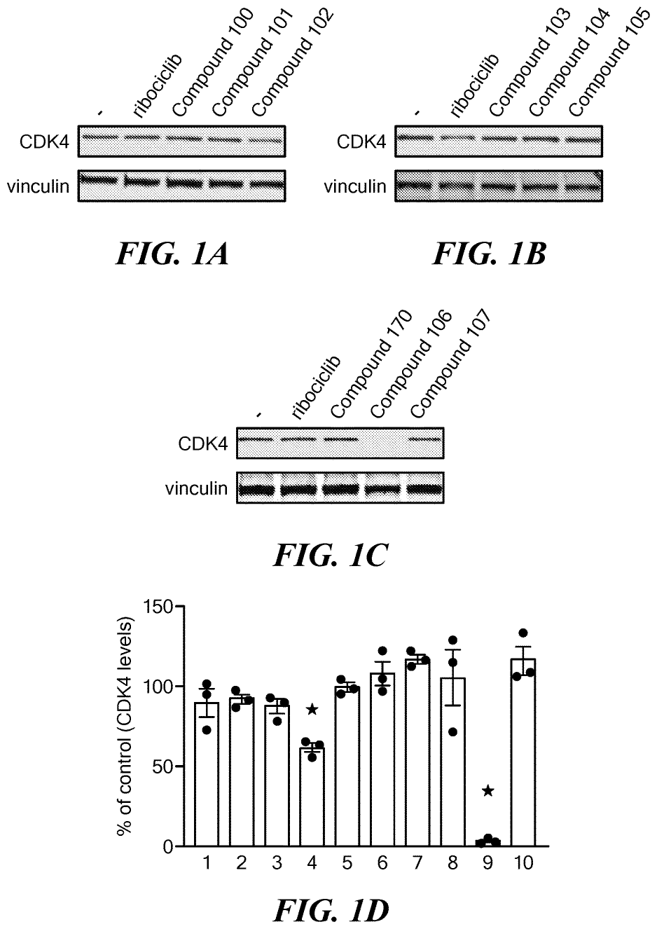

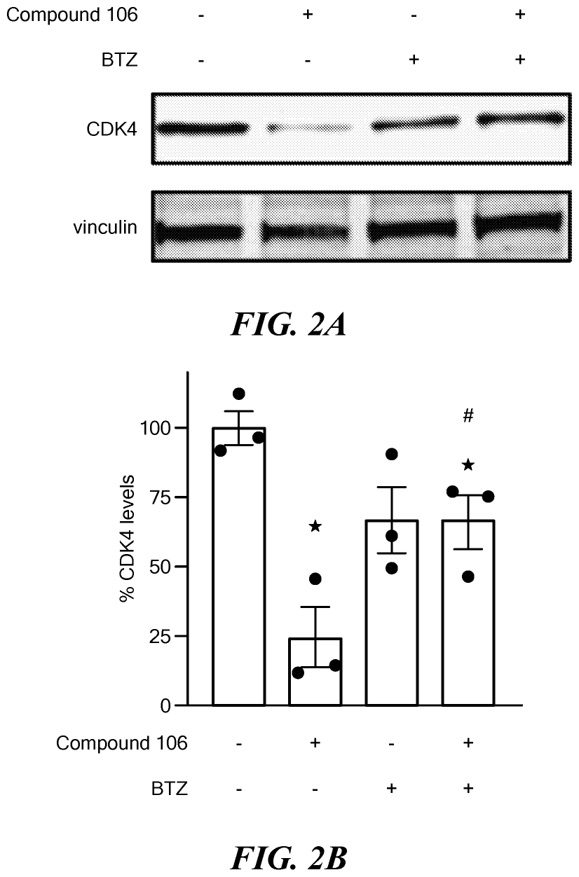

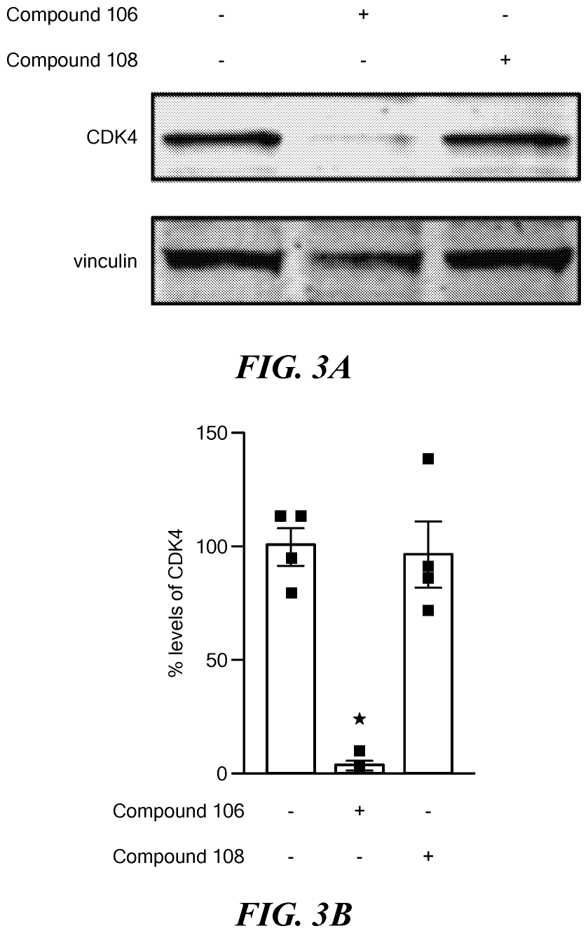

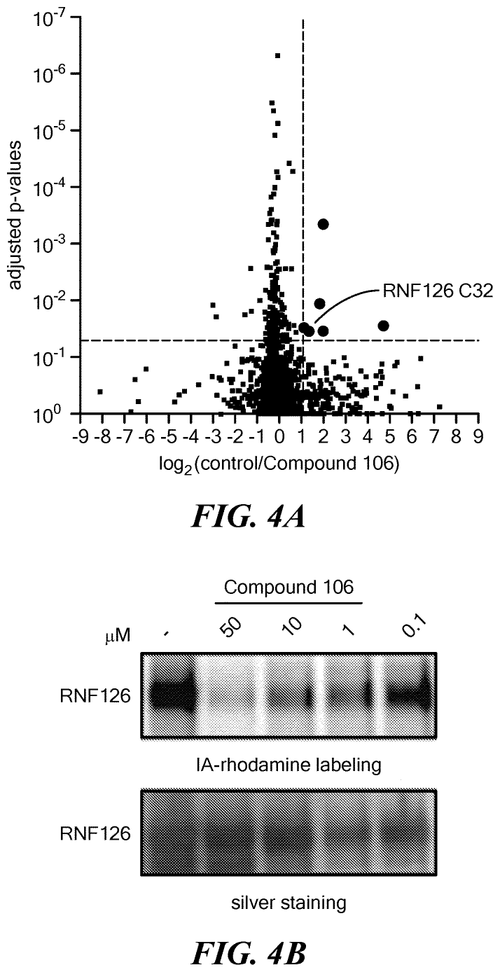

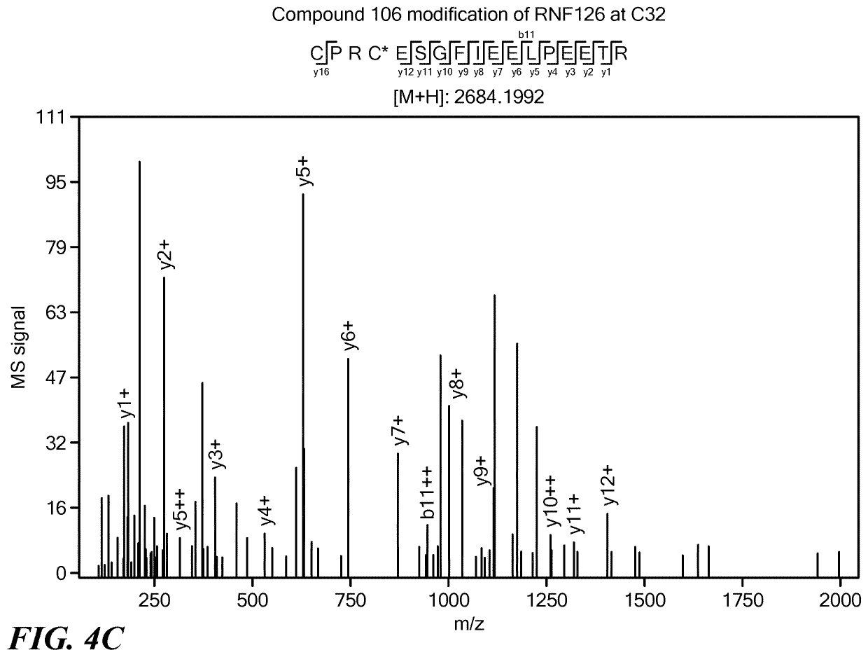

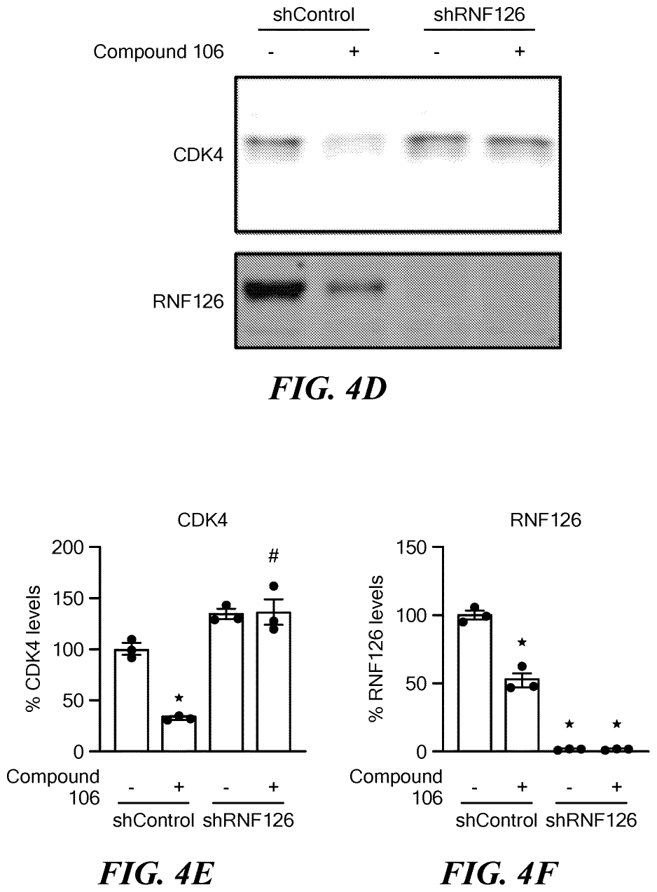

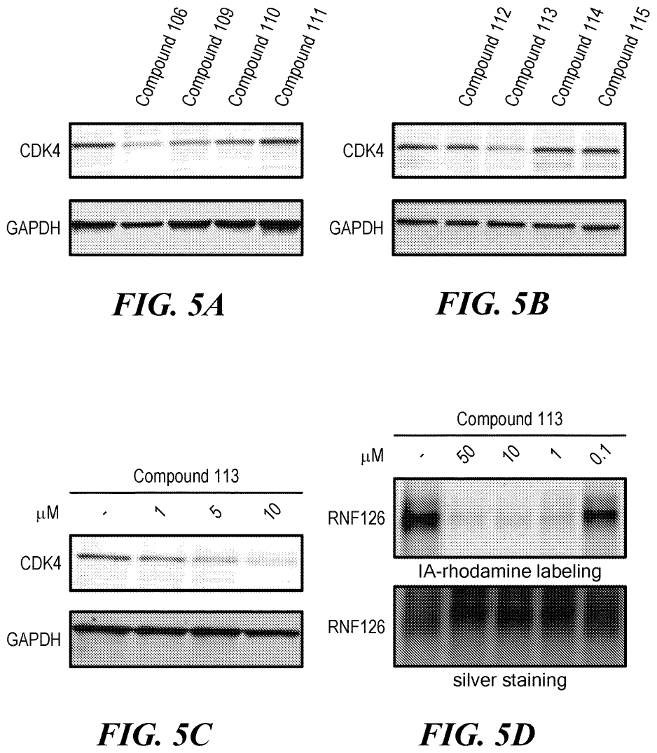

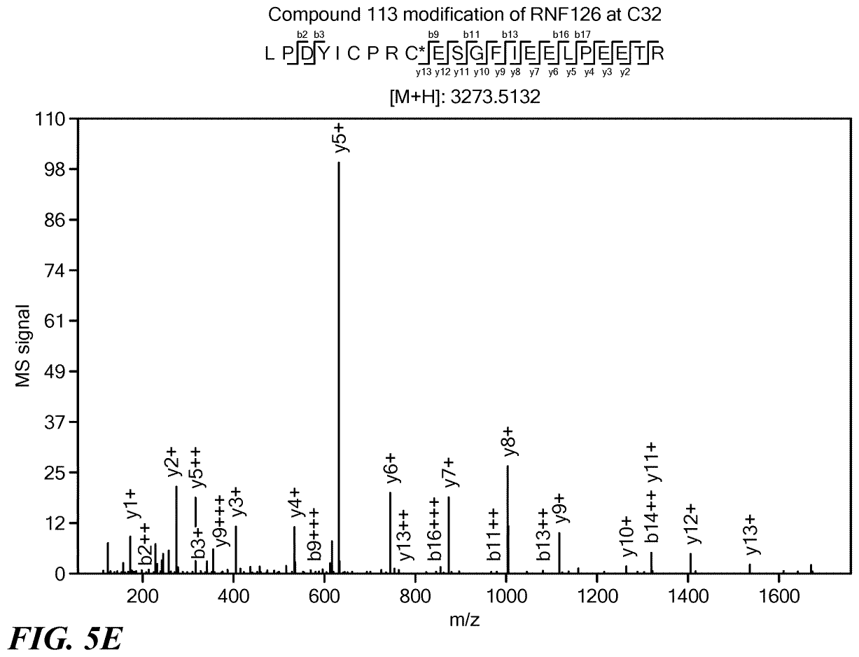

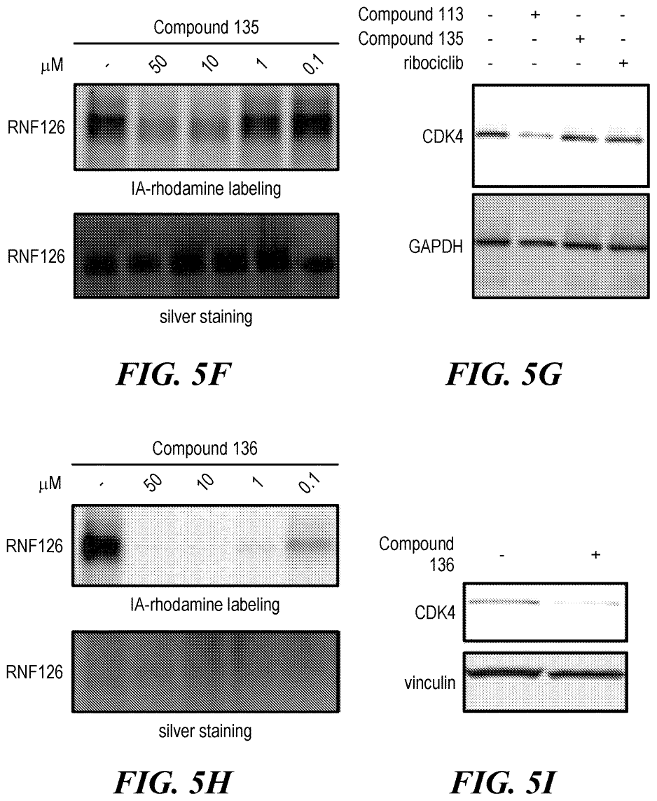

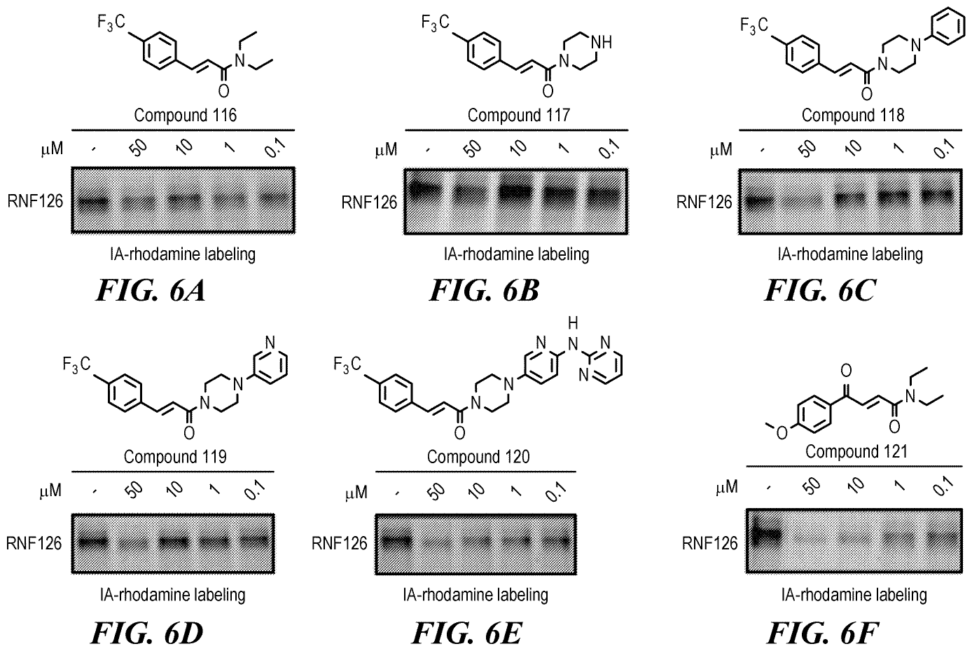

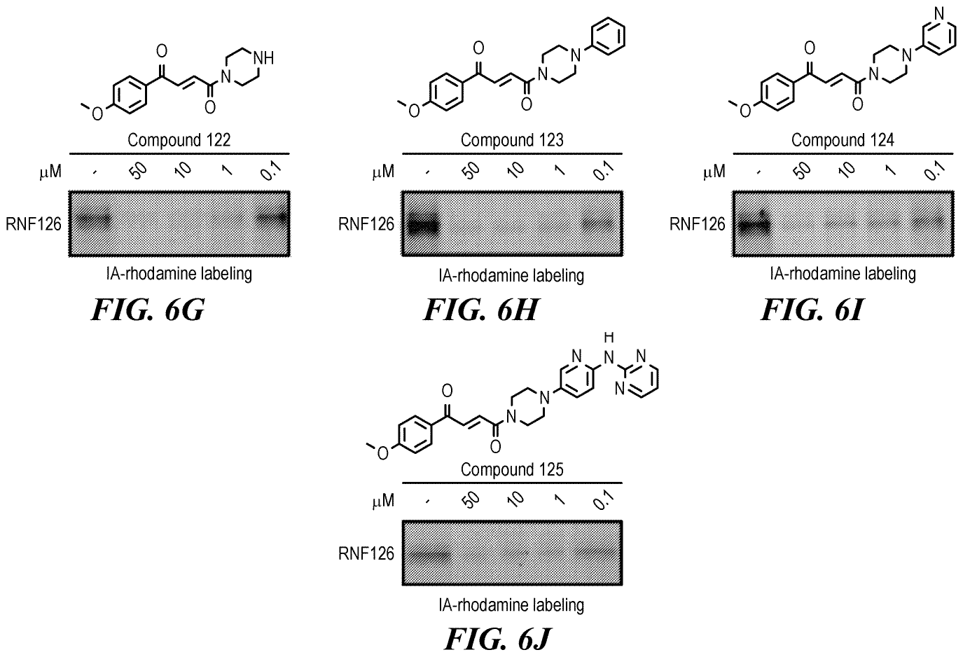

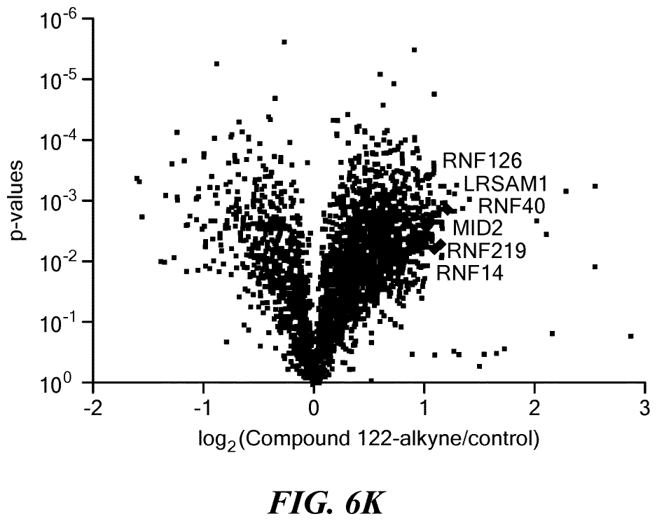

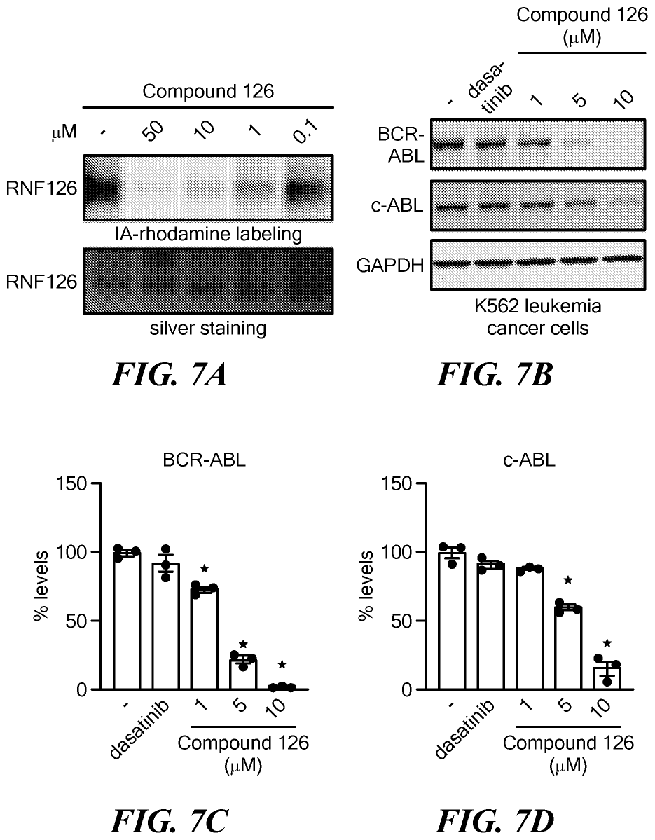

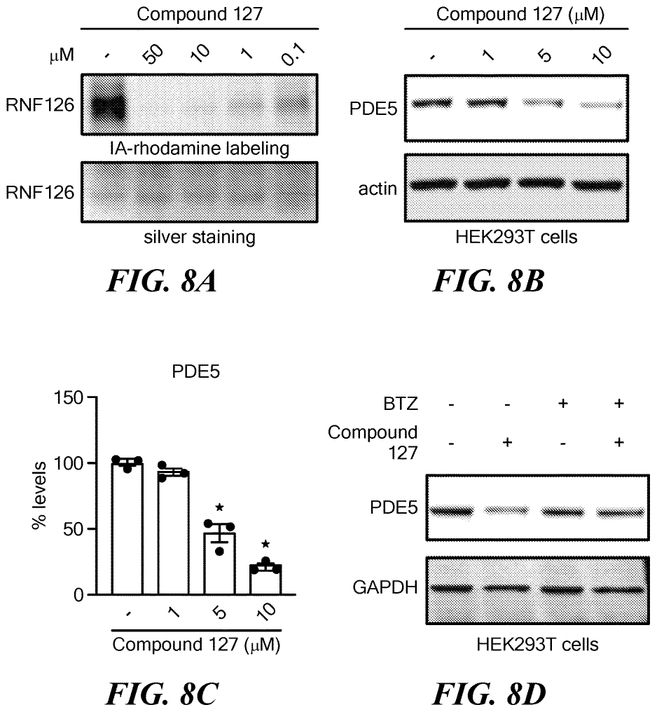

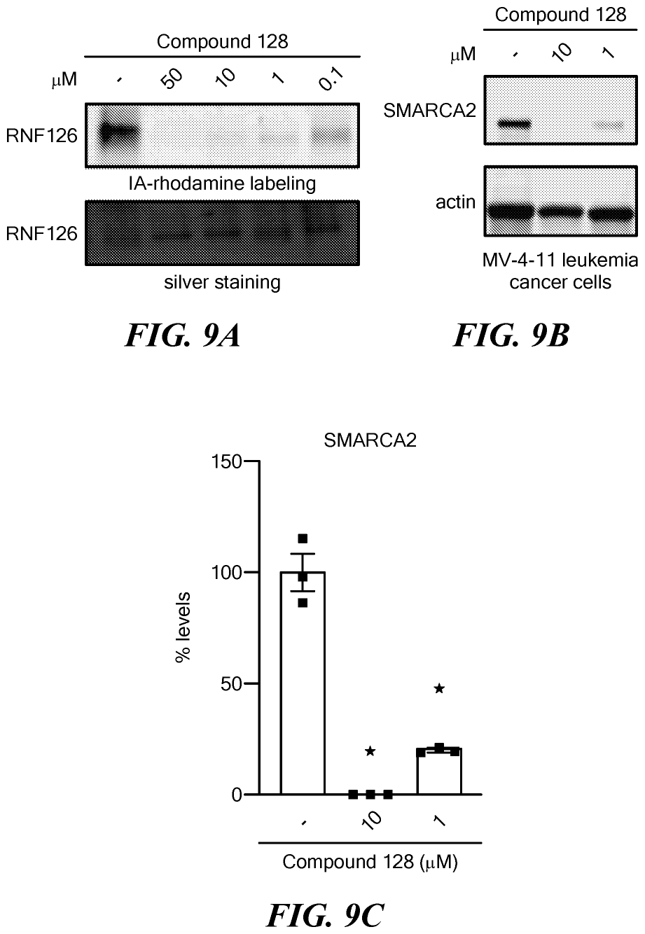

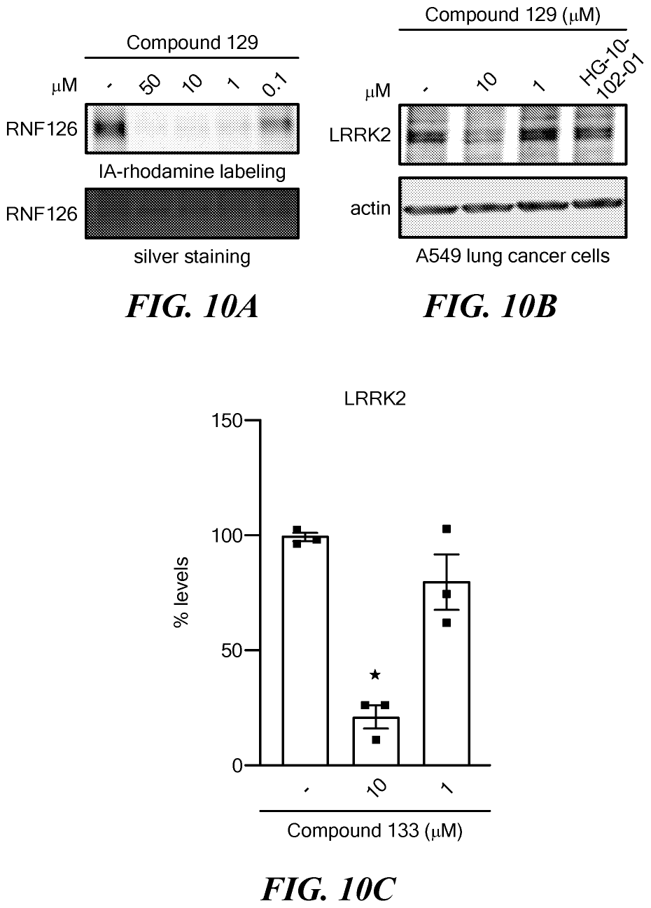

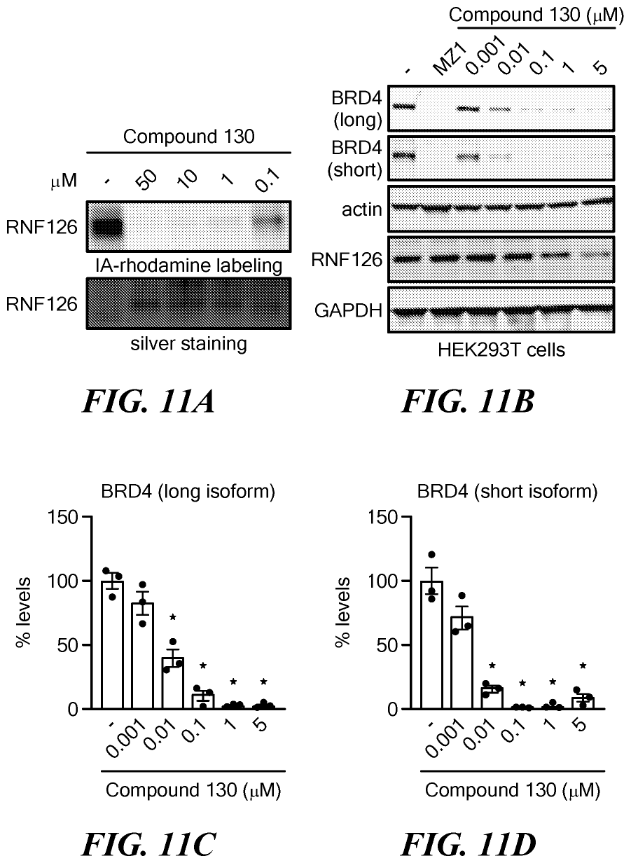

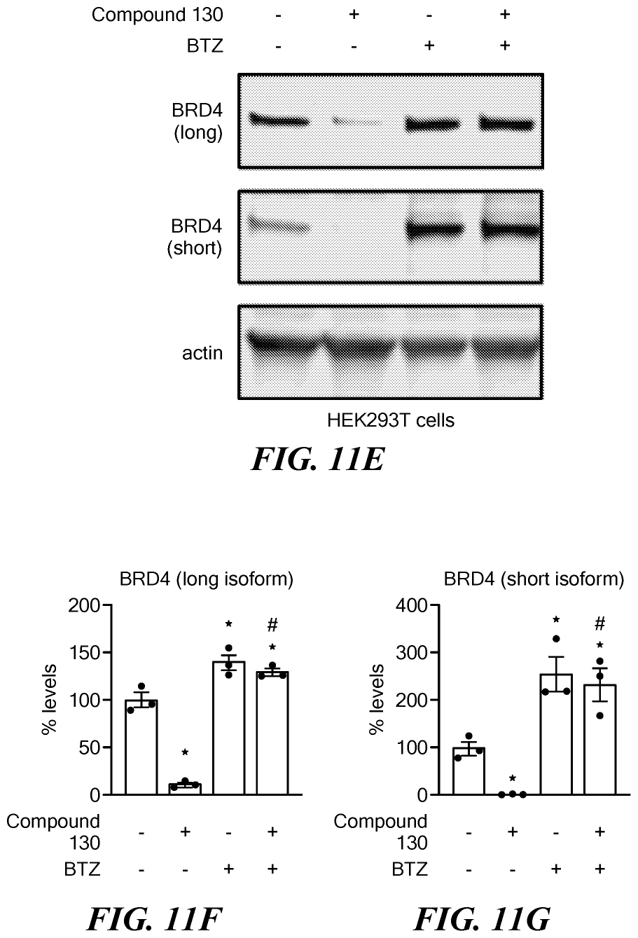

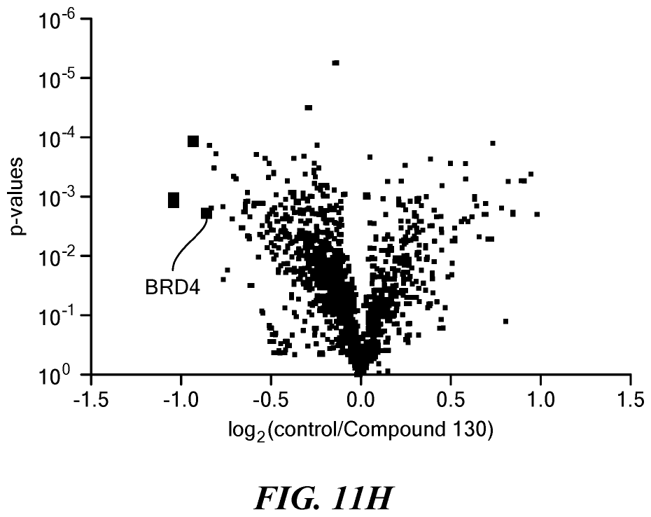

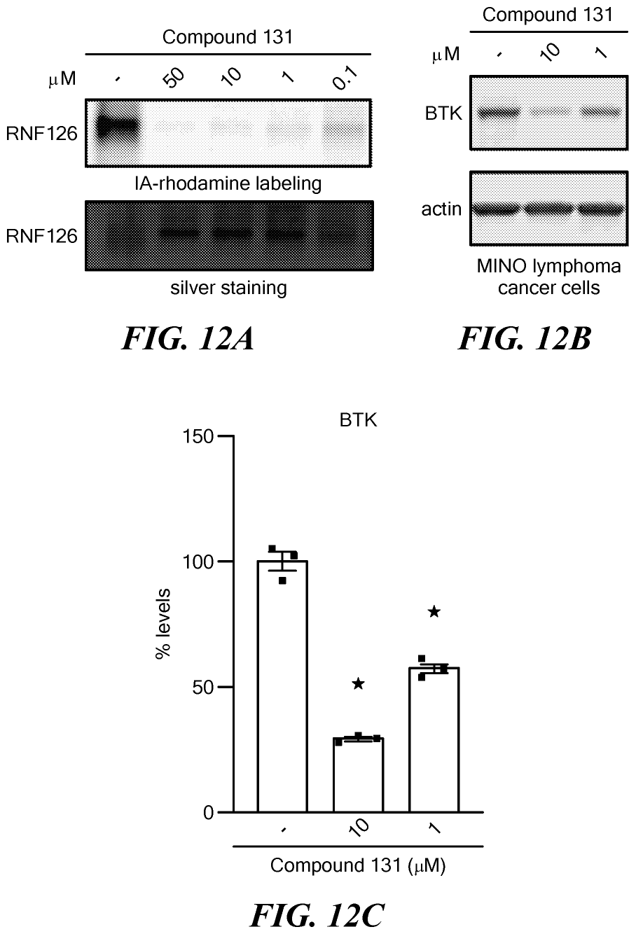

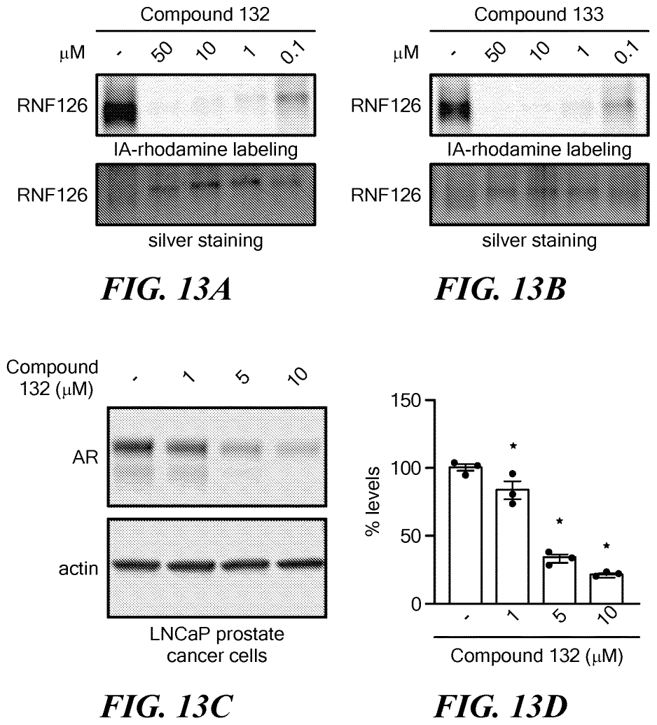

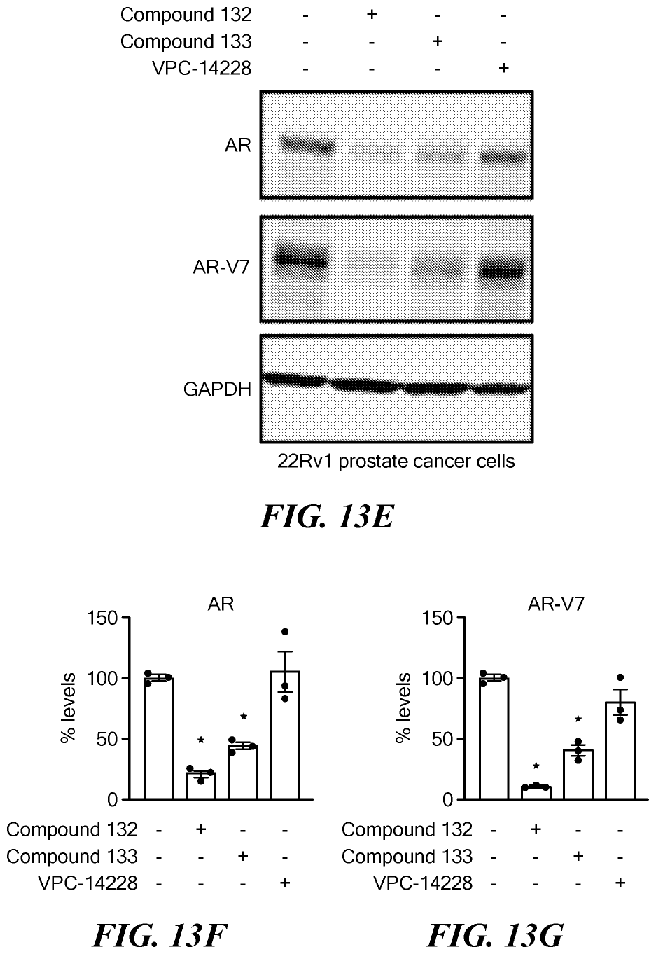

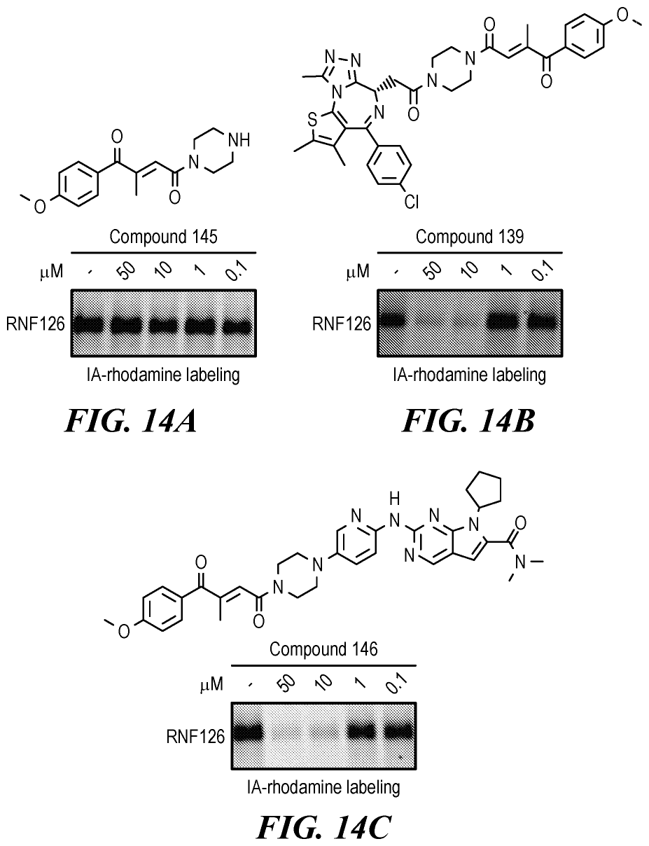

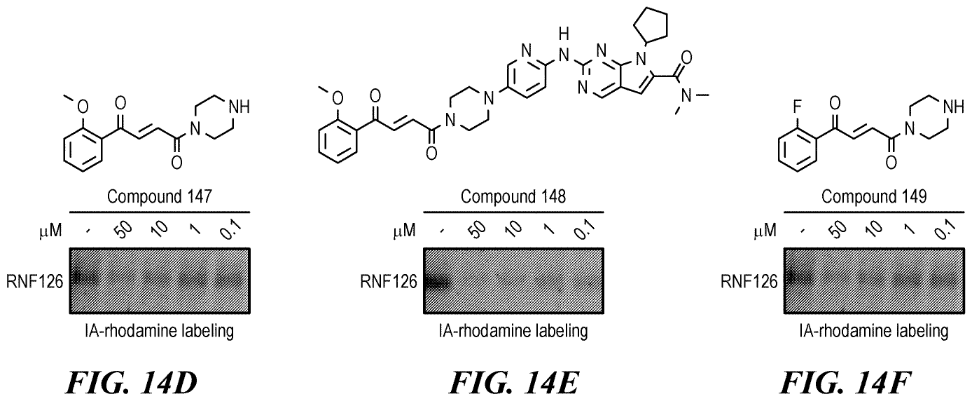

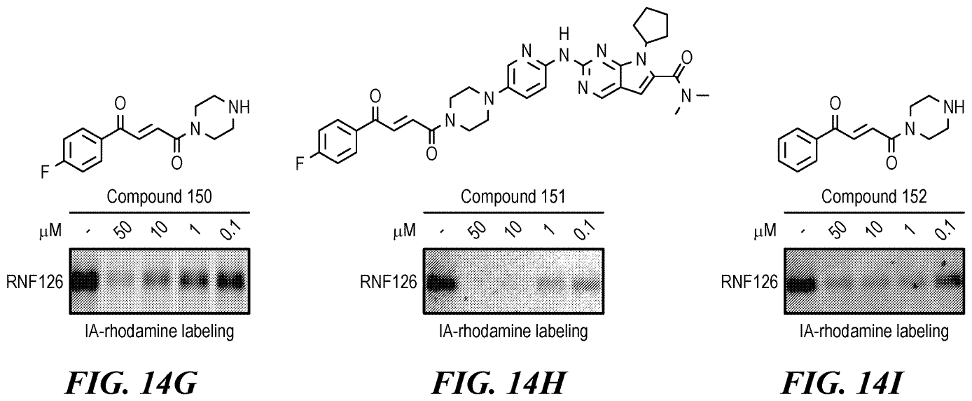

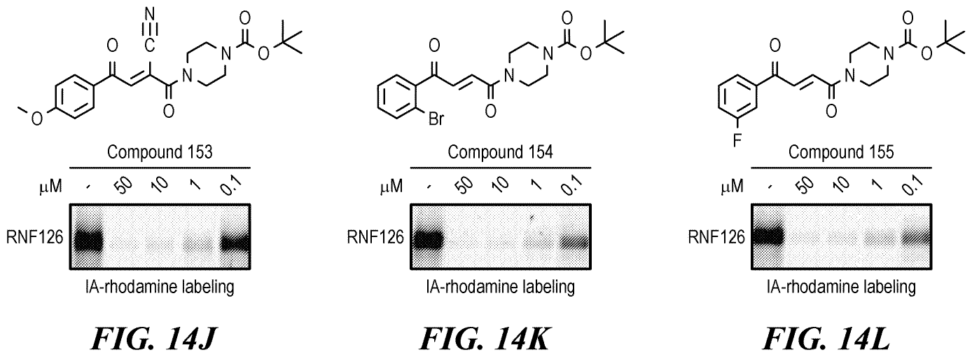

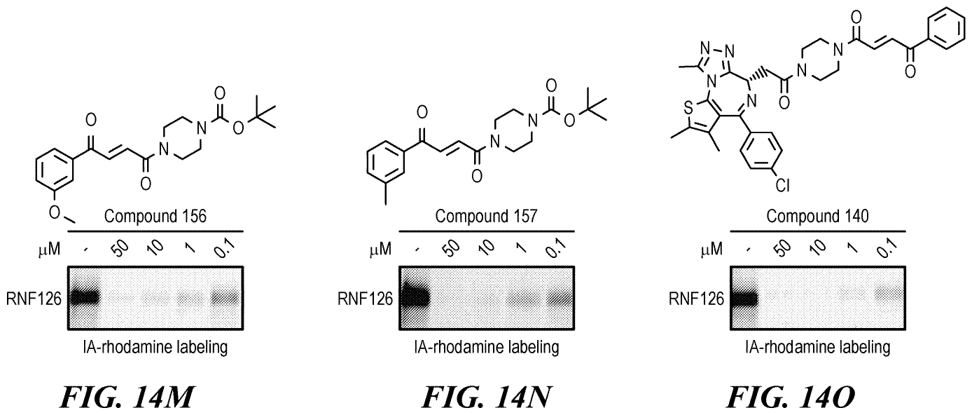

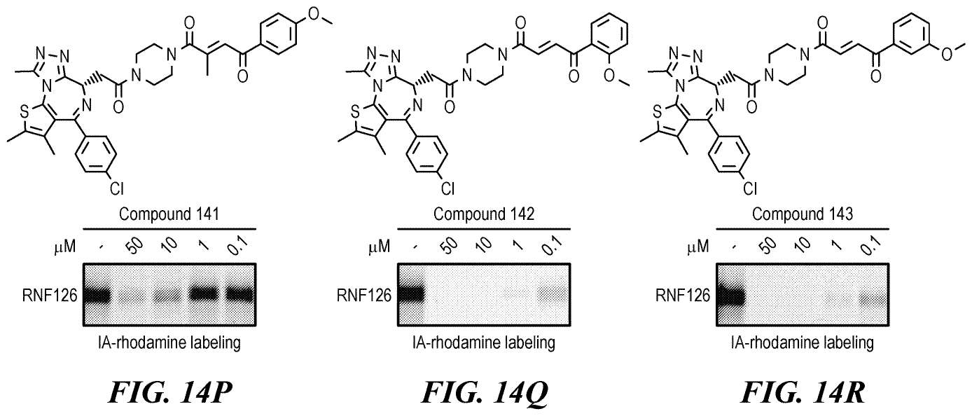

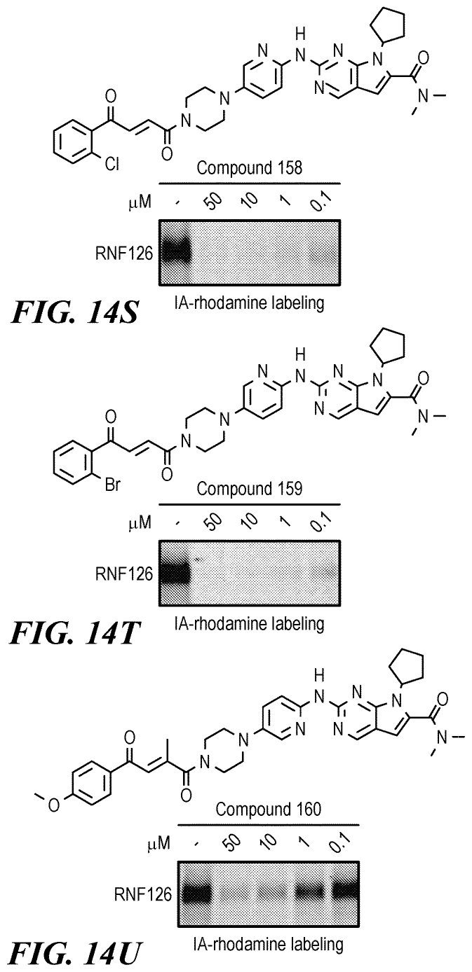

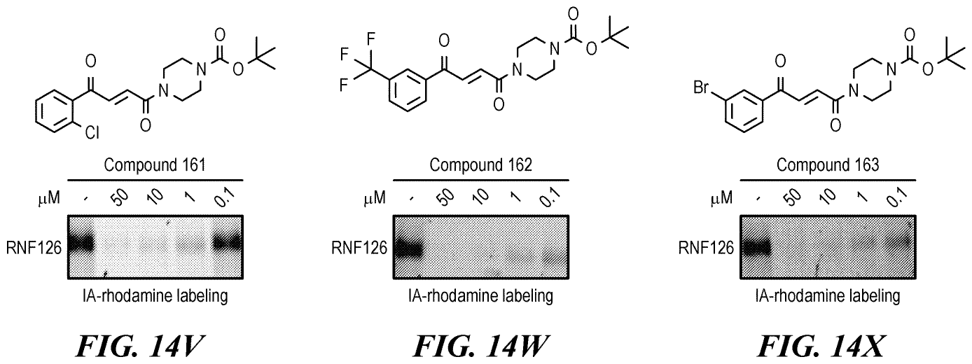

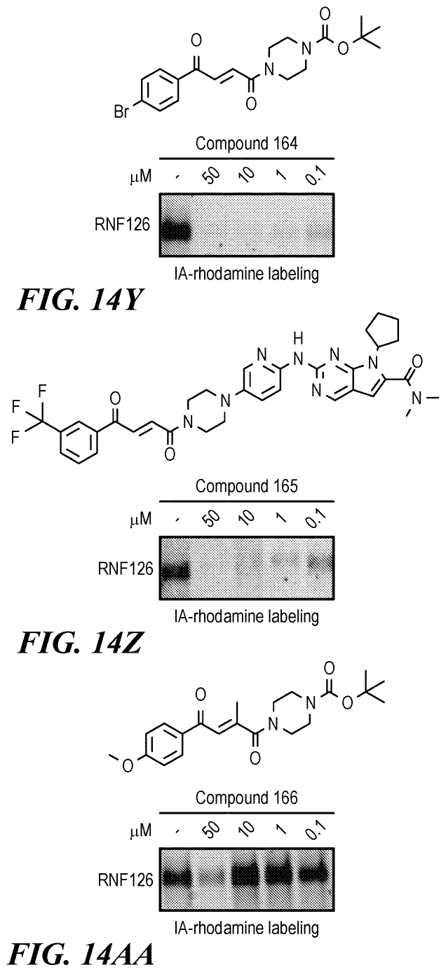

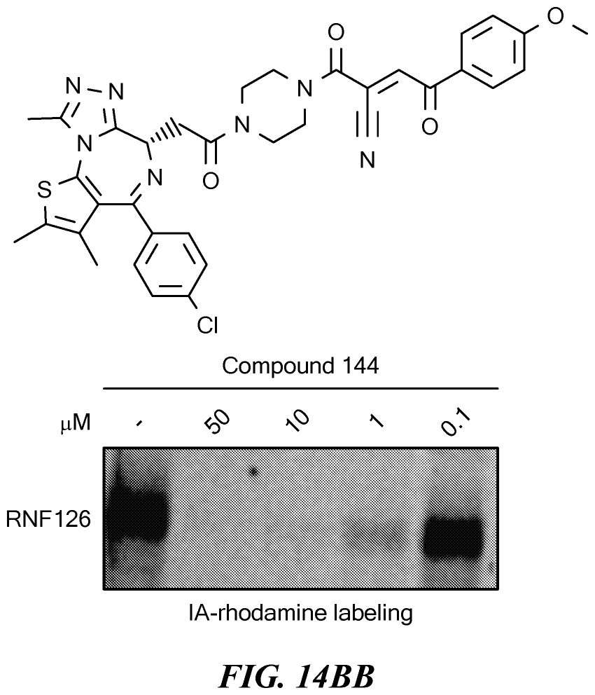

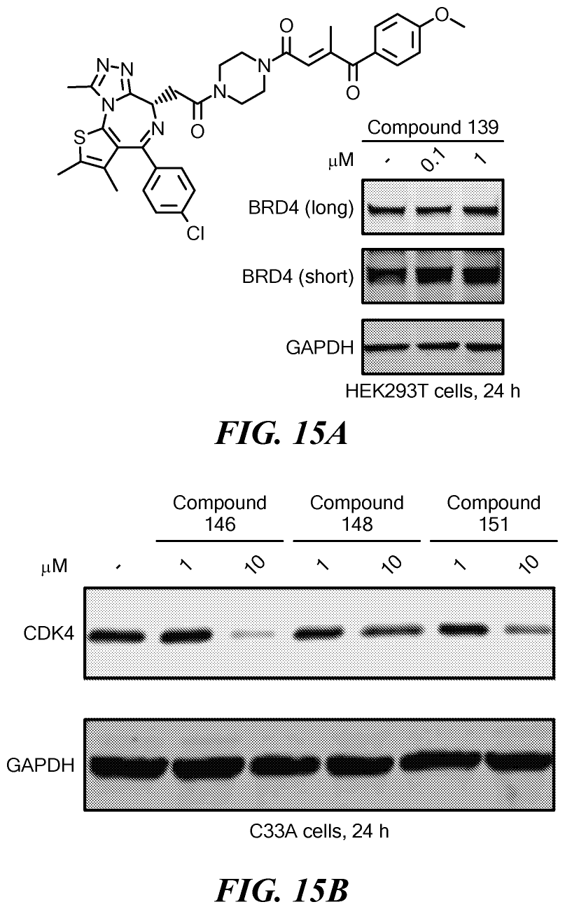

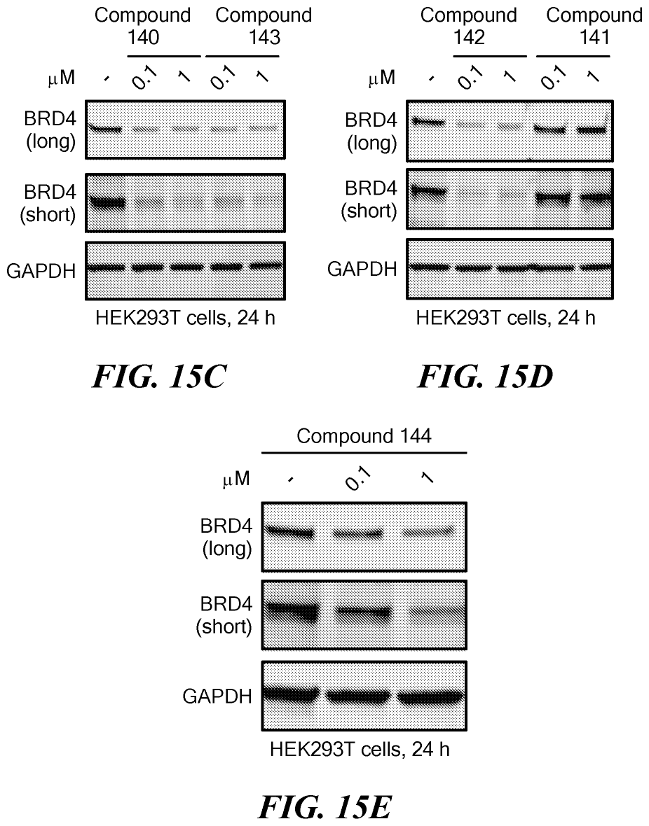

### DETAILED DESCRIPTION

Described herein are compounds and related pharmaceutically acceptable salts, hydrates, solvates, prodrugs, stereoisomers, and tautomers thereof useful as molecular glue degrader compounds. The compounds contain both a Target Ligand domain for binding to a target protein and a RING E3 Ligase Binder domain for recruitment of a RING E3 ubiquitin ligase domain to provide for degradation of the target protein. The present disclosure further features compositions of molecular glue degrader compounds as well as methods of preparation and use thereof.

### Molecular Glue Degrader Compounds

The present disclosure features a molecular glue degrader compound having the structure of Formula (I):

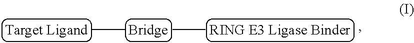

or a pharmaceutically acceptable salt, hydrate, solvate, prodrug, stereoisomer, or tautomer thereof, wherein: (i) the Target Ligand is capable of binding to a target protein; (ii) Bridge is absent or a linker; and (iii) the RING E3 Ligase Binder is capable of binding to a RING E3 ubiquitin ligase (e.g., RNF126 or BCA2). In an embodiment, the Target Ligand is a small molecule capable of binding a target protein, e.g., in a non-covalent manner. In an embodiment, Bridge is absent. In an embodiment, Bridge is a linker, e.g., a linker described herein. In an embodiment, the RING E3 Ligase Binder is capable of covalently binding to a RING E3 ubiquitin ligase. In an embodiment, the RING E3 Ligase Binder comprises an electrophilic moiety, e.g., a C₂₋₁₀ alkenylene moiety. In an embodiment, the RING E3 Ligase Binder is capable of binding to a cysteine residue within the RING E3 ubiquitin ligase, e.g., a non-catalytic cysteine residue within the RING E3 ubiquitin ligase.

In an embodiment, the molecular glue degrader compound of Formula (I) has a structure of Formula (II):

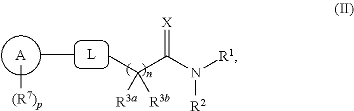

or a pharmaceutically acceptable salt, hydrate, solvate, stereoisomer, or tautomer thereof, wherein X is O or S; Ring A is selected from the group consisting of an aryl, a heteroaryl, a cycloalkyl, and a heterocycloalkyl; L is absent or a linker; R¹ is,

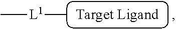

wherein L¹ is absent or a linker and the Target Ligand comprises a moiety capable of binding to a target protein; or —NR¹ is a Target Ligand; R² is selected from the group consisting of hydrogen and C₁₋₆ alkyl; or R¹ and R² together with the atoms to which they are attached form a 5- to 10-membered heteroaryl or a 3- to 12-membered heterocycloalkyl, wherein the heteroaryl or heterocycloalkyl are each substituted with one R⁸, wherein R⁸ is

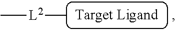

wherein L² is a linker, and the heteroaryl or heterocycloalkyl are each substituted with 0-4 occurrences of R⁹; each R⁹ is independently selected from the group consisting of —OH, halogen, C₁₋₆ alkyl, C₁₋₆ alkoxyl, C₁₋₆ haloalkyl, alkynyl, —CN, C₁₋₆ haloalkoxyl, —O-aryl, and heteroaryl; or two R⁹ on the same carbon atom together with the same carbon atom to which they are attached form a C₃₋₈ cycloalkyl or an oxo; or two R⁹ on adjacent carbon atoms together with the adjacent carbon atoms to which they are attached form a C₃₋₈ cycloalkyl; or two R⁹ on non-adjacent carbon atoms together with the non-adjacent carbon atoms to which they are attached form a bridging ring; R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; R⁴ and R⁵ are each independently selected from the group consisting of hydrogen, —OH, halogen, —CN, C₁₋₆ alkyl, and C₁₋₆ haloalkyl; R^{4′} and R^{5′} are each independently selected from the group consisting of hydrogen, —OH, halogen, —CN, C₁₋₆ alkyl, and C₁₋₆ haloalkyl; R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or R^{6a} and R^{6b}, together with the atoms to which they are attached, form an oxo group; R^{6c} is independently selected from the group consisting of hydrogen, C₁₋₆ alkyl, C₁₋₆ hydroxyalkyl, C₁₋₆ haloalkyl, and C₃₋₈ cycloalkyl; R⁷ are each independently selected from the group consisting of —OH, halogen, C₁₋₆ alkyl, C₁₋₆ alkoxyl, C₁₋₆ haloalkyl, alkynyl, —CN, C₁₋₆ haloalkoxyl, —O-aryl, and heteroaryl; or two R⁷ on the same carbon atom together with the atoms which they are attached, form an oxo group; or two R⁷ on adjacent atoms together with the atoms to which they are attached form an optionally substituted 5- to 10-membered aryl or a 3- to 12-membered heterocycloalkyl; n is 0, 1, or 2; and p is 0, 1, 2, 3, or 4.

In an embodiment, X is O. In an embodiment, X is S. In an embodiment, R² is hydrogen. In an embodiment, R² is methyl. In an embodiment, each of R^{3a} and R^{3b} is independently hydrogen or C₁₋₆ alkyl. In an embodiment, one of R^{3a} and R^{3b} is hydrogen. In an embodiment, each of R^{3a} and R^{3b} are hydrogen. In an embodiment, L is absent. In an embodiment, L is a linker. In an embodiment, L is a linker selected from the group of

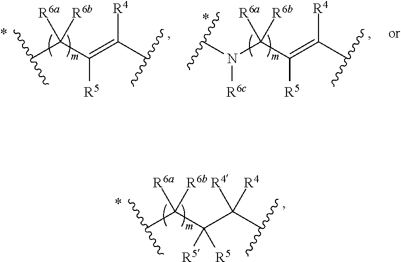

wherein * denotes where L connects to Ring A. In an embodiment, R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl. In an embodiment, R^{6a} and R^{6b}, together with the atoms to which they are attached, form an oxo group. In an embodiment, R^{6c} is independently selected from the group consisting of hydrogen, C₁₋₆ alkyl, C₁₋₆ hydroxyalkyl, C₁₋₆ haloalkyl, and C₃₋₈ cycloalkyl. In an embodiment, each of R^{6a} and R^{6b} is independently hydrogen or C₁₋₆ alkyl. In an embodiment, R⁴ and R⁵ are each independently selected from the group consisting of hydrogen, —OH, halogen, —CN, C₁₋₆ alkyl, and C₁₋₆ haloalkyl. In an embodiment, R⁴ and R⁵ are each independently selected from the group consisting of hydrogen, halogen, —CN, C₁₋₆ alkyl, and C₁₋₆ haloalkyl. In an embodiment, each of R⁴ and R⁵ is independently hydrogen or C₁₋₆ alkyl. In an embodiment, R^{4′} and R^{5′} are each independently selected from the group consisting of hydrogen, —OH, halogen, —CN, C₁₋₆ alkyl, and C₁₋₆ haloalkyl. In an embodiment, m is 1 or 2. In an embodiment, m is 0. In an embodiment, n is 1 or 2. In an embodiment, n is 0. In an embodiment, n is 1. In an embodiment, n is 2. In an embodiment, Ring A is 6- to 10-membered aryl (e.g., phenyl). In an embodiment, Ring A is 5- to 10-membered heteroaryl (e.g., pyridyl). In an embodiment, p is 0, 1, 2, or 3. In an embodiment, L¹ is absent. In an embodiment, L¹ is a linker (e.g., a linker described herein). In an embodiment, L² is absent. In an embodiment, L² is a linker (e.g., a linker described herein). In an embodiment, the Target Ligand is a kinase inhibitor, bromodomain inhibitor, or phosphodiesterase inhibitor. In an embodiment, the Target Ligand is capable of binding to CDK4, CDK6, BRD4, PDE5, BCR-ABL, c-ABL, AR, AR-V7, BTK, LRRK2, or SMARCA2. In an embodiment, the Target Ligand is selected from ribociclib, dasatinib, palbociclib, sildenafil, HG-10-102-01, JQ1, ibrutinib, or a derivative thereof.

In an embodiment, the molecular glue degrader compound of Formula (I) has a structure of Formula (II-a):

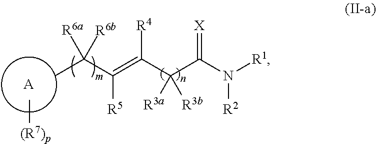

or a pharmaceutically acceptable salt, hydrate, solvate, stereoisomer, or tautomer thereof, wherein X is O or S; Ring A is selected from the group consisting of a 6- to 10-membered aryl, a 5- to 10-membered heteroaryl, a 3- to 12-membered cycloalkyl, and a 3- to 12-membered heterocycloalkyl; R¹ is

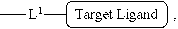

wherein L¹ is absent or a linker and the Target Ligand comprises a moiety capable of binding to a target protein; or —NR¹ is a Target Ligand; R² is selected from the group consisting of hydrogen and C₁₋₆ alkyl; or R¹ and R² together with the atoms to which they are attached form a 5- to 10-membered heteroaryl or a 3- to 12-membered heterocycloalkyl, wherein the heteroaryl or heterocycloalkyl are each substituted with one R⁸, wherein R⁸ is

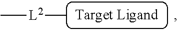

wherein L² is a linker, and the heteroaryl or heterocycloalkyl are each substituted with 0-4 occurrences of R⁹; each R⁹ is independently selected from the group consisting of —OH, halogen, C₁₋₆ alkyl, C₁₋₆ alkoxyl, C₁₋₆ haloalkyl, and C₁₋₆ haloalkoxyl; or two R⁹ on the same carbon atom together with the same carbon atom to which they are attached form a C₃₋₈ cycloalkyl or an oxo; or two R⁹ on adjacent carbon atoms together with the adjacent carbon atoms to which they are attached form a C₃₋₈ cycloalkyl; or two R⁹ on non-adjacent carbon atoms together with the non-adjacent carbon atoms to which they are attached form a bridging ring; R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; R⁴ and R⁵ are each independently selected from the group consisting of hydrogen, —OH, halogen, —CN, C₁₋₆ alkyl, and C₁₋₆ haloalkyl; R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or R^{6a} and R^{6b}, together with the atoms to which they are attached, form an oxo group; R⁷ are each independently selected from the group consisting of —OH, halogen, C₁₋₆ alkyl, C₁₋₆ alkoxyl, C₁₋₆ haloalkyl, and C₁₋₆ haloalkoxyl; m is 0, 1, or 2; n is 0, 1, or 2; and p is 0, 1, 2, 3, or 4.

In an embodiment, X is O. In an embodiment, R² is hydrogen. In an embodiment, each of R^{3a} and R^{3b} is independently hydrogen or C₁₋₆ alkyl. In an embodiment, each of R^{6a} and R^{6b} is independently hydrogen or C₁₋₆ alkyl. In an embodiment, R⁴ and R⁵ are each independently selected from the group consisting of hydrogen, halogen, —CN, C₁₋₆ alkyl, and C₁₋₆ haloalkyl. In an embodiment, each of R⁴ and R⁵ is independently hydrogen or C₁₋₆ alkyl. In an embodiment, m is 1 or 2. In an embodiment, n is 1 or 2. In an embodiment, Ring A is 6- to 10-membered aryl (e.g., phenyl). In an embodiment, Ring A is 5- to 10-membered heteroaryl (e.g., pyridyl). In an embodiment, p is 0, 1, 2, or 3. In an embodiment, Lis absent. In an embodiment, Lis a linker (e.g., a linker described herein). In an embodiment, L² is absent. In an embodiment, L² is a linker (e.g., a linker described herein). In an embodiment, the Target Ligand is a kinase inhibitor, bromodomain inhibitor, or phosphodiesterase inhibitor. In an embodiment, the Target Ligand is capable of binding to CDK4, CDK6, BRD4, PDE5, BCR-ABL, c-ABL, AR, AR-V7, BTK, LRRK2, or SMARCA2. In an embodiment, the Target Ligand is selected from ribociclib, dasatinib, palbociclib, sildenafil, HG-10-102-01, JQ1, ibrutinib, or a derivative thereof.

In an embodiment, the molecular glue degrader compound of Formula (I) has a structure of Formula (II-b):

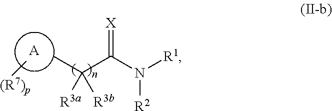

or a pharmaceutically acceptable salt, hydrate, solvate, stereoisomer, or tautomer thereof, wherein X is O or S; Ring A is selected from the group consisting of a 6- to 10-membered aryl, a 5- to 10-membered heteroaryl, a 3- to 12-membered cycloalkyl, and a 3- to 12-membered heterocycloalkyl; R¹ is

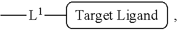

wherein L¹ is absent or a linker and the Target Ligand comprises a moiety capable of binding to a target protein; or —NR¹ is a Target Ligand; R² is selected from the group consisting of hydrogen and C₁₋₆ alkyl; or R¹ and R² together with the atoms to which they are attached form a 5- to 10-membered heteroaryl or a 3- to 12-membered heterocycloalkyl, wherein the heteroaryl or heterocycloalkyl are each substituted with one R⁸, wherein R⁸ is

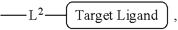

wherein L² is a linker, and the heteroaryl or heterocycloalkyl are each substituted with 0-4 occurrences of R⁹; each R⁹ is independently selected from the group consisting of —OH, halogen, C₁₋₆ alkyl, C₁₋₆ alkoxyl, C₁₋₆ haloalkyl, and C₁₋₆ haloalkoxyl; or two R⁹ on the same carbon atom together with the same carbon atom to which they are attached form a C₃₋₈ cycloalkyl or an oxo; or two R⁹ on adjacent carbon atoms together with the adjacent carbon atoms to which they are attached form a C₃₋₈ cycloalkyl; or two R⁹ on non-adjacent carbon atoms together with the non-adjacent carbon atoms to which they are attached form a bridging ring; R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; R⁷ are each independently selected from the group consisting of —OH, halogen, C₁₋₆ alkyl, C₁₋₆ alkoxyl, C₁₋₆ haloalkyl, and C₁₋₆ haloalkoxyl; n is 0, 1, or 2; and p is 0, 1, 2, 3, or 4.

In an embodiment, the molecular glue degrader compound of Formula (I) has a structure of Formula (II-c):

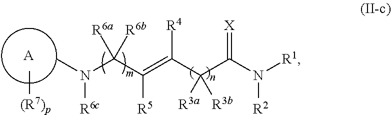

or a pharmaceutically acceptable salt, hydrate, solvate, stereoisomer, or tautomer thereof, wherein X is O or S; Ring A is selected from the group consisting of a 6- to 10-membered aryl, a 5- to 10-membered heteroaryl, a 3- to 12-membered cycloalkyl, and a 3- to 12-membered heterocycloalkyl; R¹ is

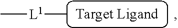

wherein L¹ is absent or a linker and the Target Ligand comprises a moiety capable of binding to a target protein; or —NR¹ is a Target Ligand; R² is selected from the group consisting of hydrogen and C₁₋₆ alkyl; or R¹ and R² together with the atoms to which they are attached form a 5- to 10-membered heteroaryl or a 3- to 12-membered heterocycloalkyl, wherein the heteroaryl or heterocycloalkyl are each substituted with one R⁸, wherein R⁸ is

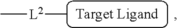

wherein L² is a linker, and the heteroaryl or heterocycloalkyl are each substituted with 0-4 occurrences of R⁹; each R⁹ is independently selected from the group consisting of —OH, halogen, C₁₋₆ alkyl, C₁₋₆ alkoxyl, C₁₋₆ haloalkyl, and C₁₋₆ haloalkoxyl; or two R⁹ on the same carbon atom together with the same carbon atom to which they are attached form a C₃₋₈ cycloalkyl or an oxo; or two R⁹ on adjacent carbon atoms together with the adjacent carbon atoms to which they are attached form a C₃₋₈ cycloalkyl; or two R⁹ on non-adjacent carbon atoms together with the non-adjacent carbon atoms to which they are attached form a bridging ring; R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; R⁴ and R⁵ are each independently selected from the group consisting of hydrogen, —OH, halogen, —CN, C₁₋₆ alkyl, and C₁₋₆ haloalkyl; R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or R^{6a} and R^{6b}, together with the atoms to which they are attached, form an oxo group; R^{6c} is independently selected from the group consisting of hydrogen, C₁₋₆ alkyl, C₁₋₆ hydroxyalkyl, C₁₋₆ haloalkyl, and C₃₋₈ cycloalkyl; R⁷ are each independently selected from the group consisting of —OH, halogen, C₁₋₆ alkyl, C₁₋₆ alkoxyl, C₁₋₆ haloalkyl, and C₁₋₆ haloalkoxyl; m is 0, 1, or 2; n is 0, 1, or 2; and p is 0, 1, 2, 3, or 4.

In an embodiment, the molecular glue degrader compound of Formula (I) has a structure of Formula (II-d):

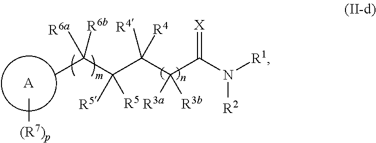

or a pharmaceutically acceptable salt, hydrate, solvate, stereoisomer, or tautomer thereof, wherein X is O or S; Ring A is selected from the group consisting of a 6- to 10-membered aryl, a 5- to 10-membered heteroaryl, a 3- to 12-membered cycloalkyl, and a 3- to 12-membered heterocycloalkyl; R¹ is

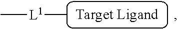

wherein L¹ is absent or a linker and the Target Ligand comprises a moiety capable of binding to a target protein; or —NR¹ is a Target Ligand; R² is selected from the group consisting of hydrogen and C₁₋₆ alkyl; or R¹ and R² together with the atoms to which they are attached form a 5- to 10-membered heteroaryl or a 3- to 12-membered heterocycloalkyl, wherein the heteroaryl or heterocycloalkyl are each substituted with one R⁸, wherein R⁸ is

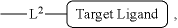

wherein L² is a linker, and the heteroaryl or heterocycloalkyl are each substituted with 0-4 occurrences of R⁹; each R⁹ is independently selected from the group consisting of —OH, halogen, C₁₋₆ alkyl, C₁₋₆ alkoxyl, C₁₋₆ haloalkyl, and C₁₋₆ haloalkoxyl; or two R⁹ on the same carbon atom together with the same carbon atom to which they are attached form a C₃₋₈ cycloalkyl or an oxo; or two R⁹ on adjacent carbon atoms together with the adjacent carbon atoms to which they are attached form a C₃₋₈ cycloalkyl; or two R⁹ on non-adjacent carbon atoms together with the non-adjacent carbon atoms to which they are attached form a bridging ring; R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; R⁴ and R⁵ are each independently selected from the group consisting of hydrogen, —OH, halogen, —CN, C₁₋₆ alkyl, and C₁₋₆ haloalkyl; R^{4′} and R^{5′} are each independently selected from the group consisting of hydrogen, —OH, halogen, —CN, C₁₋₆ alkyl, and C₁₋₆ haloalkyl; R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or R^{6a} and R^{6b}, together with the atoms to which they are attached, form an oxo group; R⁷ are each independently selected from the group consisting of —OH, halogen, C₁₋₆ alkyl, C₁₋₆ alkoxyl, C₁₋₆ haloalkyl, and C₁₋₆ haloalkoxyl; m is 0, 1, or 2; n is 0, 1, or 2; and p is 0, 1, 2, 3, or 4.

In an embodiment, the molecular glue degrader compound of Formula (I) has a structure of Formula (II-e):

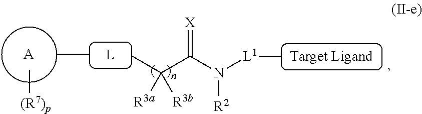

or a pharmaceutically acceptable salt, hydrate, solvate, stereoisomer, or tautomer thereof, wherein each of the Target Ligand, Ring A, L, L¹, X, R², R^{3a}, R^{3b}, R⁷, n, p, and variables therein, are as defined for Formula (II).

In an embodiment, the molecular glue degrader compound of Formula (I) has a structure of Formula (II-f):

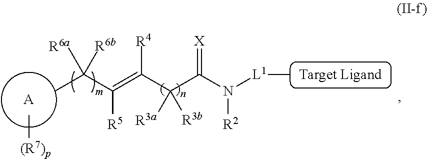

or a pharmaceutically acceptable salt, hydrate, solvate, stereoisomer, or tautomer thereof, wherein each of the Target Ligand, Ring A, L¹, X, R², R^{3a}, R^{3b}, R⁴, R⁵, R^{6a}, R^{6b}, R⁷, m, n, and p are as defined for Formula (II).

In an embodiment, X is O. In an embodiment, R² is hydrogen. In an embodiment, each of R^{3a} and R^{3b} is independently hydrogen or C₁₋₆ alkyl. In an embodiment, each of R^{6a} and R^{6b} is independently hydrogen or C₁₋₆ alkyl. In an embodiment, R⁴ and R⁵ are each independently selected from the group consisting of hydrogen, halogen, —CN, C₁₋₆ alkyl, and C₁₋₆ haloalkyl. In an embodiment, each of R⁴ and R⁵ is independently hydrogen or C₁₋₆ alkyl. In an embodiment, m is 1 or 2. In an embodiment, n is 1 or 2. In an embodiment, Ring A is 6- to 10-membered aryl (e.g., phenyl). In an embodiment, Ring A is 5- to 10-membered heteroaryl (e.g., pyridyl). In an embodiment, p is 0, 1, 2, or 3. In an embodiment, L¹ is absent. In an embodiment, L¹ is a linker (e.g., a linker described herein). In an embodiment, the Target Ligand is a kinase inhibitor, bromodomain inhibitor, or phosphodiesterase inhibitor. In an embodiment, the Target Ligand is capable of binding to CDK4, CDK6, BRD4, PDE5, BCR-ABL, c-ABL, AR, AR-V7, BTK, LRRK2, or SMARCA2. In an embodiment, the Target Ligand is selected from ribociclib, dasatinib, palbociclib, sildenafil, HG-10-102-01, JQ1, ibrutinib, or a derivative thereof.

In an embodiment, R¹ and R² together with the atoms to which they are attached form a 5- to 10-membered heteroaryl or a 3- to 12-membered heterocycloalkyl, which is referred to as Ring B.

In an embodiment, the molecular glue degrader compound of Formula (I) has a structure of Formula (II-g):

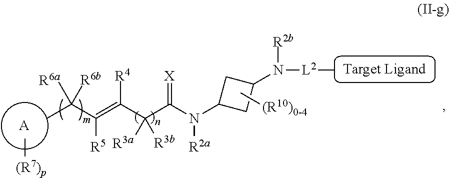

or a pharmaceutically acceptable salt, hydrate, solvate, stereoisomer, or tautomer thereof, wherein X is O or S; Ring A is selected from the group consisting of a 6- to 10-membered aryl, a 5- to 10-membered heteroaryl, a 3- to 12-membered cycloalkyl, and a 3- to 12-membered heterocycloalkyl; L² is a linker substituted with 0-4 occurrences of R⁹; Target Ligand comprises a moiety capable of binding to a target protein; R^{2a} and R^{2b} are each independently hydrogen, C₁₋₆ alkyl, and C₃₋₈ cycloalkyl; R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or R^{3a} and R^{3b}, together with the atoms to which they are attached, form an oxo group; R⁴ and R⁵ are each independently selected from the group consisting of hydrogen, —OH, halogen, —CN, C₁₋₆ alkyl, and C₁₋₆ haloalkyl; R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or R^{6a} and R^{6b}, together with the atoms to which they are attached, form an oxo group; each R⁷ is independently selected from the group consisting of —OH, halogen, C₁₋₆ alkyl, C₁₋₆ alkoxyl, C₁₋₆ haloalkyl, and C₁₋₆ haloalkoxyl; each R⁹ is independently selected from the group consisting of —OH, halogen, C₁₋₆ alkyl, C₁₋₆ alkoxyl, C₁₋₆ haloalkyl, and C₁₋₆ haloalkoxyl; or two R⁹ on the same carbon atom together with the same carbon atom to which they are attached form a C₃₋₈ cycloalkyl or an oxo; or two R⁹ on adjacent carbon atoms together with the adjacent carbon atoms to which they are attached form a C₃₋₈ cycloalkyl; or two R⁹ on non-adjacent carbon atoms together with the non-adjacent carbon atoms to which they are attached form a bridging ring; each R¹⁰ is independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; m is 0, 1, or 2; n is 0, 1, or 2; and p is 0, 1, 2, 3, or 4.

In an embodiment, the molecular glue degrader compound of Formula (I) has a structure of Formula (II-h):

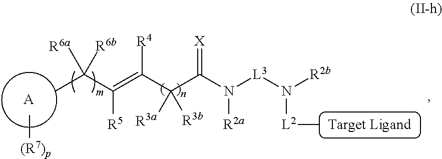

or a pharmaceutically acceptable salt, hydrate, solvate, stereoisomer, or tautomer thereof, wherein X is O or S; Ring A is selected from the group consisting of a 6- to 10-membered aryl, a 5- to 10-membered heteroaryl, a 3- to 12-membered cycloalkyl, and a 3- to 12-membered heterocycloalkyl; L³ is trans-cyclobutane-1,3-diyl or cis-cyclobutane-1,3-diyl, wherein the cyclobutane ring is optionally substituted with 1, 2, 3, or 4 occurrences of R¹⁰; L² is a linker substituted with 0-4 occurrences of R⁹; Target Ligand comprises a moiety capable of binding to a target protein; R^{2a} and R^{2b} are each independently selected from the group consisting of hydrogen, C₁₋₆ alkyl, and C₃₋₈ cycloalkyl; R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; R⁴ and R⁵ are each independently selected from the group consisting of hydrogen, —OH, halogen, —CN, C₁₋₆ alkyl, and C₁₋₆ haloalkyl; R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or R^{6a} and R^{6b}, together with the atoms to which they are attached, form an oxo group; each R⁷ is independently selected from the group consisting of —OH, halogen, C₁₋₆ alkyl, C₁₋₆ alkoxyl, C₁₋₆ haloalkyl, and C₁₋₆ haloalkoxyl; each R⁹ is independently selected from the group consisting of —OH, halogen, C₁₋₆ alkyl, C₁₋₆ alkoxyl, C₁₋₆ haloalkyl, and C₁₋₆ haloalkoxyl; or two R⁹ on the same carbon atom together with the same carbon atom to which they are attached form a C₃₋₈ cycloalkyl or an oxo; or two R⁹ on adjacent carbon atoms together with the adjacent carbon atoms to which they are attached form a C₃₋₈ cycloalkyl; or two R⁹ on non-adjacent carbon atoms together with the non-adjacent carbon atoms to which they are attached form a bridging ring; each R¹⁰ is independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; m is 0, 1, or 2; n is 0, 1, or 2; and p is 0, 1, 2, 3, or 4.

In an embodiment, the molecular glue degrader compound of Formula (I) has the structure of Formula (III):

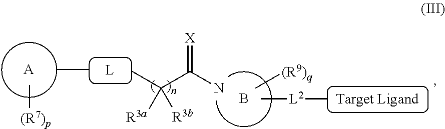

or a pharmaceutically acceptable salt, hydrate, solvate, stereoisomer, or tautomer thereof, wherein X is O or S; Ring A is selected from the group consisting of an aryl, a heteroaryl, a cycloalkyl, and a heterocycloalkyl; L is absent or a linker; the Target Ligand comprises a moiety capable of binding to a target protein; Ring B is a 5- to 10-membered heteroaryl or a 3- to 12-membered heterocycloalkyl, each optionally substituted with 0-4 occurrences of R⁹; L² is a linker; each R⁹ is independently selected from the group consisting of —OH, halogen, C₁₋₆ alkyl, C₁₋₆ alkoxyl, C₁₋₆ haloalkyl, alkynyl, —CN, C₁₋₆ haloalkoxyl, —O-aryl, and heteroaryl; or two R⁹ on the same carbon atom together with the same carbon atom to which they are attached form a C₃₋₈ cycloalkyl or an oxo; or two R⁹ on adjacent carbon atoms together with the adjacent carbon atoms to which they are attached form a C₃₋₈ cycloalkyl; or two R⁹ on non-adjacent carbon atoms together with the non-adjacent carbon atoms to which they are attached form a bridging ring; R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; R⁷ are each independently selected from the group consisting of —OH, halogen, C₁₋₆ alkyl, C₁₋₆ alkoxyl, C₁₋₆ haloalkyl, alkynyl, —CN, C₁₋₆ haloalkoxyl, —O-aryl, and heteroaryl; or two R⁷ on the same carbon atom together with the atoms which they are attached, form an oxo group; or two R⁷ on adjacent atoms together with the atoms to which they are attached form an optionally substituted 5- to 10-membered aryl or a 3- to 12-membered heterocycloalkyl; n is 0, 1, or 2; q is 0, 1, 2, 3, or 4; and p is 0, 1, 2, 3, or 4.

In an embodiment, the molecular glue degrader compound of Formula (I) has the structure of Formula (III-a):

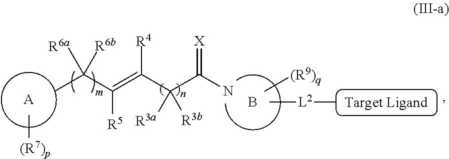

or a pharmaceutically acceptable salt, hydrate, solvate, stereoisomer, or tautomer thereof, wherein X is O or S; Ring A is selected from the group consisting of an aryl, a heteroaryl, a cycloalkyl, and a heterocycloalkyl; Ring B is a 5- to 10-membered heteroaryl or a 3- to 12-membered heterocycloalkyl, each optionally substituted with 0-4 occurrences of R⁹; L² is a linker; each R⁹ is independently selected from the group consisting of —OH, halogen, C₁₋₆ alkyl, C₁₋₆ alkoxyl, C₁₋₆ haloalkyl, alkynyl, —CN, C₁₋₆ haloalkoxyl, —O-aryl, and heteroaryl; or two R⁹ on the same carbon atom together with the same carbon atom to which they are attached form a C₃₋₈ cycloalkyl or an oxo; or two R⁹ on adjacent carbon atoms together with the adjacent carbon atoms to which they are attached form a C₃₋₈ cycloalkyl; or two R⁹ on non-adjacent carbon atoms together with the non-adjacent carbon atoms to which they are attached form a bridging ring; R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; R⁴ and R⁵ are each independently selected from the group consisting of hydrogen, —OH, halogen, —CN, C₁₋₆ alkyl, and C₁₋₆ haloalkyl; R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or R^{6a} and R^{6b}, together with the atoms to which they are attached, form an oxo group; R^{T} are each independently selected from the group consisting of —OH, halogen, C₁₋₆ alkyl, C₁₋₆ alkoxyl, C₁₋₆ haloalkyl, alkynyl, —CN, C₁₋₆ haloalkoxyl, —O-aryl, and heteroaryl; or two R⁷ on the same carbon atom together with the atoms which they are attached, form an oxo group; or two R⁷ on adjacent atoms together with the atoms to which they are attached form an optionally substituted 5- to 10-membered aryl or a 3- to 12-membered heterocycloalkyl; m is 0, 1, or 2; n is 0, 1, or 2; q is 0, 1, 2, 3, or 4; and p is 0, 1, 2, 3, or 4.

In an embodiment, X is O. In an embodiment, each of R^{3a} and R^{3b} is independently hydrogen or C₁₋₆ alkyl. In an embodiment, each of R^{6a} and R^{6b} is independently hydrogen or C₁₋₆ alkyl. In an embodiment, R⁴ and R⁵ are each independently selected from the group consisting of hydrogen, halogen, —CN, C₁₋₆ alkyl, and C₁₋₆ haloalkyl. In an embodiment, each of R⁴ and R⁵ is independently hydrogen or C₁₋₆ alkyl. In an embodiment, m is 1 or 2. In an embodiment, n is 1 or 2. In an embodiment, Ring A is 6- to 10-membered aryl (e.g., phenyl). In an embodiment, Ring A is 5- to 10-membered heteroaryl (e.g., pyridyl). In an embodiment, p is 0, 1, 2, or 3. In an embodiment, L² is absent. In an embodiment, L² is a linker (e.g., a linker described herein). In an embodiment, the Target Ligand is a kinase inhibitor, bromodomain inhibitor, or phosphodiesterase inhibitor. In an embodiment, the Target Ligand is capable of binding to CDK4, CDK6, BRD4, PDE5, BCR-ABL, c-ABL, AR, AR-V7, BTK, LRRK2, or SMARCA2. In an embodiment, the Target Ligand is selected from ribociclib, dasatinib, palbociclib, sildenafil, HG-10-102-01, JQ1, ibrutinib, or a derivative thereof.

In an embodiment, the molecular glue degrader compound of Formula (I) has the structure of Formula (III-b):

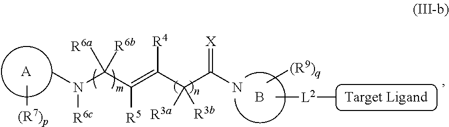

or a pharmaceutically acceptable salt, hydrate, solvate, stereoisomer, or tautomer thereof, wherein each of the Target Ligand, Ring A, L², X, R^{3a}, R^{3b}, R⁴, R⁵, R^{6a}, R^{6b}, R^{6c}, R⁷, R⁹, m, n, p, q, and variables therein, are as defined for Formula (II).

In an embodiment, the molecular glue degrader compound of Formula (I) has the structure of Formula (III-c):

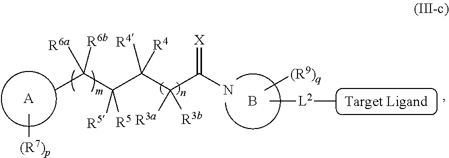

or a pharmaceutically acceptable salt, hydrate, solvate, stereoisomer, or tautomer thereof, wherein each of the Target Ligand, Ring A, L², X, R^{3a}, R^{3b}, R⁴, R⁵, R^{6a}, R^{6b}, R⁷, R⁹, m, n, p, and q are as defined for Formula (II).

In an embodiment, the molecular glue degrader compound of Formula (I) has the structure having a structure of Formula (IV):

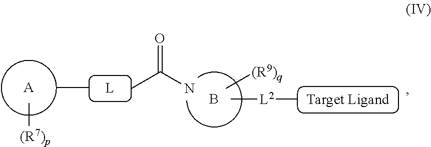

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Target Ligand, Ring A, L², R⁷, R⁹, p, and q are as defined for Formula (II).

In an embodiment, the molecular glue degrader compound of Formula (I) has the structure having a structure of Formula (IV-a):

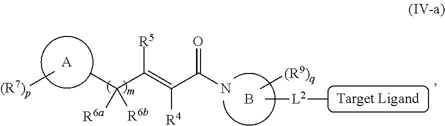

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Target Ligand, Ring A, L², R⁴, R⁵, R^{6a}, R^{6b}, R⁷, R⁹, m, p, and q are as defined for Formula (II).

In an embodiment, X is O. In an embodiment, each of R^{6a} and R^{6b} is independently hydrogen or C₁₋₆ alkyl. In an embodiment, R⁴ and R⁵ are each independently selected from the group consisting of hydrogen, halogen, —CN, C₁₋₆ alkyl, and C₁₋₆ haloalkyl. In an embodiment, each of R⁴ and R⁵ is independently hydrogen or C₁₋₆ alkyl. In an embodiment, m is 1 or 2. In an embodiment, n is 1 or 2. In an embodiment, Ring A is 6- to 10-membered aryl (e.g., phenyl). In an embodiment, Ring A is 5- to 10-membered heteroaryl (e.g., pyridyl). In an embodiment, p is 0, 1, 2, or 3. In an embodiment, L² is absent. In an embodiment, L² is a linker (e.g., a linker described herein). In an embodiment, the Target Ligand is a kinase inhibitor, bromodomain inhibitor, or phosphodiesterase inhibitor. In an embodiment, the Target Ligand is capable of binding to CDK4, CDK6, BRD4, PDE5, BCR-ABL, c-ABL, AR, AR-V7, BTK, LRRK2, or SMARCA2. In an embodiment, the Target Ligand is selected from ribociclib, dasatinib, palbociclib, sildenafil, HG-10-102-01, JQ1, ibrutinib, or a derivative thereof.

In an embodiment, the molecular glue degrader compound of Formula (I) has the structure having a structure of Formula (IV-b):

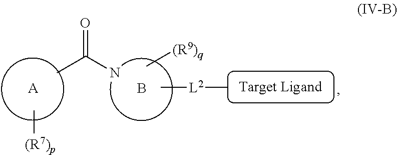

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Target Ligand, Ring A, L², R⁷, R⁹, p, and q are as defined for Formula (II).

In an embodiment, the molecular glue degrader compound of Formula (I) has the structure having a structure of Formula (V):

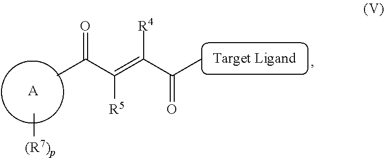

or a pharmaceutically acceptable salt, hydrate, solvate, stereoisomer, or tautomer thereof, wherein and each of the Target Ligand, Ring A, R⁴, R⁵, R⁷, and p are as defined for Formula (II).

In an embodiment, R⁴ and R⁵ are each independently selected from the group consisting of hydrogen, halogen, —CN, C₁₋₆ alkyl, and C₁₋₆ haloalkyl. In an embodiment, each of R⁴ and R⁵ is independently hydrogen or C₁₋₆ alkyl. In an embodiment, Ring A is 6- to 10-membered aryl (e.g., phenyl). In an embodiment, p is 0, 1, 2, or 3. In an embodiment, the Target Ligand is a kinase inhibitor, bromodomain inhibitor, or phosphodiesterase inhibitor. In an embodiment, the Target Ligand is capable of binding to CDK4, CDK6, BRD4, PDE5, BCR-ABL, c-ABL, AR, AR-V7, BTK, LRRK2, or SMARCA2. In an embodiment, the Target Ligand is selected from ribociclib, dasatinib, palbociclib, sildenafil, HG-10-102-01, JQ1, ibrutinib, or a derivative thereof.

In an embodiment, the molecular glue degrader compound of Formula (I) has the structure having a structure of Formula (VI):

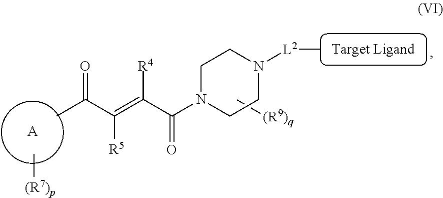

or a pharmaceutically acceptable salt, hydrate, solvate, stereoisomer, or tautomer thereof, wherein and each of the Target Ligand, Ring A, L², R⁴, R⁵, R⁷, R⁹, p and q are as defined for Formula (II).

In an embodiment, R⁴ and R⁵ are each independently selected from the group consisting of hydrogen, halogen, —CN, C₁₋₆ alkyl, and C₁₋₆ haloalkyl. In an embodiment, each of R⁴ and R⁵ is independently hydrogen or C₁₋₆ alkyl. In an embodiment, Ring A is 6- to 10-membered aryl (e.g., phenyl). In an embodiment, p is 0, 1, 2, or 3. In an embodiment, the Target Ligand is a kinase inhibitor, bromodomain inhibitor, or phosphodiesterase inhibitor. In an embodiment, the Target Ligand is capable of binding to CDK4, CDK6, BRD4, PDE5, BCR-ABL, c-ABL, AR, AR-V7, BTK, LRRK2, or SMARCA2. In an embodiment, the Target Ligand is selected from ribociclib, dasatinib, palbociclib, sildenafil, HG-10-102-01, JQ1, ibrutinib, or a derivative thereof.

In an embodiment, the molecular glue degrader compound of Formula (I) has the structure having a structure of Formula (VI-a):

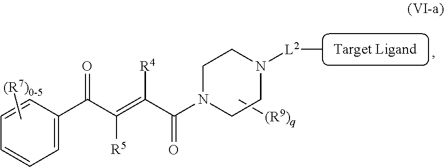

or a pharmaceutically acceptable salt, hydrate, solvate, stereoisomer, or tautomer thereof, wherein and each of the Target Ligand, L², R⁴, R⁵, R⁷, R⁹, and q are as defined for Formula (II).

In an embodiment, the molecular glue degrader compound of Formula (I) has the structure having a structure of Formula (VI-b):

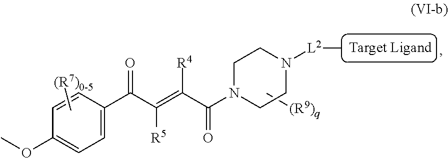

or a pharmaceutically acceptable salt, hydrate, solvate, stereoisomer, or tautomer thereof, wherein and each of the Target Ligand, L², R⁴, R⁵, R⁷, R⁹, and q are as defined for Formula (II).

In an embodiment, the molecular glue degrader compound of Formula (I) has the structure having a structure of Formula (VII):

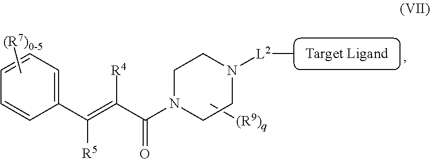

or a pharmaceutically acceptable salt, hydrate, solvate, stereoisomer, or tautomer thereof, wherein and each of the Target Ligand, L², R⁴, R⁵, R⁷, R⁹, and q are as defined for Formula (II).

In an embodiment, the molecular glue degrader compound of Formula (I) has the structure having a structure of Formula (VII-a):

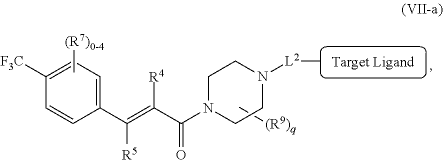

or a pharmaceutically acceptable salt, hydrate, solvate, stereoisomer, or tautomer thereof, wherein and each of the Target Ligand, L², R⁴, R⁵, R⁷, R⁹, and q are as defined for Formula (II).

In an embodiment, the molecular glue degrader compound of Formula (I) has the structure having a structure of Formula (VII-b):

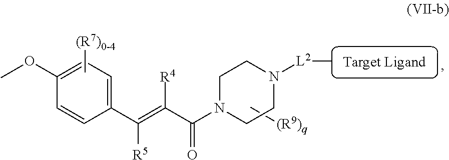

or a pharmaceutically acceptable salt, hydrate, solvate, stereoisomer, or tautomer thereof, wherein and each of the Target Ligand, L², R⁴, R⁵, R⁷, R⁹, and q are as defined for Formula (II).

In an embodiment, the molecular glue degrader compound comprises a Bridge, wherein the Bridge is a linker L¹ or L², between the Target Ligand and the RING E3 Ligase Binder. In some embodiments, the molecular glue degrader compound comprises a linker L¹ or L². In an embodiment, each of L¹ and L² is selected from absent, —C(O)—, —S(O)₂—, C₁-s alkylene, C₂₋₈ alkenylene, C₂₋₈ alkynylene, C₁₋₈ heteroalkylene, C₂₋₈ heteroalkenylene, C₂₋₈ heteroalkynylene, *C(O)—C₁₋₈ alkylene, *C(O)—C₁₋₈ heteroalkylene, *C(O)—C₁₋₈ alkylene-O, *C₁₋₈ alkylene-C(O)—, *C₁₋₈ heteroalkylene-C(O)—, *C₁₋₈ alkylene-C(O)—, *C₁₋₈ heteroalkylene-C(O)—, wherein * denotes the point of attachment to the N atom in Formula (I) and related subgenera.

In an embodiment, the molecular glue degrader compound of Formula (I) has the structure having a structure of Formula (VIII):

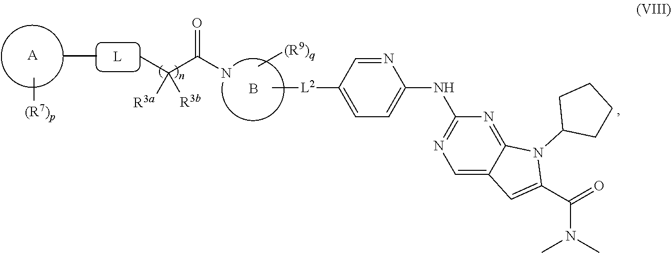

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, X is O. In an embodiment, each of R^{6a} and R^{6b} is independently hydrogen or C₁₋₆ alkyl. In an embodiment, R⁴ and R⁵ are each independently selected from the group consisting of hydrogen, halogen, —CN, C₁₋₆ alkyl, and C₁₋₆ haloalkyl. In an embodiment, each of R⁴ and R⁵ is independently hydrogen or C₁₋₆ alkyl. In an embodiment, m is 1 or 2. In an embodiment, n is 1 or 2. In an embodiment, Ring A is 6- to 10-membered aryl (e.g., phenyl). In an embodiment, p is 0, 1, 2, or 3. In an embodiment, L² is absent. In an embodiment, L² is a linker (e.g., a linker described herein).

In an embodiment, the molecular glue degrader compound of Formula (I) has the structure having a structure of Formula (VIII-a):

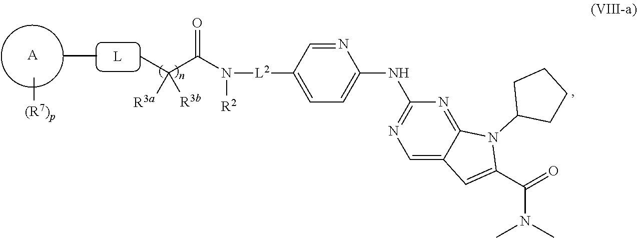

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (VIII-b):

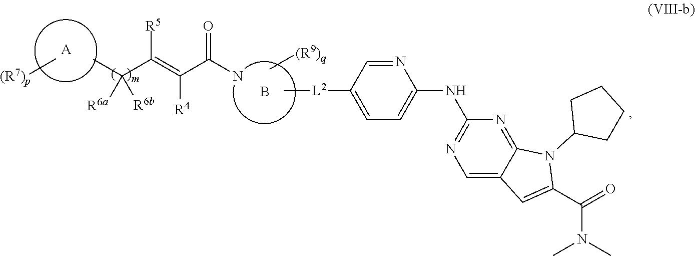

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, X is O. In an embodiment, each of R^{6a} and R^{6b} is independently hydrogen or C₁₋₆ alkyl. In an embodiment, R⁴ and R⁵ are each independently selected from the group consisting of hydrogen, halogen, —CN, C₁₋₆ alkyl, and C₁₋₆ haloalkyl. In an embodiment, each of R⁴ and R⁵ is independently hydrogen or C₁₋₆ alkyl. In an embodiment, m is 1 or 2. In an embodiment, n is 1 or 2. In an embodiment, Ring A is 6- to 10-membered aryl (e.g., phenyl). In an embodiment, p is 0, 1, 2, or 3. In an embodiment, L² is absent. In an embodiment, L² is a linker (e.g., a linker described herein).

In an embodiment, the molecular glue degrader compound of Formula (I) has the structure having a structure of Formula (VIII-c):

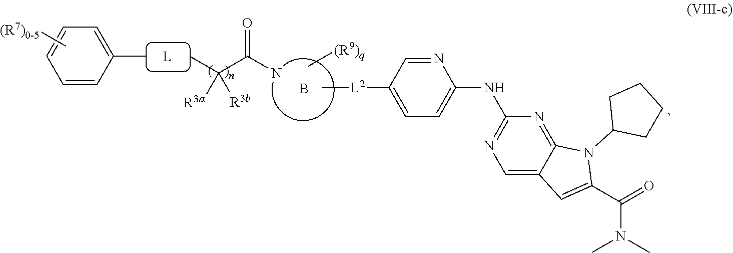

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (VIII-d):

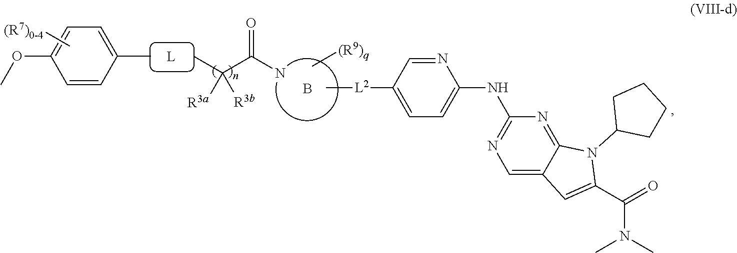

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (IX):

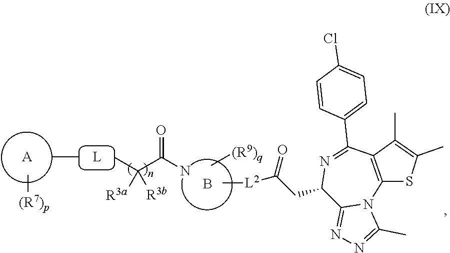

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (IX-a):

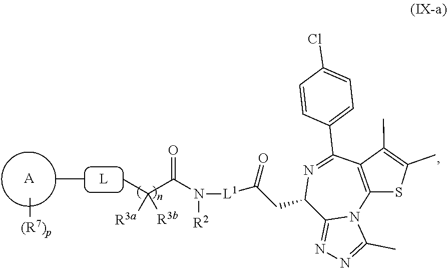

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (IX-b):

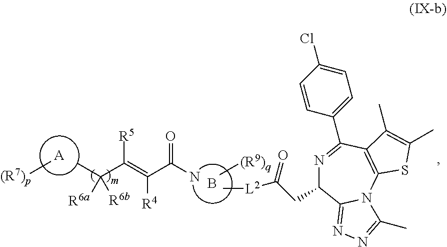

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (IX-c):

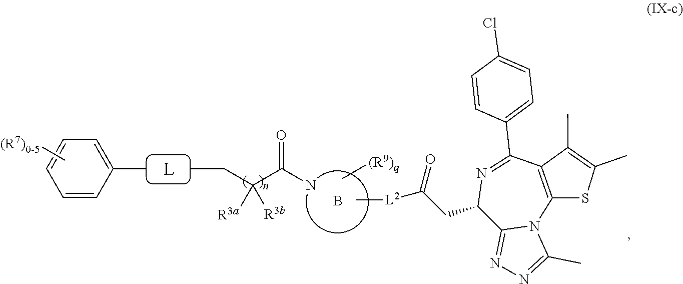

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (IX-d):

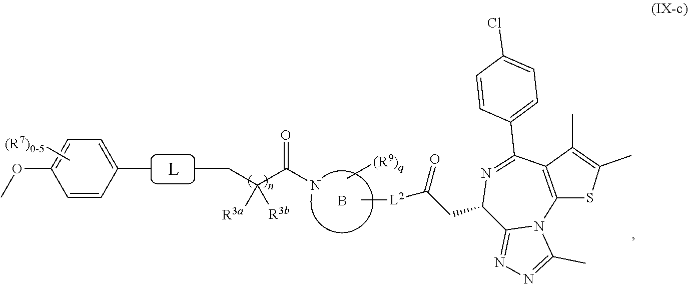

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (IX-e):

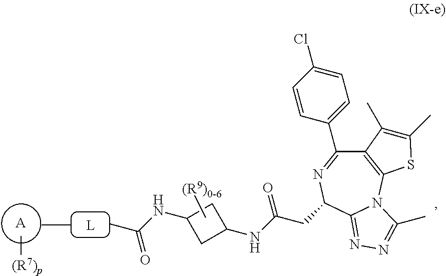

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (X):

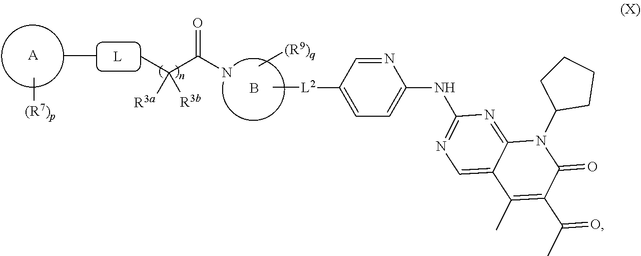

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (X-a):

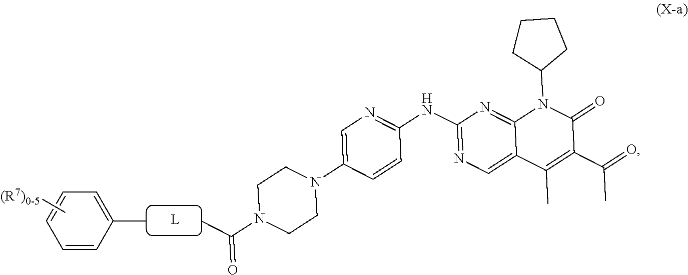

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (XI):

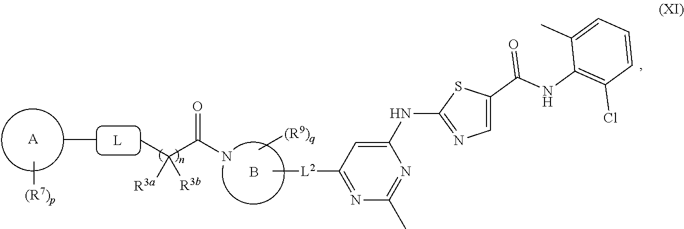

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (XII):

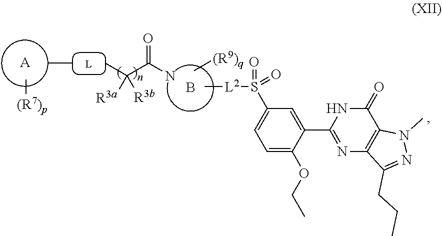

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (XIII):

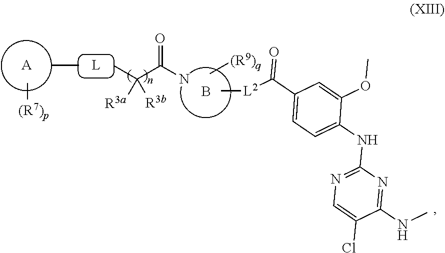

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (XIV):

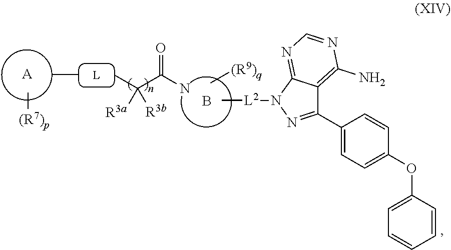

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (XV):

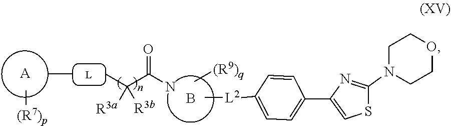

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (XV-a):

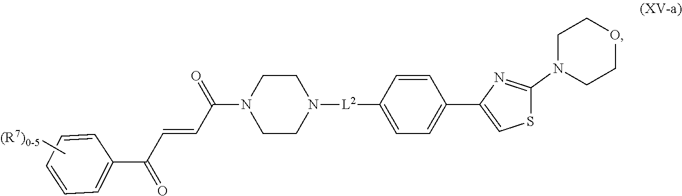

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (XVI):

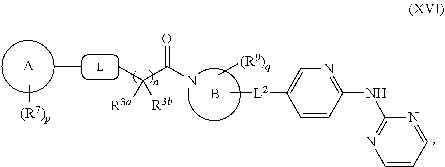

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

In an embodiment, the molecular glue degrader compound of Formula (I) has the structure having a structure of Formula (XVI-a):

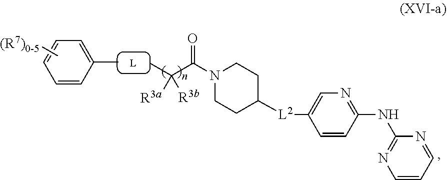

or a pharmaceutically acceptable salt, hydrate, solvate, stereoisomer, or tautomer thereof, wherein Ring B is a 5- to 10-membered heteroaryl or a 3- to 12-membered heterocycloalkyl, and each of the Ring A, L², R⁴, R⁵, R^{6a}, R^{6b}, R⁷, R⁹, m, p, and q are as defined for Formula (II).

### Ring A

As generally described herein for compounds of Formula (II)-(VIII), Ring A is selected from the group consisting of aryl, heteroaryl, cycloalkyl, and heterocycloalkyl; each of which is optionally substituted with one or more R⁷.

In some embodiments, Ring A is selected from the group consisting of 6- to 10-membered aryl, a 5- to 10-membered heteroaryl, a 3- to 12-membered cycloalkyl, and a 3- to 12-membered heterocycloalkyl; each of which is optionally substituted with one or more R⁷.

In some embodiments, Ring A is a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic).

In some embodiments, Ring A is a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, Ring A is a bicyclic ring comprising a fused, bridged, or spiro ring system.

In some embodiments, Ring A is an aryl or heteroaryl ring, optionally substituted with 1-4 occurrences of R⁷. In some embodiments, Ring A is a monocyclic aryl ring. In some embodiments, Ring A is a bicyclic aryl ring. In some embodiments, Ring A is a 6- to 10-membered aryl ring (e.g., phenyl). In some embodiments, Ring A is a 6-membered aryl ring. In some embodiments, Ring A is an aryl ring, fused with a heterocycloalkyl ring. In some embodiments, Ring A is an aryl ring, optionally substituted with two or more R⁷, wherein two R⁷ on adjacent atoms together with the atoms to which they are attached form an optionally substituted heterocycloalkyl ring. In some embodiments, Ring A is an aryl ring, optionally substituted with two or more R⁷, wherein two R⁷ on adjacent atoms together with the atoms to which they are attached form an optionally substituted heterocycloalkyl ring containing 1 or 2 O or N atoms. In some embodiments, Ring A is an aryl ring, optionally substituted with two or more R⁷, wherein two R⁷ on adjacent atoms together with the atoms to which they are attached form an optionally substituted heterocycloalkyl ring containing 1 oxygen atom. In some embodiments, Ring A is an aryl ring, optionally substituted with two or more R⁷, wherein two R⁷ on adjacent atoms together with the atoms to which they are attached form an optionally substituted heterocycloalkyl ring containing 2 O or N atoms.

In some embodiments, Ring A is a heteroaryl ring. In some embodiments, Ring A is a monocyclic heteroaryl ring. In some embodiments, Ring A is a bicyclic heteroaryl ring. In some embodiments, Ring A is a 5- to 10-membered heteroaryl ring. In some embodiments, Ring A is a 5-membered heteroaryl ring (e.g., thiophenyl). In some embodiments, Ring A is a 6-membered heteroaryl ring (e.g., pyridyl). In some embodiments, Ring A is a heteroaryl ring containing 0, S, or N. In some embodiments, Ring A is a monocyclic heteroaryl ring containing one O or S atom.

In some embodiments, Ring A is a heterocycloalkyl ring. In some embodiments, Ring A is a monocyclic heterocycloalkyl ring. In some embodiments, Ring A is a bicyclic heterocycloalkyl ring. In some embodiments, Ring A is a 3- to 12-membered heterocycloalkyl ring. In some embodiments, Ring A is a heterocycloalkyl ring, wherein the ring is fused, bridged, or spiro. In some embodiments, Ring A is a heterocycloalkyl ring, containing 1, 2, or 3 heteroatoms. In some embodiments, Ring A is a heterocycloalkyl ring, containing 1, 2, or 3 N, O, or S atoms. In some embodiments, Ring A is a heterocycloalkyl ring, containing 1, 2, or 3 N atoms. In some embodiments, Ring A is a monocyclic heterocycloalkyl ring, containing 1 or 2 N atoms. In some embodiments, Ring A is a heterocycloalkyl ring, optionally fused to an aryl ring. In some embodiments, Ring A is a heterocycloalkyl ring, containing at least one O and one N atom. In some embodiments, Ring A is a bicyclic spiro heterocycloalkyl ring.

In some embodiments, Ring A is a cycloalkyl ring. In some embodiments, Ring A is a monocyclic or bicyclic cycloalkyl ring. In some embodiments, Ring A is a monocyclic cycloalkyl ring. In some embodiments, Ring A is a 3- to 12-membered cycloalkyl ring. In some embodiments, Ring A is a 5-membered cycloalkyl ring. In some embodiments, Ring A is a cycloalkyl ring, optionally containing one double bond.

In some embodiments, Ring A is selected from

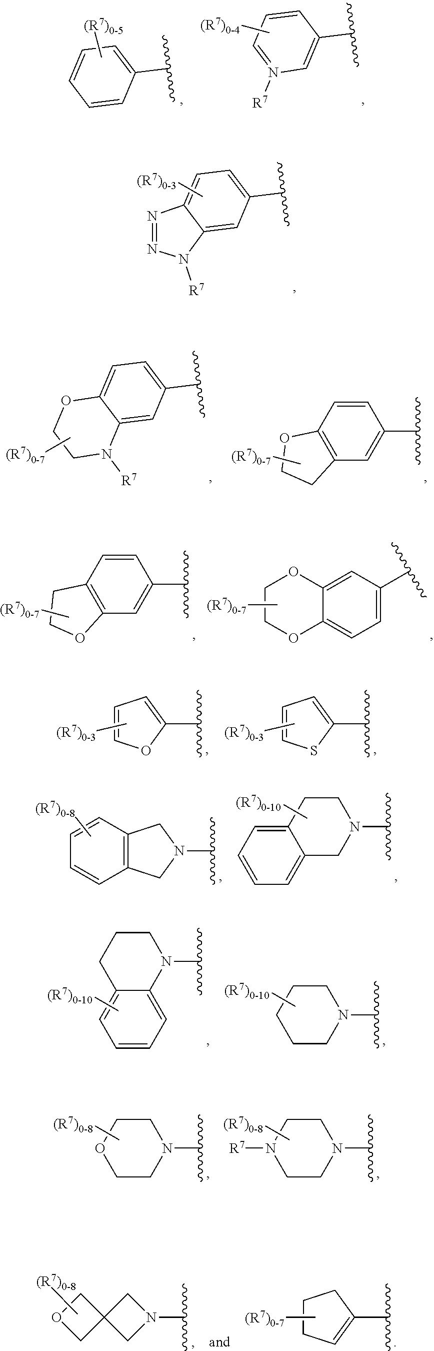

In some embodiments, Ring A is selected from

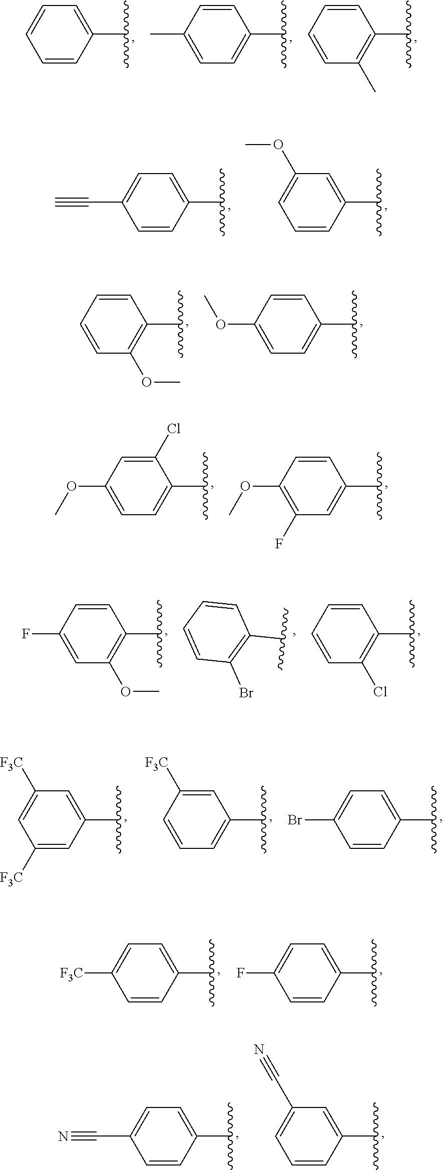

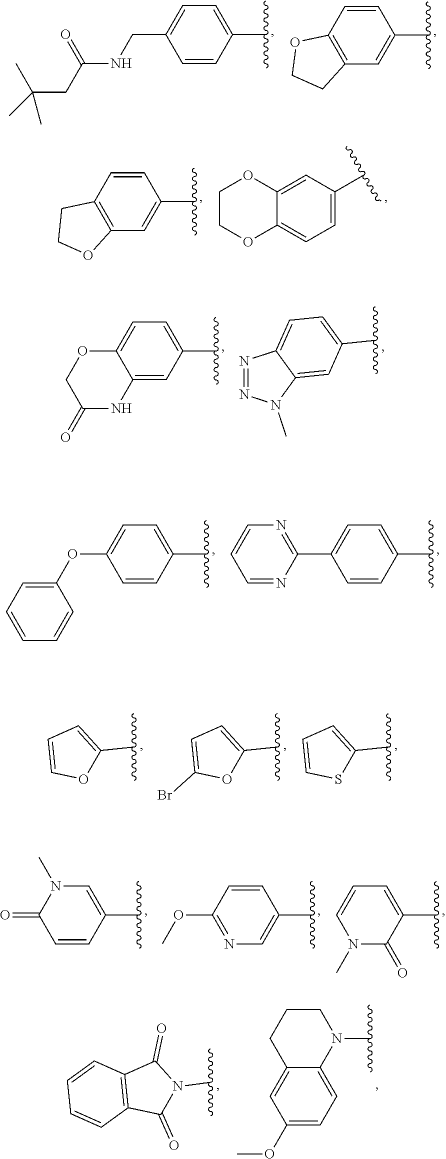

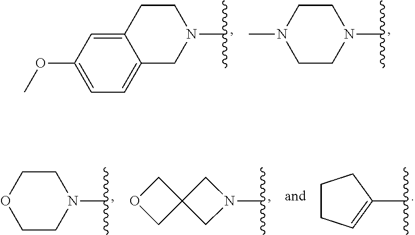

### Linkers

In an embodiment, the molecule glue degrader compound comprises a linker, termed “linker”, “L” or “L¹” herein. In an embodiment, the linker, L, or L¹ is absent. In an embodiment, the linker or L is L. In an embodiment, L¹ is absent. In an embodiment, L¹ is a linker selected from the group of alkyl, heteroalkyl, —(C₁₋₄ alkyl-NR)—, C₁₋₅ cycloalkyl, —(C₁₋₅ cycloalkyl)-NR—, wherein R is hydrogen or C₁₋₆ alkyl.

In an embodiment of Formula (II) and related subgenera, R¹ and R² together with the atoms to which they are attached form a ring, referred to hereinafter as “Ring B”, wherein Ring B is further substituted with R⁸, and 0-4 occurrences of R⁹. In an embodiment, Ring B is a heteroaryl or heterocycloalkyl ring. In an embodiment, Ring B is a 5- to 10-membered heteroaryl or a 3- to 12-membered heterocycloalkyl ring.

In an embodiment, Ring B is a heterocycloalkyl ring. In an embodiment, Ring B is a monocyclic or bicyclic heterocycloalkyl ring. In an embodiment, Ring B is a 3- to 12-membered heterocycloalkyl. In an embodiment, Ring B is a 6- or 7-membered heterocycloalkyl ring (e.g. piperazinyl). In an embodiment, Ring B is a heterocycloalkyl ring, comprising 1, 2, or 3 heteroatoms. In an embodiment, Ring B is a heterocycloalkyl ring, comprising 2 or 3 heteroatoms. In an embodiment, Ring B is a heterocycloalkyl ring, comprising 2 N atoms. In an embodiment, Ring B is a heterocycloalkyl ring, comprising 2 N atoms and one or more O atoms.

In an embodiment, Ring B is a fused, bridged, or spiro heterocycloalkyl ring. In an embodiment, Ring B is a fused bicyclic heterocycloalkyl ring. In an embodiment, Ring B is a fused bicyclic heterocycloalkyl ring, comprising 1, 2, or 3 heteroatoms. In an embodiment, Ring B is a fused bicyclic heterocycloalkyl ring, comprising 2 N atoms. In an embodiment, Ring B is a bridged heterocycloalkyl ring. In an embodiment, Ring B is a bridged heterocycloalkyl ring, comprising 1, 2, or 3 heteroatoms. In an embodiment, Ring B is a bridged heterocycloalkyl ring, comprising 2 heteroatoms. In an embodiment, Ring B is a bridged heterocycloalkyl ring, comprising 2 N atoms. In an embodiment, Ring B is a spiro heterocycloalkyl ring. In an embodiment, Ring B is a spiro heterocycloalkyl ring, comprising 1, 2, or 3 heteroatoms. In an embodiment, Ring B is a spiro heterocycloalkyl ring, comprising 2 or 3 heteroatoms. In an embodiment, Ring B is a spiro heterocycloalkyl ring, comprising 2 N atoms. In an embodiment, Ring B is a spiro heterocycloalkyl ring, comprising 2 N atoms and one or more O atoms.

In an embodiment, Ring B is selected from the group of:

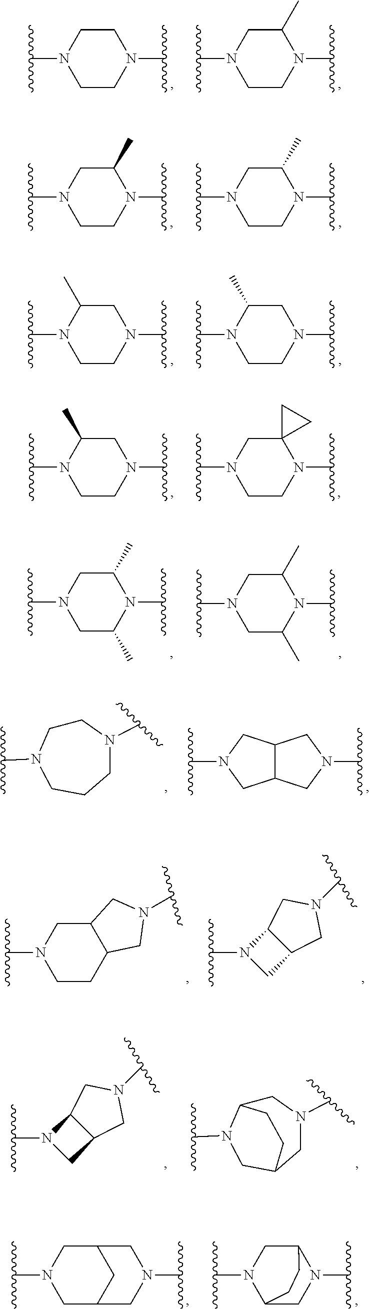

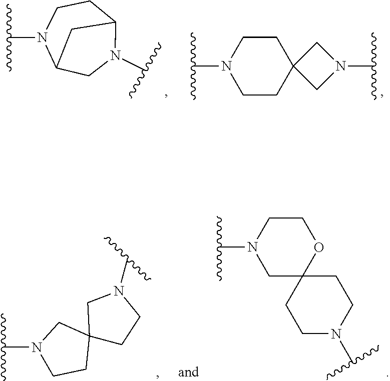 [SMILES: *N1CCC2CC1CN2* ; *N1CCC2(CC1)CN(*)C2 ; *N1CCC2(CCN(*)C2)C1 ; *N1CCC2(CC1)CN(*)CCO2]

In an embodiment, Ring B is incorporated in the Target Ligand, e.g., the Target Ligand includes Ring B.

### Target Ligands

As generally described herein, the Target Ligand comprises a moiety capable of binding to a target protein.

In an embodiment, the Target Ligand is a kinase inhibitor, bromodomain inhibitor, or phosphodiesterase inhibitor. In an embodiment, the Target Ligand is capable of binding to CDK4, CDK6, BRD4, PDE5, BCR-ABL, c-ABL, AR, AR-V7, BTK, LRRK2, or SMARCA2. In an embodiment, the Target Ligand is selected from ribociclib, dasatinib, palbociclib, sildenafil, HG-10-102-01, JQ1, ibrutinib, or a derivative thereof.

In an embodiment, the Target Ligand is selected from the group of:

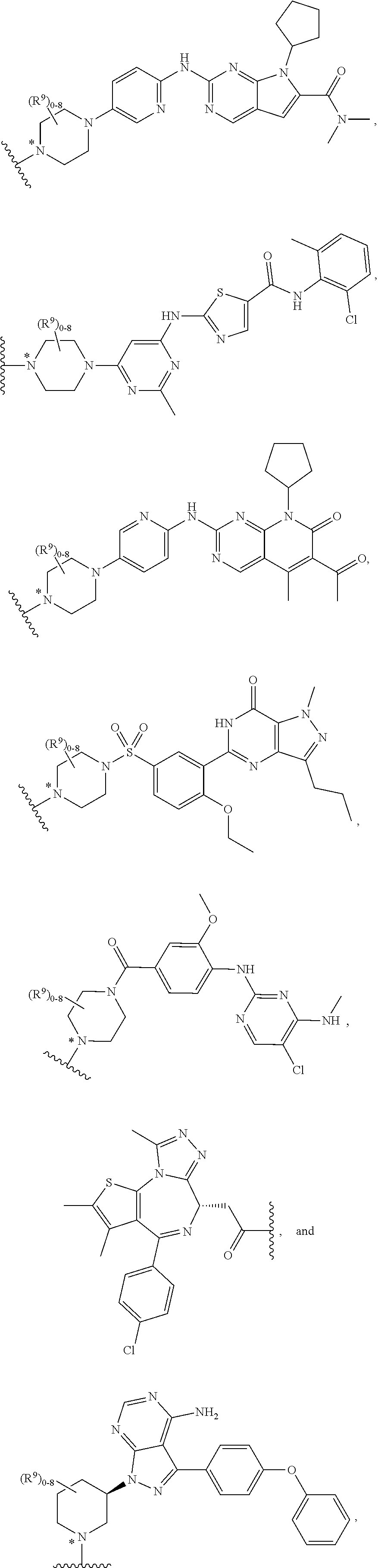

or derivatives thereof, wherein N* is the same N atom of —NR¹R² in Formulas (II)-(XV).

In an embodiment, the Target Ligand is selected from the group of:

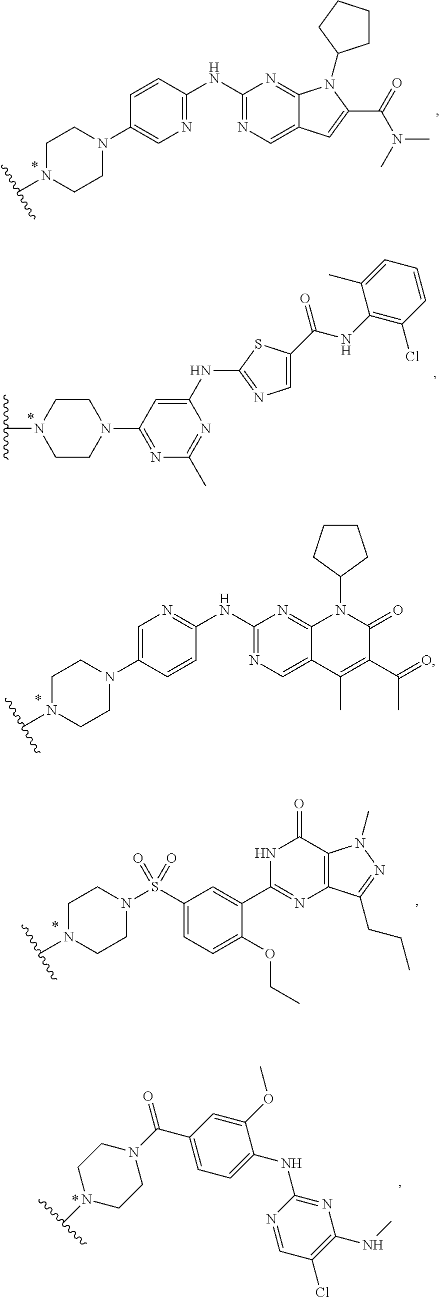 [SMILES: *N1CCN(c2ccc(Nc3ncc4cc(C(=O)N(C)C)n(C5CCCC5)c4n3)nc2)CC1 ; *N1CCN(c2cc(Nc3ncc(C(=O)Nc4c(C)cccc4Cl)s3)nc(C)n2)CC1 ; *N1CCN(c2ccc(Nc3ncc4c(C)c(C(C)=O)c(=O)n(C5CCCC5)c4n3)nc2)CC1 ; *N1CCN(S(=O)(=O)c2ccc(OCC)c(-c3nc4c(CCC)nn(C)c4c(=O)[nH]3)c2)CC1 ; *N1CCN(C(=O)c2ccc(Nc3ncc(Cl)c(NC)n3)c(OC)c2)CC1]

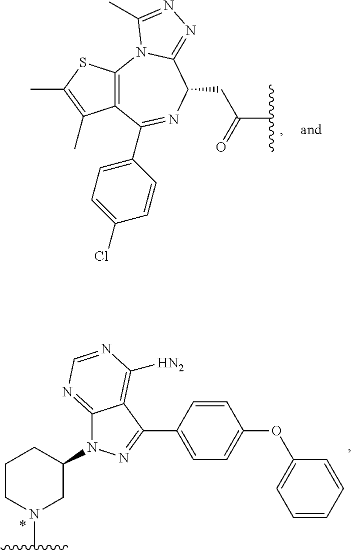

or a derivative thereof, wherein N* is the same N atom of —NR¹R² in Formulas (II)-(XV).

In an embodiment, the Target Ligand is selected from ribociclib or JQ1. In an embodiment, the Target Ligand is selected from

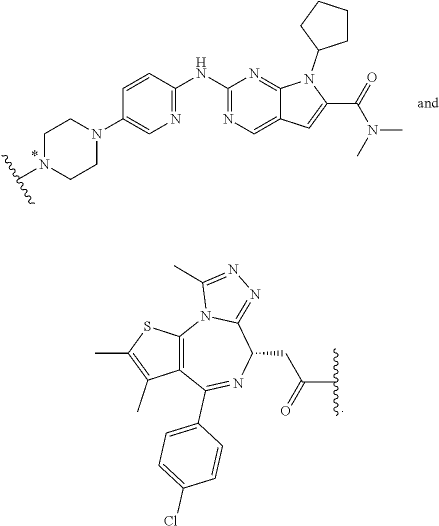

In an embodiment, the Target Ligand is selected from the group of

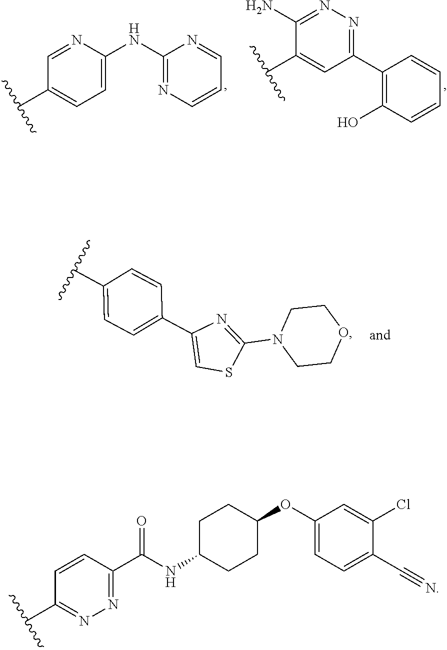 [SMILES: *c1ccc(Nc2ncccn2)nc1 ; *c1cc(-c2ccccc2O)nnc1N ; *c1ccc(-c2csc(N3CCOCC3)n2)cc1 ; *c1ccc(C(=O)N[C@H]2CC[C@H](Oc3ccc(C#N)c(Cl)c3)CC2)nn1]

In an embodiment, the Target Ligand is an aryl or heteroaryl ring. In an embodiment, the Target Ligand is an aryl ring (e.g., phenyl). In an embodiment, the Target Ligand is a heteroaryl ring (e.g., pyridyl). In an embodiments, the Target Ligand is selected from

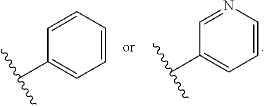 [SMILES: *c1ccccc1 ; *c1cccnc1]

In an embodiment, the molecular glue degrader compound is a compound selected from Table 1, or a pharmaceutically acceptable salt, hydrate, solvate, stereoisomer, or tautomer thereof. In an embodiment, the molecular glue degrader compound (e.g., a compound of Formula (I), e.g., a compound provided in Table 1) is prepared via an intermediate provided in Table 2, or a pharmaceutically acceptable salt, hydrate, solvate, stereoisomer, or tautomer thereof.

TABLE 1
Exemplary molecular glue degrader compounds

| Compound | |
|---|---|
| No. | Structure |
| 100 | 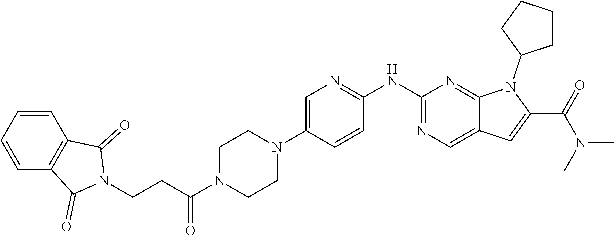 [SMILES: CN(C)C(=O)c1cc2cnc(Nc3ccc(N4CCN(C(=O)CCN5C(=O)c6ccccc6C5=O)CC4)cn3)nc2n1C1CCCC1] |
| 101 | 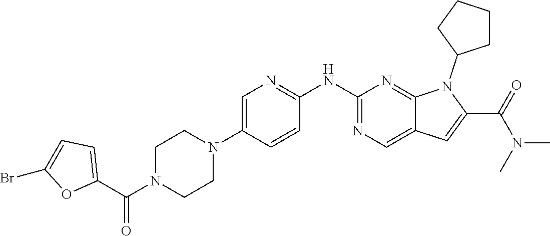 [SMILES: CN(C)C(=O)c1cc2cnc(Nc3ccc(N4CCN(C(=O)c5ccc(Br)o5)CC4)cn3)nc2n1C1CCCC1] |
| 102 | 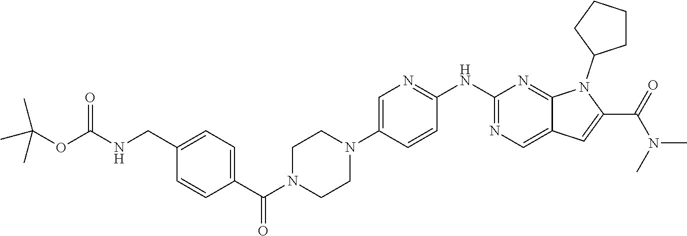 [SMILES: CN(C)C(=O)c1cc2cnc(Nc3ccc(N4CCN(C(=O)c5ccc(CNC(=O)OC(C)(C)C)cc5)CC4)cn3)nc2n1C1CCCC1] |
| 103 | 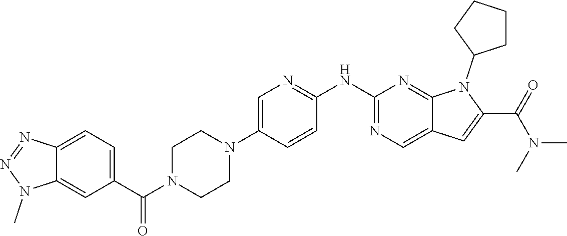 [SMILES: CN(C)C(=O)c1cc2cnc(Nc3ccc(N4CCN(C(=O)c5ccc6nnn(C)c6c5)CC4)cn3)nc2n1C1CCCC1] |
| 104 | 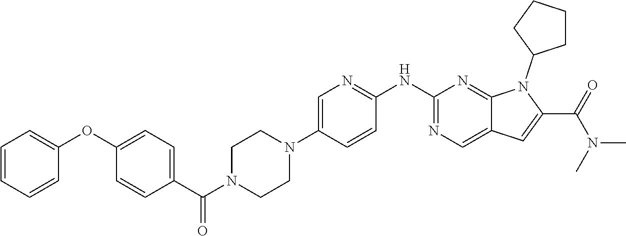 [SMILES: CN(C)C(=O)c1cc2cnc(Nc3ccc(N4CCN(C(=O)c5ccc(Oc6ccccc6)cc5)CC4)cn3)nc2n1C1CCCC1] |
| 105 | 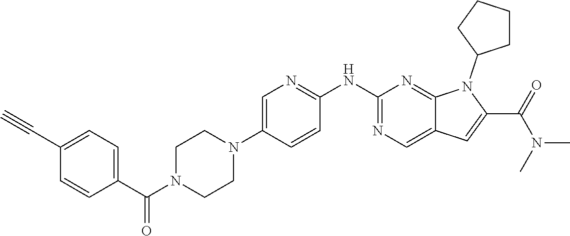 [SMILES: C#Cc1ccc(C(=O)N2CCN(c3ccc(Nc4ncc5cc(C(=O)N(C)C)n(C6CCCC6)c5n4)nc3)CC2)cc1] |
| 106 | 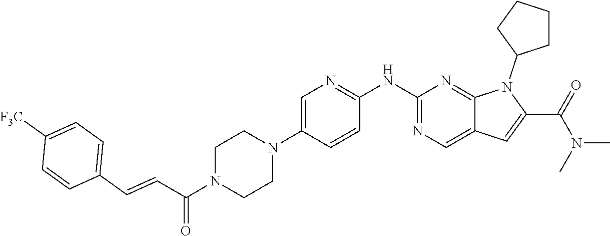 [SMILES: CN(C)C(=O)c1cc2cnc(Nc3ccc(N4CCN(C(=O)/C=C/c5ccc(C(F)(F)F)cc5)CC4)cn3)nc2n1C1CCCC1] |
| 107 | 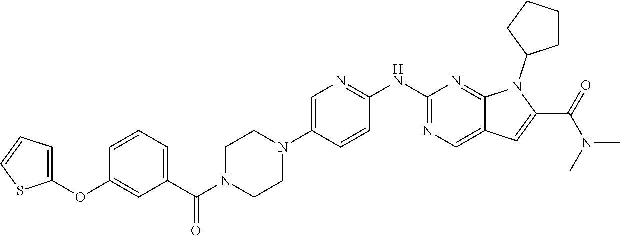 [SMILES: CN(C)C(=O)c1cc2cnc(Nc3ccc(N4CCN(C(=O)c5cccc(Oc6cccs6)c5)CC4)cn3)nc2n1C1CCCC1] |
| 108 | 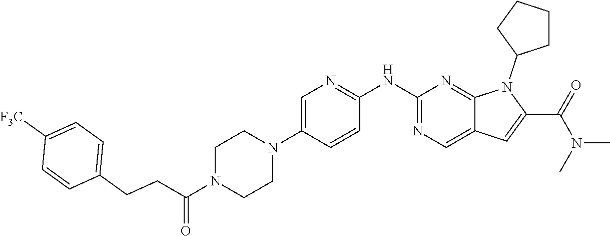 [SMILES: CN(C)C(=O)c1cc2cnc(Nc3ccc(N4CCN(C(=O)CCc5ccc(C(F)(F)F)cc5)CC4)cn3)nc2n1C1CCCC1] |
| 109 | 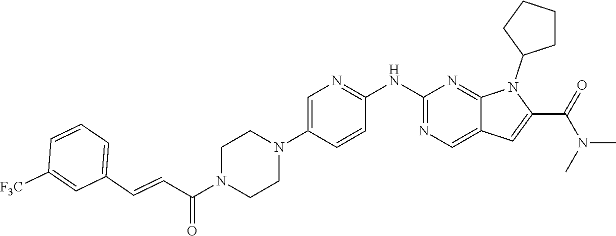 [SMILES: CN(C)C(=O)c1cc2cnc(Nc3ccc(N4CCN(C(=O)/C=C/c5cccc(C(F)(F)F)c5)CC4)cn3)nc2n1C1CCCC1] |
| 110 | 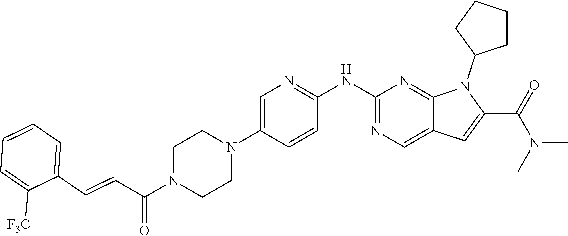 [SMILES: CN(C)C(=O)c1cc2cnc(Nc3ccc(N4CCN(C(=O)/C=C/c5ccccc5C(F)(F)F)CC4)cn3)nc2n1C1CCCC1] |
| 111 | 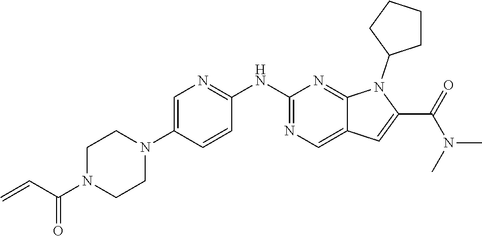 [SMILES: C=CC(=O)N1CCN(c2ccc(Nc3ncc4cc(C(=O)N(C)C)n(C5CCCC5)c4n3)nc2)CC1] |
| 112 | 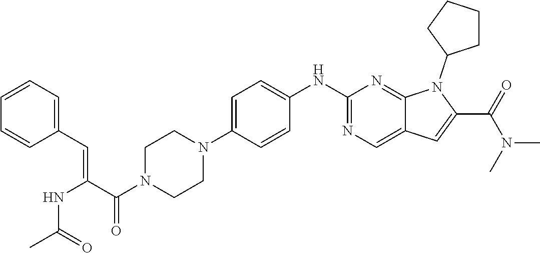 [SMILES: CC(=O)N/C(=C\c1ccccc1)C(=O)N1CCN(c2ccc(Nc3ncc4cc(C(=O)N(C)C)n(C5CCCC5)c4n3)cc2)CC1] |
| 113 | 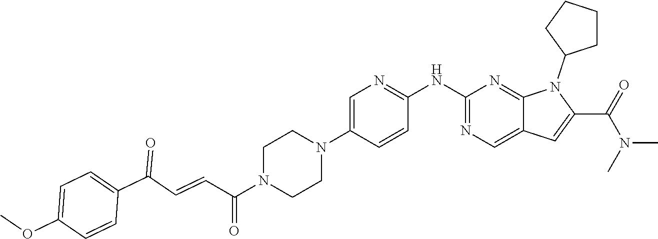 |
| 114 | 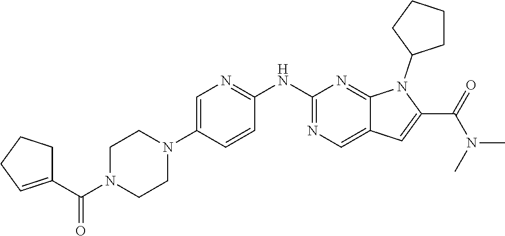 [SMILES: CN(C)C(=O)c1cc2cnc(Nc3ccc(N4CCN(C(=O)C5=CCCC5)CC4)cn3)nc2n1C1CCCC1] |
| 115 | 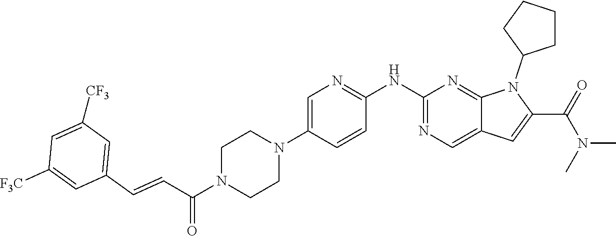 [SMILES: CN(C)C(=O)c1cc2cnc(Nc3ccc(N4CCN(C(=O)/C=C/c5cc(C(F)(F)F)cc(C(F)(F)F)c5)CC4)cn3)nc2n1C1CCCC1] |
| 120 | 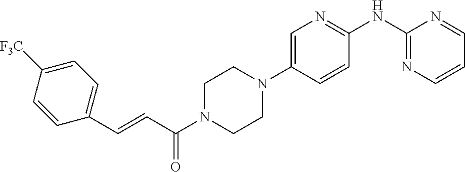 [SMILES: O=C(/C=C/c1ccc(C(F)(F)F)cc1)N1CCN(c2ccc(Nc3ncccn3)nc2)CC1] |
| 125 | 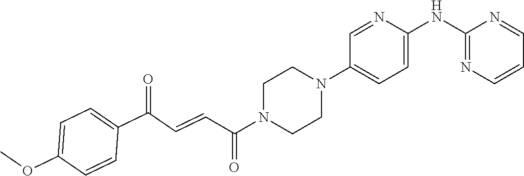 [SMILES: COc1ccc(C(=O)/C=C/C(=O)N2CCN(c3ccc(Nc4ncccn4)nc3)CC2)cc1] |
| 126 | 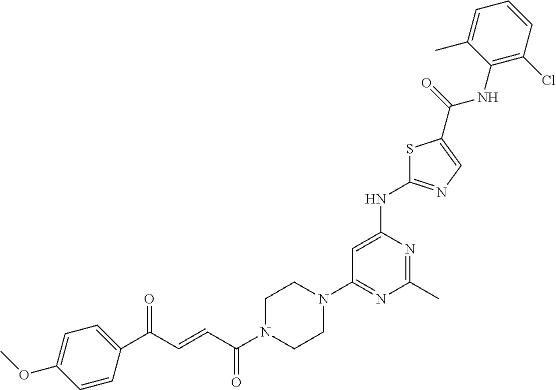 [SMILES: COc1ccc(C(=O)/C=C/C(=O)N2CCN(c3cc(Nc4ncc(C(=O)Nc5c(C)cccc5Cl)s4)nc(C)n3)CC2)cc1] |
| 127 | 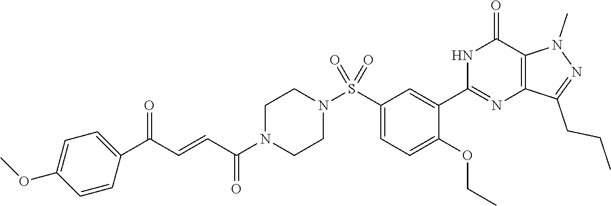 [SMILES: CCCc1nn(C)c2c(=O)[nH]c(-c3cc(S(=O)(=O)N4CCN(C(=O)/C=C/C(=O)c5ccc(OC)cc5)CC4)ccc3OCC)nc12] |
| 128 | 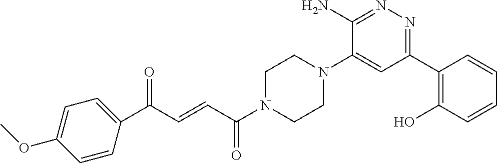 [SMILES: COc1ccc(C(=O)/C=C/C(=O)N2CCN(c3cc(-c4ccccc4O)nnc3N)CC2)cc1] |
| 129 | 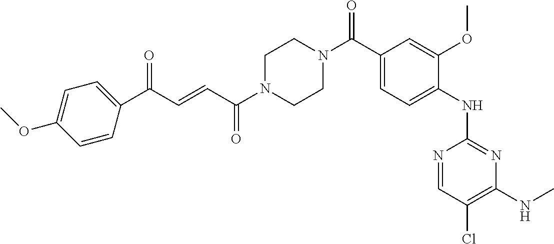 |
| 130 | 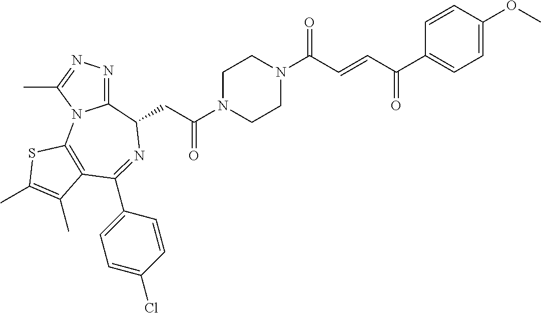 |
| 131 | 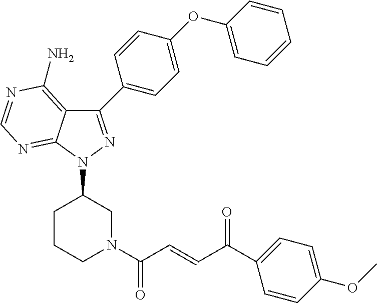 [SMILES: COc1ccc(C(=O)/C=C/C(=O)N2CCC[C@@H](n3nc(-c4ccc(Oc5ccccc5)cc4)c4c(N)ncnc43)C2)cc1] |
| 132 | 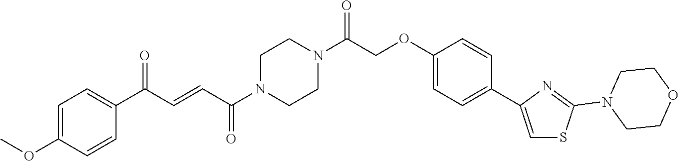 [SMILES: COc1ccc(C(=O)/C=C/C(=O)N2CCN(C(=O)COc3ccc(-c4csc(N5CCOCC5)n4)cc3)CC2)cc1] |
| 133 | 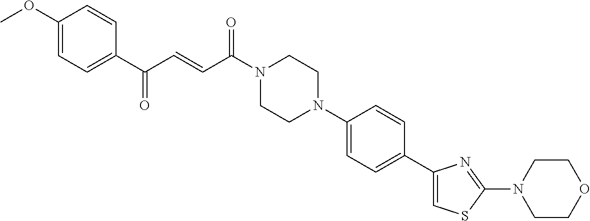 [SMILES: COc1ccc(C(=O)/C=C/C(=O)N2CCN(c3ccc(-c4csc(N5CCOCC5)n4)cc3)CC2)cc1] |
| 134 | 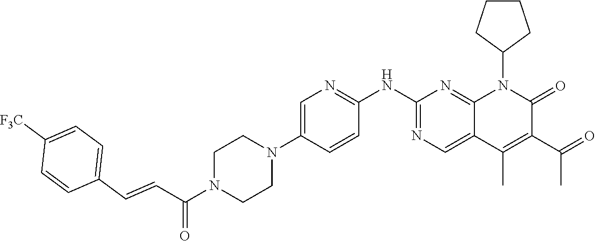 [SMILES: CC(=O)c1c(C)c2cnc(Nc3ccc(N4CCN(C(=O)/C=C/c5ccc(C(F)(F)F)cc5)CC4)cn3)nc2n(C2CCCC2)c1=O] |
| 135 | 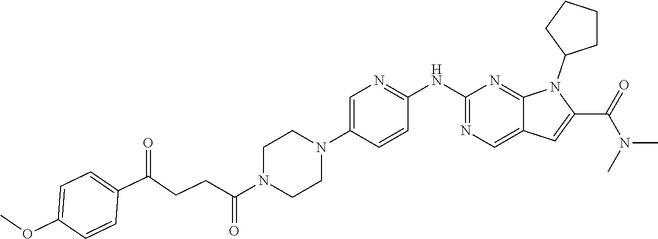 |
| 136 | 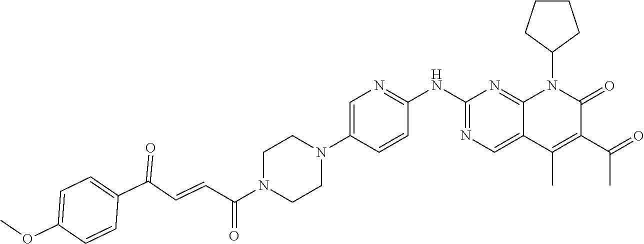 [SMILES: COc1ccc(C(=O)/C=C/C(=O)N2CCN(c3ccc(Nc4ncc5c(C)c(C(C)=O)c(=O)n(C6CCCC6)c5n4)nc3)CC2)cc1] |
| 137 | 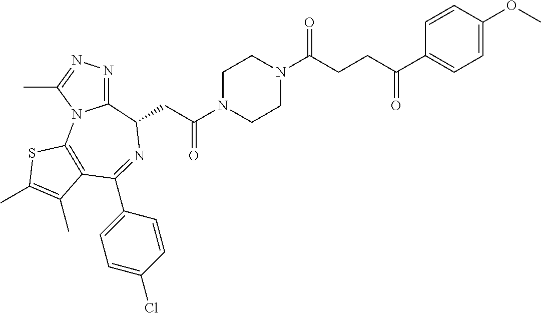 |
| 138 | 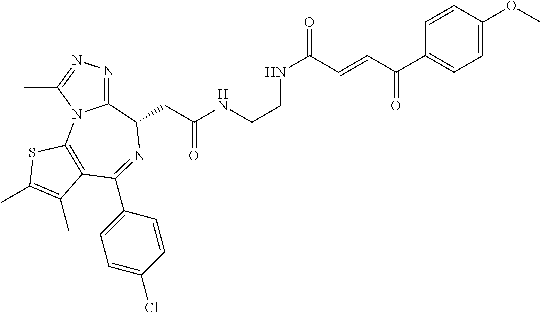 |
| 139 | 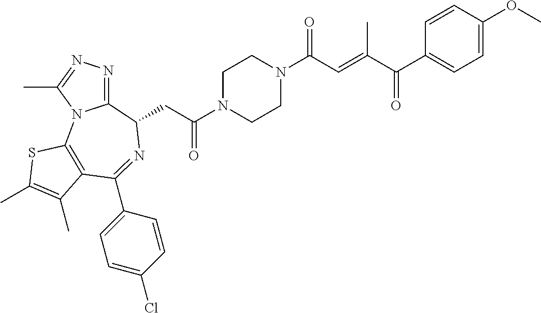 |
| 140 | 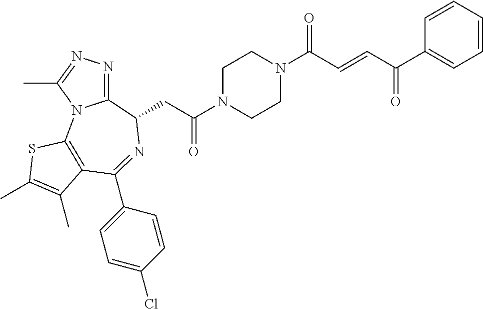 [SMILES: Cc1sc2c(c1C)C(c1ccc(Cl)cc1)=N[C@@H](CC(=O)N1CCN(C(=O)/C=C/C(=O)c3ccccc3)CC1)c1nnc(C)n1-2] |
| 141 | 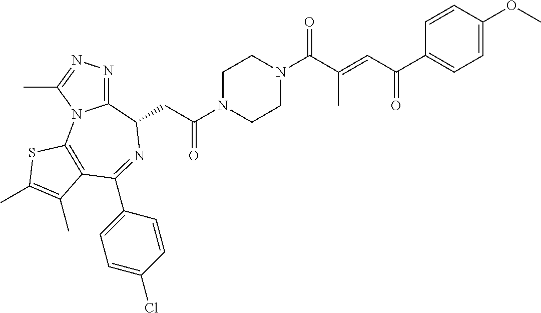 |
| 142 | 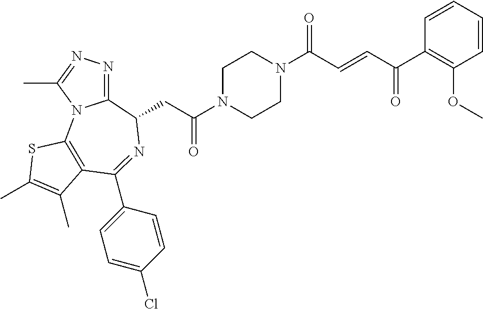 |
| 143 | 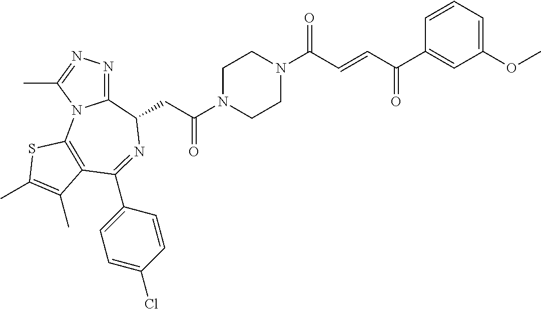 |
| 144 | 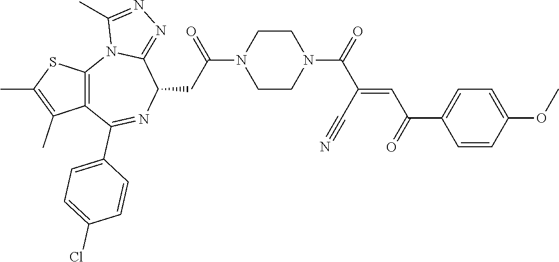 |
| 146 | 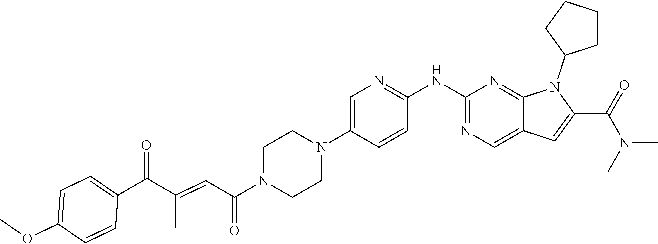 |
| 148 | 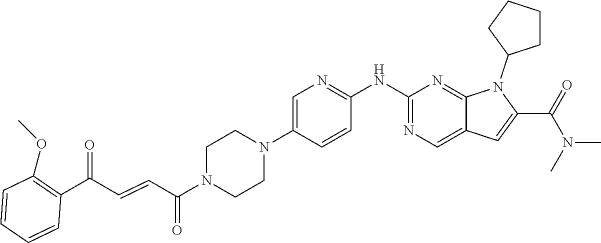 [SMILES: COc1ccccc1C(=O)/C=C/C(=O)N1CCN(c2ccc(Nc3ncc4cc(C(=O)N(C)C)n(C5CCCC5)c4n3)nc2)CC1] |
| 151 | 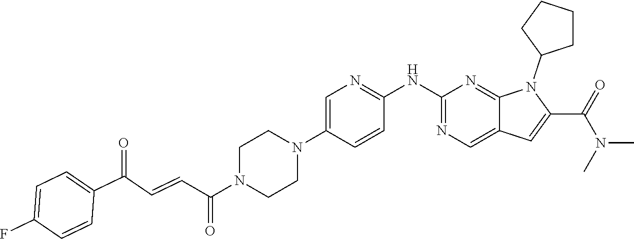 [SMILES: CN(C)C(=O)c1cc2cnc(Nc3ccc(N4CCN(C(=O)/C=C/C(=O)c5ccc(F)cc5)CC4)cn3)nc2n1C1CCCC1] |
| 158 | 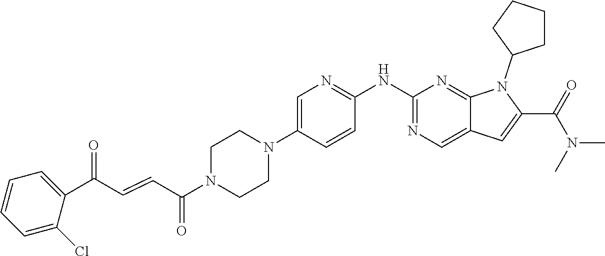 [SMILES: CN(C)C(=O)c1cc2cnc(Nc3ccc(N4CCN(C(=O)/C=C/C(=O)c5ccccc5Cl)CC4)cn3)nc2n1C1CCCC1] |
| 159 | 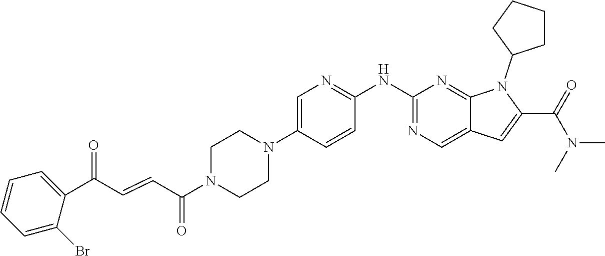 [SMILES: CN(C)C(=O)c1cc2cnc(Nc3ccc(N4CCN(C(=O)/C=C/C(=O)c5ccccc5Br)CC4)cn3)nc2n1C1CCCC1] |
| 160 | 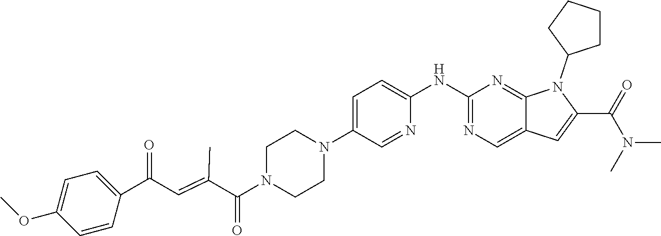 |
| 165 | 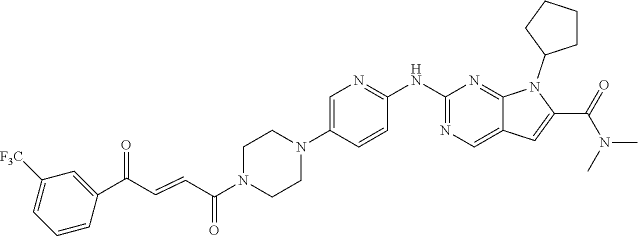 [SMILES: CN(C)C(=O)c1cc2cnc(Nc3ccc(N4CCN(C(=O)/C=C/C(=O)c5cccc(C(F)(F)F)c5)CC4)cn3)nc2n1C1CCCC1] |
| 170 | 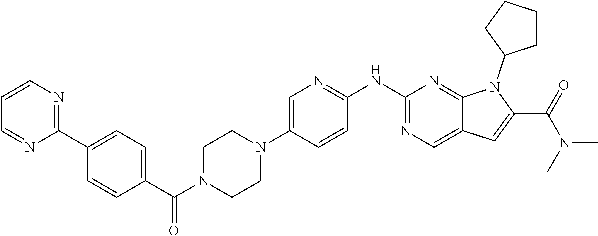 [SMILES: CN(C)C(=O)c1cc2cnc(Nc3ccc(N4CCN(C(=O)c5ccc(-c6ncccn6)cc5)CC4)cn3)nc2n1C1CCCC1] |
| 200 | 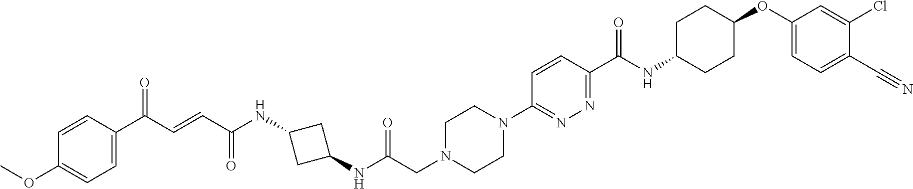 [SMILES: COc1ccc(C(=O)/C=C/C(=O)N[C@H]2C[C@H](NC(=O)CN3CCN(c4ccc(C(=O)N[C@H]5CC[C@H](Oc6ccc(C#N)c(Cl)c6)CC5)nn4)CC3)C2)cc1] |
| 201 | 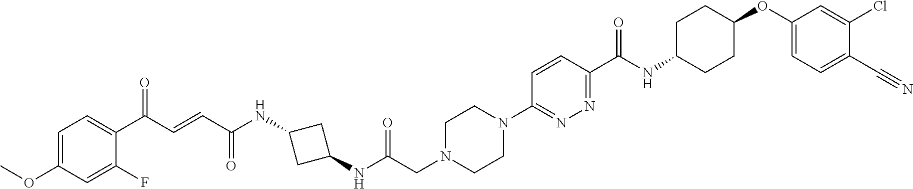 [SMILES: COc1ccc(C(=O)/C=C/C(=O)N[C@H]2C[C@H](NC(=O)CN3CCN(c4ccc(C(=O)N[C@H]5CC[C@H](Oc6ccc(C#N)c(Cl)c6)CC5)nn4)CC3)C2)c(F)c1] |
| 202 | 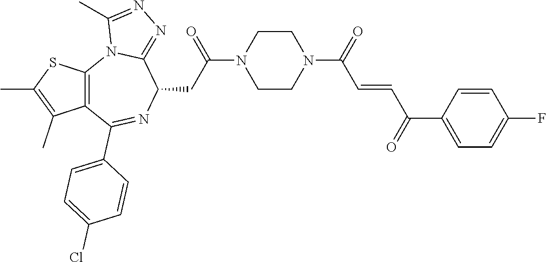 [SMILES: Cc1sc2c(c1C)C(c1ccc(Cl)cc1)=N[C@@H](CC(=O)N1CCN(C(=O)/C=C/C(=O)c3ccc(F)cc3)CC1)c1nnc(C)n1-2] |
| 203 | 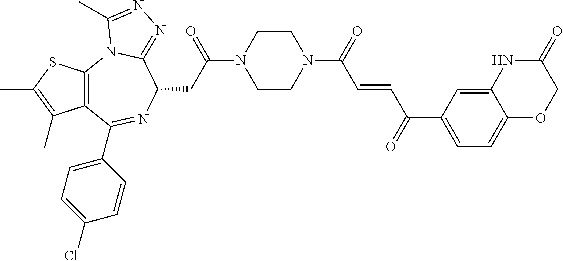 [SMILES: Cc1sc2c(c1C)C(c1ccc(Cl)cc1)=N[C@@H](CC(=O)N1CCN(C(=O)/C=C/C(=O)c3ccc4c(c3)NC(=O)CO4)CC1)c1nnc(C)n1-2] |
| 204 | 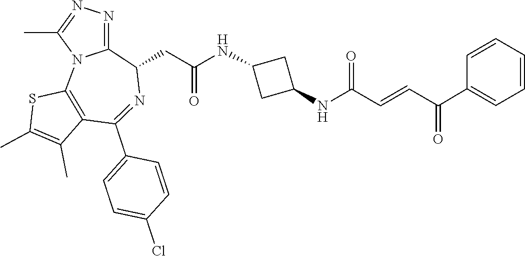 [SMILES: Cc1sc2c(c1C)C(c1ccc(Cl)cc1)=N[C@@H](CC(=O)N[C@H]1C[C@H](NC(=O)/C=C/C(=O)c3ccccc3)C1)c1nnc(C)n1-2] |
| 205 | 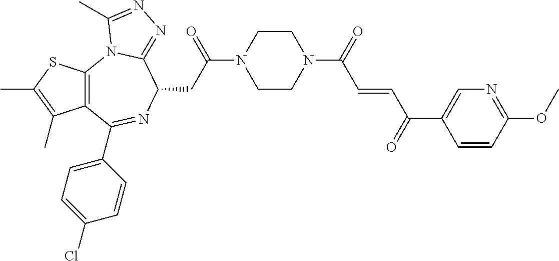 |
| 206 | 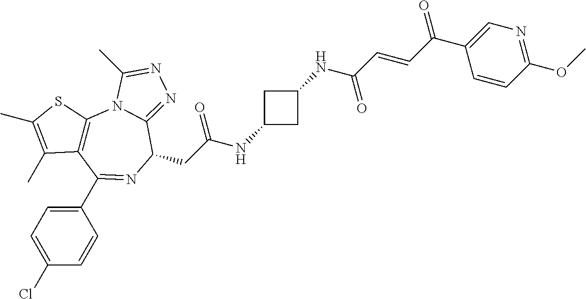 |
| 207 | 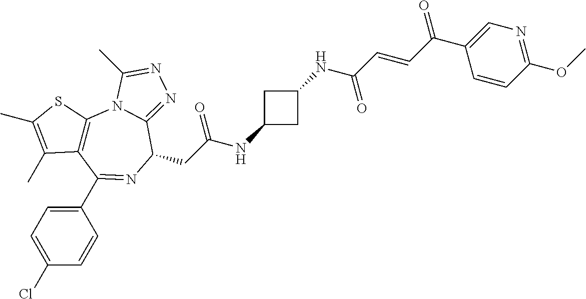 |
| 208 | 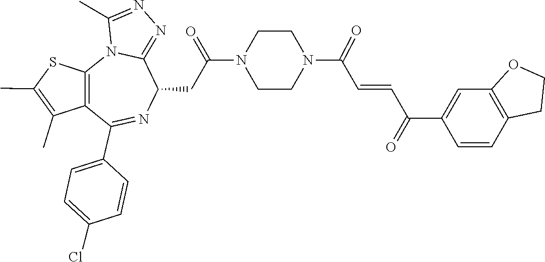 [SMILES: Cc1sc2c(c1C)C(c1ccc(Cl)cc1)=N[C@@H](CC(=O)N1CCN(C(=O)/C=C/C(=O)c3ccc4c(c3)OCC4)CC1)c1nnc(C)n1-2] |
| 209 | 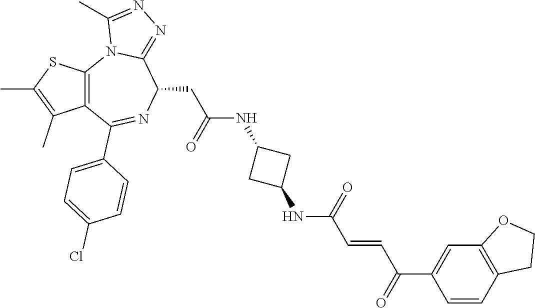 [SMILES: Cc1sc2c(c1C)C(c1ccc(Cl)cc1)=N[C@@H](CC(=O)N[C@H]1C[C@H](NC(=O)/C=C/C(=O)c3ccc4c(c3)OCC4)C1)c1nnc(C)n1-2] |
| 210 | 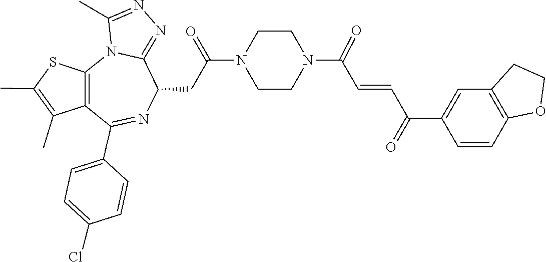 [SMILES: Cc1sc2c(c1C)C(c1ccc(Cl)cc1)=N[C@@H](CC(=O)N1CCN(C(=O)/C=C/C(=O)c3ccc4c(c3)CCO4)CC1)c1nnc(C)n1-2] |
| 211 | 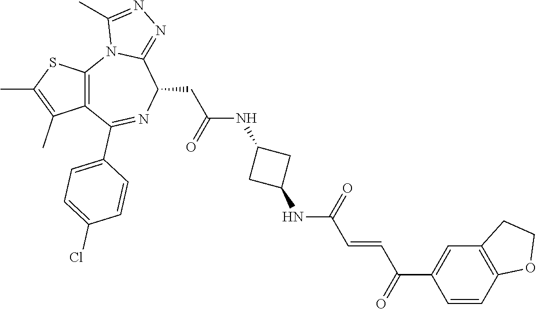 [SMILES: Cc1sc2c(c1C)C(c1ccc(Cl)cc1)=N[C@@H](CC(=O)N[C@H]1C[C@H](NC(=O)/C=C/C(=O)c3ccc4c(c3)CCO4)C1)c1nnc(C)n1-2] |
| 212 | 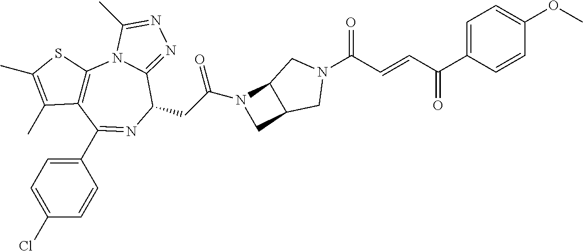 |
| 213 | 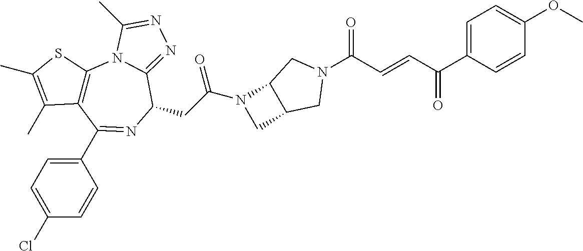 |
| 214 | 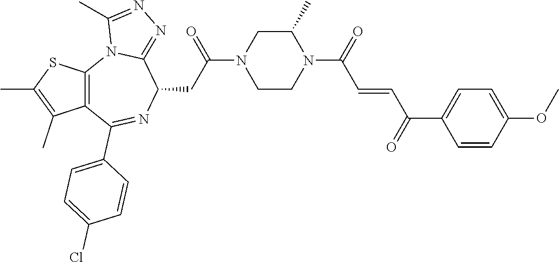 |
| 215 | 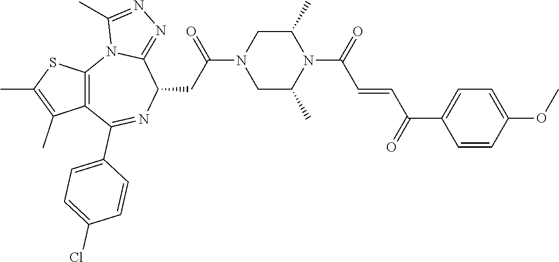 |
| 216 | 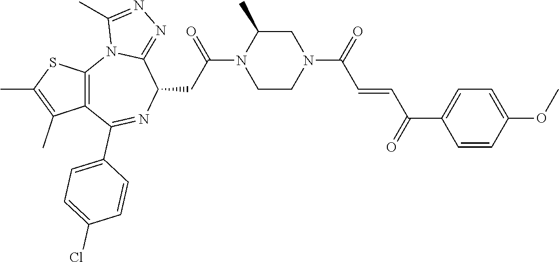 |
| 217 | 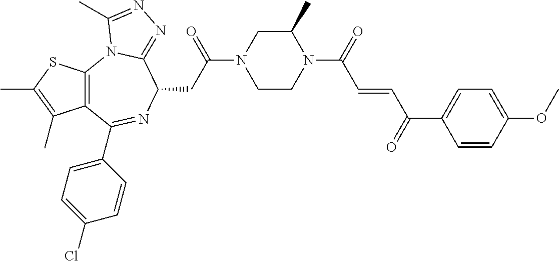 |
| 218 | 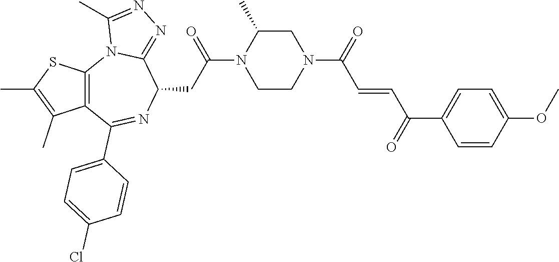 |
| 219 | 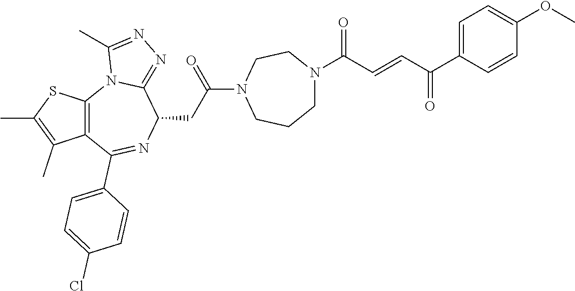 |
| 220 | 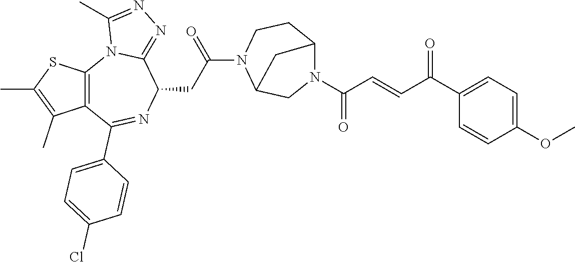 |
| 221 | 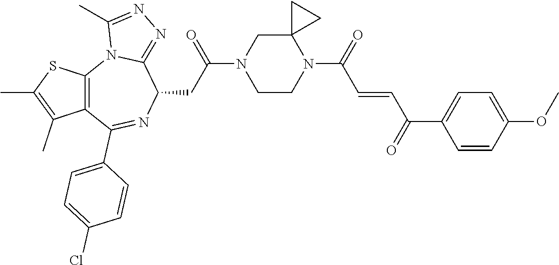 |
| 222 | 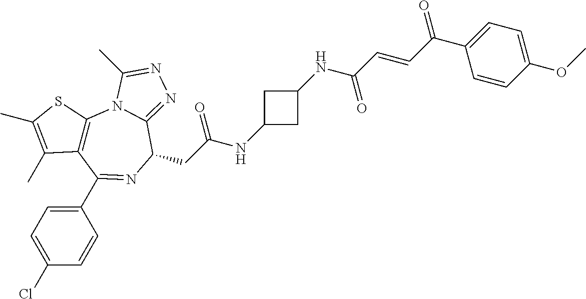 |
| 223 | 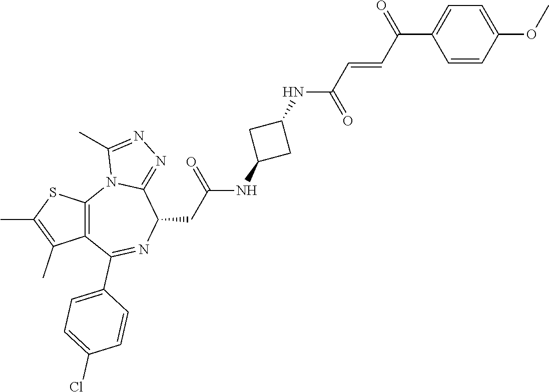 |
| 224 | 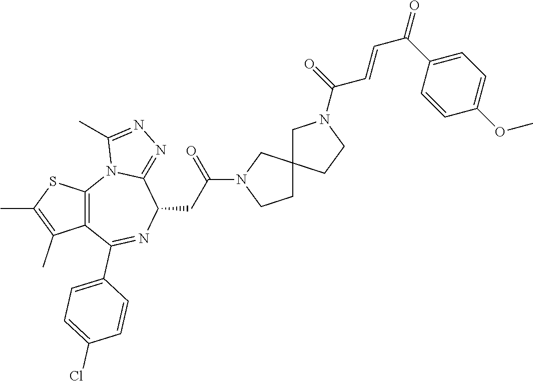 |
| 225 | 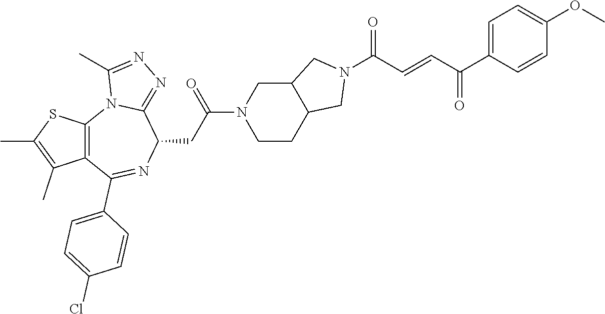 |
| 226 | 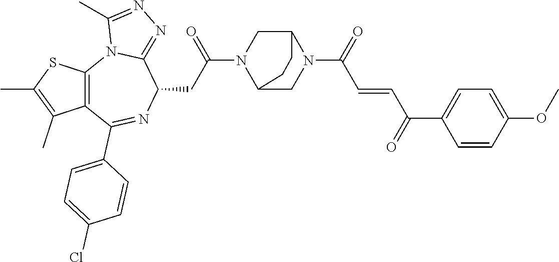 |
| 227 | 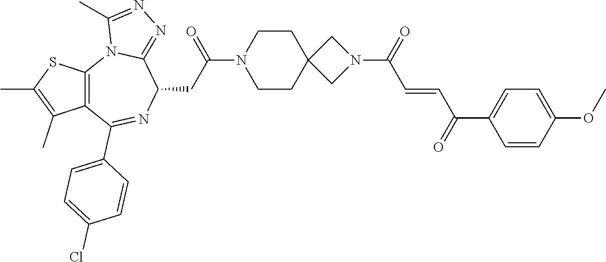 |
| 228 | 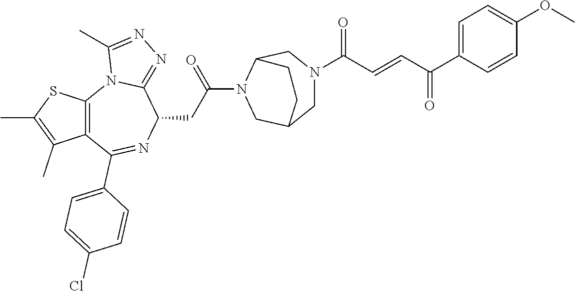 |
| 229 | 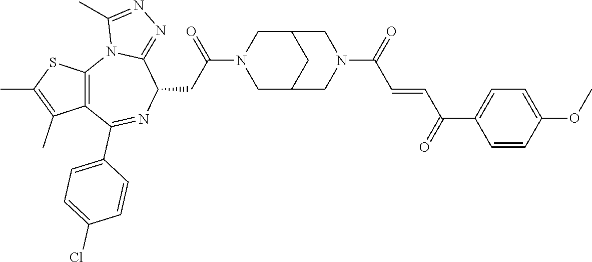 |
| 230 | 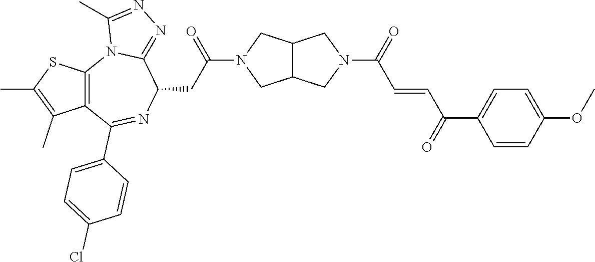 |
| 231 | 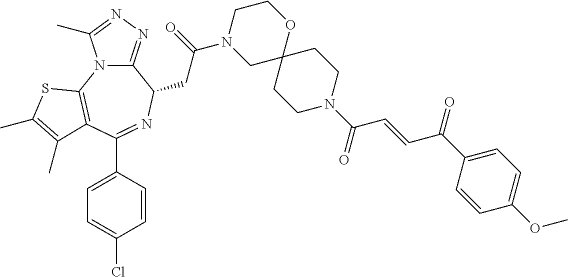 |
| 232 | 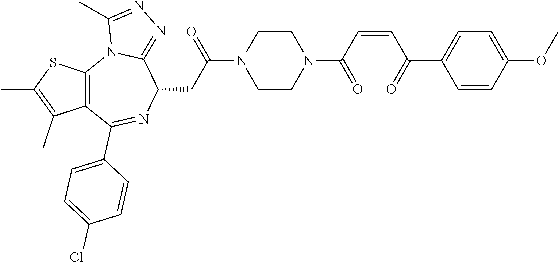 |
| 233 | 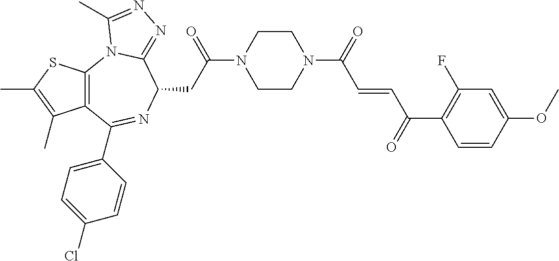 |
| 234 | 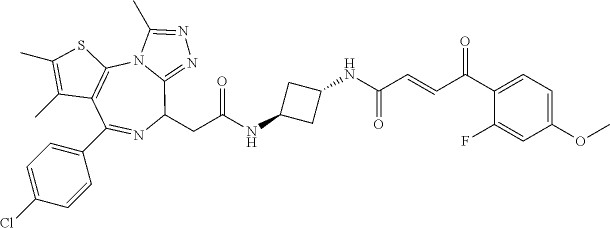 [SMILES: COc1ccc(C(=O)/C=C/C(=O)N[C@H]2C[C@H](NC(=O)CC3N=C(c4ccc(Cl)cc4)c4c(sc(C)c4C)-n4c(C)nnc43)C2)c(F)c1] |
| 235 | 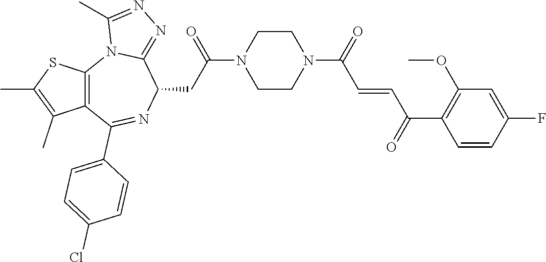 [SMILES: COc1cc(F)ccc1C(=O)/C=C/C(=O)N1CCN(C(=O)C[C@@H]2N=C(c3ccc(Cl)cc3)c3c(sc(C)c3C)-n3c(C)nnc32)CC1] |
| 236 | 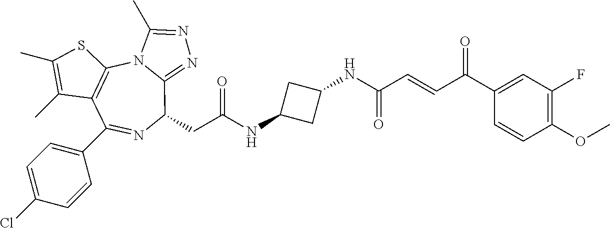 [SMILES: COc1ccc(C(=O)/C=C/C(=O)N[C@H]2C[C@H](NC(=O)C[C@@H]3N=C(c4ccc(Cl)cc4)c4c(sc(C)c4C)-n4c(C)nnc43)C2)cc1F] |
| 237 | 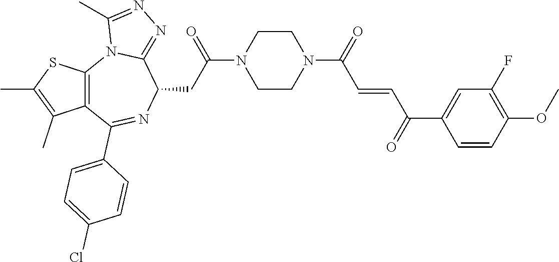 |
| 238 | 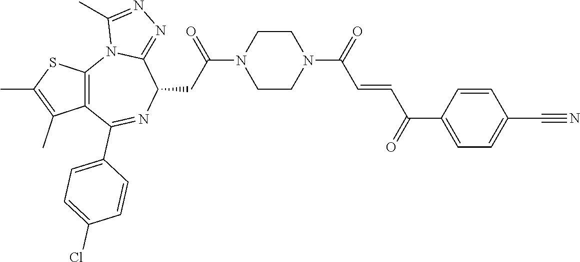 [SMILES: Cc1sc2c(c1C)C(c1ccc(Cl)cc1)=N[C@@H](CC(=O)N1CCN(C(=O)/C=C/C(=O)c3ccc(C#N)cc3)CC1)c1nnc(C)n1-2] |
| 239 | 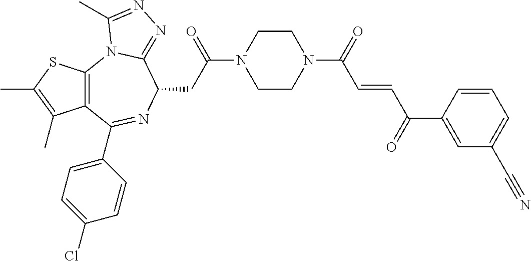 [SMILES: Cc1sc2c(c1C)C(c1ccc(Cl)cc1)=N[C@@H](CC(=O)N1CCN(C(=O)/C=C/C(=O)c3cccc(C#N)c3)CC1)c1nnc(C)n1-2] |
| 240 | 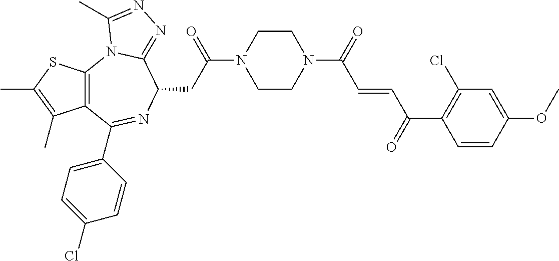 |
| 241 | 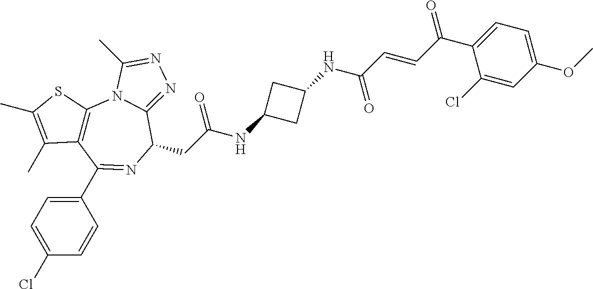 |
| 242 | 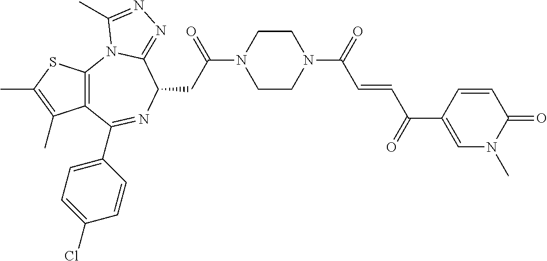 [SMILES: Cc1sc2c(c1C)C(c1ccc(Cl)cc1)=N[C@@H](CC(=O)N1CCN(C(=O)/C=C/C(=O)c3ccc(=O)n(C)c3)CC1)c1nnc(C)n1-2] |
| 243 | 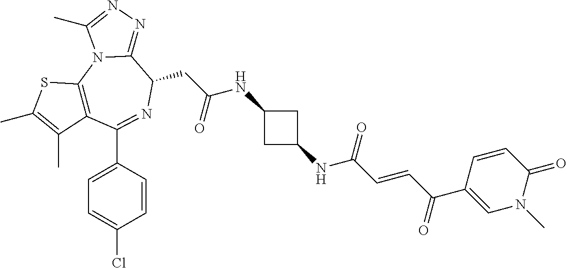 [SMILES: Cc1sc2c(c1C)C(c1ccc(Cl)cc1)=N[C@@H](CC(=O)N[C@H]1C[C@@H](NC(=O)/C=C/C(=O)c3ccc(=O)n(C)c3)C1)c1nnc(C)n1-2] |
| 244 | 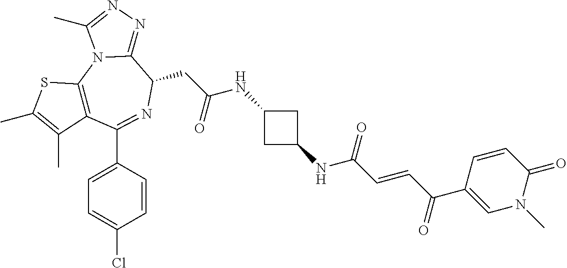 [SMILES: Cc1sc2c(c1C)C(c1ccc(Cl)cc1)=N[C@@H](CC(=O)N[C@H]1C[C@H](NC(=O)/C=C/C(=O)c3ccc(=O)n(C)c3)C1)c1nnc(C)n1-2] |
| 245 | 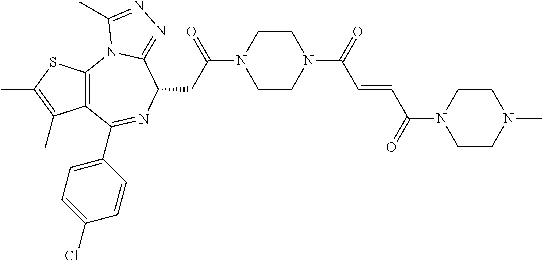 |
| 246 | 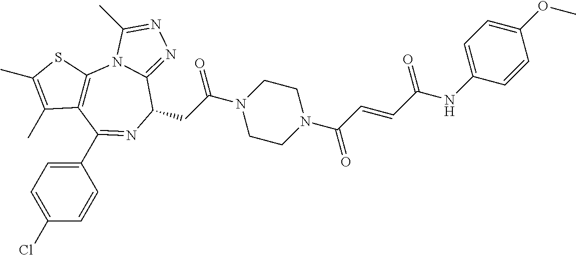 |
| 247 | 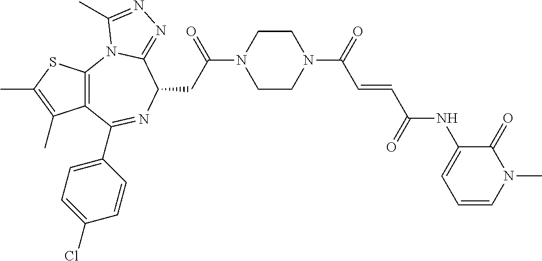 |
| 248 | 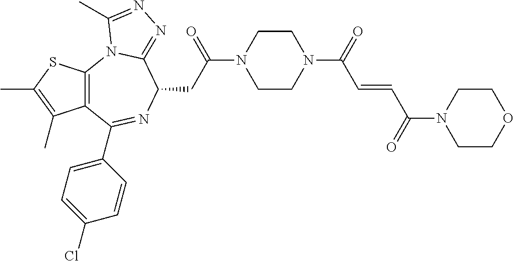 [SMILES: Cc1sc2c(c1C)C(c1ccc(Cl)cc1)=N[C@@H](CC(=O)N1CCN(C(=O)/C=C/C(=O)N3CCOCC3)CC1)c1nnc(C)n1-2] |
| 249 | 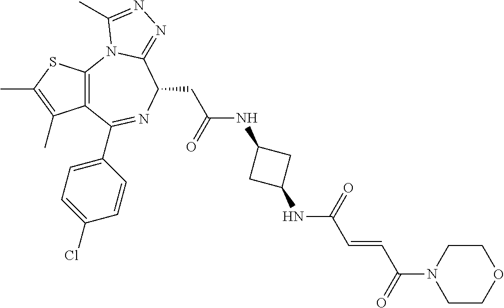 [SMILES: Cc1sc2c(c1C)C(c1ccc(Cl)cc1)=N[C@@H](CC(=O)N[C@H]1C[C@@H](NC(=O)/C=C/C(=O)N3CCOCC3)C1)c1nnc(C)n1-2] |
| 250 | 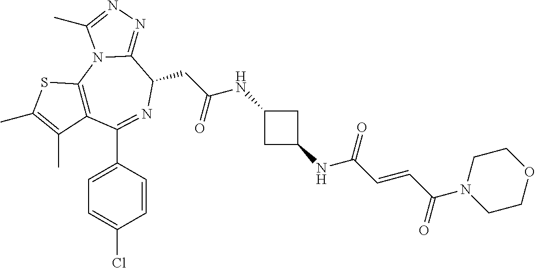 [SMILES: Cc1sc2c(c1C)C(c1ccc(Cl)cc1)=N[C@@H](CC(=O)N[C@H]1C[C@H](NC(=O)/C=C/C(=O)N3CCOCC3)C1)c1nnc(C)n1-2] |
| 251 | 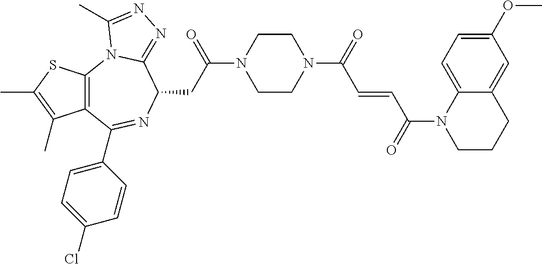 |
| 252 | 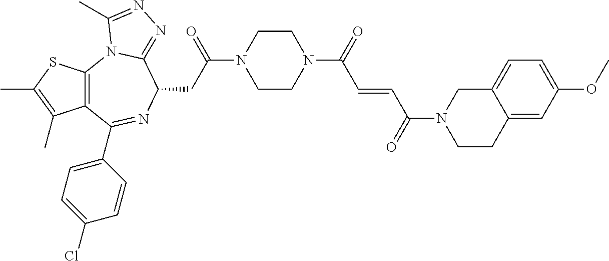 |
| 253 | 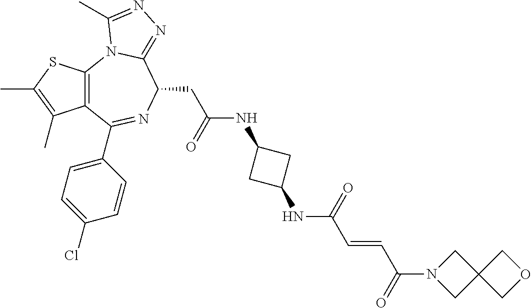 [SMILES: Cc1sc2c(c1C)C(c1ccc(Cl)cc1)=N[C@@H](CC(=O)N[C@H]1C[C@@H](NC(=O)/C=C/C(=O)N3CC4(COC4)C3)C1)c1nnc(C)n1-2] |
| 254 | 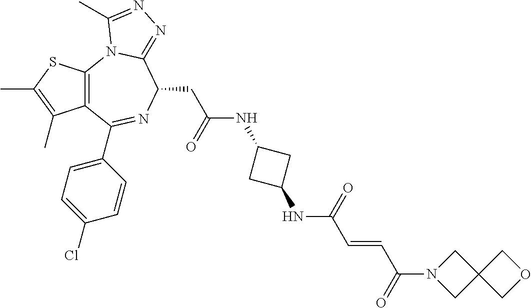 [SMILES: Cc1sc2c(c1C)C(c1ccc(Cl)cc1)=N[C@@H](CC(=O)N[C@H]1C[C@H](NC(=O)/C=C/C(=O)N3CC4(COC4)C3)C1)c1nnc(C)n1-2] |
| 255 | 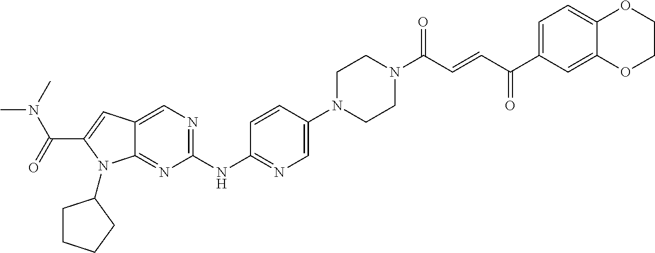 [SMILES: CN(C)C(=O)c1cc2cnc(Nc3ccc(N4CCN(C(=O)/C=C/C(=O)c5ccc6c(c5)OCCO6)CC4)cn3)nc2n1C1CCCC1] |
| 256 | 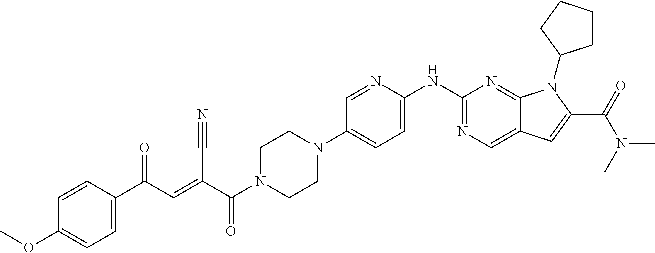 |
| 257 | 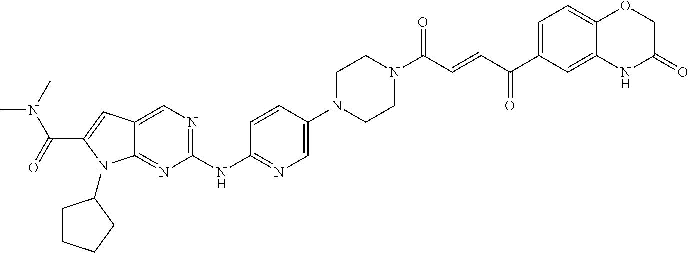 [SMILES: CN(C)C(=O)c1cc2cnc(Nc3ccc(N4CCN(C(=O)/C=C/C(=O)c5ccc6c(c5)NC(=O)CO6)CC4)cn3)nc2n1C1CCCC1] |
| 258 | 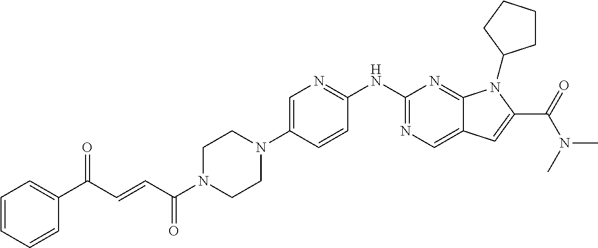 [SMILES: CN(C)C(=O)c1cc2cnc(Nc3ccc(N4CCN(C(=O)/C=C/C(=O)c5ccccc5)CC4)cn3)nc2n1C1CCCC1] |
| 259 | 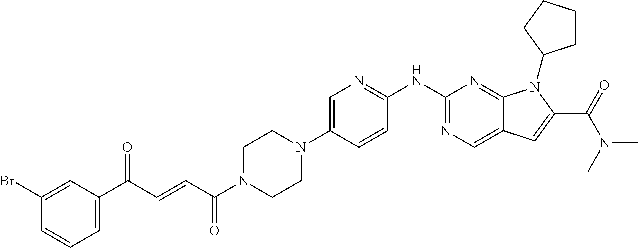 [SMILES: CN(C)C(=O)c1cc2cnc(Nc3ccc(N4CCN(C(=O)/C=C/C(=O)c5cccc(Br)c5)CC4)cn3)nc2n1C1CCCC1] |
| 260 | 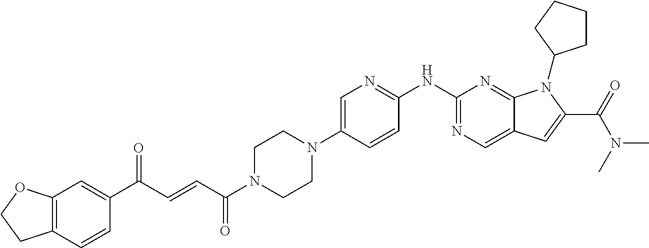 [SMILES: CN(C)C(=O)c1cc2cnc(Nc3ccc(N4CCN(C(=O)/C=C/C(=O)c5ccc6c(c5)OCC6)CC4)cn3)nc2n1C1CCCC1] |
| 261 | 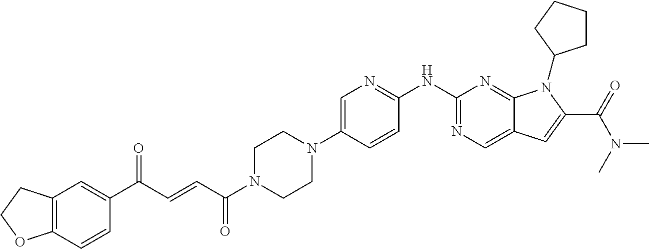 [SMILES: CN(C)C(=O)c1cc2cnc(Nc3ccc(N4CCN(C(=O)/C=C/C(=O)c5ccc6c(c5)CCO6)CC4)cn3)nc2n1C1CCCC1] |
| 262 | 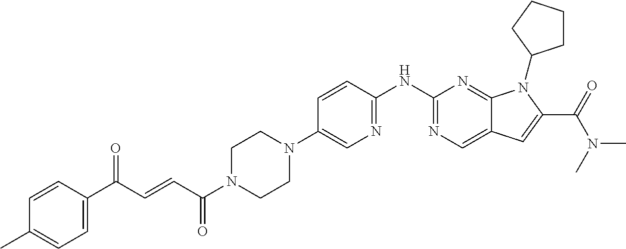 |
| 263 | 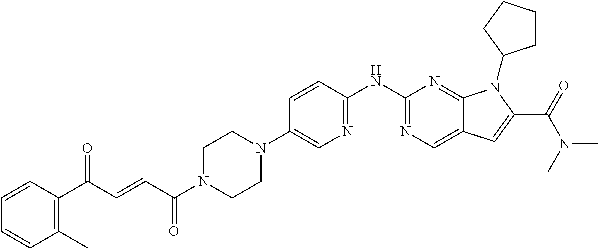 [SMILES: Cc1ccccc1C(=O)/C=C/C(=O)N1CCN(c2ccc(Nc3ncc4cc(C(=O)N(C)C)n(C5CCCC5)c4n3)nc2)CC1] |
| 264 | 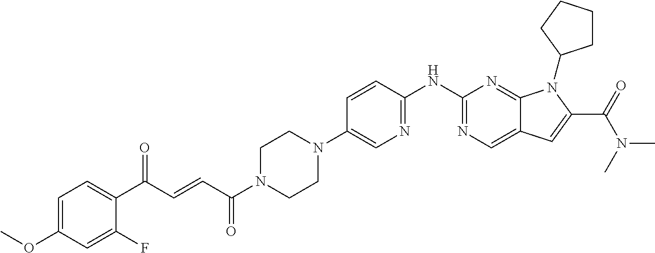 |
| 265 | 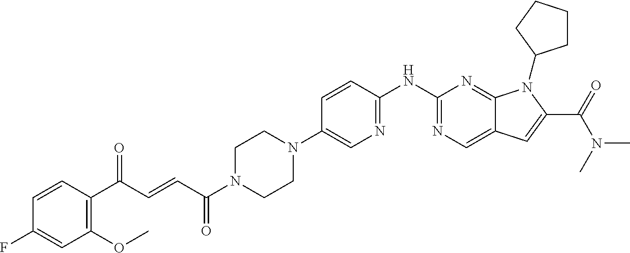 [SMILES: COc1cc(F)ccc1C(=O)/C=C/C(=O)N1CCN(c2ccc(Nc3ncc4cc(C(=O)N(C)C)n(C5CCCC5)c4n3)nc2)CC1] |
| 266 | 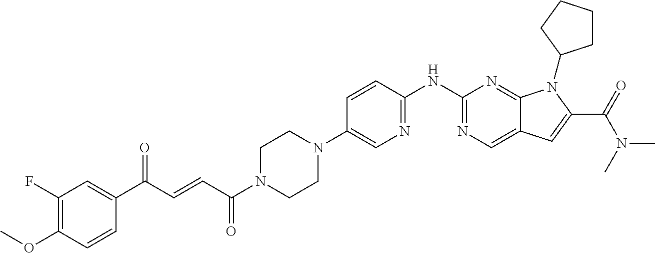 |
| 267 | 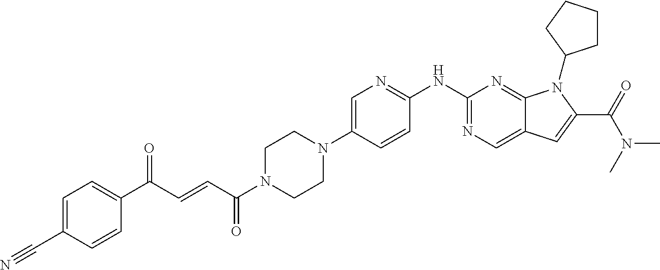 [SMILES: CN(C)C(=O)c1cc2cnc(Nc3ccc(N4CCN(C(=O)/C=C/C(=O)c5ccc(C#N)cc5)CC4)cn3)nc2n1C1CCCC1] |
| 268 | 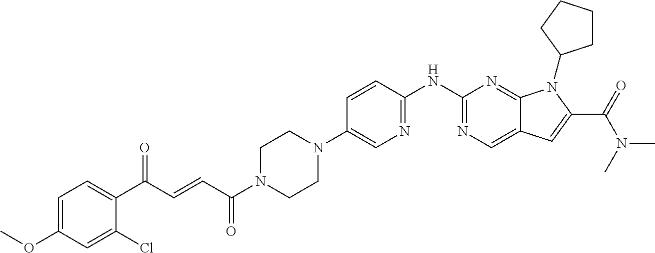 |
| 269 | 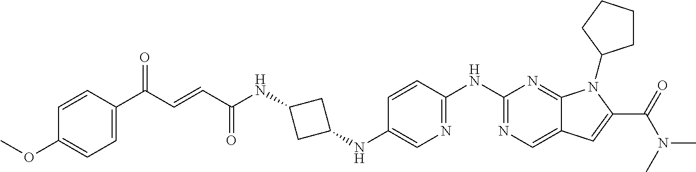 |
| 270 | 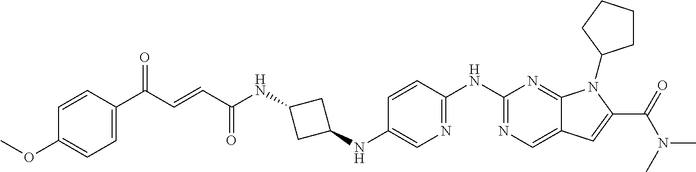 |
| 271 | 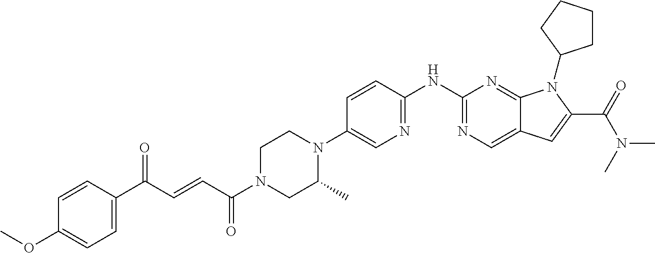 |
| 272 | 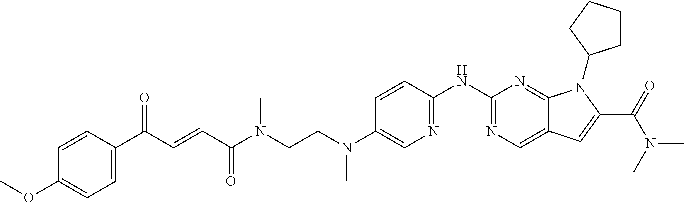 |
| 273 | 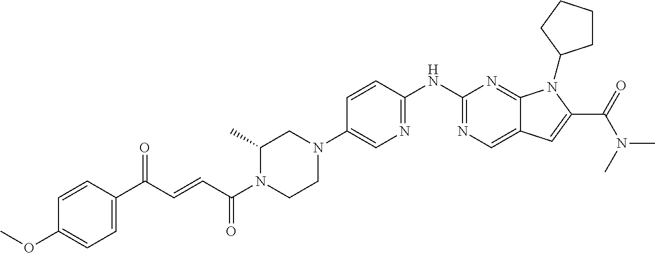 |
| 274 | 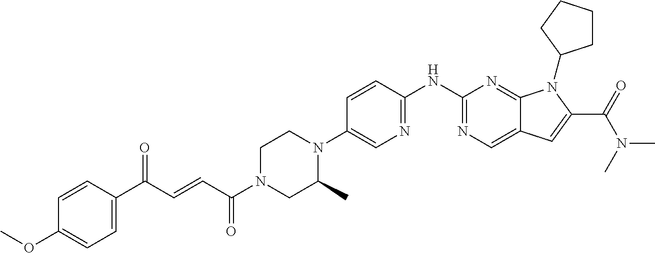 |
| 275 | 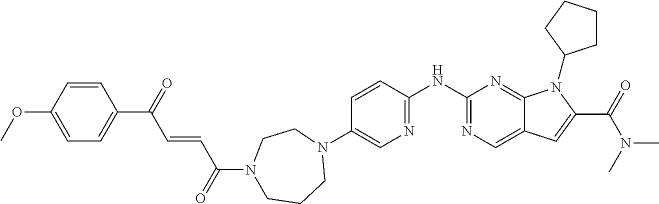 |
| 276 | 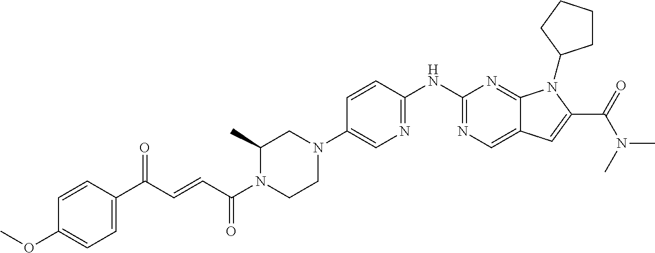 |
| 277 | 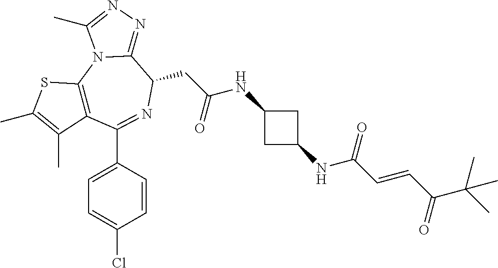 [SMILES: Cc1sc2c(c1C)C(c1ccc(Cl)cc1)=N[C@@H](CC(=O)N[C@H]1C[C@@H](NC(=O)/C=C/C(=O)C(C)(C)C)C1)c1nnc(C)n1-2] |
| 278 | 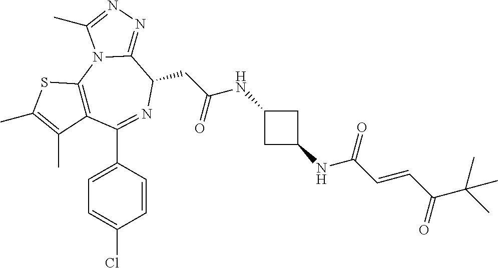 [SMILES: Cc1sc2c(c1C)C(c1ccc(Cl)cc1)=N[C@@H](CC(=O)N[C@H]1C[C@H](NC(=O)/C=C/C(=O)C(C)(C)C)C1)c1nnc(C)n1-2] |
| 279 | 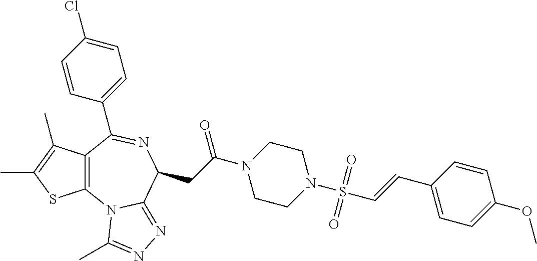 |
| 280 | 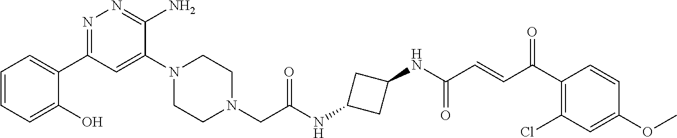 [SMILES: COc1ccc(C(=O)/C=C/C(=O)N[C@H]2C[C@H](NC(=O)CN3CCN(c4cc(-c5ccccc5O)nnc4N)CC3)C2)c(Cl)c1] |
| 281 | 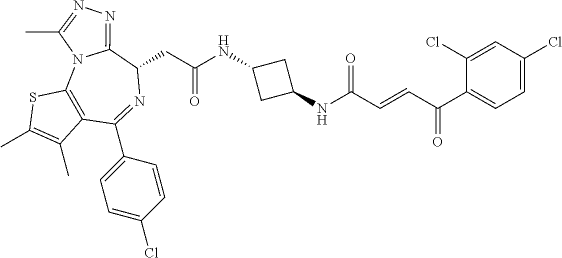 [SMILES: Cc1sc2c(c1C)C(c1ccc(Cl)cc1)=N[C@@H](CC(=O)N[C@H]1C[C@H](NC(=O)/C=C/C(=O)c3ccc(Cl)cc3Cl)C1)c1nnc(C)n1-2] |
| 282 | 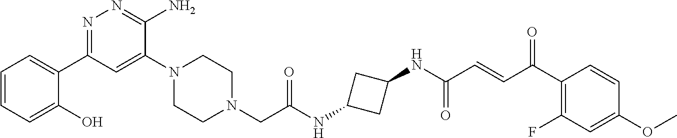 [SMILES: COc1ccc(C(=O)/C=C/C(=O)N[C@H]2C[C@H](NC(=O)CN3CCN(c4cc(-c5ccccc5O)nnc4N)CC3)C2)c(F)c1] |
| 283 | 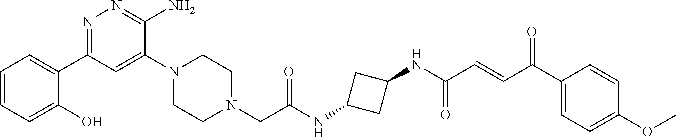 [SMILES: COc1ccc(C(=O)/C=C/C(=O)N[C@H]2C[C@H](NC(=O)CN3CCN(c4cc(-c5ccccc5O)nnc4N)CC3)C2)cc1] |
| 284 | 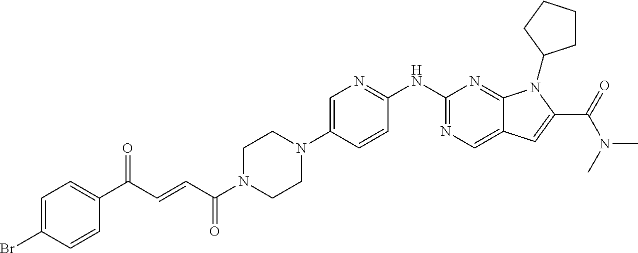 [SMILES: CN(C)C(=O)c1cc2cnc(Nc3ccc(N4CCN(C(=O)/C=C/C(=O)c5ccc(Br)cc5)CC4)cn3)nc2n1C1CCCC1] |
| 285 | 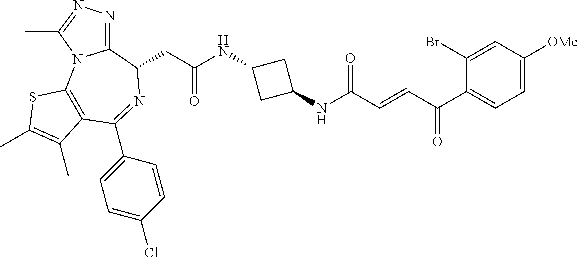 [SMILES: COc1ccc(C(=O)/C=C/C(=O)N[C@H]2C[C@H](NC(=O)C[C@@H]3N=C(c4ccc(Cl)cc4)c4c(sc(C)c4C)-n4c(C)nnc43)C2)c(Br)c1] |
| 286 | 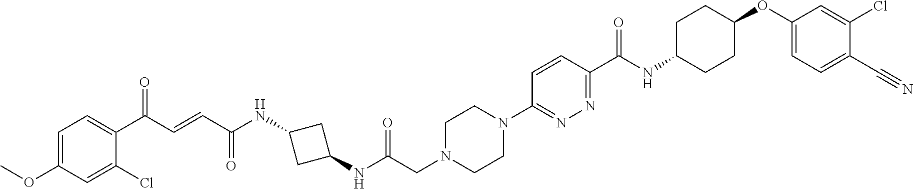 [SMILES: COc1ccc(C(=O)/C=C/C(=O)N[C@H]2C[C@H](NC(=O)CN3CCN(c4ccc(C(=O)N[C@H]5CC[C@H](Oc6ccc(C#N)c(Cl)c6)CC5)nn4)CC3)C2)c(Cl)c1] |
| 287 | 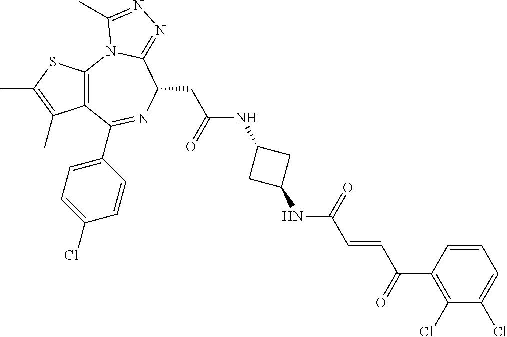 [SMILES: Cc1sc2c(c1C)C(c1ccc(Cl)cc1)=N[C@@H](CC(=O)N[C@H]1C[C@H](NC(=O)/C=C/C(=O)c3cccc(Cl)c3Cl)C1)c1nnc(C)n1-2] |
| 288 | 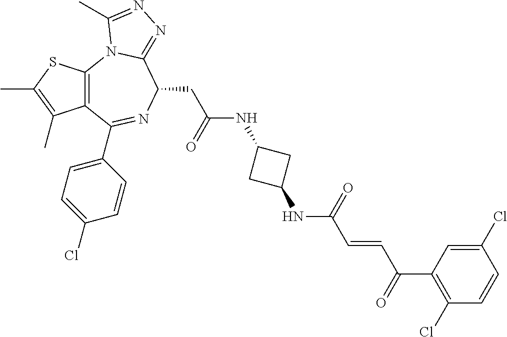 [SMILES: Cc1sc2c(c1C)C(c1ccc(Cl)cc1)=N[C@@H](CC(=O)N[C@H]1C[C@H](NC(=O)/C=C/C(=O)c3cc(Cl)ccc3Cl)C1)c1nnc(C)n1-2] |

TABLE 2
Exemplary Intermediates

| Compound | |
|---|---|
| No. | Structure |
| 116 | 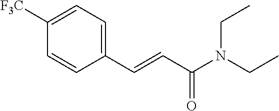 [SMILES: CCN(CC)C(=O)/C=C/c1ccc(C(F)(F)F)cc1] |
| 117 | 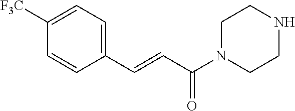 [SMILES: O=C(/C=C/c1ccc(C(F)(F)F)cc1)N1CCNCC1] |
| 118 | 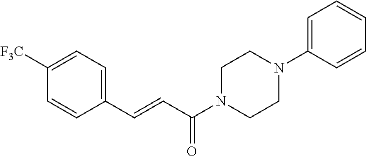 [SMILES: O=C(/C=C/c1ccc(C(F)(F)F)cc1)N1CCN(c2ccccc2)CC1] |
| 119 | 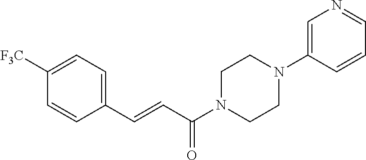 [SMILES: O=C(/C=C/c1ccc(C(F)(F)F)cc1)N1CCN(c2cccnc2)CC1] |
| 121 | 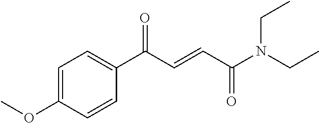 [SMILES: CCN(CC)C(=O)/C=C/C(=O)c1ccc(OC)cc1] |
| 122 | 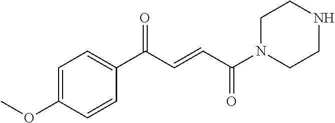 [SMILES: COc1ccc(C(=O)/C=C/C(=O)N2CCNCC2)cc1] |
| 123 | 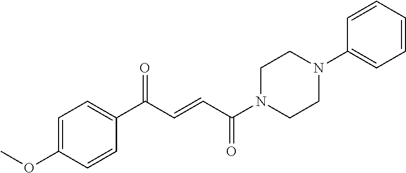 [SMILES: COc1ccc(C(=O)/C=C/C(=O)N2CCN(c3ccccc3)CC2)cc1] |
| 124 | 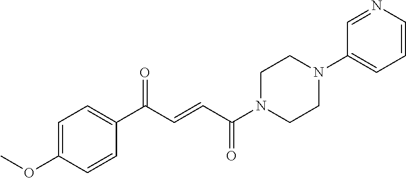 [SMILES: COc1ccc(C(=O)/C=C/C(=O)N2CCN(c3cccnc3)CC2)cc1] |
| 145 | 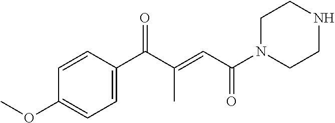 [SMILES: COc1ccc(C(=O)/C(C)=C/C(=O)N2CCNCC2)cc1] |
| 147 | 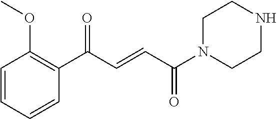 [SMILES: COc1ccccc1C(=O)/C=C/C(=O)N1CCNCC1] |
| 149 | 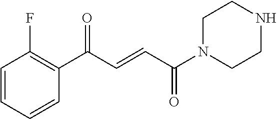 [SMILES: O=C(/C=C/C(=O)N1CCNCC1)c1ccccc1F] |
| 150 | 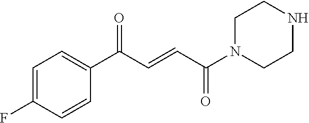 [SMILES: O=C(/C=C/C(=O)N1CCNCC1)c1ccc(F)cc1] |
| 152 | 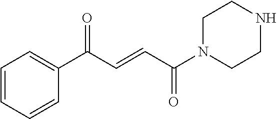 [SMILES: O=C(/C=C/C(=O)N1CCNCC1)c1ccccc1] |
| 153 | 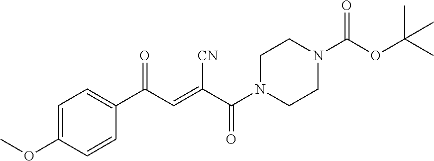 [SMILES: COc1ccc(C(=O)/C=C(\C#N)C(=O)N2CCN(C(=O)OC(C)(C)C)CC2)cc1] |
| 154 | 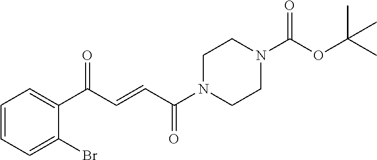 [SMILES: CC(C)(C)OC(=O)N1CCN(C(=O)/C=C/C(=O)c2ccccc2Br)CC1] |
| 155 | 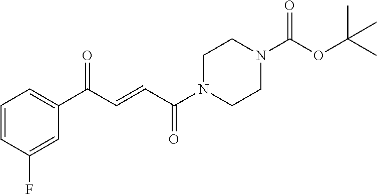 [SMILES: CC(C)(C)OC(=O)N1CCN(C(=O)/C=C/C(=O)c2cccc(F)c2)CC1] |
| 156 | 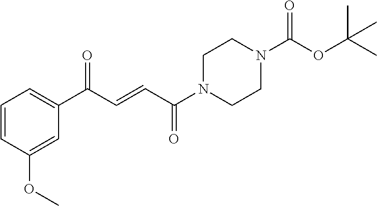 [SMILES: COc1cccc(C(=O)/C=C/C(=O)N2CCN(C(=O)OC(C)(C)C)CC2)c1] |
| 157 | 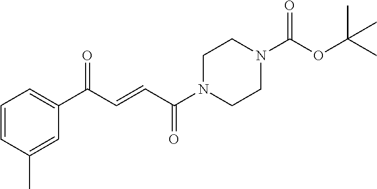 [SMILES: Cc1cccc(C(=O)/C=C/C(=O)N2CCN(C(=O)OC(C)(C)C)CC2)c1] |
| 161 | 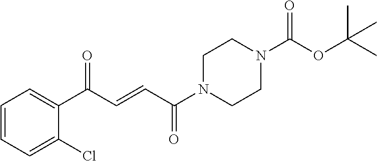 [SMILES: CC(C)(C)OC(=O)N1CCN(C(=O)/C=C/C(=O)c2ccccc2Cl)CC1] |
| 162 | 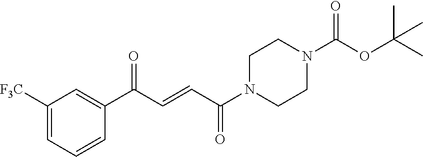 [SMILES: CC(C)(C)OC(=O)N1CCN(C(=O)/C=C/C(=O)c2cccc(C(F)(F)F)c2)CC1] |
| 163 | 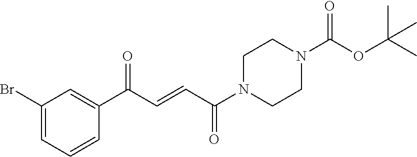 [SMILES: CC(C)(C)OC(=O)N1CCN(C(=O)/C=C/C(=O)c2cccc(Br)c2)CC1] |
| 164 | 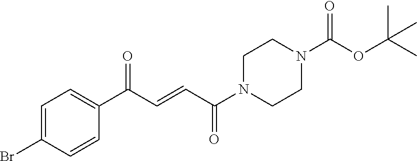 [SMILES: CC(C)(C)OC(=O)N1CCN(C(=O)/C=C/C(=O)c2ccc(Br)cc2)CC1] |
| 166 | 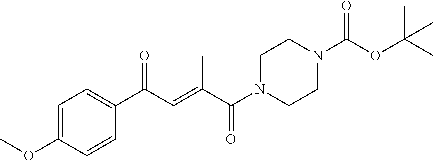 [SMILES: COc1ccc(C(=O)/C=C(\C)C(=O)N2CCN(C(=O)OC(C)(C)C)CC2)cc1] |
| 300 | 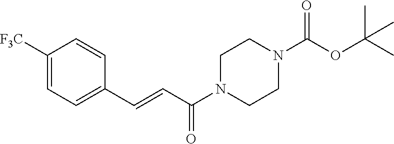 [SMILES: CC(C)(C)OC(=O)N1CCN(C(=O)/C=C/c2ccc(C(F)(F)F)cc2)CC1] |
| 301 | 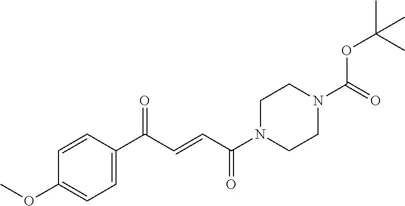 [SMILES: COc1ccc(C(=O)/C=C/C(=O)N2CCN(C(=O)OC(C)(C)C)CC2)cc1] |
| 302 | 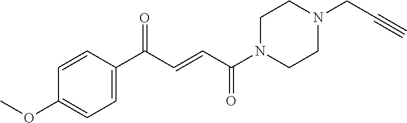 [SMILES: C#CCN1CCN(C(=O)/C=C/C(=O)c2ccc(OC)cc2)CC1] |
| 303 | 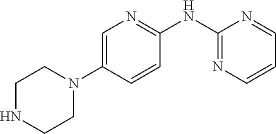 [SMILES: c1cnc(Nc2ccc(N3CCNCC3)cn2)nc1] |
| 304 | 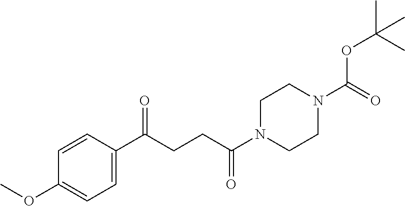 [SMILES: COc1ccc(C(=O)CCC(=O)N2CCN(C(=O)OC(C)(C)C)CC2)cc1] |
| 305 | 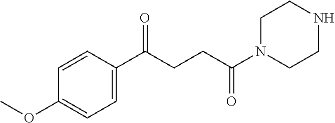 [SMILES: COc1ccc(C(=O)CCC(=O)N2CCNCC2)cc1] |
| 306 | 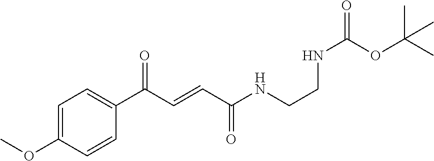 [SMILES: COc1ccc(C(=O)/C=C/C(=O)NCCNC(=O)OC(C)(C)C)cc1] |
| 307 | 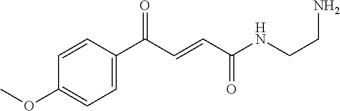 [SMILES: COc1ccc(C(=O)/C=C/C(=O)NCCN)cc1] |
| 308 | 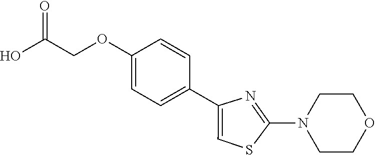 [SMILES: O=C(O)COc1ccc(-c2csc(N3CCOCC3)n2)cc1] |
| 309 | 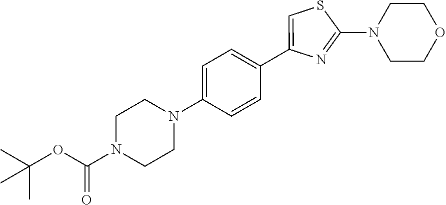 [SMILES: CC(C)(C)OC(=O)N1CCN(c2ccc(-c3csc(N4CCOCC4)n3)cc2)CC1] |
| 310 | 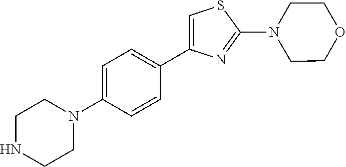 [SMILES: c1cc(N2CCNCC2)ccc1-c1csc(N2CCOCC2)n1] |
| 311 | 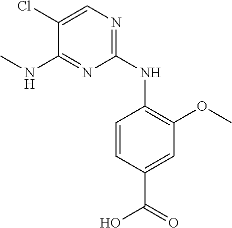 |
| 312 | 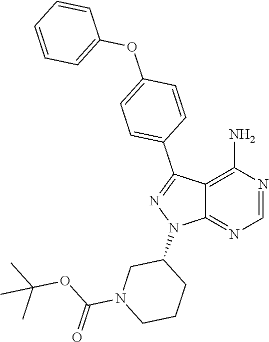 [SMILES: CC(C)(C)OC(=O)N1CCC[C@@H](n2nc(-c3ccc(Oc4ccccc4)cc3)c3c(N)ncnc32)C1] |
| 313 | 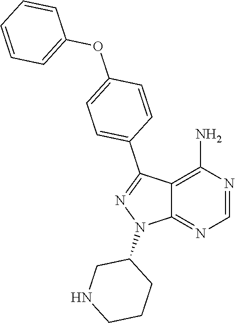 [SMILES: Nc1ncnc2c1c(-c1ccc(Oc3ccccc3)cc1)nn2[C@@H]1CCCNC1] |
| 314 | 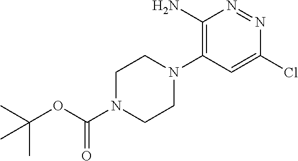 [SMILES: CC(C)(C)OC(=O)N1CCN(c2cc(Cl)nnc2N)CC1] |
| 315 | 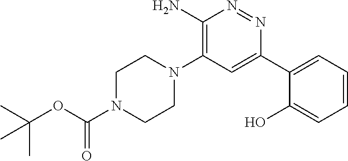 [SMILES: CC(C)(C)OC(=O)N1CCN(c2cc(-c3ccccc3O)nnc2N)CC1] |
| 316 | 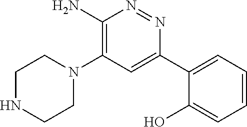 [SMILES: Nc1nnc(-c2ccccc2O)cc1N1CCNCC1] |
| 317 | 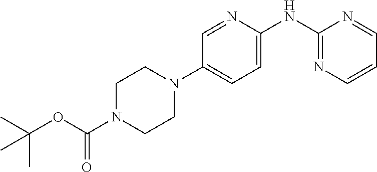 [SMILES: CC(C)(C)OC(=O)N1CCN(c2ccc(Nc3ncccn3)nc2)CC1] |
| 400 | 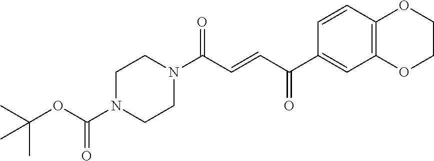 [SMILES: CC(C)(C)OC(=O)N1CCN(C(=O)/C=C/C(=O)c2ccc3c(c2)OCCO3)CC1] |
| 401 | 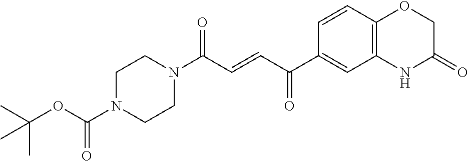 [SMILES: CC(C)(C)OC(=O)N1CCN(C(=O)/C=C/C(=O)c2ccc3c(c2)NC(=O)CO3)CC1] |
| 402 | 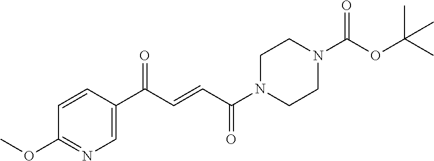 [SMILES: COc1ccc(C(=O)/C=C/C(=O)N2CCN(C(=O)OC(C)(C)C)CC2)cn1] |
| 403 | 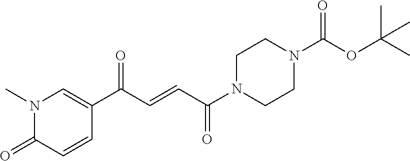 [SMILES: Cn1cc(C(=O)/C=C/C(=O)N2CCN(C(=O)OC(C)(C)C)CC2)ccc1=O] |
| 404 | 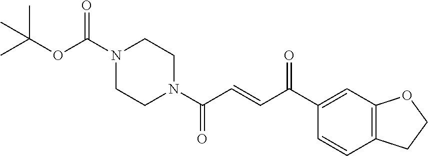 [SMILES: CC(C)(C)OC(=O)N1CCN(C(=O)/C=C/C(=O)c2ccc3c(c2)OCC3)CC1] |
| 405 | 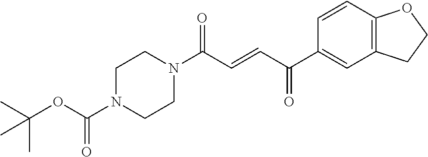 [SMILES: CC(C)(C)OC(=O)N1CCN(C(=O)/C=C/C(=O)c2ccc3c(c2)CCO3)CC1] |
| 406 | 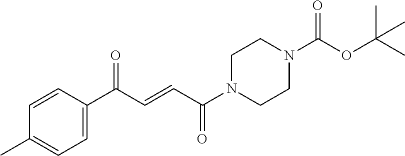 [SMILES: Cc1ccc(C(=O)/C=C/C(=O)N2CCN(C(=O)OC(C)(C)C)CC2)cc1] |
| 407 | 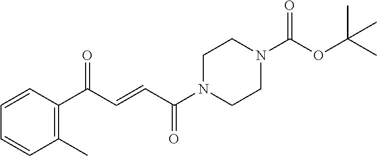 [SMILES: Cc1ccccc1C(=O)/C=C/C(=O)N1CCN(C(=O)OC(C)(C)C)CC1] |
| 408 | 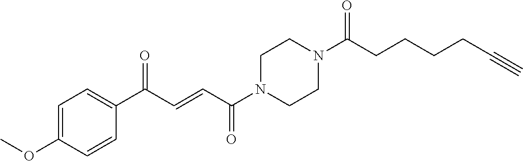 [SMILES: C#CCCCCC(=O)N1CCN(C(=O)/C=C/C(=O)c2ccc(OC)cc2)CC1] |
| 409 | 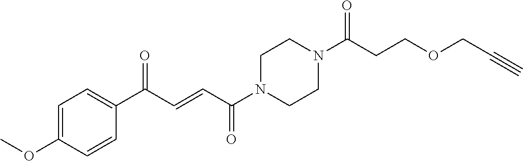 [SMILES: C#CCOCCC(=O)N1CCN(C(=O)/C=C/C(=O)c2ccc(OC)cc2)CC1] |
| 410 | 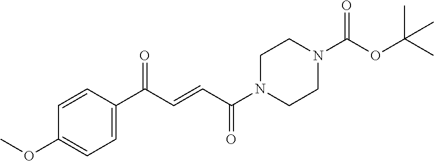 [SMILES: COc1ccc(C(=O)/C=C/C(=O)N2CCN(C(=O)OC(C)(C)C)CC2)cc1] |
| 411 | 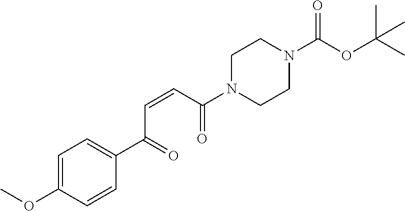 [SMILES: COc1ccc(C(=O)/C=C\C(=O)N2CCN(C(=O)OC(C)(C)C)CC2)cc1] |
| 412 | 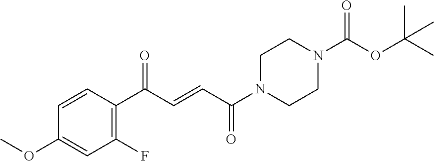 [SMILES: COc1ccc(C(=O)/C=C/C(=O)N2CCN(C(=O)OC(C)(C)C)CC2)c(F)c1] |
| 413 | 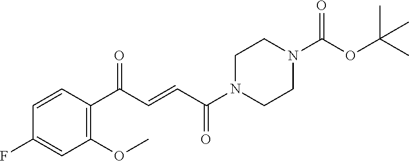 [SMILES: COc1cc(F)ccc1C(=O)/C=C/C(=O)N1CCN(C(=O)OC(C)(C)C)CC1] |
| 414 | 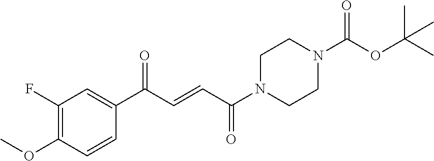 [SMILES: COc1ccc(C(=O)/C=C/C(=O)N2CCN(C(=O)OC(C)(C)C)CC2)cc1F] |
| 415 | 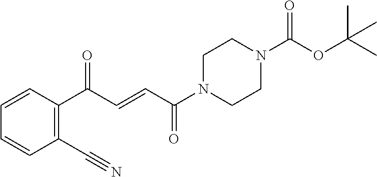 [SMILES: CC(C)(C)OC(=O)N1CCN(C(=O)/C=C/C(=O)c2ccccc2C#N)CC1] |
| 416 | 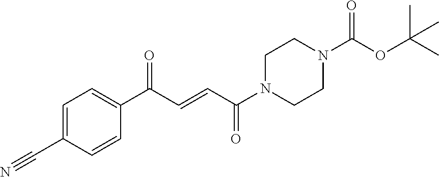 [SMILES: CC(C)(C)OC(=O)N1CCN(C(=O)/C=C/C(=O)c2ccc(C#N)cc2)CC1] |
| 417 | 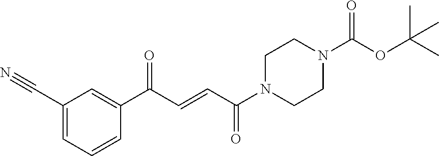 [SMILES: CC(C)(C)OC(=O)N1CCN(C(=O)/C=C/C(=O)c2cccc(C#N)c2)CC1] |
| 418 | 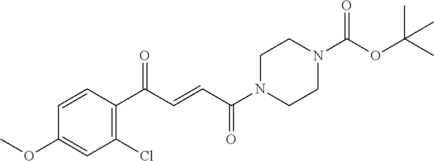 [SMILES: COc1ccc(C(=O)/C=C/C(=O)N2CCN(C(=O)OC(C)(C)C)CC2)c(Cl)c1] |
| 419 | 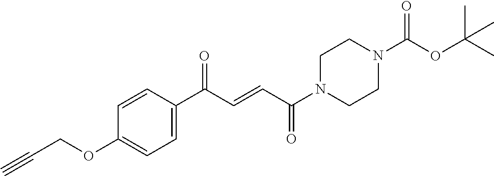 [SMILES: C#CCOc1ccc(C(=O)/C=C/C(=O)N2CCN(C(=O)OC(C)(C)C)CC2)cc1] |

In some embodiments, the molecular glue degrader compound is Compound 100 or a pharmaceutically acceptable salt, hydrate, solvate, prodrug, stereoisomer, or tautomer thereof. In some embodiments, the molecular glue degrader compound is Compound 101 or a pharmaceutically acceptable salt, hydrate, solvate, prodrug, stereoisomer, or tautomer thereof. In some embodiments, the molecular glue degrader compound is Compound 102 or a pharmaceutically acceptable salt, hydrate, solvate, prodrug, stereoisomer, or tautomer thereof. In some embodiments, the molecular glue degrader compound is Compound 103 or a pharmaceutically acceptable salt, hydrate, solvate, prodrug, stereoisomer, or tautomer thereof. In some embodiments, the molecular glue degrader compound is Compound 104 or a pharmaceutically acceptable salt, hydrate, solvate, prodrug, stereoisomer, or tautomer thereof. In some embodiments, the molecular glue degrader compound is Compound 105 or a pharmaceutically acceptable salt, hydrate, solvate, prodrug, stereoisomer, or tautomer thereof. In some embodiments, the molecular glue degrader compound is Compound 106 or a pharmaceutically acceptable salt, hydrate, solvate, prodrug, stereoisomer, or tautomer thereof. In some embodiments, the molecular glue degrader compound is Compound 107 or a pharmaceutically acceptable salt, hydrate, solvate, prodrug, stereoisomer, or tautomer thereof. In some embodiments, the molecular glue degrader compound is Compound 108 or a pharmaceutically acceptable salt, hydrate, solvate, prodrug, stereoisomer, or tautomer thereof. In some embodiments, the molecular glue degrader compound is Compound 109 or a pharmaceutically acceptable salt, hydrate, solvate, prodrug, stereoisomer, or tautomer thereof. In some embodiments, the molecular glue degrader compound is Compound 110 or a pharmaceutically acceptable salt, hydrate, solvate, prodrug, stereoisomer, or tautomer thereof. In some embodiments, the molecular glue degrader compound is Compound 111 or a pharmaceutically acceptable salt, hydrate, solvate, prodrug, stereoisomer, or tautomer thereof. In some embodiments, the molecular glue degrader compound is Compound 112 or a pharmaceutically acceptable salt, hydrate, solvate, prodrug, stereoisomer, or tautomer thereof. In some embodiments, the molecular glue degrader compound is Compound 113 or a pharmaceutically acceptable salt, hydrate, solvate, prodrug, stereoisomer, or tautomer thereof. In some embodiments, the molecular glue degrader compound is Compound 114 or a pharmaceutically acceptable salt, hydrate, solvate, prodrug, stereoisomer, or tautomer thereof. In some embodiments, the molecular glue degrader compound is Compound 115 or a pharmaceutically acceptable salt, hydrate, solvate, prodrug, stereoisomer, or tautomer thereof. In some embodiments, the molecular glue degrader compound is Compound 116 or a pharmaceutically acceptable salt, hydrate, solvate, prodrug, stereoisomer, or tautomer thereof. In some embodiments, the molecular glue degrader compound is Compound 117 or a pharmaceutically acceptable salt, hydrate, solvate, prodrug, stereoisomer, or tautomer thereof. In some embodiments, the molecular glue degrader compound is Compound 118 or a pharmaceutically acceptable salt, hydrate, solvate, prodrug, stereoisomer, or tautomer thereof. In some embodiments, the molecular glue degrader compound is Compound 119 or a pharmaceutically acceptable salt, hydrate, solvate, prodrug, stereoisomer, or tautomer thereof. In some embodiments, the molecular glue degrader compound is Compound 120 or a pharmaceutically acceptable salt, hydrate, solvate, prodrug, stereoisomer, or tautomer thereof. In some embodiments, the molecular glue degrader compound is Compound 121 or a pharmaceutically acceptable salt, hydrate, solvate, prodrug, stereoisomer, or tautomer thereof. In some embodiments, the molecular glue degrader compound is Compound 122 or a pharmaceutically acceptable salt, hydrate, solvate, prodrug, stereoisomer, or tautomer thereof. In some embodiments, the molecular glue degrader compound is Compound 123 or a pharmaceutically acceptable salt, hydrate, solvate, prodrug, stereoisomer, or tautomer thereof. In some embodiments, the molecular glue degrader compound is Compound 124 or a pharmaceutically acceptable salt, hydrate, solvate, prodrug, stereoisomer, or tautomer thereof. In some embodiments, the molecular glue degrader compound is Compound 125 or a pharmaceutically acceptable salt, hydrate, solvate, prodrug, stereoisomer, or tautomer thereof. In some embodiments, the molecular glue degrader compound is Compound 126 or a pharmaceutically acceptable salt, hydrate, solvate, prodrug, stereoisomer, or tautomer thereof. In some embodiments, the molecular glue degrader compound is Compound 127 or a pharmaceutically acceptable salt, hydrate, solvate, prodrug, stereoisomer, or tautomer thereof. In some embodiments, the molecular glue degrader compound is Compound 128 or a pharmaceutically acceptable salt, hydrate, solvate, prodrug, stereoisomer, or tautomer thereof. In some embodiments, the molecular glue degrader compound is Compound 129 or a pharmaceutically acceptable salt, hydrate, solvate, prodrug, stereoisomer, or tautomer thereof. In some embodiments, the molecular glue degrader compound is Compound 130 or a pharmaceutically acceptable salt, hydrate, solvate, prodrug, stereoisomer, or tautomer thereof. In some embodiments, the molecular glue degrader compound is Compound 131 or a pharmaceutically acceptable salt, hydrate, solvate, prodrug, stereoisomer, or tautomer thereof. In some embodiments, the molecular glue degrader compound is Compound 132 or a pharmaceutically acceptable salt, hydrate, solvate, prodrug, stereoisomer, or tautomer thereof. In some embodiments, the molecular glue degrader compound is Compound 133 or a pharmaceutically acceptable salt, hydrate, solvate, prodrug, stereoisomer, or tautomer thereof. In some embodiments, the molecular glue degrader compound is Compound 134 or a pharmaceutically acceptable salt, hydrate, solvate, prodrug, stereoisomer, or tautomer thereof. In some embodiments, the molecular glue degrader compound is Compound 135 or a pharmaceutically acceptable salt, hydrate, solvate, prodrug, stereoisomer, or tautomer thereof. In some embodiments, the molecular glue degrader compound is Compound 136 or a pharmaceutically acceptable salt, hydrate, solvate, prodrug, stereoisomer, or tautomer thereof. In some embodiments, the molecular glue degrader compound is Compound 137 or a pharmaceutically acceptable salt, hydrate, solvate, prodrug, stereoisomer, or tautomer thereof. In some embodiments, the molecular glue degrader compound is Compound 138 or a pharmaceutically acceptable salt, hydrate, solvate, prodrug, stereoisomer, or tautomer thereof. In some embodiments, the molecular glue degrader compound is Compound 139 or a pharmaceutically acceptable salt, hydrate, solvate, prodrug, stereoisomer, or tautomer thereof. In some embodiments, the molecular glue degrader compound is Compound 140 or a pharmaceutically acceptable salt, hydrate, solvate, prodrug, stereoisomer, or tautomer thereof. In some embodiments, the molecular glue degrader compound is Compound 141 or a pharmaceutically acceptable salt, hydrate, solvate, prodrug, stereoisomer, or tautomer thereof. In some embodiments, the molecular glue degrader compound is Compound 142 or a pharmaceutically acceptable salt, hydrate, solvate, prodrug, stereoisomer, or tautomer thereof. In some embodiments, the molecular glue degrader compound is Compound 143 or a pharmaceutically acceptable salt, hydrate, solvate, prodrug, stereoisomer, or tautomer thereof. In some embodiments, the molecular glue degrader compound is Compound 144 or a pharmaceutically acceptable salt, hydrate, solvate, prodrug, stereoisomer, or tautomer thereof. In some embodiments, the molecular glue degrader compound is Compound 145 or a pharmaceutically acceptable salt, hydrate, solvate, prodrug, stereoisomer, or tautomer thereof. In some embodiments, the molecular glue degrader compound is Compound 146 or a pharmaceutically acceptable salt, hydrate, solvate, prodrug, stereoisomer, or tautomer thereof. In some embodiments, the molecular glue degrader compound is Compound 147 or a pharmaceutically acceptable salt, hydrate, solvate, prodrug, stereoisomer, or tautomer thereof. In some embodiments, the molecular glue degrader compound is Compound 148 or a pharmaceutically acceptable salt, hydrate, solvate, prodrug, stereoisomer, or tautomer thereof. In some embodiments, the molecular glue degrader compound is Compound 149 or a pharmaceutically acceptable salt, hydrate, solvate, prodrug, stereoisomer, or tautomer thereof. In some embodiments, the molecular glue degrader compound is Compound 150 or a pharmaceutically acceptable salt, hydrate, solvate, prodrug, stereoisomer, or tautomer thereof. In some embodiments, the molecular glue degrader compound is Compound 151 or a pharmaceutically acceptable salt, hydrate, solvate, prodrug, stereoisomer, or tautomer thereof. In some embodiments, the molecular glue degrader compound is Compound 152 or a pharmaceutically acceptable salt, hydrate, solvate, prodrug, stereoisomer, or tautomer thereof. In some embodiments, the molecular glue degrader compound is Compound 153 or a pharmaceutically acceptable salt, hydrate, solvate, prodrug, stereoisomer, or tautomer thereof. In some embodiments, the molecular glue degrader compound is Compound 154 or a pharmaceutically acceptable salt, hydrate, solvate, prodrug, stereoisomer, or tautomer thereof. In some embodiments, the molecular glue degrader compound is Compound 155 or a pharmaceutically acceptable salt, hydrate, solvate, prodrug, stereoisomer, or tautomer thereof. In some embodiments, the molecular glue degrader compound is Compound 156 or a pharmaceutically acceptable salt, hydrate, solvate, prodrug, stereoisomer, or tautomer thereof. In some embodiments, the molecular glue degrader compound is Compound 157 or a pharmaceutically acceptable salt, hydrate, solvate, prodrug, stereoisomer, or tautomer thereof. In some embodiments, the molecular glue degrader compound is Compound 158 or a pharmaceutically acceptable salt, hydrate, solvate, prodrug, stereoisomer, or tautomer thereof. In some embodiments, the molecular glue degrader compound is Compound 159 or a pharmaceutically acceptable salt, hydrate, solvate, prodrug, stereoisomer, or tautomer thereof. In some embodiments, the molecular glue degrader compound is Compound 160 or a pharmaceutically acceptable salt, hydrate, solvate, prodrug, stereoisomer, or tautomer thereof. In some embodiments, the molecular glue degrader compound is Compound 161 or a pharmaceutically acceptable salt, hydrate, solvate, prodrug, stereoisomer, or tautomer thereof. In some embodiments, the molecular glue degrader compound is Compound 162 or a pharmaceutically acceptable salt, hydrate, solvate, prodrug, stereoisomer, or tautomer thereof. In some embodiments, the molecular glue degrader compound is Compound 163 or a pharmaceutically acceptable salt, hydrate, solvate, prodrug, stereoisomer, or tautomer thereof. In some embodiments, the molecular glue degrader compound is Compound 164 or a pharmaceutically acceptable salt, hydrate, solvate, prodrug, stereoisomer, or tautomer thereof. In some embodiments, the molecular glue degrader compound is Compound 165 or a pharmaceutically acceptable salt, hydrate, solvate, prodrug, stereoisomer, or tautomer thereof. In some embodiments, the molecular glue degrader compound is Compound 166 or a pharmaceutically acceptable salt, hydrate, solvate, prodrug, stereoisomer, or tautomer thereof. In some embodiments, the molecular glue degrader compound is Compound 200 or a pharmaceutically acceptable salt, hydrate, solvate, prodrug, stereoisomer, or tautomer thereof. In some embodiments, the molecular glue degrader compound is Compound 201 or a pharmaceutically acceptable salt, hydrate, solvate, prodrug, stereoisomer, or tautomer thereof. In some embodiments, the molecular glue degrader compound is Compound 202 or a pharmaceutically acceptable salt, hydrate, solvate, prodrug, stereoisomer, or tautomer thereof. In some embodiments, the molecular glue degrader compound is Compound 203 or a pharmaceutically acceptable salt, hydrate, solvate, prodrug, stereoisomer, or tautomer thereof. In some embodiments, the molecular glue degrader compound is Compound 204 or a pharmaceutically acceptable salt, hydrate, solvate, prodrug, stereoisomer, or tautomer thereof. In some embodiments, the molecular glue degrader compound is Compound 205 or a pharmaceutically acceptable salt, hydrate, solvate, prodrug, stereoisomer, or tautomer thereof. In some embodiments, the molecular glue degrader compound is Compound 206 or a pharmaceutically acceptable salt, hydrate, solvate, prodrug, stereoisomer, or tautomer thereof. In some embodiments, the molecular glue degrader compound is Compound 207 or a pharmaceutically acceptable salt, hydrate, solvate, prodrug, stereoisomer, or tautomer thereof. In some embodiments, the molecular glue degrader compound is Compound 208 or a pharmaceutically acceptable salt, hydrate, solvate, prodrug, stereoisomer, or tautomer thereof. In some embodiments, the molecular glue degrader compound is Compound 209 or a pharmaceutically acceptable salt, hydrate, solvate, prodrug, stereoisomer, or tautomer thereof. In some embodiments, the molecular glue degrader compound is Compound 210 or a pharmaceutically acceptable salt, hydrate, solvate, prodrug, stereoisomer, or tautomer thereof. In some embodiments, the molecular glue degrader compound is Compound 211 or a pharmaceutically acceptable salt, hydrate, solvate, prodrug, stereoisomer, or tautomer thereof. In some embodiments, the molecular glue degrader compound is Compound 212 or a pharmaceutically acceptable salt, hydrate, solvate, prodrug, stereoisomer, or tautomer thereof. In some embodiments, the molecular glue degrader compound is Compound 213 or a pharmaceutically acceptable salt, hydrate, solvate, prodrug, stereoisomer, or tautomer thereof. In some embodiments, the molecular glue degrader compound is Compound 214 or a pharmaceutically acceptable salt, hydrate, solvate, prodrug, stereoisomer, or tautomer thereof. In some embodiments, the molecular glue degrader compound is Compound 215 or a pharmaceutically acceptable salt, hydrate, solvate, prodrug, stereoisomer, or tautomer thereof. In some embodiments, the molecular glue degrader compound is Compound 216 or a pharmaceutically acceptable salt, hydrate, solvate, prodrug, stereoisomer, or tautomer thereof. In some embodiments, the molecular glue degrader compound is Compound 217 or a pharmaceutically acceptable salt, hydrate, solvate, prodrug, stereoisomer, or tautomer thereof. In some embodiments, the molecular glue degrader compound is Compound 218 or a pharmaceutically acceptable salt, hydrate, solvate, prodrug, stereoisomer, or tautomer thereof. In some embodiments, the molecular glue degrader compound is Compound 219 or a pharmaceutically acceptable salt, hydrate, solvate, prodrug, stereoisomer, or tautomer thereof. In some embodiments, the molecular glue degrader compound is Compound 220 or a pharmaceutically acceptable salt, hydrate, solvate, prodrug, stereoisomer, or tautomer thereof. In some embodiments, the molecular glue degrader compound is Compound 221 or a pharmaceutically acceptable salt, hydrate, solvate, prodrug, stereoisomer, or tautomer thereof. In some embodiments, the molecular glue degrader compound is Compound 222 or a pharmaceutically acceptable salt, hydrate, solvate, prodrug, stereoisomer, or tautomer thereof. In some embodiments, the molecular glue degrader compound is Compound 223 or a pharmaceutically acceptable salt, hydrate, solvate, prodrug, stereoisomer, or tautomer thereof. In some embodiments, the molecular glue degrader compound is Compound 224 or a pharmaceutically acceptable salt, hydrate, solvate, prodrug, stereoisomer, or tautomer thereof. In some embodiments, the molecular glue degrader compound is Compound 225 or a pharmaceutically acceptable salt, hydrate, solvate, prodrug, stereoisomer, or tautomer thereof. In some embodiments, the molecular glue degrader compound is Compound 226 or a pharmaceutically acceptable salt, hydrate, solvate, prodrug, stereoisomer, or tautomer thereof. In some embodiments, the molecular glue degrader compound is Compound 227 or a pharmaceutically acceptable salt, hydrate, solvate, prodrug, stereoisomer, or tautomer thereof. In some embodiments, the molecular glue degrader compound is Compound 228 or a pharmaceutically acceptable salt, hydrate, solvate, prodrug, stereoisomer, or tautomer thereof. In some embodiments, the molecular glue degrader compound is Compound 229 or a pharmaceutically acceptable salt, hydrate, solvate, prodrug, stereoisomer, or tautomer thereof. In some embodiments, the molecular glue degrader compound is Compound 230 or a pharmaceutically acceptable salt, hydrate, solvate, prodrug, stereoisomer, or tautomer thereof. In some embodiments, the molecular glue degrader compound is Compound 231 or a pharmaceutically acceptable salt, hydrate, solvate, prodrug, stereoisomer, or tautomer thereof. In some embodiments, the molecular glue degrader compound is Compound 232 or a pharmaceutically acceptable salt, hydrate, solvate, prodrug, stereoisomer, or tautomer thereof. In some embodiments, the molecular glue degrader compound is Compound 233 or a pharmaceutically acceptable salt, hydrate, solvate, prodrug, stereoisomer, or tautomer thereof. In some embodiments, the molecular glue degrader compound is Compound 234 or a pharmaceutically acceptable salt, hydrate, solvate, prodrug, stereoisomer, or tautomer thereof. In some embodiments, the molecular glue degrader compound is Compound 235 or a pharmaceutically acceptable salt, hydrate, solvate, prodrug, stereoisomer, or tautomer thereof. In some embodiments, the molecular glue degrader compound is Compound 236 or a pharmaceutically acceptable salt, hydrate, solvate, prodrug, stereoisomer, or tautomer thereof. In some embodiments, the molecular glue degrader compound is Compound 237 or a pharmaceutically acceptable salt, hydrate, solvate, prodrug, stereoisomer, or tautomer thereof. In some embodiments, the molecular glue degrader compound is Compound 238 or a pharmaceutically acceptable salt, hydrate, solvate, prodrug, stereoisomer, or tautomer thereof. In some embodiments, the molecular glue degrader compound is Compound 239 or a pharmaceutically acceptable salt, hydrate, solvate, prodrug, stereoisomer, or tautomer thereof. In some embodiments, the molecular glue degrader compound is Compound 240 or a pharmaceutically acceptable salt, hydrate, solvate, prodrug, stereoisomer, or tautomer thereof. In some embodiments, the molecular glue degrader compound is Compound 241 or a pharmaceutically acceptable salt, hydrate, solvate, prodrug, stereoisomer, or tautomer thereof. In some embodiments, the molecular glue degrader compound is Compound 242 or a pharmaceutically acceptable salt, hydrate, solvate, prodrug, stereoisomer, or tautomer thereof. In some embodiments, the molecular glue degrader compound is Compound 243 or a pharmaceutically acceptable salt, hydrate, solvate, prodrug, stereoisomer, or tautomer thereof. In some embodiments, the molecular glue degrader compound is Compound 244 or a pharmaceutically acceptable salt, hydrate, solvate, prodrug, stereoisomer, or tautomer thereof. In some embodiments, the molecular glue degrader compound is Compound 245 or a pharmaceutically acceptable salt, hydrate, solvate, prodrug, stereoisomer, or tautomer thereof. In some embodiments, the molecular glue degrader compound is Compound 246 or a pharmaceutically acceptable salt, hydrate, solvate, prodrug, stereoisomer, or tautomer thereof. In some embodiments, the molecular glue degrader compound is Compound 247 or a pharmaceutically acceptable salt, hydrate, solvate, prodrug, stereoisomer, or tautomer thereof. In some embodiments, the molecular glue degrader compound is Compound 248 or a pharmaceutically acceptable salt, hydrate, solvate, prodrug, stereoisomer, or tautomer thereof. In some embodiments, the molecular glue degrader compound is Compound 249 or a pharmaceutically acceptable salt, hydrate, solvate, prodrug, stereoisomer, or tautomer thereof. In some embodiments, the molecular glue degrader compound is Compound 250 or a pharmaceutically acceptable salt, hydrate, solvate, prodrug, stereoisomer, or tautomer thereof. In some embodiments, the molecular glue degrader compound is Compound 251 or a pharmaceutically acceptable salt, hydrate, solvate, prodrug, stereoisomer, or tautomer thereof. In some embodiments, the molecular glue degrader compound is Compound 252 or a pharmaceutically acceptable salt, hydrate, solvate, prodrug, stereoisomer, or tautomer thereof. In some embodiments, the molecular glue degrader compound is Compound 253 or a pharmaceutically acceptable salt, hydrate, solvate, prodrug, stereoisomer, or tautomer thereof. In some embodiments, the molecular glue degrader compound is Compound 254 or a pharmaceutically acceptable salt, hydrate, solvate, prodrug, stereoisomer, or tautomer thereof. In some embodiments, the molecular glue degrader compound is Compound 255 or a pharmaceutically acceptable salt, hydrate, solvate, prodrug, stereoisomer, or tautomer thereof. In some embodiments, the molecular glue degrader compound is Compound 256 or a pharmaceutically acceptable salt, hydrate, solvate, prodrug, stereoisomer, or tautomer thereof. In some embodiments, the molecular glue degrader compound is Compound 257 or a pharmaceutically acceptable salt, hydrate, solvate, prodrug, stereoisomer, or tautomer thereof. In some embodiments, the molecular glue degrader compound is Compound 258 or a pharmaceutically acceptable salt, hydrate, solvate, prodrug, stereoisomer, or tautomer thereof. In some embodiments, the molecular glue degrader compound is Compound 259 or a pharmaceutically acceptable salt, hydrate, solvate, prodrug, stereoisomer, or tautomer thereof. In some embodiments, the molecular glue degrader compound is Compound 260 or a pharmaceutically acceptable salt, hydrate, solvate, prodrug, stereoisomer, or tautomer thereof. In some embodiments, the molecular glue degrader compound is Compound 261 or a pharmaceutically acceptable salt, hydrate, solvate, prodrug, stereoisomer, or tautomer thereof. In some embodiments, the molecular glue degrader compound is Compound 262 or a pharmaceutically acceptable salt, hydrate, solvate, prodrug, stereoisomer, or tautomer thereof. In some embodiments, the molecular glue degrader compound is Compound 263 or a pharmaceutically acceptable salt, hydrate, solvate, prodrug, stereoisomer, or tautomer thereof. In some embodiments, the molecular glue degrader compound is Compound 264 or a pharmaceutically acceptable salt, hydrate, solvate, prodrug, stereoisomer, or tautomer thereof. In some embodiments, the molecular glue degrader compound is Compound 265 or a pharmaceutically acceptable salt, hydrate, solvate, prodrug, stereoisomer, or tautomer thereof. In some embodiments, the molecular glue degrader compound is Compound 266 or a pharmaceutically acceptable salt, hydrate, solvate, prodrug, stereoisomer, or tautomer thereof. In some embodiments, the molecular glue degrader compound is Compound 267 or a pharmaceutically acceptable salt, hydrate, solvate, prodrug, stereoisomer, or tautomer thereof. In some embodiments, the molecular glue degrader compound is Compound 268 or a pharmaceutically acceptable salt, hydrate, solvate, prodrug, stereoisomer, or tautomer thereof. In some embodiments, the molecular glue degrader compound is Compound 269 or a pharmaceutically acceptable salt, hydrate, solvate, prodrug, stereoisomer, or tautomer thereof. In some embodiments, the molecular glue degrader compound is Compound 270 or a pharmaceutically acceptable salt, hydrate, solvate, prodrug, stereoisomer, or tautomer thereof. In some embodiments, the molecular glue degrader compound is Compound 271 or a pharmaceutically acceptable salt, hydrate, solvate, prodrug, stereoisomer, or tautomer thereof. In some embodiments, the molecular glue degrader compound is Compound 272 or a pharmaceutically acceptable salt, hydrate, solvate, prodrug, stereoisomer, or tautomer thereof. In some embodiments, the molecular glue degrader compound is Compound 273 or a pharmaceutically acceptable salt, hydrate, solvate, prodrug, stereoisomer, or tautomer thereof. In some embodiments, the molecular glue degrader compound is Compound 274 or a pharmaceutically acceptable salt, hydrate, solvate, prodrug, stereoisomer, or tautomer thereof. In some embodiments, the molecular glue degrader compound is Compound 275 or a pharmaceutically acceptable salt, hydrate, solvate, prodrug, stereoisomer, or tautomer thereof. In some embodiments, the molecular glue degrader compound is Compound 276 or a pharmaceutically acceptable salt, hydrate, solvate, prodrug, stereoisomer, or tautomer thereof.

### Target Proteins

In one aspect, the disclosure provides a molecular glue degrader compound or a pharmaceutically acceptable salt, hydrate, solvate, prodrug, stereoisomer, or tautomer thereof, which recruit a targeted protein, such as a protein kinase, a phosphodiesterase, or a bromodomain-containing protein to a RING E3 ubiquitin ligase for degradation.

In an embodiment, the target protein is selected from a tyrosine kinase, a serine/threonine kinase, a bromodomain-containing protein, an epigenetic protein, and a misfolded protein. The target protein may be selected from AR, BCL-2/BCL, BCL-XL, BCR-ABL, BRD2, BRD3, BRD4, BRD9, BRDT, BTK, BUB1, BUB1B, c-ABL, CDK1, CDK2, CDK3, CDK4, CDK5, CDK6, CDK7, CDK8, CDK9, CDK10, CDK11B, CDK12, CDK13, CDK14, CDK15, CDK16, CDK17, CDK18, CDK19, CDK20, CTHEK1, CKS1B, CKS2, CSNK1A1, CSNK1E, CTNNB1, DSTYK, EEF2K, estrogen receptor (ER), ETNK1, FASTKD5, HRAS, ITPKB, KRAS, KRAS4A, KRAS41B, KRAS G12A, KRAS G12C, KRAS G12D, KRAS G12S, KRAS G12V, KRAS G13D, LRKK2, MAPKAPK2 (MIK2), MARK2, MAP3K2, MELK, MYC, MYCN, NEK6, NRAS, PANK2, PANK3, PDE5, PHKA1, PHKA2, PKN2, PLK1, PTK6, RIOK2, SKP2, SMARCA2, SMARCA4, STAT1, STAT2, STAT3, STAT4, STAT5A, STAT5B, STAT6, TK1, TTK, and UCK2. In an embodiment, the target protein is selected from a protein listed in Table 3 below.

TABLE 3
Exemplary Target Proteins

| Target Code | Target Name |
|---|---|
| AR | Androgen hormone receptor |
| AR-V7 | Androgen hormone receptor variant 7 |
| BCL-2 | BCL2 apoptosis regulator |
| BCL11A | BAF Chromatin Remodeling |
| | Complex Subunit BCL11A |
| BCL2L1 | BCL2 like 1 (also known as BCL-XL) |
| BCR-ABL | BCR-ABL protein |
| BRD2 | Bromodomain containing protein 2 |
| BRD3 | Bromodomain containing protein 3 |
| BRD4 | Bromodomain containing protein 4 |
| BRD5 | Bromodomain containing protein 5 |
| BRD7 | Bromodomain containing protein 7 |
| BRD8 | Bromodomain containing protein 8 |
| BRD9 | Bromodomain containing protein 9 |
| BRDT | bromodomain testis associated protein |
| BTK | Bruton's tyrosine kinase |
| BUB1 | Budding uninhibited by |
| | benzimidazoles 1 also known as mitotic |
| | checkpoint serine/threonine-protein |
| | kinase BUB1 |
| BUB1B | Mitotic checkpoint serine/threonine-protein |
| | kinase BUB1 beta |
| c-ABL | c-ABL tyrosine kinase |
| CDK10 | Cyclin Dependent Kinase 10 |
| CDK11B | Cyclin Dependent Kinase 11B |
| CDK12 | Cyclin Dependent Kinase 12 |
| CDK13 | Cyclin Dependent Kinase 13 |
| CDK14 | Cyclin Dependent Kinase 14 |
| CDK15 | Cyclin Dependent Kinase 15 |
| CDK16 | Cyclin Dependent Kinase 16 |
| CDK17 | Cyclin Dependent Kinase 17 |
| CDK18 | Cyclin Dependent Kinase 18 |
| CDK19 | Cyclin Dependent Kinase 19 |
| CDK20 | Cyclin Dependent Kinase 20 |
| CDK1 | Cyclin Dependent Kinase 1 |
| CDK2 | Cyclin Dependent Kinase 2 |
| CDK3 | Cyclin Dependent Kinase 3 |
| CDK4 | Cyclin Dependent Kinase 4 |
| CDK5 | Cyclin Dependent Kinase 5 |
| CDK6 | Cyclin Dependent Kinase 6 |
| CDK7 | Cyclin Dependent Kinase 7 |
| CDK8 | Cyclin Dependent Kinase 8 |
| CDK9 | Cyclin Dependent Kinase 9 |
| CHEK1 | Checkpoint kinase 1 |
| CKS2 | CDC28 protein kinase regulatory subunit 1B |
| CSNK1A1 | Casein kinase 1 alpha 1 |
| CSNK1E | Casein kinase 1 epsilon |
| CTNNB1 | Catenin beta 1 |
| CTNNB1 | catenin beta 1 |
| DSTYK | Dual serine/threonine and tyrosine protein kinase |
| EEF2K | Eukaryotic elongation factor 2 kinase |
| ER | Estrogen receptor |
| ETNK1 | Ethanolamine kinase 1 |
| FASTKD5 | FAST kinase domain 5 |
| HRAS | HRas proto-oncogene |
| ITPKB | Inositol-trisphosphate 3 kinase B |
| KRAS | K-Ras P21 Protein |
| LRRK2 | Leucine-rich repeat kinase 2 |
| MAPKAPK2 | Mitogen-activated protein kinase-activated |
| | protein kinase 2 |
| MAP3K2 | mitogen-activated protein kinase kinase kinase 2 |
| MELK | Maternal embryonic leucine zipper kinase |
| MYC | Myc Proto-Oncogene |
| MARK2 | Microtubule affinity regulating kinase 2 |
| MYCN | N-myc Proto-oncogene protein |
| NEK6 | NIMA related Kinase 6 |
| NRAS | Neuroblastoma RAS Proto-oncogene |
| PANK2 | Pantothenate Kinase 2 |
| PANK3 | Pantothenate Kinase 3 |
| PDE5 | Phosphodiesterase 5 |
| PHKA1 | Phosphorylase Kinase regulatory subunit alpha 1 |
| PHKA1 | Phosphorylase Kinase regulatory subunit alpha 2 |
| PKN2 | Protein kinase N2 |
| PLK1 | Polo Like Kinase 1 |
| PTK6 | Protein Tyrosine Kinase 6 |
| RIOK2 | Rio Kinase 2 |
| SKP2 | S-Phase kinase associated protein 2 |
| SMARCA2 | SWI/SNF related, matrix associated, |
| | actin dependent regulator of |
| | chromatin, subfamily a, member 2 |
| SMARCA4 | SWI/SNF related, matrix associated, |
| | actin dependent regulator of |
| | chromatin, subfamily a, member 4 |
| STAT1 | Signal Transducer and Activator of Transcription 1 |
| STAT2 | Signal Transducer and Activator of Transcription 2 |
| STAT3 | Signal Transducer and Activator of Transcription 3 |
| STAT4 | Signal Transducer and Activator of Transcription 4 |
| STAT5A | Signal Transducer and Activator of Transcription 1 |
| STAT5B | Signal Transducer and Activator of Transcription 1 |
| STAT6 | Signal Transducer and Activator of Transcription 6 |
| TK1 | Thymidine Kinase 1 |
| TTK | TTK protein kinase |
| UCK2 | Uridine-cytidine kinase 2 |
| WEE1 | WEE1 G2 Checkpoint Kinase |
| WDR5 | WD Repeat-Containing Protein 5 |

In an embodiment, the target protein is selected from AR-V7, BCL-2/BCL, BCR-ABL, BRD4, BTK, BUB1, BUB1B, c-ABL, CDK4, CDK11B, CHEK1, CKS1B, CKS2, CSNK1A1, CSNK1E, DSTYK, EEF2K, ETNK1, FASTKD5, ITPKB, KRAS G12D, LRKK2, MARK2, MAP3K2, MELK, NEK6, PANK2, PANK3, PDE5, PHKA1, PHKA2, PKN2, PLK1, RIOK2, SKP2, SMARCA2, TK1, TTK, and UCK2.

In an embodiment, the target protein is AR. In an embodiment, the target protein is AR-V7. In an embodiment, the target protein is Bcl-2/Bcl. In an embodiment, the target protein is BCR-ABL. In an embodiment, the target protein is BRD2. In an embodiment, the target protein is BRD2. In an embodiment, the target protein is BRD3. In an embodiment, the target protein is BRD2. In an embodiment, the target protein is BRD4. In an embodiment, the target protein is BRD4. In an embodiment, the target protein is BRD9. In an embodiment, the target protein is BRDT. In an embodiment, the target protein is BTK. In an embodiment, the target protein is BUB1. In an embodiment, the target protein is BUB1B. In an embodiment, the target protein is BRD2. In an embodiment, the target protein is BUB1. In an embodiment, the target protein is BRD2. In an embodiment, the target protein is CKD1. In an embodiment, the target protein is BRD2. In an embodiment, the target protein is CKD2. In an embodiment, the target protein is BRD2. In an embodiment, the target protein is CKD3. In an embodiment, the target protein is c-ABL. In an embodiment, the target protein is CDK4. In an embodiment, the target protein is c-ABL. In an embodiment, the target protein is CDK5. In an embodiment, the target protein is c-ABL. In an embodiment, the target protein is CDK6. In an embodiment, the target protein is c-ABL. In an embodiment, the target protein is CDK7. In an embodiment, the target protein is CDK8. In an embodiment, the target protein is CDK8. In an embodiment, the target protein is c-ABL. In an embodiment, the target protein is CDK9. In an embodiment, the target protein is c-ABL. In an embodiment, the target protein is CDK10. In an embodiment, the target protein is CDK9. In an embodiment, the target protein is CDK11B. In an embodiment, the target protein is c-ABL. In an embodiment, the target protein is CDK12. In an embodiment, the target protein is c-ABL. In an embodiment, the target protein is CDK13. In an embodiment, the target protein is c-ABL. In an embodiment, the target protein is CDK14. In an embodiment, the target protein is c-ABL. In an embodiment, the target protein is CDK15. In an embodiment, the target protein is c-ABL. In an embodiment, the target protein is CDK16. In an embodiment, the target protein is c-ABL. In an embodiment, the target protein is CDK17. In an embodiment, the target protein is c-ABL. In an embodiment, the target protein is CDK18. In an embodiment, the target protein is c-ABL. In an embodiment, the target protein is CDK19. In an embodiment, the target protein is c-ABL. In an embodiment, the target protein is CDK20. In an embodiment, the target protein is c-ABL. In an embodiment, the target protein is CHEK1. In an embodiment, the target protein is CHEK1. In an embodiment, the target protein is CKS1B. In an embodiment, the target protein is c-ABL. In an embodiment, the target protein is CKS2. In an embodiment, the target protein is CKS2. In an embodiment, the target protein is CSNK1A1. In an embodiment, the target protein is CKS2. In an embodiment, the target protein is CSNK1E. In an embodiment, the target protein is CKS2. In an embodiment, the target protein is CTNNB1. In an embodiment, the target protein is CSNK1E. In an embodiment, the target protein is DSTYK. In an embodiment, the target protein is EEF2K. In an embodiment, the target protein is estrogen receptor (ER). In an embodiment, the target protein is ETNK1. In an embodiment, the target protein is FASTKD5. In an embodiment, the target protein is HRAS. In an embodiment, the target protein is ITPKB. In an embodiment, the target protein is KRAS. In an embodiment, the target protein is KRAS4A. In an embodiment, the target protein is KRAS4B. In an embodiment, the target protein is KRAS4A. In an embodiment, the target protein is KRAS G12A. In an embodiment, the target protein is KRAS G12B. In an embodiment, the target protein is KRAS G12C. In an embodiment, the target protein is KRAS G12D. In an embodiment, the target protein is KRAS G12S. In an embodiment, the target protein is KRAS G12V. In an embodiment, the target protein is KRAS G13D. In an embodiment, the target protein is LRRK2. In an embodiment, the target protein is MAPKAPK2. In an embodiment, the target protein is MARK2. In an embodiment, the target protein is MAP3K2. In an embodiment, the target protein is MELK. In an embodiment, the target protein is MYC. In an embodiment, the target protein is MYCN. In an embodiment, the target protein is NEK6. In an embodiment, the target protein is NRAS. In an embodiment, the target protein is PANK2. In an embodiment, the target protein is PANK3. In an embodiment, the target protein is PBRM1. In an embodiment, the target protein is PDE5. In an embodiment, the target protein is PHKA1. In an embodiment, the target protein is PHKA2. In an embodiment, the target protein is PKN2. In an embodiment, the target protein is PLK1. In an embodiment, the target protein is PTK6. In an embodiment, the target protein is RIOK2. In an embodiment, the target protein is RIPK2. In an embodiment, the target protein is SKP2. In an embodiment, the target protein is SMARCA2. In an embodiment, the target protein is SMARCA4. In an embodiment, the target protein is STAT1. In an embodiment, the target protein is STAT2. In an embodiment, the target protein is STAT3. In an embodiment, the target protein is STAT4. In an embodiment, the target protein is STAT5A. In an embodiment, the target protein is STAT5B. In an embodiment, the target protein is STAT6. In an embodiment, the target protein is tau. In an embodiment, the target protein is TBK1. In an embodiment, the target protein is TK1. In an embodiment, the target protein is TTK. In an embodiment, the target protein is UCK2. In an embodiment, the target protein is WDR5. In an embodiment, the target protein is WEE1.

### Definitions

One embodiment is a compound of any of the formulae described herein, e.g., a compound of Formula (I) and subformula thereof, or a pharmaceutically acceptable salt, hydrate, solvate, prodrug, stereoisomer, or tautomer thereof, that modulates, e.g., decreases the amount of a targeted protein or protein of interest, e.g., one or more proteins from Table 3.

Another embodiment is a Formula (I) and subformula thereof, or a pharmaceutically acceptable salt, hydrate, solvate, prodrug, stereoisomer, or tautomer thereof, that degrades a targeted protein through the ubiquitin-proteasome pathway (UPP).

The formation of a viable ternary complex among the target protein, the molecular glue degrader compound, and the RING E3 ubiquitin ligase is enabled by the use of targeted molecular glue degraders, relying on two key components, the “Target Ligand” and the “RING E3 Ligase Binder, and, optionally, the joining segment, termed the “Bridge.” In an embodiment, the molecular glue degrader may act as a monovalent degrader, binding to a target protein that then ultimately binds to an RING E3 ubiquitin ligase to facilitate ubiquitination of the target protein and degradation.

The term “a therapeutically effective amount” of a compound described herein refers to an amount of the compound described herein that will elicit the biological or medical response of a subject, for example, reduction or inhibition of an enzyme or a protein activity, or ameliorate symptoms, alleviate conditions, slow or delay disease progression, or prevent a disease, etc. In one embodiment, the term “a therapeutically effective amount” refers to the amount of the compound described herein that, when administered to a subject, is effective to (1) at least partially alleviate, prevent and/or ameliorate a condition, or a disorder or a disease (i) mediated by a target protein, (ii) associated with activity of a target protein, or (iii) characterized by activity (normal or abnormal) of a target protein; or (2) reduce or inhibit the activity of a target protein; or (3) reduce or inhibit the expression of a target protein. These effects may be achieved for example by reducing the amount of a target protein by degrading of the target protein. In one embodiment, the term “a therapeutically effective amount” refers to the amount of the compound described herein that, when administered to a cell, or a tissue, or a non-cellular biological material, or a medium, is effective to at least partially reduce or inhibit the activity of target protein; or at least partially reduce or inhibit the expression of a target protein, for example by degrading a target protein.

As used herein, the term cancer refers to a neoplastic disease and includes for instance solid tumors, such as, e.g. sarcomas or carcinomas or blood cancer, such as, e.g. leukemia or myeloma, or cancers of lymphatic system such as lymphoma, or mixed types thereof.

As used herein, the terms “degrades”, “degrading”, or “degradation” refers to the partial or full breakdown of a target protein by the cellular proteasome system to an extent that reduces or eliminates the biological activity (especially aberrant activity) of target protein. Degradation may be achieved through mediation of a RING E3 ligase, in particular, E3-ligase complexes comprising the protein RNF126. As used herein, the term “modulation of target protein activity” or “modulating target activity” means the alteration of, especially reduction, suppression or elimination, of target protein's activity. This may be achieved by degrading the target protein in vivo or in vitro. The amount of target protein degraded can be measured by comparing the amount of target protein remaining after treatment with a compound described herein as compared to the initial amount or level of target protein present as measured prior to treatment with a compound described herein. In an embodiment, at least about 30% of the target protein is degraded compared to initial levels. In an embodiment, at least about 40% of the target protein is degraded compared to initial levels. In an embodiment, at least about 50% of the target protein is degraded compared to initial levels. In an embodiment, at least about 60% of the target protein is degraded compared to initial levels. In an embodiment, at least about 70% of the target protein is degraded compared to initial levels. In an embodiment, at least about 80% of the target protein is degraded compared to initial levels. In an embodiment, at least about 90% of the target protein is degraded compared to initial levels. In an embodiment, at least about 95% of the target protein is degraded compared to initial levels. In an embodiment, over 95% of the target protein is degraded compared to initial levels. In an embodiment, at least about 99% of the target protein is degraded compared to initial levels.

In an embodiment, the target protein is degraded in an amount of from about 30% to about 99% compared to initial levels. In an embodiment, the target protein is degraded in an amount of from about 40% to about 99% compared to initial levels. In an embodiment, the target protein is degraded in an amount of from about 50% to about 99% compared to initial levels. In an embodiment, the target protein is degraded in an amount of from about 60% to about 99% compared to initial levels. In an embodiment, the target protein is degraded in an amount of from about 70% to about 99% compared to initial levels. In an embodiment, the target protein is degraded in an amount of from about 80% to about 99% compared to initial levels. In an embodiment, the target protein is degraded in an amount of from about 90% to about 99% compared to initial levels. In an embodiment, the target protein is degraded in an amount of from about 95% to about 99% compared to initial levels. In an embodiment, the target protein is degraded in an amount of from about 90% to about 95% compared to initial levels.

As used herein, the term “selectivity for the target protein” means, for example, a compound described herein degrades the target protein in preference to, or to a greater extent than, another protein or proteins.

As used herein, the term “subject” refers to an animal. Typically, the animal is a mammal. A subject also refers to, for example, primates (e.g., humans, male or female), cows, sheep, goats, horses, dogs, cats, rabbits, rats, mice, fish, birds, and the like. In an embodiment, the subject is a primate. In a preferred embodiment, the subject is a human.

As used herein, the terms “inhibit”, “inhibition”, or “inhibiting” refer to the reduction or suppression of a given condition, symptom, or disorder, or disease, or a significant decrease in the baseline activity of a biological activity or process.

As used herein, the terms “treat”, “treating”, or “treatment” of any disease or disorder refer in an embodiment, to ameliorating the disease or disorder (i.e., slowing or arresting or reducing the development of the disease or at least one of the clinical symptoms thereof). In an embodiment, “treat”, “treating”, or “treatment” refers to alleviating or ameliorating at least one physical parameter including those which may not be discernible by the patient.

As used herein, the term “preventing” refers to a reduction in the frequency of, or delay in the onset of, symptoms of the condition or disease.

As used herein, a subject is “in need of” a treatment if such subject would benefit biologically, medically, or in quality of life from such treatment.

As used herein, the term “a,” “an,” “the” and similar terms used in the context of the disclosure (especially in the context of the claims) are to be construed to cover both the singular and plural unless otherwise indicated herein or clearly contradicted by the context.

The term “alkyl” refers to a radical of a straight-chain or branched saturated hydrocarbon group having from 1 to 6 carbon atoms (“C₁₋₆ alkyl”). In some embodiments, an alkyl group has 1 to 5 carbon atoms (“C₁₋₆ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tertiary amyl), and hexyl (C₆) (e.g., n-hexyl).

“Alkylene” refers to a divalent radical of an alkyl group, e.g., —CH₂—, —CH₂CH₂—, and —CH₂CH₂CH₂—.

“Heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and lor 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC1 alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

“Heteroalkylene” refers to a divalent radical of a heteroalkyl group.

“Alkoxy” or “alkoxyl” refers to an —O-alkyl radical. In some embodiments, the alkoxy groups are methoxy, ethoxy, n-propoxy, isopropoxy, n-butoxy, tert-butoxy, sec-butoxy, n-pentoxy, n-hexoxy, and 1,2-dimethylbutoxy. In some embodiments, alkoxy groups are lower alkoxy, i.e., with between 1 and 6 carbon atoms. In some embodiments, alkoxy groups have between 1 and 4 carbon atoms.

As used herein, the term “aryl” refers to a stable, aromatic, mono- or bicyclic ring radical having the specified number of ring carbon atoms. Examples of aryl groups include, but are not limited to, phenyl, 1-naphthyl, 2-naphthyl, and the like. The related term “aryl ring” likewise refers to a stable, aromatic, mono- or bicyclic ring having the specified number of ring carbon atoms.

As used herein, the term “heteroaryl” refers to a stable, aromatic, mono- or bicyclic ring radical having the specified number of ring atoms and comprising one or more heteroatoms individually selected from nitrogen, oxygen and sulfur. The heteroaryl radical may be bonded via a carbon atom or heteroatom. Examples of heteroaryl groups include, but are not limited to, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl, pyridyl, quinolinyl, isoquinolinyl, indolyl, indazolyl, oxadiazolyl, benzothiazolyl, quinoxalinyl, and the like. The related term “heteroaryl ring” likewise refers to a stable, aromatic, mono- or bicyclic ring having the specified number of ring atoms and comprising one or more heteroatoms individually selected from nitrogen, oxygen and sulfur.

As used herein, the term “cycloalkyl” refers to a stable, saturated or unsaturated, or a mixture thereof, non-aromatic, mono- or bicyclic (fused, bridged, or spiro) ring radical having the specified number of ring carbon atoms. Examples of cycloalkyl groups include, but are not limited to, the cycloalkyl groups identified above, cyclobutenyl, cyclopentenyl, cyclohexenyl, and the like. In an embodiment, the specified number is C₃-C₁₂ carbons. The related term “cycloalkyl ring” likewise refers to a stable, saturated or unsaturated, non-aromatic, mono- or bicyclic (fused, bridged, or spiro) ring having the specified number of ring carbon atoms. In an embodiment, the cycloalkyl can be substituted or unsubstituted. In an embodiment, the cycloalkyl can be substituted with 0-4 occurrences of R^{a}, wherein each R^{a} is independently selected from the group consisting of C₁₋₆ alkyl, C₁₋₆ alkoxyl, and halogen.

As used herein, the term “heterocycloalkyl” refers to a stable, saturated or unsaturated or a mixture thereof, non-aromatic, mono- or bicyclic (fused, bridged, or spiro) ring radical having the specified number of ring atoms and comprising one or more heteroatoms individually selected from nitrogen, oxygen and sulfur. The heterocyclyl radical may be bonded via a carbon atom or heteroatom. In an embodiment, the specified number is C₃-C₁₂ carbons. Examples of heterocyclyl groups include, but are not limited to, azetidinyl, oxetanyl, pyrrolinyl, pyrrolidinyl, tetrahydrofuryl, tetrahydrothienyl, piperidyl, piperazinyl, tetrahydropyranyl, morpholinyl, perhydroazepinyl, tetrahydropyridinyl, tetrahydroazepinyl, octahydropyrrolopyrrolyl, and the like. The related term “heterocycloalkyl ring” likewise refers to a stable, saturated or unsaturated, non-aromatic, mono- or bicyclic (fused, bridged, or spiro) ring having the specified number of ring atoms and comprising one or more heteroatoms individually selected from nitrogen, oxygen and sulfur. In an embodiment, the heterocycloalkyl can be substituted or unsubstituted. In an embodiment, the heterocycloalkyl can be substituted with 0-4 occurrences of R^{a}, wherein each R^{a} is independently selected from the group consisting of C₁₋₆ alkyl, C₁₋₆ alkoxyl, and halogen. “Heterocycloalkyl” also includes ring systems wherein the heterocycloalkyl ring, as defined above, is fused with one or more cycloalkyl groups wherein the point of attachment is either on the cycloalkyl or heterocycloalkyl ring, or ring systems wherein the heterocycloalkyl ring, as defined above, is fused with one or more aryl or heteroaryl groups, wherein the point of attachment is on the heterocycloalkyl ring, and in such instances, the number of ring members continue to designate the number of ring members in the heterocycloalkyl ring system.

As used herein, “halo” or “halogen” refers to fluorine (fluoro, —F), chlorine (chloro, —Cl), bromine (bromo, —Br), or iodine (iodo, —I).

As used herein, “haloalkyl” means an alkyl group substituted with one or more halogens. Examples of haloalkyl groups include, but are not limited to, trifluoromethyl, difluoromethyl, pentafluoroethyl, and trichloromethyl.

As used herein, “substituted”, whether preceded by the term “optionally” or not, means that one or more hydrogens of the designated moiety are replaced with a suitable substituent.

As used herein, the definition of each expression, e.g., alkyl, m, n, etc., when it occurs more than once in any structure, is intended to be independent of its definition elsewhere in the same structure.

Various embodiments of the disclosure are described herein. It will be recognized that features specified in each embodiment may be combined with other specified features, including as indicated in the embodiments below, to provide further embodiments of the present disclosure.

It is understood that in the following embodiments, combinations of substituents or variables of the depicted formulae are permissible only if such combinations result in stable compounds.

Definitions of specific functional groups and chemical terms are described in more detail below. The chemical elements are identified in accordance with the Periodic Table of the Elements, CAS version, Handbook of Chemistry and Physics, 75^{th} ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} ed, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} ed, Cambridge University Press, Cambridge, 1987.

Certain compounds described herein may exist in particular geometric or stereoisomeric forms. If, for instance, a particular enantiomer of a compound described herein is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Unless otherwise stated, structures depicted herein are also meant to include geometric (or conformational) forms of the structure; for example, the R and S configurations for each asymmetric center, cis and trans double bond isomers, Z and E double bond isomers, and Z and E conformational isomers. Therefore, single stereochemical isomers as well as enantiomeric, diastereomeric, and geometric (or conformational) mixtures of the disclosed compounds are within the scope of the disclosure. Unless otherwise stated, all tautomeric forms of the compounds described herein are within the scope of the disclosure. Additionally, unless otherwise stated, structures depicted herein are also meant to include compounds that differ only in the presence of one or more isotopically enriched atoms. For example, compounds having the disclosed structures including the replacement of hydrogen by deuterium or tritium, or the replacement of a carbon by a ¹³C or ¹⁴C enriched carbon are within the scope of this disclosure. Such compounds are useful, for example, as analytical tools, as probes in biological assays, or as therapeutic agents in accordance with the disclosure.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer. ee=(90−10)/100×100=80%.

Thus, a composition containing 90% of one enantiomer and 10% of the other enantiomer is said to have an enantiomeric excess of 80%. The compounds or compositions described herein may contain an enantiomeric excess of at least 50%, 75%, 90%, 95%, or 99% of one form of the compound, e.g., the S-enantiomer. In other words such compounds or compositions contain an enantiomeric excess of the S enantiomer over the R enantiomer.

Where a particular enantiomer is preferred, it may, in some embodiments be provided substantially free of the corresponding enantiomer, and may also be referred to as “optically enriched.” “Optically enriched,” as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See e.g., Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw Hill, NY, 1962); Wilen, S. H. Tables of Resolving Agents and Optical Resolutions p. 268 (E. L. Eliel, Ed., Univ. of Notre Dame Press, Notre Dame, IN 1972).

All methods described herein can be performed in any suitable order unless otherwise indicated herein or otherwise clearly contradicted by context. The use of any and all examples, or exemplary language (e.g. “such as”) provided herein is intended merely to better illuminate the disclosure and does not pose a limitation on the scope of the disclosure otherwise claimed.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds described herein into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-O-sulfonic acid. Racemic products can also be resolved by chiral chromatography, e.g., high pressure liquid chromatography (HPLC) using a chiral adsorbent.

### Pharmaceutically Acceptable Salts

Pharmaceutically acceptable salts of the compounds described herein are also contemplated for the uses described herein. As used herein, the terms “salt” or “salts” refer to an acid addition or base addition salt of a compound described herein. “Salts” include in particular “pharmaceutical acceptable salts.” The term “pharmaceutically acceptable salts” refers to salts that retain the biological effectiveness and properties of the compounds disclosed herein and, which typically are not biologically or otherwise undesirable. In many cases, the compounds disclosed herein are capable of forming acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium, and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, and tromethamine.

Another embodiment is a compound of Formula (I) or subformula thereof as an acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate, or xinafoate salt form.

### Pharmaceutical Compositions

Another embodiment is a pharmaceutical composition comprising one or more compounds described herein or a pharmaceutically acceptable salt, hydrate, solvate, prodrug, stereoisomer, or tautomer thereof, and one or more pharmaceutically acceptable carrier(s). The term “pharmaceutically acceptable carrier” refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions of the disclosure are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tween®, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax, and polyethylene glycols.

The pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

The pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. The amount of the compounds of the present disclosure that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

### Isotopically Labelled Compounds

A compound described herein or a pharmaceutically acceptable salt, hydrate, solvate, prodrug, stereoisomer, or tautomer thereof, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds described herein include isotopes of hydrogen, carbon, nitrogen, oxygen, phosphorous, fluorine, and chlorine, such as ²H, 3H, ¹¹C, ¹³C, ¹⁴C, ¹⁵N, ¹⁸F, ³¹P, ³²P, ³⁵S, ³⁶Cl, ¹²³I, ¹²⁴I, ¹²⁵I, respectively. The disclosure includes various isotopically labeled compounds as defined herein, for example, those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds described herein or a pharmaceutically acceptable salt, hydrate, solvate, prodrug, stereoisomer, or tautomer thereof, can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound described herein or a pharmaceutically acceptable salt, hydrate, solvate, prodrug, stereoisomer, or tautomer thereof. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound described herein is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Dosages

Toxicity and therapeutic efficacy of compounds described herein, including pharmaceutically acceptable salts and deuterated variants, can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. Compounds that exhibit large therapeutic indexes are preferred. While compounds that exhibit toxic side effects may be used, care should be taken to design a delivery system that targets such compounds to the site of affected tissue in order to minimize potential damage to uninfected cells and thereby reduce side effects.

Data obtained from the cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### Methods of Use

In one aspect, the present disclosure features a method of modulating a target protein, e.g., a target protein described herein, in a subject in need thereof, the method comprising administering to the subject a therapeutically effective amount of a compound of Formula (I), or a pharmaceutically acceptable salt, hydrate, solvate, prodrug, stereoisomer, or tautomer thereof. In some embodiments, the modulating comprises one or more of: (i) modulating the folding of the target protein; (ii) modulating the half-life of the target protein; (iii) modulating trafficking of the target protein to the proteasome; (iv) modulating the level of ubiquitination of the target protein; (v) modulating degradation (e.g., proteasomal degradation) of the target protein; (vi) modulating target protein signaling; (vii) modulating target protein localization; (viii) modulating trafficking of the target protein to the lysosome; and (ix) modulating target protein interactions with another protein.

In another aspect, the present disclosure features a method of degrading a target protein, e.g., a target protein described herein, in a subject in need thereof, the method comprising administering to the subject a therapeutically effective amount of a compound of Formula (I), or a pharmaceutically acceptable salt, hydrate, solvate, prodrug, stereoisomer, or tautomer thereof. In some embodiments, the degrading comprises decreasing the half-life of a target protein or facilitating the addition of a Ubl onto a target protein, e.g., compared to a reference standard. In some embodiments, the degrading comprises reducing the function of a target protein.

In another aspect, the present disclosure features a method of forming a protein complex comprising a RING E3 ligase, e.g., a RING E3 ligase described herein, and a target protein, upon administration of a compound of Formula (I) or a pharmaceutically acceptable salt, hydrate, solvate, prodrug, stereoisomer, or tautomer thereof. In some embodiments, the protein complex is formed in vitro (e.g., in a sample) or in vivo (e.g., in a cell or tissue, e.g., in a subject). Formulation of the protein complex may be observed and characterized by any method known in the art, e.g., mass spectrometry (native mass spectrometry) or SDS PAGE. In some embodiments, forming the protein complex modulates the level of a target protein, e.g., decreases the half-life of the target protein, e.g., compared to a reference standard. In some embodiments, forming the protein facilitates addition of a Ubl onto a target protein, e.g., compared to a reference standard. In some embodiments, the RING E3 ubiquitin ligase is RNF126.

Another embodiment is a method for adding a Ubl (e.g., a ubiquitin or ubiquitin-like protein) onto a target protein, e.g., a target protein described herein, the method comprising contacting a target protein with an effective amount of a compound of Formula (I), or a pharmaceutically acceptable salt, hydrate, solvate, prodrug, stereoisomer, or tautomer thereof.

In another aspect, the present disclosure provides a method of reducing or inhibiting the activity of a target protein, e.g., a target protein described herein, the method comprising contacting a target protein with an effective amount of a compound of Formula (I), or a pharmaceutically acceptable salt, hydrate, solvate, prodrug, stereoisomer, or tautomer thereof.

In an embodiment, reducing or inhibiting the activity of a target protein comprises binding to a RING E3 ubiquitin ligase with the molecular glue compound described herein, e.g., a compound of Formula (I), forming a ternary complex of the target protein, the molecular glue compound, and the RING E3 ubiquitin ligase, to thereby reduce or inhibit the activity of the target protein.

In another aspect, the present disclosure features a method of treating or preventing a disease, disorder or condition mediated by a target protein, e.g., a target protein described herein, the method comprising administering to the subject a therapeutically effective amount of a compound of Formula (I), or a pharmaceutically acceptable salt, hydrate, solvate, prodrug, stereoisomer, or tautomer thereof. In some embodiments, the disease, disorder, or condition is selected from the group consisting of a respiratory disorder, a proliferative disorder, an autoimmune disorder, an autoinflammatory disorder, an inflammatory disorder, a metabolic disorder, a neurological disorder, and an infectious disease. In some embodiments, the disease, disorder, or condition is selected from the group consisting of a respiratory disorder, a proliferative disorder, an autoimmune disorder, an autoinflammatory disorder, an inflammatory disorder, a neurological disorder, and an infectious disease. In some embodiments, the disease, disorder, or condition comprises a respiratory disorder. In some embodiments, the disease, disorder, or condition comprises a proliferative disorder. In some embodiments, the disease, disorder, or condition comprises an autoinflammatory disorder. In some embodiments, the disease, disorder, or condition comprises an inflammatory disorder. In some embodiments, the disease, disorder, or condition comprises a metabolic disorder. In some embodiments, the disease, disorder, or condition comprises a neurological disorder. In some embodiments, the disease, disorder, or condition comprises an infectious disease.

In some embodiments, the proliferative disorder is cancer. As used herein, the term “cancer” refers to a malignant neoplasm (Stedman's Medical Dictionary, 25th ed.; Hensyl ed.; Williams & Wilkins: Philadelphia, 1990). The cancer may involve any organ, tissue, or cell in the body. All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the proliferative disorder is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the target protein is BRD4 and the cancer is selected from diffuse large B cell lymphoma, acute myeloid leukemia (AML) (e.g., B-cell AML, T-cell AML), prostate cancer (e.g., prostate adenocarcinoma), and breast cancer. In some embodiments, the target protein is BCR-Abl and the cancer is selected from chronic myelogenous leukemia (CML), acute myeloid leukemia (AML) (e.g., B-cell AML, T-cell AML), and acute lymphoblastic leukemia (ALL). In some embodiments, the target protein is c-ABL and the cancer is chronic myelogenous leukemia (CML). In some embodiments, the target protein is PDE5 and the disease, disorder, or condition is selected from a cardiovascular disease (e.g., hypertension), metabolic disease (e.g., diabetes), cancer, or erectile dysfunction. In some embodiments, the target protein is AR or AR-v7 and the disease, disorder, or condition is related to sex development in a subject or cancer (e.g., prostate cancer or breast cancer). In some embodiments, the target protein is BTK and the disease, disorder, or condition is selected from cancer (mantle cell lymphoma, chronic lymphocytic leukemia (CLL), B-cell lymphoma), or multiple sclerosis. In some embodiments, the target protein is LRRK2 and the disease, disorder, or condition is selected from a neurodegenerative disease, such as Parkinson's disease. In some embodiments, the target protein is SMARCA2 and the disease, disorder, or condition is selected from a cancer.

In another aspect, the disclosure provides a compound of Formula (I) or a pharmaceutically acceptable salt, hydrate, solvate, prodrug, stereoisomer, or tautomer thereof, for use in inhibiting or modulating a target protein in a subject in need thereof.

Another embodiment is a use of a compound of Formula (I), or a pharmaceutically acceptable salt, hydrate, solvate, prodrug, stereoisomer, or tautomer thereof, in the manufacture of a medicament for treating or preventing a respiratory disorder, a proliferative disorder, an autoimmune disorder, an autoinflammatory disorder, an inflammatory disorder, a neurological disorder, and an infectious disease or disorder in a subject in need thereof.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Compounds of the present disclosure may be prepared by methods known in the art of organic synthesis. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (1999) Protective Groups in Organic Synthesis, 3rd edition, John Wiley & Sons). These groups are removed at a convenient stage of the compound synthesis using methods that are readily apparent to those skilled in the art.

### Synthetic Methods and Characterization

All chemical reactions were carried out under a nitrogen atmosphere with dry solvents under anhydrous conditions, unless otherwise noted. Reagents were purchased at the highest commercial quality and used without further purification, unless otherwise stated. Reactions were stirred magnetically and monitored by thin layer chromatography (TLC) carried out on Merck glass silica gel plates (60 F254) using UV light as a visualizing agent and iodine and/or phosphomolybdic acid stain as developing agents. Solvents were removed in vacuo using either a Buchi R-300 Rotavapor (equipped with an I-300 Pro Interface, B-300 Base Heating Bath, Welch 2037B-01 DryFast pump, and VWR AD15R-40-V11B Circulating Bath). Solvents for silica gel chromatography were used as supplied by Sigma-Aldrich. Automated flash chromatography was performed on a Biotage Isolera instrument, equipped with a UV detector. Chromatograms were recorded at 254 and 280 nm.

MS: Low-resolution mass spectra were obtained using Agilent 6460 Triple Quad LC/MS instrument. High-resolution mass spectra (FIRMS) were obtained at the Catalysis Center at the College of Chemistry, University of California, Berkeley.

NMR: ¹H and ¹³C nuclear magnetic resonance (NMR) spectra were recorded on Nuclear Magnetic Resonance (NMR) spectra were recorded on BRUKER AV (600 MHz and 300 MHz), AVB (400 MHz), AVQ (400 MHz) and NEO (500 MHz) spectrometers. Measurements were carried out at ambient temperature. Chemical shifts (6) are reported in ppm with the residual solvent signal as internal standard (chloroform at 7.26 and 77.00 ppm for ¹H NMR and ¹³C NMR spectroscopy, respectively). The data is reported as (s=singlet, d=doublet, t=triplet, q=quartet, p=quintet, m=multiplet or unresolved, br=broad signal, coupling constant(s) in Hz, integration). ¹³C NMR spectra were recorded with broadband ¹H decoupling.

### General Procedures

### Amide Couplings

### General Procedure A

A mixture of the corresponding carboxylic acid (1.1 equiv.) and HATU (1.2 equiv.) was purged with N₂ for 5 minutes. The mixture was dissolved in N,N-dimethylformamide (DMF) (0.1 M), N,N-diisopropylethylamine (DIPEA) (3 equiv.) was added and the reaction mixture was allowed to stir at ambient temperature for 30 minutes. The corresponding amine (1 equiv.) was dissolved in DMF (0.1 M) then added dropwise and the reaction mixture was stirred at ambient temperature overnight. The reaction was quenched with 5 times the reaction volume of 5% LiCl_{(aq)} and extracted 3 times with dichloromethane (DCM) or ethyl acetate (EtOAc). The organic extracts were dried over Na₂SO₄, vacuum filtered, and concentrated in vacuo. The resultant residue was purified by silica gel flash chromatography to afford the title compound.

### General Procedure B

The corresponding carboxylic acid (1.1 equiv.) was added to a vessel and purged with N₂ for 5 minutes. The acid was then dissolved in DCM (0.1 M) and cooled to 0° C. in an ice bath. Oxalyl chloride (1.2 equiv.) was added dropwise at 0° C. A few drops of DMF were added and the reaction mixture was allowed to stir and come to ambient temperature over 2 hours. The volatiles were removed in vacuo and the resultant residue was redissolved in DCM (0.1 M) and cooled to 0° C. in an ice bath. The corresponding amine (1.1 equiv.) was dissolved in DCM (0.1M). DIPEA (3 equiv.) was added, and the mixture was stirred for 5 minutes at ambient temperature before being added to the acyl chloride dropwise at 0° C. The reaction mixture was allowed to stir and come to ambient temperature overnight. The reaction was quenched with water and extracted 3 times with EtOAc. The organic extracts were dried over Na₂SO₄, vacuum filtered, and concentrated in vacuo. The resultant residue was purified by silica gel flash chromatography to afford the title compound.

### General Procedure C

The corresponding carboxylic acid (1.1 equiv.) was added to a vessel and purged with N₂ for 5 minutes. The acid was dissolved in acetonitrile (0.1 M) and DIPEA (2 equiv.) was added. Pentafluoropyridine (1.1 equiv.) was added dropwise, and the reaction mixture was stirred at ambient temperature for 30 minutes. The corresponding amine was dissolved in acetonitrile (0.1 M) and added to acyl fluoride. The reaction mixture was stirred at 100 0° C. overnight. The volatiles were removed in vacuo, and the resultant residue was purified by silica gel flash chromatography to afford the title compound.

### General Procedure D

The corresponding carboxylic acid (1.0 equiv.) was added to a vessel and purged with N₂ for 5 minutes. The acid was dissolved in DMF (0.1 M) and DIPEA (3 equiv.) was added. A >50% wt. solution of propylphosphonic anhydride (T3P) in EtOAc (1.5 equiv.) was added dropwise, and the reaction mixture was stirred at ambient temperature for 30 minutes. The corresponding amine (1.2 equiv.) was dissolved in DMF (0.1 M) then added dropwise and the reaction mixture was stirred at ambient temperature overnight. The reaction was quenched with 5 times the reaction volume of 5% LiCl_{(aq)} and extracted 3 times with EtOAc. The organic extracts were washed once with 5% LiCl_{(aq)}, dried over Na₂SO₄, vacuum filtered, and concentrated in vacuo. The resultant residue was purified by silica gel flash chromatography to afford the title compound.

### Tert-butyloxycarbonyl Deprotection

### General Procedure E

The corresponding tert-butyloxycarbonyl protected amine (1 equiv.) was dissolved in DCM (0.1 M). Trifluoroacetic acid (32 equiv.) was added, and the reaction mixture was stirred at ambient temperature for 30 minutes to overnight. The volatiles were removed in vacuo and the crude residue was used without further purification, unless otherwise noted.

### Example 1: Synthesis of Compounds 100-107, 109-112, 114-116, 135-136

### Synthesis of Compound 100

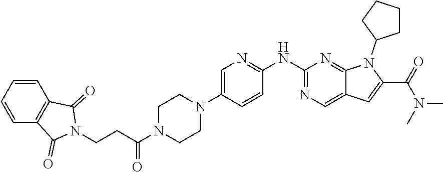 [SMILES: CN(C)C(=O)c1cc2cnc(Nc3ccc(N4CCN(C(=O)CCN5C(=O)c6ccccc6C5=O)CC4)cn3)nc2n1C1CCCC1]

General Procedure A was followed with 3-phthalimidopropionic acid (21.6 mg, 0.06 mmol), HATU (43.8 mg, 0.12 mmol), DIPEA (0.04 mL, 0.23 mmol), and ribociclib (25.0 mg, 0.06 mmol). The crude residue was purified by silica gel chromatography (0-10% DCM in MeOH) to afford 7-cyclopentyl-2-((5-(4-(3-(1,3-dioxoisoindolin-2-yl)propanoyl)piperazin-1-yl)pyridin-2-yl)amino)-N,N-dimethyl-7H-pyrrolo[2,3-d]pyrimidine-6-carboxamide (Compound 100, 14.9 mg, 41%) as a film. ¹H NMR (500 MHz, CDCl₃) δ 8.70 (s, 1H), 8.37 (d, J=9.0 Hz, 1H), 8.03-7.95 (m, 2H), 7.89-7.81 (m, 2H), 7.75-7.68 (m, 2H), 7.32 (dd, J=9.1, 3.0 Hz, 1H), 6.44 (s, 1H), 4.79 (p, J=9.0 Hz, 1H), 4.09-4.03 (m, 2H), 3.80 (t, J=5.2 Hz, 2H), 3.64 (t, J=5.1 Hz, 2H), 3.15 (s, 6H), 3.14-3.08 (m, 4H), 2.85-2.78 (m, 2H), 2.65-2.52 (m, 2H), 2.06 (m 4H), 1.77-1.70 (m, 2H). ¹³C NMR (151 MHz, CDCl₃) δ 168.4, 168.2, 164.1, 154.5, 151.9, 151.8, 147.6, 142.1, 137.6, 134.0, 132.1, 132.1, 127.4, 123.3, 112.7, 112.4, 101.0, 57.9, 50.6, 50.3, 45.4, 41.5, 34.3, 31.6, 30.2, 24.7. LRMS (ESI) m/z calcd for [C³⁴H³⁷N⁹O⁴+H]⁺=636.3, found 636.4.

An analogous procedure was followed to obtain the following compounds:

| | Starting | |
|---|---|---|
| Compound No. and Structure | Material | Characterization |
| Compound 101 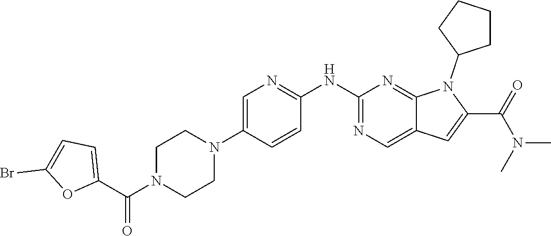 [SMILES: CN(C)C(=O)c1cc2cnc(Nc3ccc(N4CCN(C(=O)c5ccc(Br)o5)CC4)cn3)nc2n1C1CCCC1] | 5-Bromo-2- furoic acid (11 mg, 0.06 mmol) | The crude residue was purified by silica gel chromatography (0-15% DCM in MeOH) to afford 24 mg (63%) of Compound 101 as a film. ¹H NMR (500 MHz, CDCl₃) δ 8.72 (s, 1H), 8.39 (d, J = 9.0 Hz, 1H), 8.25 (s, 1H), 8.06 (d, J = 3.0 Hz, 1H), 7.34 (dd, J = 9.1, 3.0 Hz, 1H), 7.03 (d, J = 3.5 Hz, 1H), 6.48-6.42 (m, 2H), 4.79 (p, J = 8.9 Hz, 1H), 3.98 (s, 4H), 3.24-3.18 (m, 4H), 3.15 (s, 6H), 2.58 (m, 2H), 2.11- 2.01 (m, 4H), 1.76 - 1.68 (m, 2H). ¹³C NMR (151 MHz, CDCl₃) δ 164.1, 157.8, 154.6, 151.9, 151.9, 149.5, 147.7, 142.0, 137.5, 132.0, 127.3, 124.4, 119.3, 113.5, 112.6, |
| | | 112.5, 101.0, 57.9, 50.6, 30.2, 24.7. |
| | | LRMS (ESI) m/z calcd for |
| | | [C28H31BrN8O3 + H]⁺ = 607.3, |
| | | found 607.2. |
| Compound 102 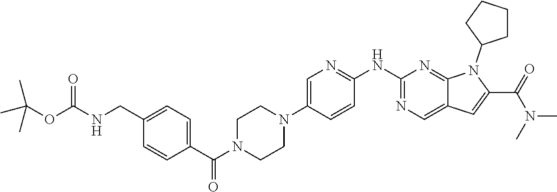 [SMILES: CN(C)C(=O)c1cc2cnc(Nc3ccc(N4CCN(C(=O)c5ccc(CNC(=O)OC(C)(C)C)cc5)CC4)cn3)nc2n1C1CCCC1] | 4-(Boc- aminomethyl) benzoic acid (15 mg, 0.06 mmol) | The crude residue was purified by silica gel chromatography (0-15% DCM in MeOH) to afford 30 mg (77%) of Compound 102 as a film. ¹H NMR (500 MHz, CDCl₃) δ 8.72 (s, 1H), 8.37 (d, J = 9.1 Hz, 1H), 8.26 (s, 1H), 8.04 (d, J = 2.9 Hz, 1H), 7.41 (d, J = 7.9 Hz, 2H), 7.36- 7.30 (m, 3H), 6.44 (s, 1H), 4.99 (d, J = 6.5 Hz, 1H), 4.79 (p, J = 9.0 Hz, 1H), 4.35 (d, J = 6.1 Hz, 2H), 3.94 (s, 2H), 3.62 (s, 2H), 3.20 (br s, 2H), 3.15 (s, 6H), 3.09 (br s, 2H), 2.58 (m, 2H), 2.11-2.00 (m, 4H), 1.76- |
| | | 1.67 (m, 2H), 1.46 (s, 9H). ¹³C |
| | | NMR (151 MHz, CDCl₃) δ 170.2, |
| | | 164.1, 156.0, 154.6, 151.9, 151.9, |
| | | 147.7, 142.0, 141.1, 137.6, 134.4, |
| | | 132.0, 127.5, 127.4, 112.7, 112.5, |
| | | 101.0, 79.7, 57.9, 50.6, 44.3, 39.4, |
| | | 35.2, 30.2, 28.4, 24.7. LRMS (ESI) |
| | | m/z calcd for [C₃₆H₄₅N₉O₄ + H]⁺ += |
| | | 668.4, found 668.5. |
| Compound 103 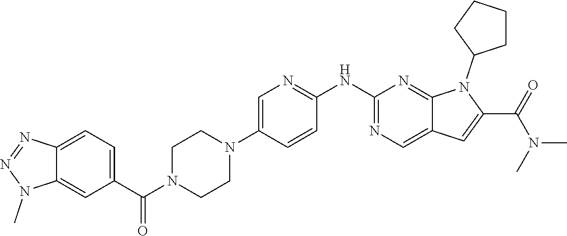 [SMILES: CN(C)C(=O)c1cc2cnc(Nc3ccc(N4CCN(C(=O)c5ccc6nnn(C)c6c5)CC4)cn3)nc2n1C1CCCC1] | 1-Methyl-1h- 1,2,3- benzotriazole- 6-carboxylic acid (10 mg, 0.06 mmol) | The crude residue was purified by silica gel chromatography (0-15% DCM in MeOH) to afford 24 mg (69%) of Compound 103 as a film. ¹H NMR (500 MHz, CDCl₃) δ 8.72 (s, 1H), 8.34 (s, 1H), 8.21 (s, 1H), 8.15 (d, J = 1.1 Hz, 1H), 8.04 (d, J = 2.9 Hz, 1H), 7.68-7.58 (m, 2H), 7.35 (dd, J = 9.0, 2.9 Hz, 1H), 6.44 (s, 1H), 4.79 (p, J = 8.9 Hz, 1H), 4.34 (s, 3H), 3.94 (br s, 2H), 3.70 (br s, 2H), 3.15 (m, 10H), 2.57 (m, 2H), 2.12-1.99 (m, 4H), 1.71 (d, J = 4.1 Hz, 2H). ¹³C NMR (151 MHz, CDCl₃) δ 169.7, 164.0, 154.4, 151.9, 145.3, 142.0, 134.1, 131.3, 127.0, 119.2, 112.8, 112.6, 109.9, |
| | | 101.0, 57.9, 50.6, 34.4, 31.9, 30.2, |
| | | 29.4, 24.7, 22.7, 14.2, 14.1. LRMS |
| | | (ESI) m/z calcd for [C₃₁H₃₅N₁₁O₂ + |
| | | H]⁺ = 594.3, found 594.3 |
| Compound 104 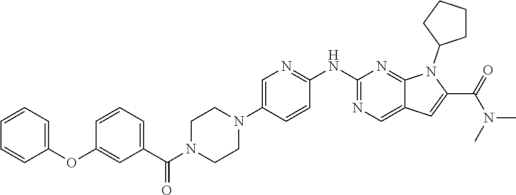 [SMILES: CN(C)C(=O)c1cc2cnc(Nc3ccc(N4CCN(C(=O)c5cccc(Oc6ccccc6)c5)CC4)cn3)nc2n1C1CCCC1] | 3-phenoxy benzoic acid (12 mg, 0.06 mmol) | The crude residue was purified by silica gel chromatography (0-15% DCM in MeOH) to afford 30 mg (83%) of Compound 104 as a film. ¹H NMR (500 MHz, CDCl₃) δ 8.72 (s, 1H), 8.39 (d, J = 9.0 Hz, 1H), 8.30 (s, 1H), 8.05 (d, J = 2.9 Hz, 1H), 7.47-7.42 (m, 2H), 7.42- 7.31 (m, 3H), 7.19-7.12 (m, 1H), 7.04 (ddd, J = 13.1, 7.6, 1.6 Hz, 4H), 6.44 (s, 1H), 4.79 (p, J = 8.9 Hz, 1H), 3.96-3.62 (m, 4H), 3.15 (m, 10H), 2.64-2.53 (m, 2H), 2.11- 2.00 (m, 4H), 1.76-1.65 (m, 2H). |
| | | ¹³C NMR (151 MHz, CDCl₃) δ |
| | | 170.1, 164.1, 159.1, 156.2, 154.6, |
| | | 151.9, 151.9, 147.7, 142.1, 137.6, |
| | | 132.0, 130.0, 129.8, 129.3, 127.4, |
| | | 124.1, 119.6, 118.1, 112.6, 112.5, |
| | | 101.1, 57.9, 50.7, 39.4, 35.2, 33.7, |
| | | 31.9, 30.2, 29.4, 26.7, 24.7, 23.2, |
| | | 22.7, 14.2, 14.1. (Mixture of |
| | | rotamers). LRMS (ESI) m/z calcd |
| | | for [C₃₆H₃₈N₈O₃ + H]⁺ = 631.3, |
| | | found 631.3. |
| Compound 105 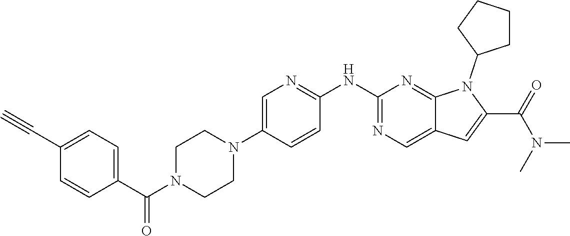 [SMILES: C#Cc1ccc(C(=O)N2CCN(c3ccc(Nc4ncc5cc(C(=O)N(C)C)n(C6CCCC6)c5n4)nc3)CC2)cc1] | 4-Ethynyl benzoic acid (8 mg, 0.06 mmol) | The crude residue was purified by silica gel chromatography (0-15% DCM in MeOH) to afford 14 mg (42%) of Compound 105 as a film. ¹H NMR (500 MHz, CDCl₃) δ 8.70 (s, 1H), 8.35 (d, J = 9.1 Hz, 1H), 8.16 (s, 1H), 7.99 (d, J = 2.9 Hz, 1H), 7.58-7.53 (m, 2H), 7.44-7.39 (m, 2H), 7.35 (dd, J = 9.1, 3.0 Hz, 1H), 6.45 (s, 1H), 4.79 (p, J = 9.0, 9.0, 9.0, 9.0 Hz, 1H), 3.93 (s, 2H), 3.61 (s, 2H), 3.16 (d, J = 3.9 Hz, 10H), 2.61 (s, 1H), 2.56 (dt, J = 16.4, 8.3, 8.3 Hz, 2H), 2.11-2.01 (m, 4H), 1.74-1.68 (m, 2H). ¹³C NMR (151 MHz, CDCl₃) δ 169.7, 164.0, 154.3, 151.9, 151.6, 142.0, |
| | | 135.6, 132.3, 127.2, 123.9, 113.9, |
| | | 112.7, 101.0, 82.8, 78.9, 57.9, 41.0, |
| | | 39.4, 31.9, 30.2, 29.4, 24.7, 22.7, |
| | | 14.1. LRMS (ESI) m/z calcd for |
| | | [C₃₂H₃₄N₈O₂ + H]⁺ = 563.3, found |
| | | 563.3. |
| Compound 106 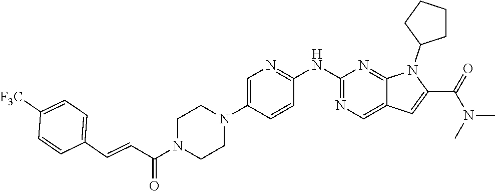 [SMILES: CN(C)C(=O)c1cc2cnc(Nc3ccc(N4CCN(C(=O)/C=C/c5ccc(C(F)(F)F)cc5)CC4)cn3)nc2n1C1CCCC1] | trans-4- (trifluoro- methyl) cinnamic acid (12 mg, 0.06 mmol) | The crude residue was purified by silica gel chromatography (0-15% DCM in MeOH) to afford 25 mg (68%) of Compound 106 as a film. ¹H NMR (500 MHz, CDCl₃) δ 8.73 (s, 1H), 8.17 (s, 1H), 8.00 (d, J = 2.9 Hz, 1H), 7.71 (d, J = 15.4 Hz, 1H), 7.64 (s, 4H), 7.44-7.37 (m, 1H), 7.01 (d, J = 15.5 Hz, 1H), 6.46 (s, 1H), 4.77 (q, J = 8.9 Hz, 1H), 3.99- 3.78 (m, 4H), 3.19 (br s, 4H), 3.15 (s, 6H), 2.53 (m, 2H), 2.10-1.97 (m, 4H), 1.69 (m, 2H). ¹³C NMR (151 MHz, CDCl₃) δ 165.0, 163.8, |
| | | 151.9, 142.1, 141.5, 138.5, 131.3 (q, |
| | | ⁴J_{CF} = 32.5 Hz), 129.5, 128.0, 125.8 |
| | | (q, ³J_{CF} = 3.7 Hz), 123.9 (q, ¹J_{CF} = |
| | | 272.2 Hz), 119.3, 113.2, 101.1, |
| | | 58.0, 45.7, 42.1, 39.4, 35.2, 33.7, |
| | | 31.9, 30.3, 30.2, 29.4, 24.7, 22.7. |
| | | HRMS (ESI) m/z calcd for |
| | | [C₃₃H₃₅F₃N₈O₂ + H]⁺ = 633.2835, |
| | | found 633.2906. |
| Compound 107 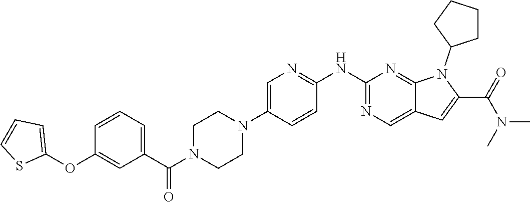 [SMILES: CN(C)C(=O)c1cc2cnc(Nc3ccc(N4CCN(C(=O)c5cccc(Oc6cccs6)c5)CC4)cn3)nc2n1C1CCCC1] | 4-(2-thienyl methyl)benzo- ic acid (13 mg, 0.06 mmol) | The crude residue was purified by silica gel chromatography (0-15% DCM in MeOH) to afford 32 mg (88%) of Compound 107 as a film. ¹H NMR (500 MHz, CDCl₃) δ 8.65 (s, 1H), 8.35 (d, J = 9.1 Hz, 1H), 7.89 (d, J = 2.9 Hz, 1H), 7.35-7.28 (m, 3H), 7.27-7.21 (m, 2H), 7.10 (dd, J = 5.2, 1.2 Hz, 1H), 6.87 (dd, J = 5.2, 3.4 Hz, 1H), 6.75 (dd, J = 3.3, 1.2 Hz, 1H), 6.38 (s, 1H), 4.73 (p, J = 8.8 Hz, 1H), 4.12 (s, 2H), 3.94-3.50 (m, 4H), 3.08 (s, 10H), 2.54-2.43 (m, 2H), 2.06-1.91 (m, 4H), 1.70-1.60 (m, 2H). ¹³C NMR |
| | | (151 MHz, CDCl₃) δ 170.4, 163.9, |
| | | 154.0, 151.9, 151.3, 147.1, 143.0, |
| | | 142.5, 142.1, 133.6, 132.6, 128.8, |
| | | 128.4, 127.5, 126.9, 125.5, 124.2, |
| | | 113.1, 113.0, 100.9, 57.9, 50.4, |
| | | 39.4, 35.8, 35.2, 31.9, 30.3, 30.2, |
| | | 29.4, 24.8, 23.2, 22.7. LRMS (ESI) |
| | | m/z calcd for [C₃₅H₃₈N₈O₂S + H]⁺ = |
| | | 635.3, found 635.4. |
| Compound 109 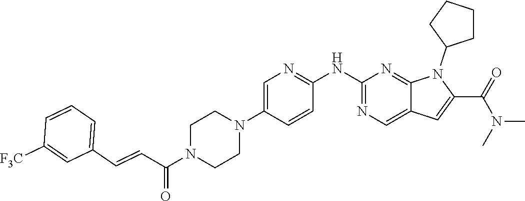 [SMILES: CN(C)C(=O)c1cc2cnc(Nc3ccc(N4CCN(C(=O)/C=C/c5cccc(C(F)(F)F)c5)CC4)cn3)nc2n1C1CCCC1] | 3-(trifluoro- methyl) cinnamic acid (12 mg, 0.06 mmol) | The crude residue was purified by silica gel chromatography (0-10% DCM in MeOH) to afford 30 mg (83%) of Compound 109 as a film. ¹H NMR (500 MHz, CDCl₃) δ 8.65 (s, 1H), 8.32 (d, J = 9.0 Hz, 1H), 8.12 (s, 1H), 7.97 (d, J = 2.9 Hz, 1H), 7.72 (d, J = 2.1 Hz, 1H), 7.66 (d, J = 15.4 Hz, 1H), 7.62 (d, J = 7.7 Hz, 1H), 7.55 (d, J = 7.8 Hz, 1H), 7.45 (t, J = 7.8, 7.8 Hz, 1H), 7.29 (dd, J = 9.1, 3.0 Hz, 1H), 6.92 (d, J = 15.4 Hz, 1H), 6.38 (s, 1H), 4.73 (p, J = 9.0, 9.0, 8.9, 8.9 Hz, 1H), 3.91-3.76 (m, 4H), 3.13 (t, J = 5.0, |
| | | 5.0 Hz, 4H), 3.09 (s, 6H), 2.57-2.46 |
| | | (m, 2H), 2.05-1.95 (m, 4H), 1.69- |
| | | 1.62 (m, 2H). ¹³C NMR (151 MHz, |
| | | CDCl₃) δ 164.9, 164.1, 154.5, |
| | | 151.9, 151.8, 147.6, 142.0, 141.5, |
| | | 136.0, 132.1, 131.4 (q, ²J_{CF} = 31.7 |
| | | Hz), 131.3, 129.4, 127.5, 126.1 (q, |
| | | ³J_{CF} = 3.7 Hz), 123.9 (q, ³J_{CF} = 3.8 |
| | | Hz), 123.9 (q, ¹J_{CF} = 272.6 Hz), |
| | | 118.7, 112.7, 112.5, 101.0, 57.9, |
| | | 50.8, 50.4, 45.9, 42.2, 39.5, 35.2, |
| | | 31.9, 30.2, 29.4, 24.7, 22.7, 14.1. |
| | | HRMS (ESI) m/z calcd for |
| | | [C₃₃H₃₅F₃N₈O₂ + H]⁺ = 633.2835, |
| | | found 633.2906. |
| Compound 110 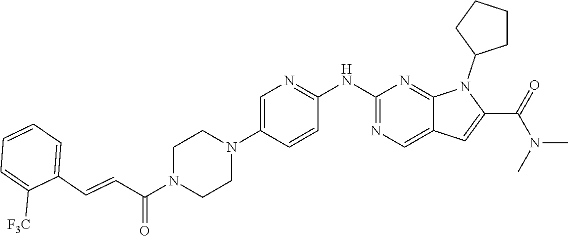 [SMILES: CN(C)C(=O)c1cc2cnc(Nc3ccc(N4CCN(C(=O)/C=C/c5ccccc5C(F)(F)F)CC4)cn3)nc2n1C1CCCC1] | trans-2- (trifluoro- methyl) cinnamic acid (12 mg, 0.06 mmol) | The crude residue was purified by silica gel chromatography (0-10% DCM in MeOH) to afford 26 mg (72%) of Compound 110 as a film. ¹H NMR (500 MHz, CDCl₃) δ 8.65 (s, 1H), 8.36 (d, J = 9.2 Hz, 1H), 7.96-7.87 (m, 2H), 7.66-7.60 (m, 2H), 7.50 (t, J = 7.6 Hz, 1H), 7.43- 7.32 (m, 2H), 6.76 (d, J = 15.3 Hz, 1H), 6.39 (s, 1H), 4.73 (p, J = 8.7 Hz, 1H), 3.92-3.71 (m, 4H), 3.12 (t, J = 5.1 Hz, 4H), 3.08 (s, 6H), 2.48 (m, 2H), 2.06-1.95 (m, 4H), 1.71- 1.59 (m, 2H). ¹³C NMR (151 MHz, CDCl₃) δ 165.0, 163.9, 154.0, 151.9, 151.3, 147.1, 142.0, 138.4, 134.4, 132.6, 132.0, 129.1, 128.7 (q, |
| | | ²J_{CF} = 30.3 Hz), 127.9, 126.2 (q, |
| | | ³J_{CF} = 5.5 Hz), 124.0 (d, ¹J_{CF} = |
| | | 274.0 Hz), 121.9, 113.2, 113.0, |
| | | 100.9, 58.0, 31.9, 30.3, 24.8, 22.7, |
| | | 14.1. HRMS (ESI) m/z calcd for |
| | | [C₃₃H₃₅F₃N₈O₂ + H] = 633.2835, |
| | | found 633.2906. |
| Compound 111 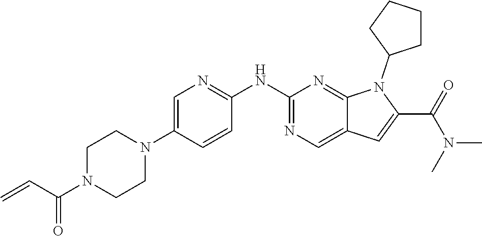 [SMILES: C=CC(=O)N1CCN(c2ccc(Nc3ncc4cc(C(=O)N(C)C)n(C5CCCC5)c4n3)nc2)CC1] | acrylic acid (0.004 mL, 0.06 mmol) | The crude residue was purified by silica gel chromatography (0-10% DCM in MeOH) to afford 15 mg (54%) of Compound 111 as a film. ¹H NMR (700 MHz, CDCl₃) δ 8.7 (s, 1H), 7.9 (br s, 2H), 7.6-7.4 (m, 1H), 6.6 (dd, J = 16.8, 10.6 Hz, 1H), 6.5 (s, 1H), 6.2 (d, J = 16.8 Hz, 1H), 5.7 (d, J = 10.6 Hz, 1H), 4.7 (p, J = 9.0 Hz, 1H), 3.8 (t, J = 5.2 Hz, 2H), 3.7 (q, J = 9.4 Hz, 2H), 3.1-3.1 (m, 8H), 3.0-2.9 (m, 2H), 2.4 (p, J = 8.3 Hz, 2H), 2.0-2.0 (m, 2H), 2.0 (q, J = 7.7 Hz, 2H), 1.6 (h, J = 8.0 Hz, 2H). ¹³C NMR (126 MHz, CDCl₃) δ 165.9, 163.8, 151.9, 149.9, 145.9, 142.0, 128.7, 127.0, |
| | | 113.8, 101.2, 58.1, 49.7, 49.3, 45.5, |
| | | 41.7, 39.4, 35.2, 30.3, 29.6, 24.6. |
| | | LRMS (ESI) m/z calcd for |
| | | [C₂₆H₃₂N₈O₂ + H]⁺ = 489.3, found |
| | | 489.4. |
| Compound 112 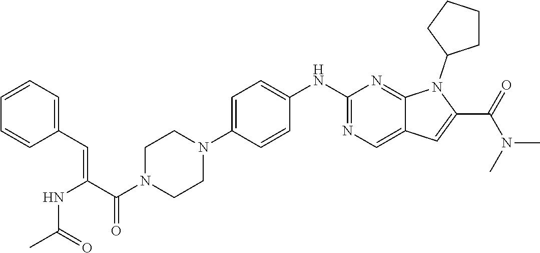 [SMILES: CC(=O)N/C(=C\c1ccccc1)C(=O)N1CCN(c2ccc(Nc3ncc4cc(C(=O)N(C)C)n(C5CCCC5)c4n3)cc2)CC1] | 2-(acetyl amino)-3- phenyl-2- propenoic acid (13 mg, 0.06 mmol) | The crude residue was purified by silica gel chromatography (0-10% DCM in MeOH) to afford 34 mg (96%) of Compound 112 as a film. ¹H NMR (700 MHz, DMSO) δ 10.84 (s, 1H), 10.11 (s, 1H), 8.93 (s, 1H), 7.95 (d, J = 2.6 Hz, 2H), 7.75 (d, J = 8.9 Hz, 1H), 7.68 (d, J = 8.4 Hz, 2H), 7.63 (d, J = 8.3 Hz, 2H), 7.49 (d, J = 15.2 Hz, 1H), 7.23 (d, J = 15.3 Hz, 1H), 6.76 (s, 1H), 4.80 (p, J = 8.9 Hz, 1H), 3.91 (s, 2H), 3.77 (s, 2H), 3.22 (s, 4H), 3.06 (s, 6H), 2.40-2.31 (m, 2H), 2.07 (s, 3H), 2.05-2.00 (m, 2H), 1.99- 1.93 (m, 2H), 1.66 (m, 2H). ¹³C NMR (151 MHz, DMSO) & 169.0, 165.2, 162.9, 153.0, 152.0, 149.7, |
| | | 145.1, 142.4, 142.1, 141.1, 134.9, |
| | | 130.2, 129.3, 119.3, 116.5, 115.0, |
| | | 113.9, 101.4, 79.6, 57.5, 49.5, 49.1, |
| | | 48.8, 45.1, 41.7, 39.2, 35.0, 30.5, |
| | | 24.7, 24.5. LRMS (ESI) m/z calcd |
| | | for [C₃₅H₄₀N₈O₃ + H]⁺ = 621.8, |
| | | found 622.3. |
| Compound 114 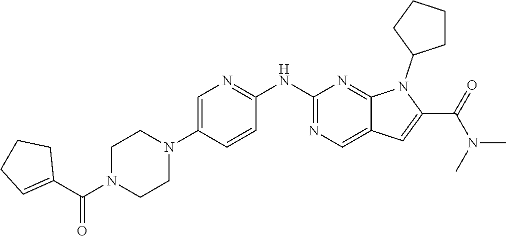 [SMILES: CN(C)C(=O)c1cc2cnc(Nc3ccc(N4CCN(C(=O)C5=CCCC5)CC4)cn3)nc2n1C1CCCC1] | 1-cyclo- pentene carboxylic acid (7 mg, 0.06 mmol) | The crude residue was purified by silica gel chromatography (0-10% DCM in MeOH) to afford 23 mg (77%) of Compound 114 as a powder. ¹H NMR (500 MHz, CDCl₃) δ 8.75 (s, 1H), 8.02 (s, 1H), 7.94 (d, J = 2.9 Hz, 1H), 7.50-7.42 (m, 1H), 6.47 (s, 1H), 5.93 (p, J = 2.2 Hz, 1H), 4.77 (p, J = 8.9 Hz, 1H), 3.78 (s, 4H), 3.21-3.08 (m, 10H), 2.63 (tq, J = 7.5, 2.4 Hz, 2H), 2.54-2.42 (m, 4H), 2.12-1.99 (m, 4H), 1.96 (p, J = 7.6 Hz, 2H), 1.67 (h, J = 8.4 Hz, 2H). ¹³C NMR (151 MHz, CDCl₃) δ 168.6, 163.6, 151.9, 142.2, 137.9, 133.4, 113.6, 101.1, 58.0, 54.0, 49.8, 39.4, 35.2, 34.5, |
| | | 33.3, 30.4, 24.7, 22.8, 18.6, 17.3, |
| | | 14.1. LRMS (ESI) m/z calcd for |
| | | [C₂₉H₃₆N₈O₂ + H] = 529.3, found |
| | | 529.4. |
| Compound 115 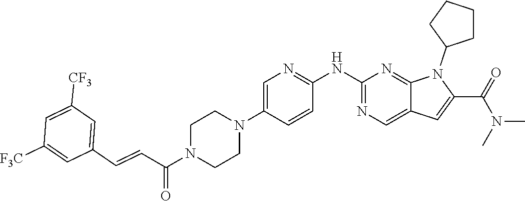 [SMILES: CN(C)C(=O)c1cc2cnc(Nc3ccc(N4CCN(C(=O)/C=C/c5cc(C(F)(F)F)cc(C(F)(F)F)c5)CC4)cn3)nc2n1C1CCCC1] | trans-3,5- bis(trifluoro- methyl) cinnamic acid (15 mg, 0.06 mmol) | The crude residue was purified by silica gel chromatography (0-10% DCM in MeOH) to afford 15 mg (40%) of Compound 115 as a film. ¹H NMR (500 MHz, CDCl₃) δ 8.65 (s, 1H), 8.28 (s, 1H), 7.97 (d, J = 3.0 Hz, 2H), 7.87 (s, 2H), 7.79 (s, 1H), 7.68 (d, J = 15.4 Hz, 1H), 7.30 (dd, J = 9.2, 2.9 Hz, 1H), 6.99 (d, J = 15.4 Hz, 1H), 6.38 (s, 1H), 4.73 (t, J = 8.9 Hz, 1H), 3.88 (s, 2H), 3.82 (d, J = 13.7 Hz, 2H), 3.13 (d, J = 8.6 Hz, 4H), 3.09 (s, 6H), 2.50 (dq, J = 15.6, 7.3 Hz, 2H), 2.06-1.92 (m, 4H), 1.65 (dt, J = 12.6, 5.1 Hz, 2H). |
| | | ¹³C NMR (151 MHz, CDCl₃) δ |
| | | 164.3, 164.0, 151.9, 142.0, 139.8, |
| | | 137.3, 132.4 (q, ²J_{CF} = 33.4 Hz), |
| | | 127.4, 123.1 (q, ¹J_{CF} = 273.0 Hz), |
| | | 122.9, 120.8, 112.9, 112.6, 101.0, |
| | | 57.9, 50.3, 45.9, 42.3, 31.9, 30.2, |
| | | 29.4, 24.7, 22.7, 14.1. LRMS (ESI) |
| | | m/z calcd for [C₃₄H₃₄F₆N₈O₂ + H] = |
| | | 701.3, found 701.4. |
| Compound 116 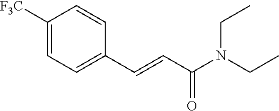 [SMILES: CCN(CC)C(=O)/C=C/c1ccc(C(F)(F)F)cc1] | trans-4- (trifluoro- methyl) cinnamic acid (100 mg, 0.06 mmol) | Reaction ran with N,N-diethylamine (0.05, 0.48 mmol), in place of ribociclib. The crude residue was purified by silica gel chromatography (0-60% EtOAC in Hexanes) to afford 66 mg (50%) of Compound 116 as a film. ¹H NMR (500 MHz, CDCl₃) δ 7.63 (d, J = 15.4 Hz, 1H), 7.54 (s, 4H), |
| | | 6.83 (d, J = 15.4 Hz, 1H), 3.42 (dq, |
| | | J = 9.8, 7.1 Hz, 4H), 1.19 (t, J = 7.2 |
| | | Hz, 3H), 1.12 (t, J = 7.1 Hz, 3H). |
| | | ¹³C NMR (126 MHz, CDCl₃) δ |
| | | 165.1, 140.5, 138.9 (d, ⁵J_{CF} = 1.4 |
| | | Hz), 131.0 (q, ²J_{CF} = 32.5 Hz), |
| | | 127.9, 125.7 (q, ³J_{CF} = 3.8 Hz), |
| | | 123.9 (q, ¹J_{CF} = 272.1 Hz), 120.3, |
| | | 42.4, 41.2, 15.1, 13.1. LRMS (ESI) |
| | | m/z calcd for [C₁₄H₁₆F₃NO + H]⁺ = |
| | | 272.1, found 272.2. |
| Compound 135 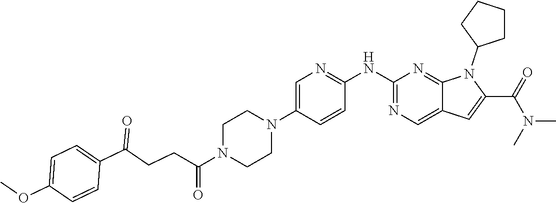 | 3-(4-Meth- oxybenzoyl) propionic acid (16 mg, 0.08 mmol), HATU (26 mg, 0.07 mmol), DIPEA (0.04 mL, 0.23 mmol), and ribociclib (28 mg, 0.06 | A precipitate had formed during the reaction which was vacuum filtered and washed with hexanes. The precipitate was dissolved in DCM, dry loaded onto silica gel and was purified by silica gel chromatography (0-7% MeOH in DCM) to afford 29 mg (73%) of Compound 135 as a powder. ¹H NMR (700 MHz, CDCl₃) δ 8.74 (s, 1H), 8.43 (s, 1H), 8.38 (d, J = 9.0 Hz, 1H), 8.07 (d, J = 2.9 Hz, 1H), 8.02-7.98 (m, 2H), 7.33 (dd, J = 9.1, 2.9 Hz, 1H), 6.95-6.91 (m, 2H), 6.44 (s, 1H), 4.79 (p, J = 8.9 |
| | mmol). | Hz, 1H), 3.86 (s, 3H), 3.81 (t, J = 5.1 |
| | | Hz, 2H), 3.76 (t, J = 5.1 Hz, 2H), |
| | | 3.34 (t, J = 6.6 Hz, 2H), 3.18 (t, J = |
| | | 5.1 Hz, 2H), 3.15 (s, 6H), 3.11 (t, J = |
| | | 5.2 Hz, 2H), 2.82 (t, J = 6.6 Hz, |
| | | 2H), 2.62-2.54 (m, 2H), 2.11- |
| | | 2.00 (m, 4H), 1.75-1.67 (m, 2H). |
| | | ¹³C NMR (151 MHz, CDCl₃) δ |
| | | 197.6, 170.4, 164.1, 163.5, 154.6, |
| | | 152.0, 151.9, 147.6, 142.1, 137.5, |
| | | 132.0, 130.4, 129.9, 127.3, 113.7, |
| | | 112.6, 112.4, 101.0, 57.9, 55.5, |
| | | 50.6, 50.3, 45.4, 41.7, 33.2, 30.2, |
| | | 27.1, 24.7. HRMS (ESI) m/z calcd |
| | | for [C₃₄H₄₀N₈O₄ + H]⁺ = 625.3173, |
| | | found 625.3242. |
| Compound 136 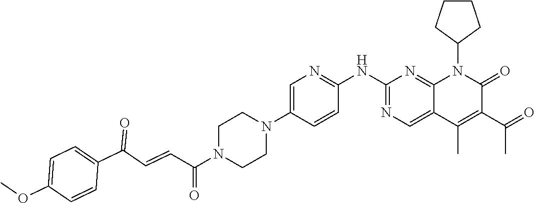 [SMILES: COc1ccc(C(=O)/C=C/C(=O)N2CCN(c3ccc(Nc4ncc5c(C)c(C(C)=O)c(=O)n(C6CCCC6)c5n4)nc3)CC2)cc1] | 4-methoxy cinnamic acid (14 mg, 0.06 mmol), HATU (43 mg, 0.11 mmol), DIPEA (0.04 mL, 0.23 mmol), and Palbociclib (25 mg, | The crude residue was purified by silica gel chromatography (0-10% MeOH in DCM) to afford 26 mg (74%) of Compound 136 as a film. ¹H NMR (500 MHz, CDCl₃) δ 8.86 (s, 1H), 8.22 (d, J = 9.0 Hz, 1H), 8.09 (d, J = 2.9 Hz, 1H), 8.08-8.02 (m, 2H), 8.00 (d, J = 14.9 Hz, 1H), 7.52 (d, J = 14.8 Hz, 1H), 7.35 (dd, J = 9.1, 3.0 Hz, 1H), 7.01-6.95 (m, 2H), 5.88 (p, J = 8.9 Hz, 1H), 3.94 (t, J= 5.2 Hz, 2H), 3.89 (s, 3H), 3.85 (t, J = 5.1 Hz, 2H), 3.22 (t, J = 5.2 Hz, 4H), 2.54 (s, 3H), 2.38 (s, 3H), 2.36-2.30 (m, 1H), 2.12-2.01 (m, |
| | 0.06 | 2H), 1.89 (dddd, J = 15.4, 13.0, 7.6, |
| | mmol). | 4.7 Hz, 2H), 1.75-1.64 (m, 3H). |
| | | ¹³C NMR (151 MHz, CDCl₃) δ |
| | | 202.6, 187.6, 164.3, 164.1, 161.4, |
| | | 158.0, 157.2, 155.5, 145.8, 143.0, |
| | | 141.7, 137.3, 134.9, 131.3, 131.1, |
| | | 130.9, 129.9, 126.9, 114.2, 113.6, |
| | | 107.9, 55.6, 54.1, 53.4, 50.4, 49.8, |
| | | 45.8, 42.1, 31.5, 28.1, 25.8, 22.7, |
| | | 14.0. HRMS (ESI) m/z calcd for |
| | | [C₃₅H₃₇N₇O₅ + H]⁺ = 636.2929, |
| | | found 636.2935. |
| Compound 170 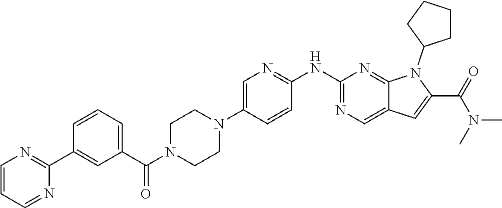 [SMILES: CN(C)C(=O)c1cc2cnc(Nc3ccc(N4CCN(C(=O)c5cccc(-c6ncccn6)c5)CC4)cn3)nc2n1C1CCCC1] | 3-(2- pyrimidinyl) benzoic acid (12 mg, 0.06 mmol) | The crude residue was purified by silica gel chromatography (0-15% DCM in MeOH) to afford 12 mg (34%) of Compound 170 as a film. ¹H NMR (500 MHz, CDCl₃) δ 8.83 (d, J = 4.8 Hz, 2H), 8.69 (s, 1H), 8.54 (dt, J = 8.9, 1.7 Hz, 2H), 8.38 (d, J = 9.1 Hz, 1H), 8.01 (d, J = 2.9 Hz, 1H), 7.92 (s, 1H), 7.63-7.54 (m, 2H), 7.35 (dd, J = 9.1, 2.9 Hz, 1H), 7.23 (t, J = 4.8 Hz, 1H), 6.44 (s, 1H), 4.79 (p, J = 8.9 Hz, 1H), 4.01 (br s, 2H), 3.69 (br s, 2H), 3.25 (br s, 2H), 3.15 (s, 6H), 3.13 (br s, 2H), 2.64-2.52 (m, 2H), 2.12- |
| | | 1.99 (m, 4H), 1.77-1.69 (m, 2H). |
| | | ¹³C NMR (151 MHz, CDCl₃) δ |
| | | 170.1, 164.1, 163.9, 157.4, 154.5, |
| | | 151.9, 151.8, 147.6, 142.2, 137.9, |
| | | 135.9, 132.1, 129.5, 129.5, 129.1, |
| | | 127.4, 126.8, 119.5, 112.7, 112.5, |
| | | 101.0, 57.9, 31.9, 30.2, 29.4, 24.7, |
| | | 22.7, 14.1. LRMS (ESI) m/z calcd |
| | | for [C₃₄H₃₆N₁₀O₂ + H]⁺ = 617.3, |
| | | found 617.3. |

### Example 2: Synthesis of Compound 108

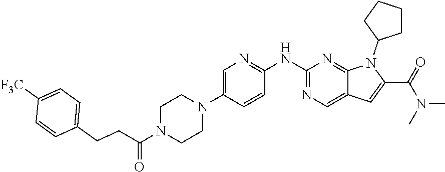 [SMILES: CN(C)C(=O)c1cc2cnc(Nc3ccc(N4CCN(C(=O)CCc5ccc(C(F)(F)F)cc5)CC4)cn3)nc2n1C1CCCC1]

General Procedure A was followed with 4-(trifluoromethyl)hydrocinnamic acid (100 mg, 0.46 mmol), HATU (350 mg, 0.92 mmol), DIPEA (0.32 mL, 1.4 mmol), and ribociclib (200 mg, 0.46 mmol). The crude residue was purified by reverse phase silica gel chromatography (595 MeCN in H₂O) to afford 133 mg (460%) of the Compound 108 as a film. ¹H NMR (500 MHz, CDCl₃) δ 9.93 (s, 1H), 8.76 (s, 1H), 7.79 (s, 1H), 7.54 (d, J=8.2 Hz, 2H), 7.47 (d, J=7.9 Hz, 2H), 7.32 (d, J=7.9 Hz, 2H), 6.48 (s, 1H), 4.73 (p, J=8.8 Hz, 1H), 3.74 (t, J=5.0 Hz, 2H), 3.58 (t, J=4.6 Hz, 2H), 3.12 (d, J=13.6 Hz, 6H), 3.08-3.00 (m, 6H), 2.71 (t, J=7.5 Hz, 2H), 2.38 (m, 2H), 2.05-1.91 (m, 4H), 1.64-1.56 (m, 2H). ¹³C NMR (151 MHz, CDCl₃) δ 170.5, 163.2, 152.4, 151.8, 145.3, 142.1, 128.9, 128.4 (q, ²J_{CF}=32.2 Hz), 125.4 (q, ³J_{CF}=3.7 Hz), 124.3 (q, ¹J_{CF}=271.8 Hz), 114.7, 114.3, 101.1, 67.1, 58.1, 48.8, 45.0, 41.2, 39.3, 35.1, 34.1, 30.9, 30.5, 24.7. HRMS (ESI) m/z calcd for [C₃₃H₃₇F₃N₈O₂+H]⁺=635.2992, found 635.3061.

### Example 3: Synthesis of Compound 113

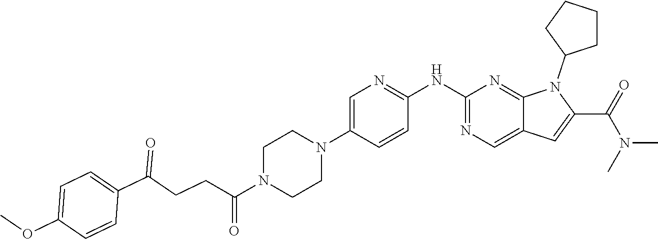

General Procedure D was followed with trans-3-(4-methoxybenzoyl)acrylic acid (100 mg, 0.5 mmol), T3P (0.5 mL, 0.7 mmol), DIPEA (0.25 mL, 1.5 mmol), and ribociclib (216 mg, 0.6 mmol). The crude residue was purified by silica gel chromatography (0-10% DCM in MeOH). The resultant oil was triturated in diethyl ether to afford (E)-7-cyclopentyl-2-((5-(4-(4-(4-methoxyphenyl)-4-oxobut-2-enoyl)piperazin-1-yl)pyridin-2-yl)-amino)-N,N-dimethyl-7H-pyrrolo[2,3-d]pyrimidine-6-carboxamide (Compound 113, 199 mg, 83%) as a powder. HRMS (ESI) m/z calcd for [C₃₄H₃₈N₈O₄+H]⁺=623.3016, found 623.3086. ¹H NMR (500 MHz, CDCl₃) δ 8.75 (s, 2H), 8.28 (d, J=8.1 Hz, 1H), 8.06-8.01 (m, 3H), 7.98 (d, J=14.9 Hz, 1H), 7.50 (d, J=15.0 Hz, 1H), 7.36 (dd, J=9.1, 2.9 Hz, 1H), 7.00-6.93 (m, 2H), 6.45 (s, 1H), 4.78 (p, J=8.9 Hz, 1H), 3.92 (t, J=5.2 Hz, 2H), 3.88 (s, 3H), 3.82 (t, J=5.1 Hz, 2H), 3.17 (t, J=5.2 Hz, 4H), 3.14 (s, 6H), 2.60-2.49 (m, 2H), 2.11-1.97 (m, 4H), 1.73-1.63 (m, 2H). ¹³C NMR (126 MHz, CDCl₃) δ 187.6, 164.2, 164.1, 164.1, 154.7, 152.0, 151.9, 148.0, 141.8, 137.7, 134.7, 131.9, 131.3, 129.9, 127.4, 114.1, 112.6, 112.5, 101.1, 457.9, 55.6, 50.9, 50.3, 46.0, 42.2, 30.1, 24.7.

### Example 4: Synthesis of Compound 134

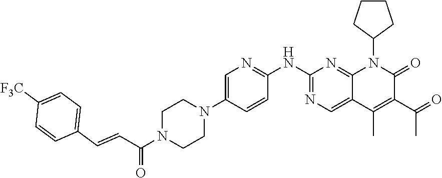 [SMILES: CC(=O)c1c(C)c2cnc(Nc3ccc(N4CCN(C(=O)/C=C/c5ccc(C(F)(F)F)cc5)CC4)cn3)nc2n(C2CCCC2)c1=O]

General Procedure A was followed with trans-4-(trifluoromethyl)cinnamic acid (15 mg, 0.07 mmol), HATU (43 mg, 0.11 mmol), DIPEA (0.04 mL, 0.23 mmol), and palbociclib (0.25, 0.06 mmol). The crude residue was purified by silica gel chromatography (0-100% EtOAc in Hexanes) to afford 23 mg (63%) of Compound 134 as a film. ¹H NMR (500 MHz, CDCl₃) δ 8.87 (s, 1H), 8.74 (s, 1H), 8.22 (d, J=9.1 Hz, 1H), 8.09 (d, J=2.9 Hz, 1H), 7.72 (d, J=15.4 Hz, 1H), 7.64 (s, 4H), 7.37 (dd, J=9.1, 3.0 Hz, 1H), 7.00 (d, J=15.5 Hz, 1H), 5.88 (p, J=8.9 Hz, 1H), 4.01-3.81 (m, 4H), 3.23 (t, J=5.1 Hz, 4H), 2.54 (s, 3H), 2.38 (s, 3H), 2.37-2.31 (m, 2H), 2.07 (tdd, J=11.9, 9.9, 5.2 Hz, 2H), 1.92-1.84 (m, 2H), 1.69 (tdd, J=10.9, 6.6, 4.0 Hz, 2H). ¹³C NMR (126 MHz, CDCl₃) δ 202.6, 164.9, 161.4, 158.0, 157.2, 155.6, 145.8, 143.1, 141.8, 141.6, 138.5 (d, ⁵J_{CF}=1.4 Hz), 137.1, 131.4 (q, ²J_{CF}=32.7 Hz), 130.9, 128.0, 126.9, 125.8 (q, ³J_{CF}=3.8 Hz), 123.9 (d, ¹J_{CF}=272.1 Hz), 119.2, 113.7, 107.9, 54.1, 50.3, 49.8, 45.7, 42.1, 31.5, 28.1, 25.8, 14.0. HRMS (ESI) m/z calcd for [C₃₄H₃₄F₃N₇O₃+H]⁺=646.2748, found 646.2741.

### Example 5: Synthesis of Compound 117

### Synthesis of Compound 300

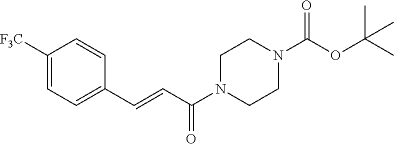 [SMILES: CC(C)(C)OC(=O)N1CCN(C(=O)/C=C/c2ccc(C(F)(F)F)cc2)CC1]

General Procedure A was followed with trans-4-(trifluoromethyl)cinnamic acid (100 mg, 0.5 mmol), HATU (44 mg, 0.12 mmol), DIPEA (0.04 mL, 0.23 mmol), and 1-boc-piperazine (95 mg, 0.5 mmol). The crude residue was purified by silica gel chromatography (0-70% EtOAc in Hexanes) to afford 73 mg (41%) of Compound 300 as a powder. ¹H NMR (500 MHz, CDCl₃) δ 7.62 (d, J=15.4 Hz, 1H), 7.55 (s, 4H), 6.87 (d, J=15.4 Hz, 1H), 3.60 (s, 4H), 3.42 (dd, J=6.6, 3.9 Hz, 4H), 1.41 (s, 9H). ¹³C NMR (151 MHz, CDCl₃) δ 165.0, 154.5, 141.4, 138.5, 131.3 (q, ²J_{CF}=32.6 Hz), 127.9, 125.8 (q, ³J_{CF}=3.7 Hz), 123.9 (q, ¹J_{CF}=272.3 Hz), 119.3, 80.4, 45.7, 42.0, 28.4. LRMS (ESI) m/z calcd for [C₁₉H₂₃F₃N₂O₃+Na]⁺=407.2, found 407.2.

### Synthesis of Compound 117

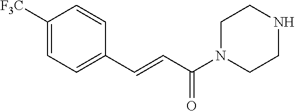 [SMILES: O=C(/C=C/c1ccc(C(F)(F)F)cc1)N1CCNCC1]

General Procedure E was followed with Compound 300 (47 mg, 0.12 mmol), and TFA (0.3 mL, 3.9 mmol) for 2 hours. The crude residue was purified by silica gel chromatography (0-17% DCM in MeOH) using a Biotage® Sfar KP-Amino D cartridge to afford 15 mg (41%) of Compound 117 as a film. ¹H NMR (500 MHz, CDCl₃) δ: 7.64 (d, J=15.5 Hz, 1H), 7.59 (s, 4H), 6.93 (d, J=15.4 Hz, 1H), 3.72-3.66 (m, 2H), 3.64-3.58 (m, 2H), 2.89 (t, J=5.0, 5.0 Hz, 4H), 1.80 (s, 1H). ¹³C NMR (151 MHz, CDCl₃) δ: 164.9, 140.8, 138.7, 131.1 (q, ²J_{CF}=32.6 Hz), 127.8, 125.7 (q, ³J_{CF}=3.8 Hz), 123.9 (q, ¹J_{CF}=272.0 Hz), 119.8, 47.2, 46.6, 45.9, 43.4. LRMS (ESI): m/z calcd for [C₁₄H₁₅F₃N₂O+H]⁺=285.1, found 285.2.

### Example 6: Synthesis of Compound 119

### Synthesis of Compound 118

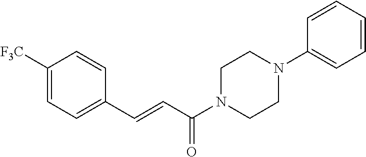 [SMILES: O=C(/C=C/c1ccc(C(F)(F)F)cc1)N1CCN(c2ccccc2)CC1]

General Procedure A was followed with trans-4-(trifluoromethyl)cinnamic acid (200.0 mg, 0.9 mmol), HATU (704 mg, 1.9 mmol), DIPEA (0.6 mL, 3.7 mmol), and 1-phenylpiperazine (150 mg, 0.9 mmol). The crude residue was purified by silica gel chromatography (15-55% EtOAc in Hexanes) to afford 125 mg (37%) of Compound 118 as a powder. ¹H NMR (500 MHz, CDCl₃) δ 7.62 (d, J=15.4 Hz, 1H), 7.53 (s, 4H), 7.23-7.16 (m, 2H), 6.91 (d, J=15.4 Hz, 1H), 6.83 (dd, J=16.5, 7.9 Hz, 3H), 3.80 (br s, 2H), 3.72 (br s, 2H), 3.13 (m, 4H). ¹³C NMR (126 MHz, CDCl₃) δ 164.9, 150.9, 141.3, 138.7, 131.2 (q, ²J_{CF}=32.5 Hz), 129.3, 128.0, 125.8 (q, ³J_{CF}=3.8 Hz), 124.0 (q, ¹J_{CF}=272.2 Hz), 120.6, 119.6, 116.7, 49.9, 49.4, 45.9, 42.2. LRMS (ESI) m/z calcd for [C₂₀H₁₉F₃N₂O+H]⁺=361.1, found 361.2.

### Synthesis of Compound 119

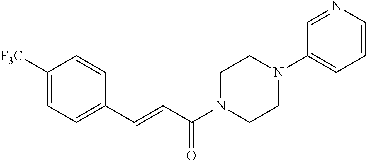 [SMILES: O=C(/C=C/c1ccc(C(F)(F)F)cc1)N1CCN(c2cccnc2)CC1]

General Procedure A was followed with trans-4-(trifluoromethyl)cinnamic acid (200 mg, 0.9 mmol), HATU (704 mg, 1.9 mmol), DIPEA (0.64 mL, 3.7 mmol), and 1-(3-pyridinyl)piperazine (151 mg, 0.9 mmol). The crude residue was purified by silica gel chromatography (0-10% DCM in MeOH) to afford 53 mg (16%) of Compound 119 as a powder. ¹H NMR (500 MHz, CDCl₃) δ: 8.34-8.29 (m, 1H), 8.14 (dd, J=3.9, 2.1 Hz, 1H), 7.70 (d, J=15.4 Hz, 1H), 7.61 (s, 4H), 7.23-7.14 (m, 2H), 6.98 (d, J=15.5 Hz, 1H), 3.90 (br s, 2H), 3.83 (br s, 2H), 3.25 (m, 4H). ¹³C NMR (126 MHz, CDCl₃) δ: 164.9, 146.5, 141.7, 141.5, 139.1, 138.5, 131.3 (q, ²J_{CF}=32.6 Hz), 128.0, 125.8 (q, ³J_{CF}=3.8 Hz), 123.9 (q, ¹J_{CF}=272.2 Hz), 123.6, 123.1, 119.2, 49.2, 48.7, 45.6, 41.9. LRMS (ESI): m/z calcd for [C₁₉H₁₈F₃N₃O+H]⁺=362.1, found 362.1.

### Example 7: Synthesis of Compounds 120, 125

### Synthesis of Compound 317

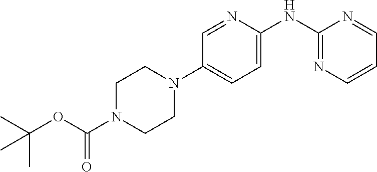 [SMILES: CC(C)(C)OC(=O)N1CCN(c2ccc(Nc3ncccn3)nc2)CC1]

A combination of 2-chloropyrimidine (100 mg, 0.95 mmol), tert-butyl 4-(6-aminopyridin-3-yl)piperazine-1-carboxylate (265 mg, 0.95 mmol), palladium (II) acetate (21 mg, 0.10 mmol), caesium carbonate (466 mg, 1.4 mmol), and Xantphos (83 mg, 0.14 mmol) was suspended in dioxane (10 mL) and heated to 120° C. overnight. The resulting mixture was concentrated in vacuo, and the crude residue was purified by silica gel chromatography (0-5% MeOH in DCM) to afford 282 mg (91%) of Compound 317 as a solid. ¹H NMR (700 MHz, CDCl₃) δ 8.50 (d, J=4.8 Hz, 2H), 8.30 (d, J=9.0 Hz, 1H), 8.19 (s, 1H), 8.07 (d, J=2.9 Hz, 1H), 7.35 (dd, J=9.1, 3.0 Hz, 1H), 6.78 (t, J=4.8 Hz, 1H), 3.63 (t, J=5.1 Hz, 4H), 3.11 (t, J=5.2 Hz, 4H), 1.51 (s, 9H). ¹³C NMR (151 MHz, CDCl₃) δ 159.3, 158.0, 154.7, 146.6, 143.0, 137.4, 127.2, 113.0, 112.9, 80.0, 50.1, 28.4. LRMS (ESI) m/z calcd for [C₁₈H₂₄N₆O₂+H]⁺=375.2, found 375.3.

### Synthesis of Compound 303

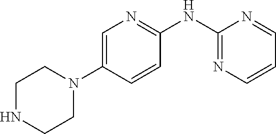 [SMILES: c1cnc(Nc2ccc(N3CCNCC3)cn2)nc1]

General Procedure E was performed with Compound 317 (152 mg, 0.43 mmol), and TFA (1.1 mL, 14 mmol) for 2 hours. The crude material (Compound 303) was used without further purification.

### Synthesis of Compound 120

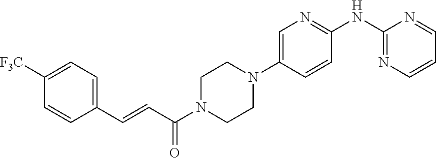 [SMILES: O=C(/C=C/c1ccc(C(F)(F)F)cc1)N1CCN(c2ccc(Nc3ncccn3)nc2)CC1]

General Procedure A was followed with trans-4-(trifluoromethyl)cinnamic acid (43 mg, 0.2 mmol), HATU (83 mg, 0.22 mmol), DIPEA (0.09 mL, 0.5 mmol), and Compound 303 (46 mg, 0.18 mmol). The crude residue was purified by silica gel chromatography (50-100% EtOAc in Hexanes followed by 0-5% MeOH in DCM) to afford 13 mg (16%, 2 steps) of Compound 120 as a residue. ¹H NMR (700 MHz, CDCl₃) δ 8.48 (dd, J=4.8, 1.6 Hz, 2H), 8.30 (d, J=8.9 Hz, 1H), 8.14 (s, 1H), 8.05 (t, J=2.2 Hz, 1H), 7.72 (dd, J=15.5, 1.7 Hz, 1H), 7.64 (d, J=1.7 Hz, 3H), 7.35 (dt, J=9.1, 2.3 Hz, 1H), 7.00 (dd, J=15.5, 1.6 Hz, 1H), 6.77 (td, J=4.8, 1.7 Hz, 1H), 3.94 (br s, 2H), 3.85 (br s, 2H), 3.19 (in, 3H). ¹³C NMR (126 MHz, CDCl₃) δ 164.9, 159.2, 158.0, 147.0, 142.5, 141.5, 138.6 (d, ⁵J_{CF}=1.3 Hz), 137.5, 131.3 (q, ²J_{CF}=32.8), 127.9, 127.4, 125.8 (q, ³J_{CF}=3.8 Hz), 123.9 (q, ¹J_{CF}=272.2 Hz), 119.3, 113.1, 113.0, 50.7, 50.1, 45.8, 42.1, 29.7, 28.4. LRMS (ESI) m/z calcd for [C₂₃H₂₁F₃N₆O+H]⁺=455.2, found 455.2.

An analogous procedure was followed to obtain the following compound.

| Compound No. | Starting Material | Characterization |
|---|---|---|
| Compound 125 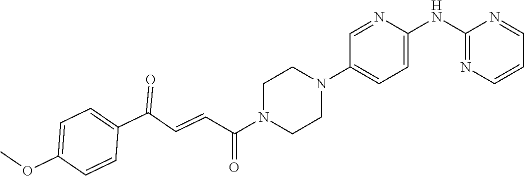 [SMILES: COc1ccc(C(=O)/C=C/C(=O)N2CCN(c3ccc(Nc4ncccn4)nc3)CC2)cc1] | trans-3-(4- methoxybenzoyl) acrylic acid (39 mg, 0.2 mmol) | The crude residue was purified by silica gel chromatography (50-100% EtOAc in Hexanes followed by 0-5% MeOH in DCM) to afford 38 mg (49%) of Compound 125 as a residue. ¹H NMR (700 MHz, CDCl₃) δ 8.77 (s, 1H), 8.50 (d, J = 4.8 Hz, 2H), 8.31 (d, J = 9.1 Hz, 1H), 8.10 (d, J = 2.9 Hz, 1H), 8.05 (d, J = 8.4 Hz, 2H), |
| | | 7.99 (d, J = 14.8 Hz, 1H), |
| | | 7.52 (d, J = 14.6 Hz, 1H), |
| | | 7.34 (dd, J = 9.0, 2.9 Hz, |
| | | 1H), 6.98 (d, J = 8.4 Hz, |
| | | 2H), 6.76 (t, J = 4.9 Hz, 1H), |
| | | 3.93 (t, J = 5.4 Hz, 2H), 3.89 |
| | | (s, 3H), 3.83 (t, J = 5.1 Hz, |
| | | 2H), 3.18 (q, J = 4.5 Hz, |
| | | 4H). ¹³C NMR (126 MHz, |
| | | CDCl₃) δ 187.6, 164.2, |
| | | 164.1, 159.3, 158.0, 147.2, |
| | | 142.3, 137.5, 134.7, 131.3, |
| | | 131.3, 130.0, 127.5, 114.1, |
| | | 113.2, 112.9, 55.6, 50.7, |
| | | 50.1, 45.9, 42.2, 29.7, 28.4. |
| | | LRMS (ESI) m/z calcd for |
| | | [C₂₄H₂₄N₆O₃ + H]⁺ = 445.2, |
| | | found 445.2. |

### Example 8: Synthesis of Compound 121

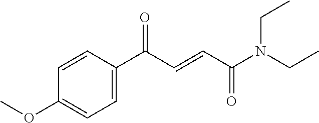 [SMILES: CCN(CC)C(=O)/C=C/C(=O)c1ccc(OC)cc1]

General Procedure A was followed with trans-3-(4-methoxybenzoyl)acrylic acid (100 mg, 0.47 mmol), HATU (176 mg, 0.48 mmol), DIPEA (0.08 mL, 0.46 mmol), and N,N-diethylamine (25 mg, 0.06 mmol). The crude residue was purified by silica gel chromatography (0-55% EtOAc in Hexanes) to afford 16 mg (13%) of Compound 121 as a film. ¹H NMR (700 MHz, CDCl₃) δ 8.06-8.04 (m, 2H), 7.99 (d, J=14.8 Hz, 1H), 7.43 (d, J=14.8 Hz, 1H), 6.99-6.95 (m, 2H), 3.89 (s, 3H), 3.52-3.45 (m, 4H), 1.24 (t, J=7.2 Hz, 3H), 1.19 (t, J=7.1 Hz, 3H). ¹³C NMR (126 MHz, CDCl₃) δ 187.9, 164.5, 164.1, 134.0, 132.2, 131.3, 131.0, 130.1, 114.1, 55.6, 42.5, 41.2, 15.1, 13.0. LRMS (ESI) m/z calcd for [C₁₅H₁₉NO₃+H]⁺=262.1, found 262.3.

### Example 9: Synthesis of Compound 302

### Synthesis of Compound 301

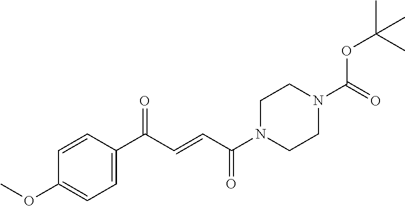 [SMILES: COc1ccc(C(=O)/C=C/C(=O)N2CCN(C(=O)OC(C)(C)C)CC2)cc1]

General Procedure A was followed with trans-3-(4-methoxybenzoyl)acrylic acid (977 mg, 7 mmol), HATU (3 g, 8 mmol), DIPEA (3.8 mL, 22 mmol), and 1-boc-piperazine (1.5 g, 8 mmol). The crude residue was purified by silica gel chromatography (0-50% EtOAc in Hexanes) to afford 1.6 g (58%) of Compound 301 as a solid. ¹H NMR (500 MHz, CDCl₃) δ 8.04 (d, J=8.6 Hz, 2H), 7.96 (dd, J=15.4, 3.9 Hz, 1H), 7.46 (d, J=14.9 Hz, 1H), 6.98 (d, J=8.7 Hz, 2H), 3.89 (s, 3H), 3.72 (t, J=5.4, 5.4 Hz, 2H), 3.62 (t, J=5.2, 5.2 Hz, 2H), 3.49 (t, J=5.2, 5.2 Hz, 4H), 1.48 (s, 9H). ¹³C NMR (126 MHz, CDCl₃) δ 187.6, 164.2, 154.5, 134.8, 131.3, 131.2, 131.1, 129.9, 114.1, 114.0, 80.5, 55.6, 45.9, 42.1, 28.4. LRMS (ESI) m/z calcd for [C₂₀H₂₆N₂O₅+Na]⁺=397.2, found 397.2.

### Synthesis of Compound 122

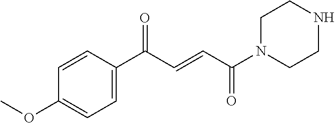 [SMILES: COc1ccc(C(=O)/C=C/C(=O)N2CCNCC2)cc1]

General Procedure E was followed with Compound 301 (789 mg, 0.2 mmol), and TFA (0.5 mL, 6.7 mmol) for 1.5 hours. The reaction mixture was concentrated in vacuo, redissolved in DCM, and stirred with a saturated solution of NaHCO₃ (2 mL) for 30 min. The organic layer was separated, washed with brine, dried over Na₂SO₄, and concentrated in vacuo to afford 21 mg (81%) of Compound 122 as an oil. LRMS (ESI) m/z calcd for [C₁₅H₁₈N₂O₃+H]⁺=275.1, found 275.1.

### Synthesis of Compound 302

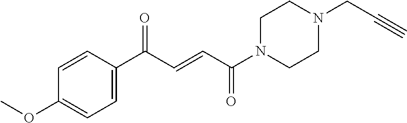 [SMILES: C#CCN1CCN(C(=O)/C=C/C(=O)c2ccc(OC)cc2)CC1]

A combination of Compound 122 (26 mg, 0.09 mmol) and potassium carbonate (16 mg, 0.11 mmol) was suspended in DMF (4 mL) and stirred at ambient temperature for 5 minutes. Propargyl bromide (0.01 mL, 0.13 mmol) was added and the reaction mixture was heated to 90° C. overnight. The reaction mixture was concentrated in vacuo, and the crude residue was purified by silica gel chromatography (0-10% MeOH in DCM) to afford 12 mg (40%) of Compound 302 as an oil. ¹H NMR (700 MHz, CDCl₃) δ 8.05-8.02 (m, 2H), 7.95 (d, J=14.9 Hz, 1H), 7.47 (d, J=14.9 Hz, 1H), 6.99-6.95 (m, 2H), 3.89 (s, 3H), 3.79 (t, J=5.1 Hz, 2H), 3.68 (t, J=5.1 Hz, 2H), 3.35 (d, J=2.4 Hz, 2H), 2.60 (q, J=4.6 Hz, 4H), 2.28 (d, J=2.4 Hz, 1H). ¹³C NMR (151 MHz, CDCl₃) δ 187.7, 164.2, 164.0, 134.4, 131.5, 131.3, 130.0, 114.1, 78.0, 73.8, 55.6, 51.9, 51.3, 46.8, 45.9, 42.1. LRMS (ESI) m/z calcd for [C₁₈H₂₀N₂O₃+H]⁺=213.1, found 213.2.

### Example 10: Synthesis of Compounds 124, 123

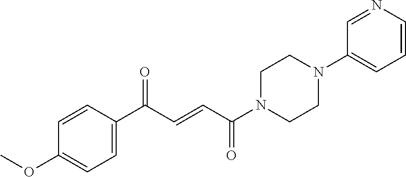 [SMILES: COc1ccc(C(=O)/C=C/C(=O)N2CCN(c3cccnc3)CC2)cc1]

General Procedure A was followed with trans-3-(4-methoxybenzoyl)acrylic acid (200 mg, 0.97 mmol), HATU (740 mg, 1.96 mmol), DIPEA (0.68 mL, 3.9 mmol), and 1-(3-pyridinyl)piperazine (158 mg, 0.97 mmol). The crude residue was purified by silica gel chromatography (0-8% DCM in MeOH) to afford 40 mg (12%) of Compound 124 as a film. 1H NMR (500 MHz, CDCl₃) δ 8.33-8.27 (m, 1H), 8.14 (dd, J=3.8, 2.2 Hz, 1H), 8.06-8.00 (m, 2H), 7.97 (d, J=14.9 Hz, 1H), 7.49 (d, J=14.8 Hz, 1H), 7.22-7.14 (m, 2H), 6.99-6.92 (m, 2H), 3.90 (m, 2H), 3.86 (s, 3H), 3.80 (t, J=5.2 Hz, 2H), 3.27-3.21 (m, 4H). ¹³C NMR (126 MHz, CDCl₃) δ 187.6, 164.2, 164.1, 146.5, 141.7, 139.2, 134.8, 131.3, 131.1, 129.9, 123.6, 123.1, 114.1, 55.6, 49.2, 48.7, 45.7, 42.0. LRMS (ESI) m/z calcd for [C₂₀H₂₁N₃O₃+H]⁺=352.2, found 352.1.

An analogous procedure was followed to obtain the following compound.

| Compound No. | Starting Material | Characterization |
|---|---|---|
| Compound 123 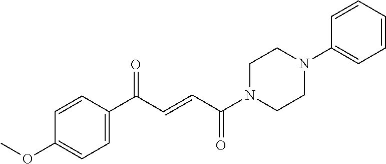 [SMILES: COc1ccc(C(=O)/C=C/C(=O)N2CCN(c3ccccc3)CC2)cc1] | 1-phenyl piperazine (25 mg, 0.06 mmol) | The crude residue was purified by silica gel chromatography (15-55% EtOAc in Hexanes) to afford 256 mg (75%) of Compound 123 as a film. ¹H NMR (500 MHz, CDCl₃) δ 8.05 (d, J = 8.6 Hz, 2H), 7.99 (d, J = 14.9 Hz, 1H), 7.52 (d, J = 14.9 Hz, 1H), 7.30 (t, J = 7.7 Hz, 2H), 7.01- 6.89 (m, 5H), 3.92 (m, 2H), 3.90 (s, 3H), 3.81 (t, J = 5.1 Hz, 2H), 3.23 (m, 4H). ¹³C NMR (126 MHz, CDCl₃) δ 187.7, 164.2, 164.1, 150.8, 134.6, 131.4, 131.3, 130.0, 129.3, 120.8, 116.8, 114.1, 55.6, 50.0, |
| | | 49.4, 46.0, 42.2. LRMS (ESI) m/z calcd |
| | | for [C₂₁H₂₂N₂O₃ + H]⁺ = 351.2, found |
| | | 351.1. |

### Example 11: Synthesis of Compound 127

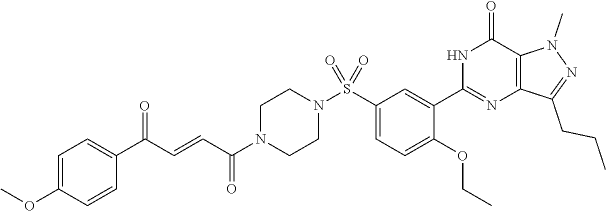 [SMILES: CCCc1nn(C)c2c(=O)[nH]c(-c3cc(S(=O)(=O)N4CCN(C(=O)/C=C/C(=O)c5ccc(OC)cc5)CC4)ccc3OCC)nc12]

The secondary amine Compound 122 (23 mg, 0.08 mmol) was dissolved in DCM (1 mL). DIPEA (0.1 mL, 0.5 mmol) was added, and the reaction mixture was stirred at ambient temperature for 5 minutes. A solution of 5-(5-Chlorosulfonyl-2-ethoxyphenyl)-1-methyl-3-propyl-1,6-dihydro-7H-pyrazolo[4,3-d]pyrimidin-7-one (35 mg, 0.08 mmol) in DCM (0.5 mL) was added dropwise to the reaction mixture and allowed to stir at ambient temperature for 2 hours. The resulting mixture was concentrated in vacuo, and the crude residue was purified by silica gel chromatography (25-100% EtOAc in Hexanes) to afford 50 mg (90%) of Compound 127 as an oil. ¹H NMR (500 MHz, CDCl₃) δ: 10.73 (s, 1H), 8.73 (d, J=2.4 Hz, 1H), 7.92-7.89 (m, 2H), 7.82 (d, J=14.9 Hz, 1H), 7.75 (dd, J=8.7, 2.4 Hz, 1H), 7.27 (d, J=14.8 Hz, 1H), 7.09 (d, J=8.8 Hz, 1H), 6.89-6.84 (m, 2H), 4.30 (q, J=7.0 Hz, 2H), 4.19 (s, 3H), 3.80 (s, 3H), 3.79-3.76 (m, 2H), 3.68 (t, J=5.0 Hz, 2H), 3.05 (q, J=5.4 Hz, 4H), 2.86 (t, J=7.6 Hz, 2H), 1.79 (h, J=7.4 Hz, 2H), 1.57 (t, J=7.0 Hz, 3H), 0.96 (t, J=7.4 Hz, 3H). ¹³C NMR (126 MHz, CDCl₃) δ:187.3, 164.3, 164.0, 159.6, 153.6, 147.0, 146.2, 138.3, 135.2, 131.5, 131.3, 131.1, 130.5, 129.7, 128.5, 124.5, 121.4, 114.1, 113.3, 77.3, 66.2, 55.6, 46.4, 45.8, 45.4, 41.6, 38.2, 27.7, 22.3, 14.5, 14.1. HRMS (ESI) m/z calcd for [C₃₂H₃₆N₆O₇S+H]⁺=649.2366, found 649.2450.

### Example 12: Synthesis of Compound 130

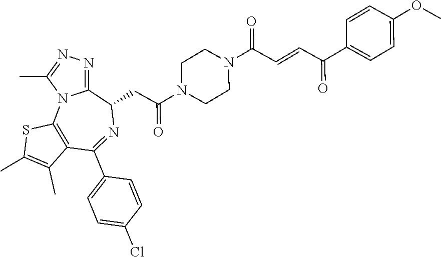

General Procedure A was followed with (6S)-4-(4-chlorophenyl)-2,3,9-trimethyl-6H-thieno[3,2-f][1,2,4]triazolo[4,3-a][1,4]diazepine-6-acetic acid (JQ1-Acid) (93 mg, 0.23 mmol), HATU (93 mg, 0.24 mmol), DIPEA (0.1 mL, 0.6 mmol), and Compound 122 (55 mg, 0.2 mmol). The crude residue was purified by silica gel chromatography (0-5% MeOH in DCM) to afford 109 mg (83%, 2 steps) of Compound 130 as a powder. ¹H NMR (700 MHz, CDCl₃) δ 8.08 (d, J=8.6 Hz, 2H), 8.02 (dd, J=14.9, 6.5 Hz, 1H), 7.51 (dd, J=14.9, 6.7 Hz, 1H), 7.42 (d, J=7.8 Hz, 2H), 7.39-7.34 (m, 2H), 7.01 (d, J=8.5 Hz, 2H), 4.83 (td, J=6.9, 2.9 Hz, 1H), 4.08-3.95 (m, 2H), 3.93 (s, 3H), 3.91-3.70 (m, 5H), 3.71-3.49 (m, 3H), 2.69 (s, 3H), 2.43 (s, 3H), 1.70 (s, 3H). ¹³C NMR (151 MHz, CDCl₃) δ 187.8, 187.7, 169.3, 169.2, 164.5, 164.2, 164.2, 164.1, 155.6, 150.0, 136.7, 136.5, 134.9, 131.9, 131.3, 131.0, 131.0, 131.0, 130.9, 130.5, 129.7, 129.7, 128.6, 114.1, 114.0, 55.5, 54.2, 54.1, 45.8, 45.8, 45.6, 45.2, 42.1, 42.0, 41.8, 41.4, 35.1, 29.6, 14.2, 13.0, 11.6. HRMS (ESI) m/z calcd for [C₃₄H₃₃ClN₆O₄S+H]⁺=657.1973, found 657.2054.

### Example 13: Synthesis of Compound 307

### Synthesis of Compound 306

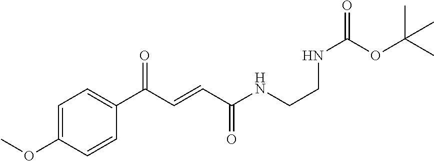 [SMILES: COc1ccc(C(=O)/C=C/C(=O)NCCNC(=O)OC(C)(C)C)cc1]

General Procedure A was followed with trans-3-(4-methoxybenzoyl)acrylic acid (54 mg, 0.26 mmol), HATU (124 mg, 0.33 mmol), DIPEA (0.13 mL, 0.8 mmol), and N-boc-ethylenediamine (0.1 mL, 0.6 mmol). The crude residue was purified by silica gel chromatography (0-5% MeOH in DCM followed by 0-100% EtOAc in Hexanes) to afford 38 mg (42%) of Compound 306 as a residue, and 102 mg of a double addition byproduct. LRMS (ESI) of byproduct m/z calcd for [C₂₅H₄₀N₄O₇+H]⁺=509.3, found 509.3. ¹H NMR (700 MHz, CDCl₃) δ 8.03 (d, J=8.4 Hz, 2H), 7.94 (d, J=14.9 Hz, 1H), 6.98-6.93 (m, 3H), 4.99 (t, J=6.2 Hz, 1H), 3.89 (s, 3H), 3.51 (q, J=5.6 Hz, 2H), 3.36 (q, J=5.9 Hz, 2H), 2.80 (s, 1H), 1.43 (s, 9H). ¹³C NMR (151 MHz, CDCl₃) δ 187.9, 164.9, 164.2, 134.5, 133.2, 131.3, 130.0, 114.1, 80.0, 55.6, 41.5, 40.1, 38.6, 37.1, 32.8, 31.9, 30.0, 29.7, 29.7, 29.4, 28.4, 27.1, 22.7, 19.7, 14.1. LRMS (ESI) m z calcd for [C₁₈H₂₄N₂O₅+Na]=371.2, found 371.1.

### Synthesis of Compound 307

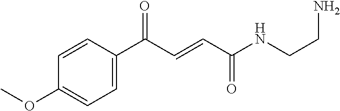 [SMILES: COc1ccc(C(=O)/C=C/C(=O)NCCN)cc1]

General Procedure E was followed with Compound 306 (19 mg, 0.05 mmol), and TFA (0.13 mL, 1.8 mmol) for 40 minutes. The crude material was used without further purification.

### Example 14: Synthesis of Compounds 137, 138

### Synthesis of Compound 304

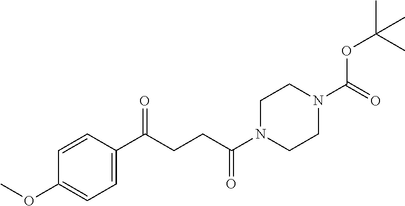 [SMILES: COc1ccc(C(=O)CCC(=O)N2CCN(C(=O)OC(C)(C)C)CC2)cc1]

General Procedure A was followed with 3-(4-methoxybenzoyl)propionic acid (101 mg, 0.48 mmol), HATU (207 mg, 0.54 mmol), DIPEA (0.25 mL, 1.45 mmol), and 1-boc-piperazine (105 mg, 0.56 mmol). The crude residue was purified by silica gel chromatography (0-100% EtOAc in Hexanes) to afford 133 mg (73%) of Compound 304 as a powder. ¹H NMR (700 MHz, CDCl₃) δ 7.93 (d, J=8.6 Hz, 2H), 6.87 (d, J=8.5 Hz, 2H), 3.80 (s, 3H), 3.58-3.52 (m, 2H), 3.52-3.47 (m, 2H), 3.44 (d, J=7.5 Hz, 2H), 3.35 (t, J=5.5 Hz, 2H), 3.26 (t, J=6.5 Hz, 2H), 2.71 (t, J=6.6 Hz, 2H), 1.43 (s, 9H). ¹³C NMR (151 MHz, CDCl₃) δ 197.5, 170.6, 163.5, 154.5, 130.3, 129.8, 113.7, 80.2, 55.4, 45.2, 41.6, 33.1, 29.7, 28.4, 27.1. LRMS (ESI) m/z calcd for [C₂₀H₂₈N₂O₅+H]⁺=377.5, found 377.2.

### Synthesis of Compound 305

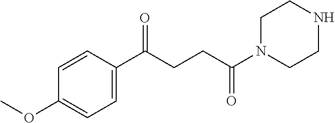 [SMILES: COc1ccc(C(=O)CCC(=O)N2CCNCC2)cc1]

General Procedure E was followed with Compound 304 (24 mg, 0.06 mmol), and TFA (0.13 mL, 1.7 mmol) for 2.5 hours. The crude material was used without further purification.

### Synthesis of Compound 137

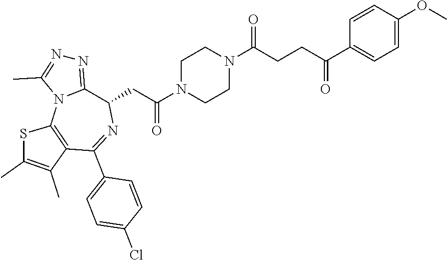

General Procedure A was followed with (6S)-4-(4-chlorophenyl)-2,3,9-trimethyl-6H-thieno[3,2-f][1,2,4]triazolo[4,3-a][1,4]diazepine-6-acetic acid (JQ1-Acid) (26 mg, 0.07 mmol), HATU (27 mg, 0.07 mmol), DIPEA (0.4 mL, 0.19 mmol), and Compound 305 (17 mg, 0.06 mmol). The crude residue was purified by silica gel chromatography (0-7% MeOH in DCM) to afford 39 mg (94%, 2 steps) of Compound 137 as a foam. ¹H NMR (700 MHz, CDCl₃) δ 7.99 (d, J=8.8 Hz, 2H), 7.39 (d, J=8.2 Hz, 2H), 7.32 (dd, J=8.6, 4.2 Hz, 2H), 6.95-6.91 (m, 2H), 4.79 (q, J=6.7 Hz, 1H), 4.01-3.94 (m, 1H), 3.86 (s, 3H), 3.85-3.80 (m, 2H), 3.79-3.69 (m, 4H), 3.69-3.63 (m, 1H), 3.60-3.55 (m, 1H), 3.54-3.48 (m, 1H), 3.42-3.35 (m, 1H), 3.32-3.26 (m, 1H), 2.84 (tt, J=16.0, 6.5 Hz, 1H), 2.78-2.73 (m, 1H), 2.65 (d, J=2.8 Hz, 3H), 2.39 (s, 3H), 1.67 (s, 3H). ¹³C NMR (151 MHz, CDCl₃) δ 197.5, 170.7, 169.4, 169.2, 163.9, 163.8, 163.6, 155.8, 149.9, 149.9, 136.8, 136.7, 132.2, 130.9, 130.9, 130.7, 130.7, 130.5, 130.4, 129.9, 129.8, 128.7, 113.7, 55.5, 54.6, 54.4, 45.9, 45.6, 45.5, 45.2, 41.8, 41.7, 41.6, 35.4, 35.3, 33.2, 33.2, 31.9, 29.7, 27.2, 27.1, 22.7, 14.4, 14.1, 13.1, 11.8. HRMS (ESI) m/z calcd for [C₃₄H₃₅ClN₆O₄S+H]⁺=659.2129, found 659.2207.

An analogous procedure was followed to obtain the following compound.

| Compound No. | SM | Characeterization |
|---|---|---|
| Compound 138 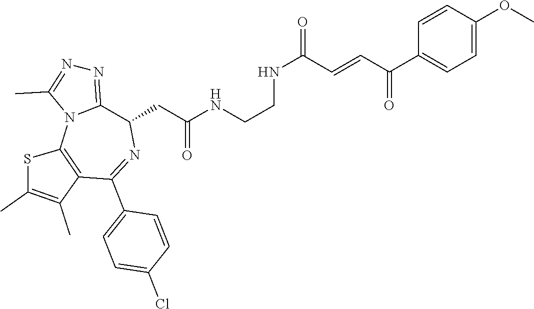 | Compound 307 (14 mg, 0.05 mmol) | The crude residue was purified by silica gel chromatography (0-6% MeOH in DCM) to afford 17 mg (51%, 2 steps) of Compound 138 as a foam. ¹H NMR (600 MHz, CDCl₃) δ 8.02-7.98 (m, 2H), 7.95 (t, J = 5.6 Hz, 1H), 7.88 (s, 1H), 7.82 (q, J = 5.4 Hz, 1H), 7.39- 7.36 (m, 2H), 7.29 (d, J = 7.4 Hz, 2H), 6.96-6.91 (m, 2H), 6.89 (d, J = 15.0 Hz, 1H), 4.71 (dd, J = 7.9, 6.2 Hz, 1H), 3.87 (s, 3H), 3.55 (qdt, J = 10.9, 6.8, 3.5 Hz, 5H), 3.46 (dd, J = 14.7, 6.2 Hz, 1H), 2.73 (s, 3H), 2.39 (s, 3H), 1.66 (s, 3H). ¹³C NMR (151 MHz, CDCl₃) δ 188.0, 171.3, 164.8, 164.3, 164.0, 156.0, 150.4, 136.9, 136.4, 135.1, 132.6, 132.0, 131.2, 131.2, 131.0, 130.6, 130.2, 129.9, 128.7, 114.0, 55.5, 54.2, 40.3, 39.0, 38.9, 29.7, |
| | | 14.4, 13.1, 11.8. HRMS (ESI) m/z |
| | | calcd for [C₃₂H₃₁ClN₆O₄S + H]⁺ = |
| | | 631.1816, found 631.1899. |

### Example 15: Synthesis of Compound 132

### Synthesis of Compound 308

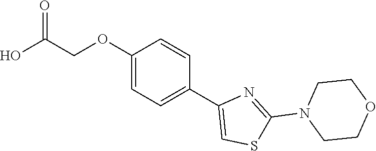 [SMILES: O=C(O)COc1ccc(-c2csc(N3CCOCC3)n2)cc1]

A solution of tert-butyl 2-(4-(2-morpholinothiazol-4-yl)phenoxy)acetate (40 mg, 0.11 mmol) in 4 M hydrochloric acid in dioxane (0.7 mL) was stirred at ambient temperature overnight. The reaction mixture was diluted with toluene and concentrated in vacuo to yield a solid which was used without further purification.

### Synthesis of Compound 132

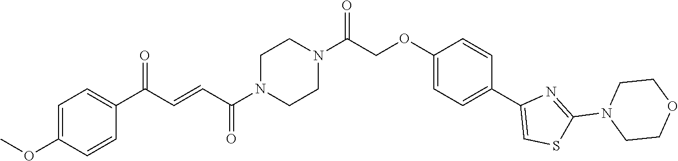 [SMILES: COc1ccc(C(=O)/C=C/C(=O)N2CCN(C(=O)COc3ccc(-c4csc(N5CCOCC5)n4)cc3)CC2)cc1]

General Procedure A was followed with 2-(4-(2-morpholinothiazol-4-yl)phenoxy)acetic acid (Compound 308, 35 mg, 0.11 mmol), HATU (50 mg, 0.13 mmol), DIPEA (0.06 mL, 0.33 mmol), and Compound 122 (37 mg, 0.13 mmol). The crude residue was purified by silica gel chromatography (0-5% MeOH in DCM) to afford 35 mg (55%, 2 steps) of Compound 132 as a powder. ¹H NMR (700 MHz, CDCl₃) δ 8.03 (d, J=8.6 Hz, 2H), 7.96 (dd, J=14.9, 4.6 Hz, 1H), 7.79-7.76 (m, 2H), 7.43 (d, J=14.7 Hz, 1H), 6.99-6.94 (m, 4H), 6.68 (s, 1H), 4.76 (d, J=3.3 Hz, 2H), 3.89 (s, 3H), 3.85-3.82 (m, 4H), 3.73 (q, J=6.3 Hz, 1H), 3.68 (p, J=5.5 Hz, 5H), 3.64-3.59 (m, 2H), 3.54-3.50 (m, 4H). ¹³C NMR (151 MHz, CDCl₃) δ 187.5, 171.2, 166.9, 164.4, 164.3, 157.1, 151.2, 135.1, 131.3, 130.8, 129.9, 129.3, 127.6, 114.5, 114.1, 100.5, 68.2, 68.0, 66.2, 55.6, 48.6, 46.1, 45.7, 45.2, 42.4, 42.0. HRMS (ESI) m/z calcd for [C₃₀H₃₂N₄O₆S+H]⁺=577.2043, found 577.2122.

### Example 16: Synthesis of Compound 133

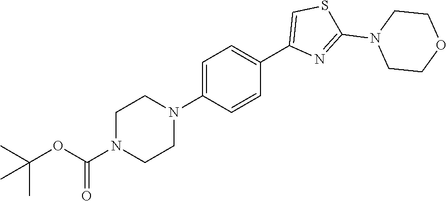 [SMILES: CC(C)(C)OC(=O)N1CCN(c2ccc(-c3csc(N4CCOCC4)n3)cc2)CC1]

A mixture of 4-(4-bromo-2-thiazolyl)morpholine (120 mg, 0.51 mmol), 4-(4-tert-butoxycarbonylpiperazinyl)phenylboronic acid pinacol ester (211, 0.54 mmol), caesium carbonate (486 mg, 1.5 mmol), and tetrakis(triphenylphosphine)palladium(0) (66 mg, 0.05 mmol) was suspended in DMF (12 mL) and stirred at 100° C. overnight. The reaction was quenched with 5% LiCl_{(aq)} (60 mL) and extracted 3 times with DCM. The organic extracts were washed again with 5% LiCl_{(aq)} and the organic extract was dried over Na₂SO₄, vacuum filtered, and concentrated in vacuo. The crude residue was purified by silica gel chromatography (0-50% EtOAc in Hexanes) to afford 29 mg (32%, BRSM) of Compound 309 as a solid. ¹H NMR (700 MHz, CDCl₃) δ 7.75-7.72 (m, 2H), 6.93-6.90 (m, 2H), 6.65 (s, 1H), 3.85-3.81 (m, 4H), 3.58 (t, J=5.2 Hz, 4H), 3.52 (dd, J=5.9, 3.9 Hz, 4H), 3.16 (t, J=5.1 Hz, 4H), 1.49 (s, 9H). ¹³C NMR (126 MHz, CDCl₃) δ 171.1, 151.8, 150.7, 127.3, 127.2, 127.0, 116.8, 116.3, 99.7, 79.9, 66.3, 49.2, 48.6, 28.5. LRMS (ESI) m/z calcd for [C₂₂H₃₀N₄O₃S+H]⁺=431.6, found 431.2.

### Synthesis of Compound 310

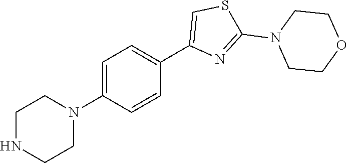 [SMILES: c1cc(N2CCNCC2)ccc1-c1csc(N2CCOCC2)n1]

General Procedure E was followed with Compound 309 (34 mg, 0.07 mmol), and TFA (0.18 mL, 2.4 mmol) for 30 minutes. The crude material was used without further purification.

### Synthesis of Compound 133

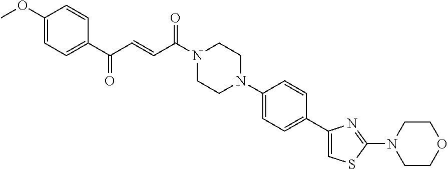 [SMILES: COc1ccc(C(=O)/C=C/C(=O)N2CCN(c3ccc(-c4csc(N5CCOCC5)n4)cc3)CC2)cc1]

General Procedure A was followed with trans-3-(4-methoxybenzoyl)acrylic acid (19 mg, 0.09 mmol), HATU (44 mg, 0.1 mmol), DIPEA (0.04 mL, 0.23 mmol), and Compound 310 (23 mg, 0.07 mmol). The crude residue was purified by silica gel chromatography (0-100% EtOAc in Hexanes) to afford 19 mg (51%, 2 steps) of Compound 133 as a foam. ¹H NMR (700 MHz, CDCl₃) δ 8.07-8.03 (m, 2H), 7.99 (d, J=14.8 Hz, 1H), 7.77-7.73 (m, 2H), 7.54-7.50 (m, 1H), 7.00-6.96 (m, 2H), 6.95-6.91 (m, 2H), 6.66 (s, 1H), 3.91 (t, J=5.2 Hz, 2H), 3.89 (s, 3H), 3.83 (t, J=4.9 Hz, 4H), 3.81 (d, J=5.1 Hz, 2H), 3.52 (t, J=4.9 Hz, 4H), 3.26 (q, J=4.9 Hz, 4H). ¹³C NMR (126 MHz, CDCl₃) δ 187.7, 171.2, 164.2, 164.1, 151.6, 150.1, 134.6, 131.3, 130.0, 127.7, 127.1, 116.4, 114.1, 99.9, 66.3, 55.6, 49.7, 49.1, 48.6, 45.9, 42.1. HRMS (ESI) m/z calcd for [C₂₈H₃₀N₄O₄S+H]⁺=519.1988, found 519.2067.

### Example 17: Synthesis of Compound 126

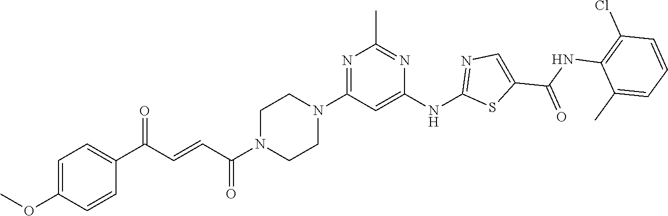 [SMILES: COc1ccc(C(=O)/C=C/C(=O)N2CCN(c3cc(Nc4ncc(C(=O)Nc5c(C)cccc5Cl)s4)nc(C)n3)CC2)cc1]

General Procedure A was followed with trans-3-(4-methoxybenzoyl)acrylic acid (7 mg, 0.03 mmol), HATU (13 mg, 0.03 mmol), DIPEA (0.02 mL, 0.13 mmol), and N-deshydroxyethyl dasatinib (10 mg, 0.02 mmol). The crude residue was purified by silica gel chromatography (0-6% MeOH in DCM) to afford 13 mg (90%) of Compound 126 as a solid. ¹H NMR (700 MHz, CDCl₃) δ 8.00-7.95 (m, 3H), 7.92 (d, J=14.9 Hz, 1H), 7.42 (d, J=14.9 Hz, 1H), 7.24 (d, J=7.7 Hz, 1H), 7.15-7.07 (m, 2H), 6.95-6.91 (m, 2H), 5.87 (s, 1H), 3.83 (s, 3H), 3.77 (t, J=5.3 Hz, 2H), 3.70 (p, J=5.2 Hz, 4H), 3.62 (d, J=5.5 Hz, 2H), 2.45 (s, 3H), 2.26 (s, 3H). ¹³C NMR (126 MHz, CDCl₃) δ 188.0, 166.5, 164.6, 164.4, 162.8, 156.9, 140.4, 138.6, 135.0, 132.4, 131.4, 131.0, 129.7, 129.1, 128.1, 127.1, 125.6, 114.2, 83.1, 55.5, 45.5, 43.9, 43.6, 41.8, 29.6, 25.4, 18.7. HRMS (ESI) m/z calcd for [C₃₁H₃₀ClN₇O₄S+H]⁺=632.1769, found 632.1848.

### Example 18: Synthesis of Compound 129

### Synthesis of Compound 311

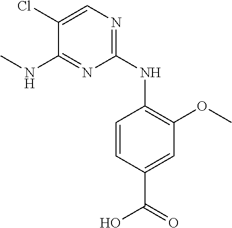

A mixture of 2,5-dichloro-N-methyl pyrimidin-4-amine (106 mg, 0.6 mmol) and 4-amino-3-methoxybenzoic acid (112 mg, 0.67 mmol) was dissolved in a 1:1 mixture of Dioxane:H₂O (4 mL). A solution of 4 M HCl in dioxane (0.15 mL) was added and the reaction mixture was stirred at 100° C. overnight. A white precipitate formed upon cooling which was filtered and washed with H₂O to afford 79 mg (43%) of Compound 311 as a powder which product was used without further purification. ¹H NMR (500 MHz, DMSO) δ 9.17 (s, 1H), 8.55 (d, J=5.8 Hz, 1H), 8.29 (d, J=8.5 Hz, 1H), 8.18 (s, 1H), 7.63 (d, J=8.5 Hz, 1H), 7.55 (s, 1H), 3.93 (s, 3H), 2.98 (d, J=4.6 Hz, 3H).

### Synthesis of Compound 129

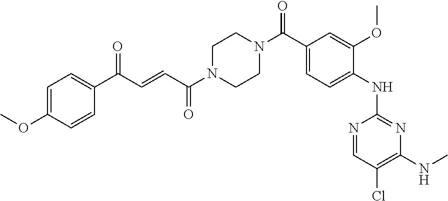

General Procedure A was followed with Compound 311 (21 mg, 0.07 mmol), HATU (34 mg, 0.07 mmol), DIPEA (0.05 mL, 0.3 mmol), and Compound 122 (21 mg, 0.07 mmol). The crude residue was purified by silica gel chromatography (0-8% MeOH in DCM) to afford 23 mg (60%, 2 steps) of Compound 129 as a film. ¹H NMR (700 MHz, CDCl₃) δ 8.58 (d, J=8.2 Hz, 1H), 8.06-8.02 (m, 2H), 7.99 (d, J=14.9 Hz, 1H), 7.93 (s, 1H), 7.67 (s, 1H), 7.47 (d, J=14.9 Hz, 1H), 7.04 (d, J=1.8 Hz, 1H), 7.02 (dd, J=8.3, 1.8 Hz, 1H), 7.00-6.96 (m, 2H), 5.34 (q, J=4.9 Hz, 1H), 3.93 (s, 3H), 3.89 (s, 3H), 3.81-3.59 (m, 8H), 3.11 (d, J=4.9 Hz, 3H). ¹³C NMR (151 MHz, CDCl₃) δ 187.5, 170.9, 164.3, 164.3, 158.6, 157.7, 152.6, 147.5, 135.0, 131.9, 131.3, 130.9, 129.9, 126.7, 120.3, 116.7, 114.2, 114.1, 109.6, 105.7, 55.9, 55.6, 46.0, 42.4, 28.1. HRMS (ESI) m/z calcd for [C₂₈H₂₉ClN₆O₅+H]⁺=565.1888, found 565.1968.

### Example 19: Synthesis of Compound 131

### Synthesis of Compound 312

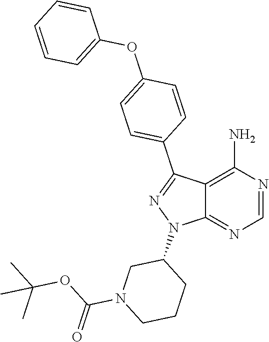 [SMILES: CC(C)(C)OC(=O)N1CCC[C@@H](n2nc(-c3ccc(Oc4ccccc4)cc3)c3c(N)ncnc32)C1]

A mixture of (4-phenoxyphenyl)boronic acid (61 mg, 0.28 mmol), tert-butyl (R)-3-(4-amino-3-iodo-1H-pyrazolo[3,4-d]pyrimidin-1-yl)piperidine-1-carboxylate (117 mg, 0.26 mmol), tetrakis(triphenylphosphine)palladium (28 mg, 0.03 mmol), and potassium carbonate (105 mg, 0.8 mmol) was suspended in a 1:1 mixture of Dioxane:H₂O (4 mL) and the reaction mixture was stirred at 90° C. for 5 hours. The resulting mixture was concentrated in vacuo, and the crude residue was purified by silica gel chromatography (0-70% EtOAc in Hexanes) to afford 127 mg (99%) of Compound 312 as a foam. ¹H NMR (500 MHz, CDCl₃) δ 8.37 (s, 1H), 7.65 (dd, J=8.1, 6.1 Hz, 2H), 7.41-7.36 (m, 2H), 7.15 (dd, J=8.4, 6.4 Hz, 3H), 7.08 (d, J=7.9 Hz, 2H), 5.64 (s, 2H), 4.84 (tt, J=10.9, 4.4 Hz, 1H), 4.47-4.03 (m, 2H), 2.85 (td, J=13.0, 3.0 Hz, 1H), 2.32-2.14 (m, 2H), 1.88 (d, J=7.9 Hz, 1H), 1.75-1.63 (m, 1H), 1.44 (s, 9H).

### Synthesis of Compound 313

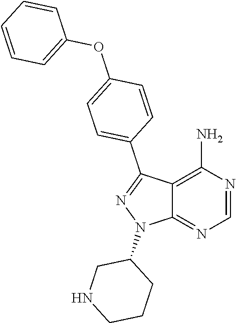 [SMILES: Nc1ncnc2c1c(-c1ccc(Oc3ccccc3)cc1)nn2[C@@H]1CCCNC1]

General Procedure E was followed with Compound 312 (37 mg, 0.08 mmol), and TFA (0.2 mL, 2.5 mmol) for 30 minutes. The crude material was used without further purification.

### Synthesis of Compound 131

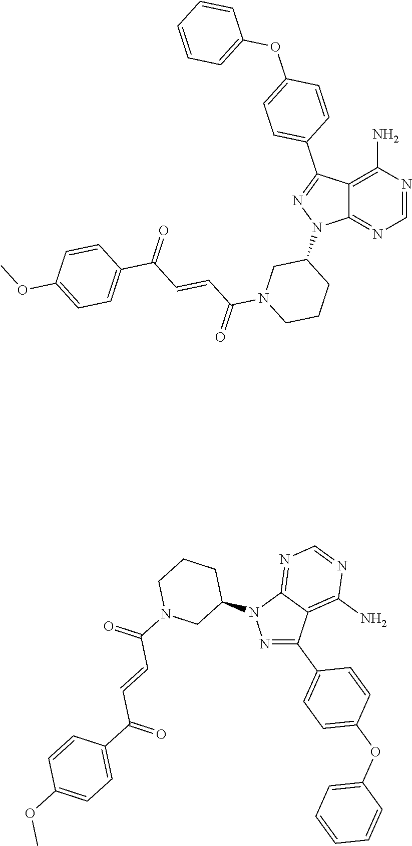 [SMILES: COc1ccc(C(=O)/C=C/C(=O)N2CCC[C@@H](n3nc(-c4ccc(Oc5ccccc5)cc4)c4c(N)ncnc43)C2)cc1 ; COc1ccc(C(=O)/C=C/C(=O)N2CCC[C@@H](n3nc(-c4ccc(Oc5ccccc5)cc4)c4c(N)ncnc43)C2)cc1]

General Procedure A was followed with trans-3-(4-methoxybenzoyl)acrylic acid (25 mg, 0.09 mmol), HATU (41 mg, 0.11 mmol), DIPEA (0.05 mL, 0.3 mmol), and Compound 313 (28 mg, 0.07 mmol). The crude residue was purified by silica gel chromatography (0-5% MeOH in DCM) and then by reverse phase silica gel chromatography (5-95% MeCN in H₂O) to afford 17 mg (41%, 2 steps) of Compound 131 as a solid. ¹H NMR (700 MHz, CDCl₃) δ 8.38-8.31 (m, 1H), 8.07-8.03 (m, 1H), 8.00-7.97 (m, 1H), 7.97-7.85 (m, 1H), 7.67-7.62 (m, 2H), 7.55-7.41 (m, 1H), 7.40-7.36 (m, 2H), 7.17 (t, J=7.3 Hz, 1H), 7.16-7.13 (m, 2H), 7.08 (d, J=8.0 Hz, 2H), 7.00-6.96 (m, 1H), 6.96-6.92 (m, 1H), 5.64 (s, 2H), 4.96-4.87 (m, 2H), 4.55-4.49 (m, 1H), 4.22 (dd, J=13.6, 4.1 Hz, 1H), 4.11 (d, J=13.8 Hz, 1H), 3.92 (dd, J=13.4, 9.9 Hz, 1H), 3.88 (d, J=13.3 Hz, 3H), 3.48 (dd, J=12.6, 10.6 Hz, 1H), 3.28 (td, J=13.2, 2.9 Hz, 1H), 3.08 (ddd, J=13.7, 11.1, 3.2 Hz, 1H), 2.45-2.33 (m, 1H), 2.28 (dd, J=13.7, 4.1 Hz, 1H), 2.05 (ddt, J=12.1, 7.5, 3.7 Hz, 1H), 1.81-1.71 (m, 1H). ¹³C NMR (151 MHz, CDCl₃) δ 187.7, 187.6, 164.7, 164.4, 164.2, 164.1, 158.6, 158.6, 157.8, 156.4, 156.3, 155.8, 155.7, 154.3, 154.3, 144.1, 144.0, 134.4, 134.1, 132.0, 131.9, 131.3, 131.3, 130.0, 127.8, 127.6, 124.1, 119.6, 119.1, 114.1, 114.0, 98.6, 98.6, 55.6, 55.6, 53.4, 52.4, 50.1, 46.3, 46.2, 42.5, 30.3, 30.0, 25.3, 23.7. HRMS (ESI) m/z calcd for [C₃₃H₃₀N₆O₄+H]⁺=575.2329, found 575.2406.

### Example 20: Synthesis of Compound 128

### Synthesis of Compound 314

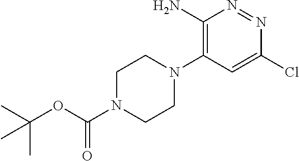 [SMILES: CC(C)(C)OC(=O)N1CCN(c2cc(Cl)nnc2N)CC1]

A mixture of 3-amino-4-bromo-6-chloropyridazine (109 mg, 0.52 mmol) and 1-boc-piperazine (462 mg, 2.5 mmol) was dissolved in a THF (1 mL) and the reaction mixture was stirred at 80° C. for overnight. The solvent was removed in vacuo, and the crude residue was purified by silica gel chromatography (0-100% EtOAc in Hexanes) to afford 147 mg (90%) of Compound 314 as a foam. ¹H NMR (500 MHz, CDCl₃) δ 6.73 (s, 1H), 5.12 (s, 2H), 3.60-3.55 (m, 4H), 3.00 (t, J=5.0 Hz, 4H), 1.48 (s, 9H).

### Synthesis of Compound 315

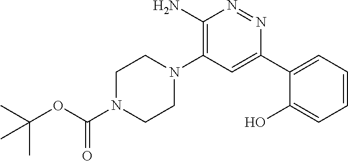 [SMILES: CC(C)(C)OC(=O)N1CCN(c2cc(-c3ccccc3O)nnc2N)CC1]

A mixture of Compound 314 (122 mg, 0.39 mmol), 2-hydroxyphenyl boronic acid (138 mg, 1 mmol), [1,1′-Bis(diphenylphosphino)ferrocene]dichloropalladium(II) (35 mg, 0.05 mmol), and potassium carbonate (186 mg, 1.35 mmol) was suspended in a 1:1 mixture of MeCN:H₂O (2.5 mL) in a sealed tube, and the reaction mixture was stirred at 120° C. for 45 minutes. The solvent was removed in vacuo, and the crude residue was purified by silica gel chromatography (0-100% EtOAc in Hexanes) to afford 21 mg (15%) of Compound 315 as a powder. ¹H NMR (500 MHz, CDCl₃) δ 7.57 (dd, J=8.0, 1.6 Hz, 1H), 7.29 (ddd, J=8.5, 7.2, 1.6 Hz, 1H), 7.05 (dd, J=8.2, 1.2 Hz, 1H), 6.91 (td, J=7.6, 1.3 Hz, 1H), 4.88 (s, 2H), 3.65 (t, J=5.0 Hz, 4H), 3.10 (t, J=5.0 Hz, 4H), 1.50 (s, 9H).

### Synthesis of Compound 316

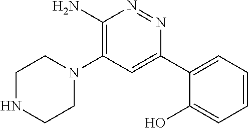 [SMILES: Nc1nnc(-c2ccccc2O)cc1N1CCNCC1]

General Procedure E was followed with Compound 315 (11 mg, 0.03 mmol), and TFA (0.09 mL, 0.96 mmol) for 1 hour. The solvent was removed in vacuo, and the crude residue was purified by silica gel chromatography (0-20% MeOH in DCM with 0.1% Et₃N) to afford 6 mg (76%) of Compound 316 as an oil. ¹H NMR (600 MHz, DMSO) δ 7.92 (dd, J=8.3, 1.6 Hz, 1H), 7.48 (s, 1H), 7.24 (ddd, J=8.4, 7.3, 1.6 Hz, 1H), 6.89 (dtd, J=8.2, 3.6, 1.2 Hz, 2H), 6.23 (d, J=6.0 Hz, 1H), 4.01 (s, 1H), 3.04 (t, J=5.0 Hz, 4H), 2.98-2.91 (m, 4H). LRMS (ESI) m/z calcd for [C₁₄H₁₇N₅O+H]⁺=272.3, found 272.1.

### Synthesis of Compound 128

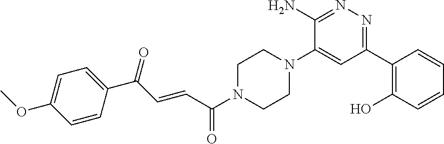 [SMILES: COc1ccc(C(=O)/C=C/C(=O)N2CCN(c3cc(-c4ccccc4O)nnc3N)CC2)cc1]

General Procedure A was followed with trans-3-(4-methoxybenzoyl)acrylic acid (13 mg, 0.06 mmol), HATU (28 mg, 0.07 mmol), DIPEA (0.05 mL, 0.29 mmol), and Compound 316 (16 mg, 0.06 mmol). The crude residue was purified by silica gel chromatography (0-10% MeOH in DCM) to afford 26 mg (92%, 2 steps) of Compound 128 as a powder. ¹H NMR (700 MHz, CDCl₃) δ 8.02 (d, J=8.5 Hz, 2H), 7.97 (d, J=14.9 Hz, 1H), 7.54 (d, J=8.0 Hz, 1H), 7.48 (d, J=14.9 Hz, 1H), 7.30 (s, 1H), 7.26 (t, J=1.7 Hz, 1H), 7.00 (d, J=8.1 Hz, 1H), 6.96 (d, J=8.6 Hz, 2H), 6.89 (t, J=7.5 Hz, 1H), 3.92 (t, J=5.0 Hz, 2H), 3.87 (s, 3H), 3.86-3.83 (m, 2H), 3.16 (t, J=5.2 Hz, 4H), 2.47 (br s, 2H). ¹³C NMR (151 MHz, CDCl₃) δ 187.8, 164.6, 164.5, 158.7, 155.0, 154.1, 140.6, 135.3, 131.5, 131.1, 130.9, 129.8, 125.3, 119.1, 118.4, 117.5, 114.3, 111.6, 55.7, 49.6, 49.1, 49.1, 45.9, 42.1. HRMS (ESI) m/z calcd for [C₂₅H₂₅N₅O₄+H]⁺=460.1979, found 460.1983.

Analogous methods to those provided in Examples 1-20 above were followed to obtain the compounds of Tables 4 and 5 below.

TABLE 4
Characterization of Exemplary Intermediates

| Compound | | | LC-MS |
|---|---|---|---|
| No. | Structure and Name | ¹H NMR | (min; m/z) |
| Compound 145 | 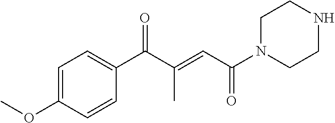 [SMILES: COc1ccc(C(=O)/C(C)=C/C(=O)N2CCNCC2)cc1] (E)-1-(4-methoxyphenyl)-2-methyl-4- | ¹H NMR (400 MHz, DMSO) δ 8.24-8.18 (m, 1H), 7.79-7.67 (m, 2H), 7.12-6.98 (m, 2H), 6.64 (d, J = 1.8 Hz, 1H), 3.83 (s, 3H), 3.24 (s, 2H), 2.74 (s, 1H), 2.58 (s, 1H), 2.00 (d, J = 1.5 Hz, 3H). | Rt = 1.52; 289.1 [M + H]+ |
| | (piperazin-1-yl)but-2-ene-1,4-dione | | |
| Compound 147 | 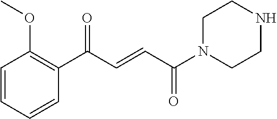 [SMILES: COc1ccccc1C(=O)/C=C/C(=O)N1CCNCC1] | ¹H NMR (400 MHz, DMSO) δ 9.40 (s, 1H), 7.68-7.46 (m, 3H), 7.35- 7.19 (m, 2H), 7.08 (td, J = 7.5, 1.0 Hz, 1H), 3.90- 3.75 (m, 6H), 3.14 (s, 2H). | Rt = 1.59; 275.1 [M + H]+ |
| | (E)-1-(2-methoxyphenyl)-4- | | |
| | (piperazin-1-yl)but-2-ene-1,4-dione | | |
| Compound 149 | 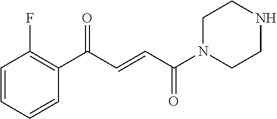 [SMILES: O=C(/C=C/C(=O)N1CCNCC1)c1ccccc1F] (E)-1-(2-fluorophenyl)-4-(piperazin-1- | ¹H NMR (400 MHz, DMSO) δ 9.32 (s, 1H), 7.82 (td, J = 7.7, 1.8 Hz, 1H), 7.73 (dddd, J = 9.3, 7.2, 5.2, 1.8 Hz, 1H), 7.50 (dd, J = 15.3, 3.1 Hz, 1H), 7.45-7.32 (m, 3H), 3.81 (dt, J = 15.4, 5.3 Hz, 4H) 3.13 (q, J = 6.4 Hz, 4H) | Rt = 1.49; 263.1 [M + H]⁺ |
| | yl)but-2-ene-1,4-dione | | |
| Compound 150 | 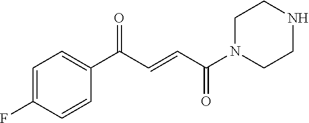 [SMILES: O=C(/C=C/C(=O)N1CCNCC1)c1ccc(F)cc1] (E)-1-(4-fluorophenyl)-4-(piperazin-1- yl)but-2-ene-1,4-dione | ¹H NMR (400 MHz, DMSO) δ 9.42 (s, 1H), 8.18-8.09 (m, 2H), 7.79 (d, J = 15.2 Hz, 1H), 7.48- 7.36 (m, 3H), 3.84-3.79 (m, 4H), 3.14 (s, 4H); 19F NMR (376 MHz, DMSO) δ −104.8 | Rt = 1.58; 263.1 [M + H]+ |
| Compound 152 | 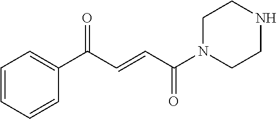 [SMILES: O=C(/C=C/C(=O)N1CCNCC1)c1ccccc1] (E)-1-phenyl-4-(piperazin-1-yl)but-2- ene-1,4-dione | ¹H NMR (400 MHz, DMSO) δ 9.35 (s, 1H), 8.08-7.99 (m, 2H), 7.80 (d, J = 15.3 Hz, 1H), 7.76- 7.67 (m, 1H), 7.59 (dd, J = 8.3, 7.2 Hz, 2H), 7.44 (d, J = 15.3 Hz, 1H), 3.82 (dt, J = 13.2, 5.0 Hz, 4H), 3.14 (p, J = 5.0 Hz, 4H). | Rt = 1.50; 245.1 [M + H]+ |
| Compound 153 | 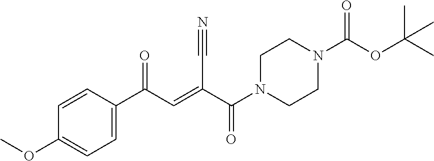 [SMILES: COc1ccc(C(=O)/C=C(\C#N)C(=O)N2CCN(C(=O)OC(C)(C)C)CC2)cc1] | ¹H NMR (400 MHz, CDCl₃) δ 7.94 (d, J = 8.4 Hz, 2H), 7.65 (s, 1H), 7.28 (s, 1H), 7.01 (d, J = 8.4 Hz, 2H), 3.93 (s, 3H), 3.68 (s, 1H), 3.57 (d, J = 5.8 Hz, 4H), 3.50-3.45 (m, 2H), 1.50 (s, 8H). | Rt = 1.92; 422.1 [M + Na]+ |
| | tert-butyl (E)-4-(2-cyano-4-(4- | | |
| | methoxyphenyl)-4-oxobut-2- | | |
| | enoyl)piperazine-1-carboxylate | | |
| Compound 154 | 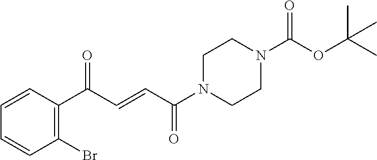 [SMILES: CC(C)(C)OC(=O)N1CCN(C(=O)/C=C/C(=O)c2ccccc2Br)CC1] | ¹H NMR (400 MHz, DMSO) δ 7.77 (dd, J = 7.5, 1.4 Hz, 1H), 7.62- 7.46 (m, 3H), 7.23 (d, J = 15.7 Hz, 1H), 7.15 (d, J = 15.6 Hz, 1H), 3.52 (dd, J = 11.9, 8.0 Hz, 4H), 3.40- 3.33 (m, 4H), 1.41 (s, 9H). | Rt = 2.68; 446.4 [M + Na]+ |
| | tert-butyl (E)-4-(4-(2-bromophenyl)-4- | | |
| | oxobut-2-enoyl)piperazine-1- | | |
| | carboxylate | | |
| Compound 155 | 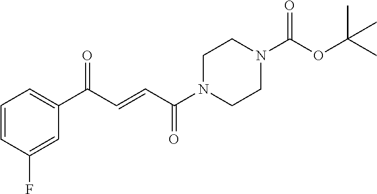 [SMILES: CC(C)(C)OC(=O)N1CCN(C(=O)/C=C/C(=O)c2cccc(F)c2)CC1] tert-butyl (E)-4-(4-(3-fluorophenyl)-4- oxobut-2-enoyl)piperazine-1- carboxylate | ¹H NMR (400 MHz, CDCl₃) δ 7.83 (d, J = 15.0 Hz, 1H), 7.75 (dt, J = 7.8, 1.3 Hz, 1H), 7.65 (ddd, J = 9.3, 2.7, 1.6 Hz, 1H), 7.49- 7.38 (m, 2H), 7.25 (tdd, J = 8.3, 2.6, 1.0 Hz, 1H), 3.69-3.62 (m, 2H), 3.58- 3.51 (m, 2H), 3.42 (t, J = 5.4 Hz, 4H), 1.46 (s, 1H), 1.41 (s, 9H). | Rt = 2.90; 385.1 [M + Na]+ |
| Compound 156 | 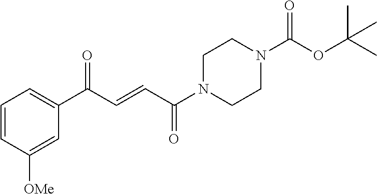 [SMILES: COc1cccc(C(=O)/C=C/C(=O)N2CCN(C(=O)OC(C)(C)C)CC2)c1] | ¹H NMR (400 MHz, DMSO) δ 7.76 (d, J = 15.3 Hz, 1H), 7.64 (dd, J = 7.7, 1.4 Hz, 1H), 7.55-7.40 (m, 3H), 7.28 (dd, J = 8.2, 2.7 Hz, 1H), 3.85 (s, 3H), 3.57 (q, J = 5.2 Hz, 4H), 3.38 (t, J = 5.2 Hz, 4H), 1.42 (s, 9H). | Rt = 2.62; 397.2 [M + Na]+ |
| | tert-butyl (E)-4-(4-(3- | | |
| | methoxyphenyl)-4-oxobut-2- | | |
| | enoyl)piperazine-1-carboxylate | | |
| Compound 157 | 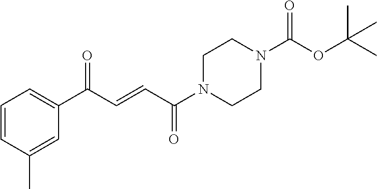 [SMILES: Cc1cccc(C(=O)/C=C/C(=O)N2CCN(C(=O)OC(C)(C)C)CC2)c1] | ¹H NMR (400 MHz, CDCl₃) δ 7.99 (d, J = 14.9 Hz, 1H), 7.86 (d, J = 9.0 Hz, 2H), 7.53-7.38 (m, 3H), 3.75 (t, J = 5.3 Hz, 2H), 3.64 (d, J = 5.6 Hz, 2H), 3.52 (t, J = 5.3 Hz, 4H), 2.46 (s, 3H), 1.51 (s, 9H). | Rt = 2.98; 381.1 [M + Na]+ |
| | tert-butyl (E)-4-(4-oxo-4-(m-toly1)but- | | |
| | 2-enoyl)piperazine-1-carboxylate | | |
| Compound 161 | 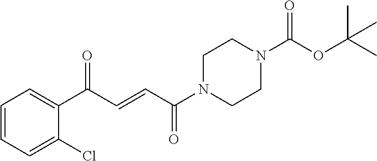 [SMILES: CC(C)(C)OC(=O)N1CCN(C(=O)/C=C/C(=O)c2ccccc2Cl)CC1] | ¹H NMR (400 MHz, DMSO) δ 7.67-7.56 (m, 3H), 7.51 (ddd, J = 7.4, 5.5, 3.1 Hz, 1H), 7.31- 7.16 (m, 2H), 3.58-3.50 (m, 4H), 3.30 (m, 4H), 1.42 (s, 9H). | Rt = 2.66; 401.1 [M + Na]+ |
| | tert-butyl (E)-4-(4-(2-chlorophenyl)-4- | | |
| | oxobut-2-enoyl)piperazine-1- | | |
| | carboxylate | | |
| Compound 162 | 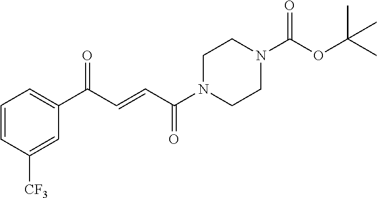 [SMILES: CC(C)(C)OC(=O)N1CCN(C(=O)/C=C/C(=O)c2cccc(C(F)(F)F)c2)CC1] | ¹H NMR (400 MHz, CDCl₃) δ 8.31 (s, 1H), 8.24 (d, J = 7.9 Hz, 1H), 7.98 (d, J = 14.9 Hz, 1H), 7.90 (d, J = 7.8 Hz, 1H), 7.69 (t, J = 7.8 Hz, 1H), 7.56 (d, J = 14.9 Hz, 1H), 3.76 (t, J = 5.3 Hz, 2H), 3.68-3.61 (m, 2H), 3.53 (q, J = 3.8 Hz, 4H), 1.51 (s, 9H). | Rt = 2.72; 435.1 [M + Na]+ |
| | tert-butyl (E)-4-(4-oxo-4-(3- | | |
| | (trifluoromethyl)phenyl)but-2- | | |
| | enoyl)piperazine-1-carboxylate | | |
| Compound 163 | 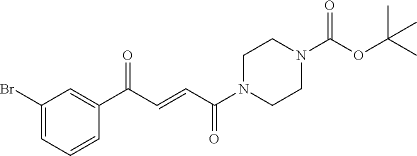 [SMILES: CC(C)(C)OC(=O)N1CCN(C(=O)/C=C/C(=O)c2cccc(Br)c2)CC1] tert-butyl (E)-4-(4-(3-bromophenyl)-4- oxobut-2-enoyl)piperazine-1- | ¹H NMR (400 MHz, CDCl₃) δ 8.19 (t, J = 1.8 Hz, 1H), 7.97 (dt, J = 7.8, 1.4 Hz, 1H), 7.92 (d, J = 14.9 Hz, 1H), 7.76 (ddd, J = 7.9, 2.0, 1.0 Hz, 1H), 7.51 (d, J = 14.9 Hz, 1H), 7.42 (t, J = 7.9 Hz, 1H), 3.74 (t, J = 5.3 Hz, 2H), 3.63 (dd, J = 6.7, 3.9 Hz, 2H), 3.51 (t, J = 5.2 Hz, 4H), 1.50 (s, 9H). | Rt = 2.70; 446.1 [M + Na]+ |
| | carboxylate | | |
| Compound 164 | 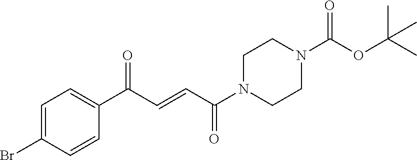 [SMILES: CC(C)(C)OC(=O)N1CCN(C(=O)/C=C/C(=O)c2ccc(Br)cc2)CC1] | ¹H NMR (400 MHz, CDCl₃) δ 7.88-7.80 (m, 3H), 7.59 (d, J = 8.8 Hz, 2H), 7.42 (d, J = 14.9 Hz, 1H), 3.65 (t, J = 5.2 Hz, 2H), 3.60-3.50 (m, 2H), 3.42 (t, J = 5.3 Hz, 4H), 1.41 (s, 9H). | Rt = 2.80; 445.1 [M + Na]+ |
| | tert-butyl (E)-4-(4-(4-bromophenyl)-4- | | |
| | oxobut-2-enoyl)piperazine-1- | | |
| | carboxylate | | |
| Compound 166 | 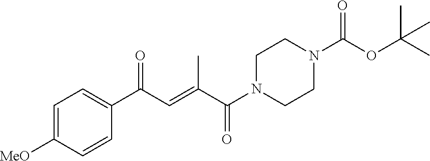 [SMILES: COc1ccc(C(=O)/C=C(\C)C(=O)N2CCN(C(=O)OC(C)(C)C)CC2)cc1] | ¹H NMR (400 MHz, DMSO) δ 8.01-7.93 (m, 2H), 7.11-7.03 (m, 2H), 7.00 (s, 1H), 3.86 (s, 3H), 3.51 (s, 4H), 3.46-3.37 (m, 4H), 2.18 (d, J = 1.6 Hz, 3H), 1.42 (s, 9H). | Rt = 2.62; 411.2 [M + Na]+ |
| | tert-butyl (E)-4-(4-(4- | | |
| | methoxyphenyl)-2-methyl-4-oxobut-2- | | |
| | enoyl)piperazine-1-carboxylate | | |
| Compound 400 | 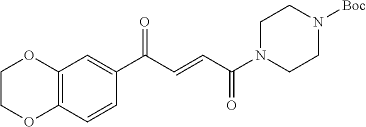 [SMILES: CC(C)(C)OC(=O)N1CCN(C(=O)/C=C/C(=O)c2ccc3c(c2)OCCO3)CC1] | ¹H NMR (400 MHz, DMSO) δ 7.73 (d, J = 15.2 Hz, 1H), 7.60 (dd, J = 8.5, 2.2 Hz, 1H), 7.52 (d, J = 2.1 Hz, 1H), 7.39 (d, J = 15.2 Hz, 1H), 7.03 (d, J = 8.4 Hz, 1H), 4.39-4.28 (m, 4H), 3.61-3.52 (m, | Rt = 2.55; 425.1 [M + Na]+ |
| | tert-butyl (E)-4-(4-(2,3- | 4H), 3.38 (t, J = 5.2 Hz, | |
| | dihydrobenzo[b][1,4]dioxin-6-y1)-4- | 4H), 1.42 (s, 9H). | |
| | oxobut-2-enoy1)piperazine-1- | | |
| | carboxylate | | |
| Compound 401 | 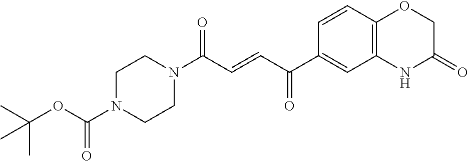 [SMILES: CC(C)(C)OC(=O)N1CCN(C(=O)/C=C/C(=O)c2ccc3c(c2)NC(=O)CO3)CC1] | ¹H NMR (400 MHz, DMSO) δ 10.89 (s, 1H), 7.77-7.66 (m, 2H), 7.57 (d, J = 2.1 Hz, 1H), 7.43 (d, J = 15.1 Hz, 1H), 7.09 (d, J = 8.4 Hz, 1H), 4.73 (s, 2H), 3.57 (t, J = 6.9 Hz, 4H), 3.38 (q, J = 5.4 Hz, 4H), 1.42 (s, 9H). | Rt = 2.21; 438.2 [M + Na]+ |
| | tert-butyl (E)-4-(4-oxo-4-(3-oxo-3,4- | | |
| | dihydro-2H-benzo[b][1,4]oxazin-6- | | |
| | yl)but-2-enoyl)piperazine-1- | | |
| | carboxylate | | |
| Compound 402 | 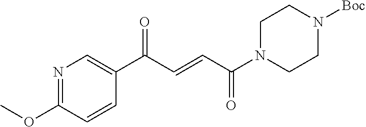 [SMILES: COc1ccc(C(=O)/C=C/C(=O)N2CCN(C(=O)OC(C)(C)C)CC2)cn1] | ¹H NMR (400 MHz, DMSO) δ 8.93 (d, J = 2.4 Hz, 1H), 8.29 (dd, J = 8.8, 2.5 Hz, 1H), 7.75 (d, J = 15.2 Hz, 1H), 7.44 (d, J = 15.2 Hz, 1H), 6.99 (d, J = 8.8 Hz, 1H), 3.97 (s, 3H), 3.57 (q, J = 4.8 Hz, 4H), | Rt = 2.47; 376.1 [M + H]+ |
| | tert-butyl (E)-4-(4-(6-methoxypyridin- | 3.38 (t, J = 5.3 Hz, 4H), | |
| | 3-yl)-4-oxobut-2-enoyl)piperazine-1- | 1.42 (s, 9H). | |
| | carboxylate | | |
| Compound 403 | 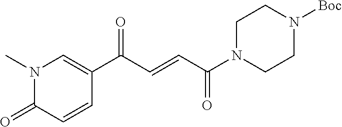 [SMILES: Cn1cc(C(=O)/C=C/C(=O)N2CCN(C(=O)OC(C)(C)C)CC2)ccc1=O] | ¹H NMR (400 MHz, DMSO) δ 8.94 (d, J = 2.6 Hz, 1H), 7.94 (dd, J = 9.5, 2.7 Hz, 1H), 7.73 (d, J = 15.1 Hz, 1H), 7.44 (d, J = 15.0 Hz, 1H), 6.50 (d, J = 9.5 Hz, 1H), 3.56 (d, J = 13.1 Hz, 7H), 3.37 (d, J = | Rt = 2.16; 398.1 [M + Na]+ |
| | tert-butyl (E)-4-(4-(1-methyl-6-oxo- | 4.7 Hz, 4H), 1.42 (s, 9H). | |
| | 1,6-dihydropyridin-3-y1)-4-oxobut-2- | | |
| | enoyl)piperazine-1-carboxylate | | |
| Compound 404 | 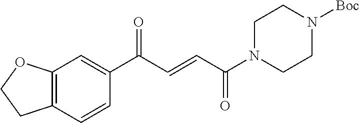 [SMILES: CC(C)(C)OC(=O)N1CCN(C(=O)/C=C/C(=O)c2ccc3c(c2)OCC3)CC1] | ¹H NMR (400 MHz, CDCl₃) δ 7.83 (d, J = 14.9 Hz, 1H), 7.49 (dd, J = 7.7, 1.5 Hz, 1H), 7.42-7.32 (m, 2H), 7.23 (d, J = 7.7 Hz, 1H), 4.57 (t, J = 8.7 Hz, 2H), 3.65 (t, J = 5.4 | Rt = 2.57; 409.1 [M + Na]+ |
| | tert-butyl (E)-4-(4-(2,3- | Hz, 2H), 3.53 (d, J = 5.7 | |
| | dihydrobenzofuran-6-yl)-4-oxobut-2- | Hz, 2H), 3.42 (dd, J = 6.5, | |
| | enoyl)piperazine-1-carboxylate | 3.9 Hz, 4H), 3.25-3.16 | |
| | | (m, 2H), 1.41 (s, 9H). | |
| Compound 405 | 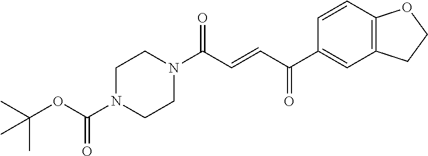 [SMILES: CC(C)(C)OC(=O)N1CCN(C(=O)/C=C/C(=O)c2ccc3c(c2)CCO3)CC1] | ¹H NMR (400 MHz, CDCl₃) δ 8.02-7.90 (m, 3H), 7.47 (d, J = 14.9 Hz, 1H), 6.87 (d, J = 8.4 Hz, 1H), 4.72 (t, J = 8.8 Hz, 2H), 3.74 (t, J = 5.3 Hz, 2H), 3.63 (d, J = 5.5 Hz, 2H), 3.51 (t, J = 5.2 Hz, 3H), 3.30 (t, J = 8.8 Hz, 2H), 1.51 (s, 9H) | Rt = 2.54; 409.2 [M + Na]+ |
| | tert-butyl (E)-4-(4-(2,3- | | |
| | dihydrobenzofuran-5-y1)-4-oxobut-2- | | |
| | enoyl)piperazine-1-carboxylate | | |
| Compound 406 | 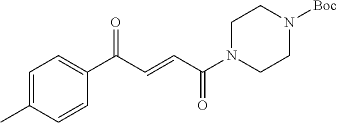 [SMILES: Cc1ccc(C(=O)/C=C/C(=O)N2CCN(C(=O)OC(C)(C)C)CC2)cc1] | ¹H NMR (400 MHz, DMSO) δ 7.99-7.92 (m, 2H), 7.77 (d, J = 15.2 Hz, 1H), 7.47-7.36 (m, 3H), 3.58 (q, J = 5.5 Hz, 4H), 3.38 (q, J = 5.3 Hz, 4H), 2.41 (s, 3H), 1.42 (s, 9H). | Rt = 2.67; 381.1 [M + Na]+ |
| | tert-butyl (E)-4-(4-oxo-4-(p-tolyl)but- | | |
| | 2-enoyl)piperazine-1-carboxylate | | |
| Compound 407 | 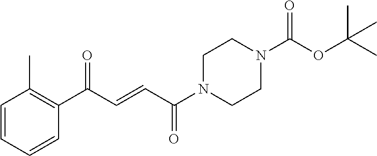 [SMILES: Cc1ccccc1C(=O)/C=C/C(=O)N1CCN(C(=O)OC(C)(C)C)CC1] | ¹H NMR (400 MHz, CDCl₃) δ 7.62-7.53 (m, 2H), 7.40-7.31 (m, 1H), 7.27-7.17 (m, 6H), 3.63 (d, J = 5.5 Hz, 2H), 3.50 (d, J = 5.8 Hz, 2H), 3.41 (t, J = 5.3 Hz, 4H), 2.45 (s, 3H), 1.41 (d, J = 1.1 Hz, 9H) | Rt = 2.68; 381.2 [M + Na]+ |
| | tert-butyl (E)-4-(4-oxo-4-(o-tolyl)but- | | |
| | 2-enoyl)piperazine-1-carboxylate | | |
| Compound 408 | 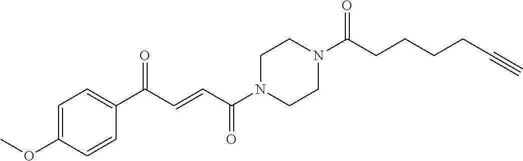 | ¹H NMR (400 MHz, CDCl₃) δ 8.04 (d, J = 8.6 Hz, 2H), 8.02-7.94 (m, 1H), 7.46 (d, J = 14.8 Hz, 1H), 7.02-6.95 (m, 2H), 3.90 (s, 3H), 3.77 (d, J = 6.0 Hz, 2H), 3.67 (d, J = 12.6 Hz, 4H), 3.54 (s, 2H), 2.39 (t, J = 7.8 Hz, 2H), | Rt = 2.17; 672.3 [M + H]+ |
| | (E)-1-(4-(hept-6-ynoyl)piperazin-1- | 2.25 (td, J = 7.0, 2.7 Hz, | |
| | y1)-4-(4-methoxyphenyl)but-2-ene- | 2H), 1.96 (t, J = 2.6 Hz, | |
| | 1,4-dione | 1H), 1.79 (p, J = 7.5 Hz, | |
| | | 2H), 1.61 (q, J = 7.2 Hz, | |
| | | 2H). | |
| Compound 409 | 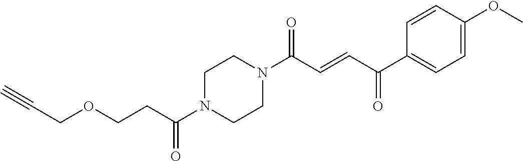 [SMILES: C#CCOCCC(=O)N1CCN(C(=O)/C=C/C(=O)c2ccc(OC)cc2)CC1] | ¹H NMR (400 MHz, CDCl₃) δ 8.08 (d, J = 8.4 Hz, 2H), 8.02 (d, J = 15.0 Hz, 1H), 7.50 (d, J = 14.9 Hz, 1H), 7.30 (s, 4H), 7.02 (d, J = 8.6 Hz, 2H), 4.20 (s, 2H), 3.92 (d, J = 10.7 Hz, 4H), 3.81 (s, 1H), 3.75 (s, 2H), 3.69 (s, 1H), 3.62 | Rt = 2.08; 385.2 [M + H]+ |
| | (E)-1-(4-methoxyphenyl)-4-(4-(3- | (s, 2H), 2.72 (s, 2H), 2.48 | |
| | (prop-2-yn-1- | (d, J = 2.8 Hz, 1H) | |
| | yloxy)propanoyl)piperazin-1-yl)but-2- | | |
| | ene-1,4-dione | | |
| Compound 410 | 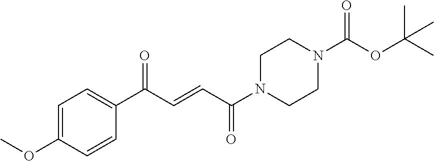 [SMILES: COc1ccc(C(=O)/C=C/C(=O)N2CCN(C(=O)OC(C)(C)C)CC2)cc1] | ¹H NMR (400 MHz, CDCl₃) δ 8.11-8.04 (m, 2H), 8.01 (dd, J= 14.9, 1.4 Hz, 1H), 7.50 (dd, J = 14.9, 1.5 Hz, 1H), 7.30 (s, 1H), 7.05-6.98 (m, 2H), 3.93 (d, J = 1.5 Hz, 3H), 3.76 (t, J = 5.3 Hz, 2H), 3.65 (t, J = 5.2 Hz, 2H), 3.52 (t, J = 5.2 Hz, 4H), | Rt = 2.57; 397.1 [M + Na]+ |
| | tert-butyl (E)-4-(4-(4- | 1.52 (s, 9H) | |
| | methoxyphenyl)-4-oxobut-2- | | |
| | enoyl)piperazine-1-carboxylate | | |
| Compound 411 | 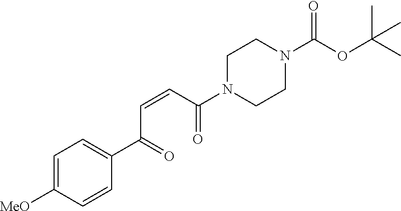 [SMILES: COc1ccc(C(=O)/C=C\C(=O)N2CCN(C(=O)OC(C)(C)C)CC2)cc1] | ¹H NMR (400 MHz, CDCl₃) δ 7.91-7.83 (m, 2H), 6.95 (d, J = 11.9 Hz, 1H), 6.91-6.84 (m, 2H), 6.49 (d, J = 12.0 Hz, 1H), 3.81 (s, 3H), 3.57 (s, 2H), 3.38 (s, 4H), 1.40 (s, 9H). | Rt = 2.46; 375.1 [M + H]+ |
| | tert-butyl (Z)-4-(4-(4- | | |
| | methoxyphenyl)-4-oxobut-2- | | |
| | enoyl)piperazine-1-carboxylate | | |
| Compound 412 | 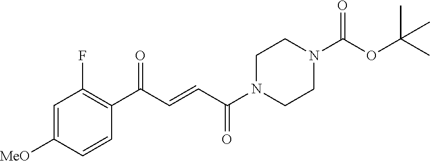 [SMILES: COc1ccc(C(=O)/C=C/C(=O)N2CCN(C(=O)OC(C)(C)C)CC2)c(F)c1] | ¹H NMR (400 MHz, CDCl₃) δ 7.90 (t, J = 8.7 Hz, 1H), 7.80 (dd, J = 15.0, 3.3 Hz, 1H), 7.42 (dd, J = 15.0, 1.6 Hz, 1H), 6.81 (dd, J = 8.9, 2.4 Hz, 1H), 6.67 (dd, J= 13.1, 2.4 Hz, 1H), 3.90 (s, 2H), 3.73 (t, J = 5.3 Hz, 2H), 3.62 (t, J = 5.1 Hz, 2H), 3.50 (t, J = | Rt = 2.48; 415.1 [M + Na]+ |
| | tert-butyl (E)-4-(4-(2-fluoro-4- | 5.3 Hz, 3H), 1.50 (s, | |
| | methoxyphenyl)-4-oxobut-2- | 9H). | |
| | enoyl)piperazine-1-carboxylate | | |
| Compound 413 | 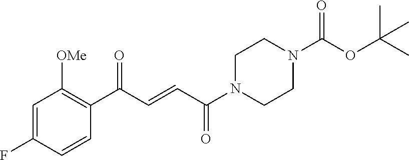 [SMILES: COc1cc(F)ccc1C(=O)/C=C/C(=O)N1CCN(C(=O)OC(C)(C)C)CC1] | ¹H NMR (400 MHz, CDCl₃) δ 7.87-7.75 (m, 2H), 7.36 (d, J = 15.0 Hz, 1H), 6.81-6.67 (m, 2H), 3.94 (s, 3H), 3.72 (t, J = 5.2 Hz, 2H), 3.66-3.59 (m, 2H), 3.54-3.47 (m, 4H), 1.50 (s, 9H); 19F NMR (376 MHz, CDCl₃) δ −102.2 | Rt = 2.63; 415.1 [M + Na]+ |
| | tert-butyl (E)-4-(4-(4-fluoro-2- | | |
| | methoxyphenyl)-4-oxobut-2- | | |
| | enoyl)piperazine-1-carboxylate | | |
| Compound 414 | 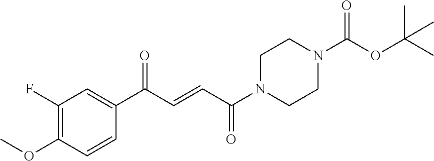 [SMILES: COc1ccc(C(=O)/C=C/C(=O)N2CCN(C(=O)OC(C)(C)C)CC2)cc1F] | ¹H NMR (400 MHz, CDCl₃) δ 7.85 (d, J = 14.9 Hz, 1H), 7.81-7.69 (m, 2H), 7.41 (d, J = 14.8 Hz, 1H), 6.97 (t, J = 8.3 Hz, 1H), 3.91 (s, 3H), 3.65 (t, J = 5.3 Hz, 2H), 3.55 (s, 2H), 3.46-3.38 (m, 4H), 1.41 (s, 9H). | Rt = 2.26; 415.2 [M + Na]+ |
| | tert-butyl (E)-4-(4-(3-fluoro-4- | | |
| | methoxyphenyl)-4-oxobut-2- | | |
| | enoyl)piperazine-1-carboxylate | | |
| Compound 415 | 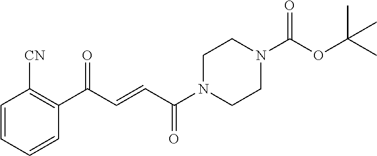 [SMILES: CC(C)(C)OC(=O)N1CCN(C(=O)/C=C/C(=O)c2ccccc2C#N)CC1] | ¹H NMR (400 MHz, CDCl₃) δ 7.91 (d, J = 7.6 Hz, 1H), 7.80 (d, J = 7.2 Hz, 1H), 7.75-7.59 (m, 3H), 7.47 (dd, J = 15.1, 1.9 Hz, 1H), 7.19 (s, 1H), 3.64 (s, 2H), 3.53 (d, J = 5.1 Hz, 2H), 3.42 (d, J = 6.3 Hz, 4H), 1.41 (d, J = 1.9 Hz, 9H). | Rt = 2.46; 392.1 [M + Na]+ |
| | tert-butyl (E)-4-(4-(2-cyanophenyl)-4- | | |
| | oxobut-2-enoyl)piperazine-1- | | |
| | carboxylate | | |
| Compound 416 | 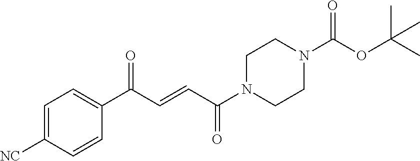 [SMILES: CC(C)(C)OC(=O)N1CCN(C(=O)/C=C/C(=O)c2ccc(C#N)cc2)CC1] | ¹H NMR (400 MHz, CDCl₃) δ 8.08-8.01 (m, 2H), 7.85 (d, J = 14.9 Hz, 1H), 7.79-7.72 (m, 2H), 7.46 (d, J = 14.9 Hz, 1H), 3.66 (t, J = 5.2 Hz, 2H), 3.54 (d, J = 5.7 Hz, 2H), 3.43 (d, J = 6.0 Hz, 4H), 1.41 (s, 9H) | Rt = 2.53; 392.1 [M + Na]+ |
| | tert-butyl (E)-4-(4-(4-cyanophenyl)-4- | | |
| | oxobut-2-enoyl)piperazine-1- | | |
| | carboxylate | | |
| Compound 417 | 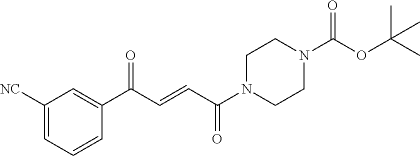 [SMILES: CC(C)(C)OC(=O)N1CCN(C(=O)/C=C/C(=O)c2cccc(C#N)c2)CC1] | ¹H NMR (400 MHz, CDCl₃) δ 8.34 (t, J = 1.8 Hz, 1H), 8.27 (dt, J = 7.9, 1.5 Hz, 1H), 7.97-7.88 (m, 2H), 7.69 (t, J = 7.8 Hz, 1H), 7.56 (d, J = 14.9 Hz, 1H), 3.75 (t, J = 5.3 Hz, 2H), 3.67-3.60 (m, 2H), 3.52 (dt, J = 7.0, 3.2 Hz, 4H), 1.50 (s, 9H). | Rt = 2.38; 392.2 [M + Na]+ |
| | tert-butyl (E)-4-(4-(3-cyanophenyl)-4- | | |
| | oxobut-2-enoyl)piperazine-1- | | |
| | carboxylate | | |
| Compound 418 | 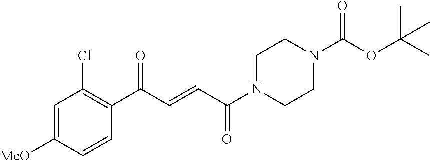 [SMILES: COc1ccc(C(=O)/C=C/C(=O)N2CCN(C(=O)OC(C)(C)C)CC2)c(Cl)c1] | ¹H NMR (400 MHz, CDCl₃) δ 7.63-7.53 (m, 2H), 7.24 (d, J = 15.1 Hz, 1H), 6.90 (d, J = 2.4 Hz, 1H), 6.81 (dd, J = 8.7, 2.5 Hz, 1H), 3.80 (s, 4H), 3.63 (t, J = 5.4 Hz, 3H), 3.56- 3.49 (m, 3H), 3.44-3.37 (m, 5H), 1.41 (s, 9H). | Rt = 2.54; 431.1 [M + Na]+ |
| | tert-butyl (E)-4-(4-(2-chloro-4- | | |
| | methoxyphenyl)-4-oxobut-2- | | |
| | enoyl)piperazine-1-carboxylate | | |
| Compound 419 | 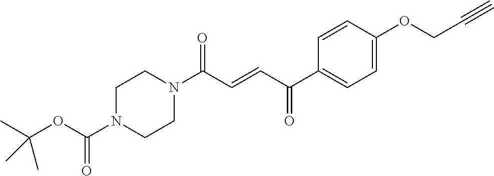 [SMILES: C#CCOc1ccc(C(=O)/C=C/C(=O)N2CCN(C(=O)OC(C)(C)C)CC2)cc1] | ¹H NMR (400 MHz, CDCl₃) δ 8.11-8.04 (m, 2H), 7.99 (d, J = 14.9 Hz, 1H), 7.49 (d, J = 14.9 Hz, 1H), 7.13-7.06 (m, 2H), 4.81 (d, J = 2.4 Hz, 2H), 3.74 (t, J = 5.2 Hz, 2H), 3.64 (d, J = 6.0 Hz, 2H), 3.51 (t, J = 5.4 Hz, 4H), 2.59 (t, J = 2.4 Hz, 1H), 1.51 (s, 9H). | Rt = 2.49; 421.2 [M + Na]+ |
| | tert-butyl (E)-4-(4-oxo-4-(4-(prop-2- | | |
| | yn-1-yloxy)phenyl)but-2- | | |
| | enoyl)piperazine-1-carboxylate | | |

TABLE 5
Characterization of Exemplary Compounds.

| Compound | | | LC-MS |
|---|---|---|---|
| No. | Structure and Name | ¹H NMR | (min; m/z) |
| Compound 113 | 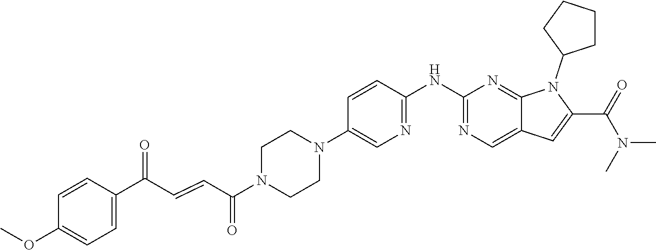 | ¹H NMR (400 MHz, DMSO) δ 9.34 (s, 1H), 8.76 (s, 1H), 8.18 (d, J = 9.1 Hz, 1H), 8.11-8.01 (m, 3H), 7.82 (d, J = 15.2 Hz, 1H), 7.53-7.44 (m, 2H), 7.15- 7.07 (m, 2H), 6.60 (s, 1H), 4.78-4.70 (m, 1H), 3.88 (s, 3H), 3.77 (s, 3H), 3.18 (d, J = 5.5 Hz, 4H), 3.06 (s, 5H), 2.44 (s, 1H), 1.99 (s, 3H), 1.65 (s, 2H). | Rt = 2.32; 623.3 [M + H]+ |
| | (E)-7-cyclopentyl-2-((5-(4-(4-(4- | | |
| | methoxyphenyl)-4-oxobut-2- | | |
| | enoyl)piperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 130 | 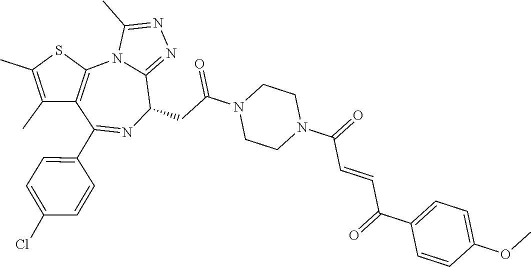 | ¹H NMR (400 MHz, DMSO) δ 8.07 (dd, J = 8.9, 2.4 Hz, 2H), 7.83 (d, J = 15.2 Hz, 1H), 7.53-7.40 (m, 5H), 7.11 (d, J = 8.5 Hz, 2H), 4.60 (t, J = 6.7 Hz, 1H), 3.88 (s, 3H), 3.75 (d, J = 6.1 Hz, 3H), 3.73-3.54 (m, 5H), 3.45 (dd, J = 18.1, 6.1 Hz, 1H), 2.61 (s, 3H), 2.45-2.40 (m, 3H), 1.66- 1.62 (m, 3H). | Rt = 2.57; 657.2 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(4- | | |
| | methoxyphenyl)but-2-ene-1,4-dione | | |
| Compound 139 | 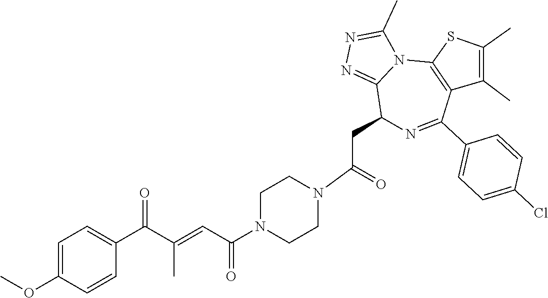 [SMILES: COc1ccc(C(=O)/C(C)=C/C(=O)N2CCN(C(=O)C[C@@H]3N=C(c4ccc(Cl)cc4)c4c(sc(C)c4C)-n4c(C)nnc43)CC2)cc1] | H NMR (400 MHz, CDCl₃) δ 7.94-7.86 (m, 2H), 7.47 (d, J = 8.2 Hz, 2H), 7.39 (d, J = 8.1 Hz, 2H), 7.02-6.94 (m, 2H), 6.35 (d, J = 1.9 Hz, 1H), 4.89 (s, 1H), 3.89 (s, 3H), 3.74 (s, 2H), 3.66 (d, J = 11.3 Hz, 2H), 2.77 (s, 3H), 2.45 (s, 3H), 2.16 (d, J = 1.5 Hz, 3H), 1.71 (s, 3H). | Rt = 2.50; 670.1 [M + H]+ |
| | (S,E)-4-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-1-(4- | | |
| | methoxyphenyl)-2-methylbut-2-ene-1,4- | | |
| | dione | | |
| Compound 140 | 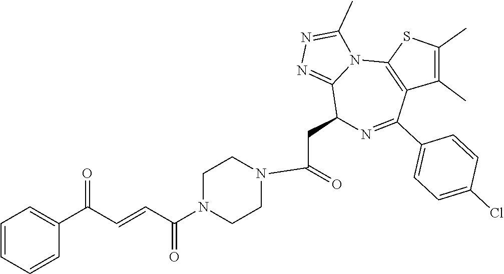 [SMILES: Cc1sc2c(c1C)C(c1ccc(Cl)cc1)=N[C@@H](CC(=O)N1CCN(C(=O)/C=C/C(=O)c3ccccc3)CC1)c1nnc(C)n1-2] | H NMR (400 MHz, CDCl₃) δ 8.08 (d, J = 7.8 Hz, 2H), 8.06-7.98 (m, 1H), 7.66 (t, J = 7.3 Hz, 1H), 7.60-7.49 (m, 3H), 7.44 (d, J = 8.2 Hz, 2H), 7.40-7.34 (m, 2H), 7.29 (s, 1H), 4.85 (t, J = 6.9 Hz, 1H), 4.07-3.95 (m, 2H), 3.86 (d, J = 6.4 Hz, 2H), 3.83-3.74 (m, 2H), 3.70-3.63 (m, 1H), 3.61 (s, 1H), 2.73-2.68 (m, 3H), 2.44 (s, 3H) | Rt = 2.63; 627.2 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-phenylbut-2- | | |
| | ene-1,4-dione | | |
| Compound 141 | 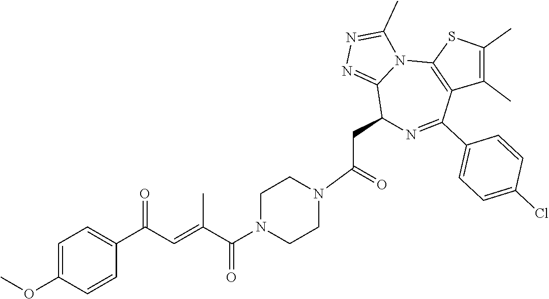 [SMILES: COc1ccc(C(=O)/C=C(\C)C(=O)N2CCN(C(=O)C[C@@H]3N=C(c4ccc(Cl)cc4)c4c(sc(C)c4C)-n4c(C)nnc43)CC2)cc1] | ¹H NMR (400 MHz, DMSO) δ 7.99 (d, J = 8.5 Hz, 1H), 7.54-7.41 (m, 3H), 7.08 (d, J = 8.9 Hz, 2H), 4.60 (t, J = 6.7 Hz, 1H), 3.86 (s, 2H), 3.78 (s, 1H), 3.67 (s, 1H), 3.58 (s, 1H), 3.47 (s, 1H), 2.61 (s, 2H), 2.43 (s, 2H), 2.22 (s, 2H), 1.64 (s, 2H), 1.24 (s,1H). | Rt = 2.66; 671.3 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(4- | | |
| | methoxyphenyl)-2-methylbut-2-ene-1,4- | | |
| | dione | | |
| Compound 142 | 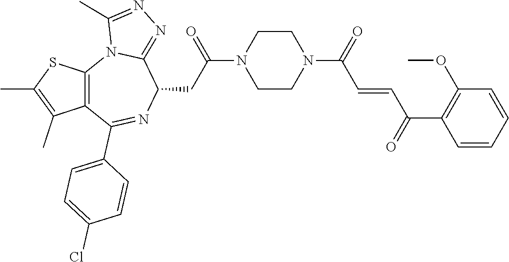 [SMILES: COc1ccccc1C(=O)/C=C/C(=O)N1CCN(C(=O)C[C@@H]2N=C(c3ccc(Cl)cc3)c3c(sc(C)c3C)-n3c(C)nnc32)CC1] | ¹H NMR (400 MHz, DMSO) δ 7.65-7.54 (m, 2H), 7.54-7.41 (m, 5H), 7.32 (dd, J = 15.3, 6.5 Hz, 1H), 7.21 (dd, J = 15.7, 8.4 Hz, 1H), 7.06 (dt, J = 19.7, 7.4 Hz, 1H), 4.60 (t, J = 6.7 Hz, 1H), 3.93-3.87 (m, 3H), 3.75 (d, J = 13.5 Hz, 4H), 3.66 (t, J = 8.0 Hz, 2H), 3.57 (d, J = 10.0 Hz, 4H), 3.45 (dd, J = 16.2, 6.3 Hz, 3H), 2.61 (s, 3H), 2.43 (s, 3H), 1.64 (s, 3H). | Rt = 2.69; 657.2 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(2- | | |
| | methoxyphenyl)but-2-ene-1,4-dione | | |
| Compound 143 | 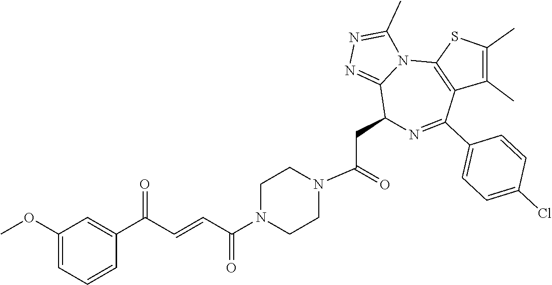 [SMILES: COc1cccc(C(=O)/C=C/C(=O)N2CCN(C(=O)C[C@@H]3N=C(c4ccc(Cl)cc4)c4c(sc(C)c4C)-n4c(C)nnc43)CC2)c1] | ¹H NMR (400 MHz, DMSO) δ 7.79 (d, J = 15.2 Hz, 1H), 7.66 (d, J = 7.7 Hz, 1H), 7.54-7.41 (m, 7H), 7.29 (d, J = 8.0 Hz, 1H), 4.60 (t, J = 6.7 Hz, 1H), 3.86 (s, 3H), 3.75 (s, 3H), 3.67 (dt, J = 15.2, 7.2 Hz, 2H), 3.61-3.55 (m, 3H), 3.45 (d, J = 16.5 Hz, 1H), 2.61 (s, 3H), 2.43 (s, 3H), 1.64 (s, 3H). | Rt = 2.66; 657.3 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(3- | | |
| | methoxyphenyl)but-2-ene-1,4-dione | | |
| Compound 144 | 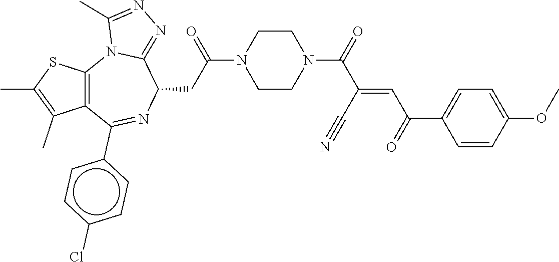 | ¹H NMR (400 MHz, CDCl₃) δ 7.96 (d, J = 8.6 Hz, 2H), 7.69 (s, 1H), 7.46- 7.34 (m, 4H), 7.02 (d, J = 8.8 Hz, 2H), 4.82 (t, J = 6.7 Hz, 1H), 4.02 (s, 1H), 3.93 (s, 3H), 3.92-3.82 (m, 2H), 3.70 (s, 1H), 3.64 (m, 2H), 3.56 (s, 1H), 2.70 (s, 3H), 2.43 (s, 3H), 1.71 (s, 3H). | Rt = 2.39; 683.0 [M + H]+ |
| | (S,E)-2-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazine-1-carbonyl)-4-(4- | | |
| | methoxyphenyl)-4-oxobut-2-enenitrile | | |
| Compound 146 | 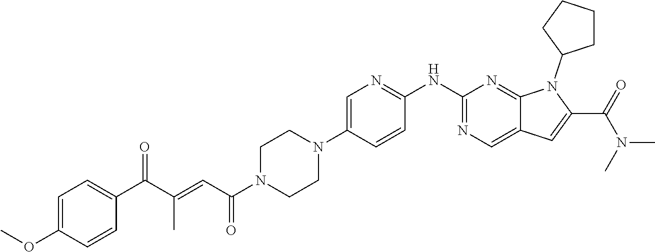 | ¹H NMR (400 MHz, DMSO) δ 9.33 (s, 1H), 8.18 (d, J = 9.0 Hz, 1H), 8.02 (d, J = 3.0 Hz, 1H), 7.79 (d, J = 8.8 Hz, 2H), 7.47 (dd, J = 9.1, 3.0 Hz, 1H), 7.10 (d, J = 8.8 Hz, 2H), 6.60 (s, 1H), 6.54 (d, J = 1.5 Hz, 1H), 4.78-4.69 (m, 1H), 3.87 (s, 3H), 3.72 (s, 2H), 3.58 (s, 2H), 3.36 (s, 28H), 3.15 (s, 3H), 3.06 (s, 5H), 2.44 (s, 1H), 2.03 (d, J = 1.3 Hz, 4H), 1.98 (s, 3H), 1.65 (s, 1H) | Rt = 2.29; 637.3 [M + H]+ |
| | (E)-7-cyclopentyl-2-((5-(4-(4-(4- | | |
| | methoxyphenyl)-3-methyl-4-oxobut-2- | | |
| | enoyl)piperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 148 | 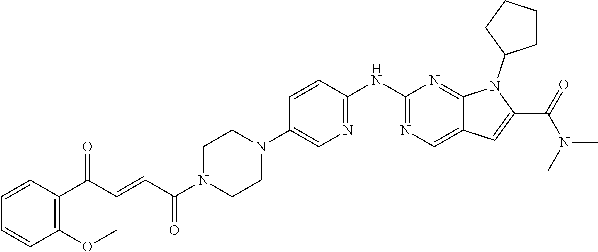 [SMILES: COc1ccccc1C(=O)/C=C/C(=O)N1CCN(c2ccc(Nc3ncc4cc(C(=O)N(C)C)n(C5CCCC5)c4n3)nc2)CC1] | ¹H NMR (400 MHz, DMSO) δ 9.34 (s, 1H), 8.76 (s, 1H), 8.18 (d, J = 9.0 Hz, 1H), 8.06-8.01 (m, 1H), 7.65-7.54 (m, 2H), 7.52- 7.44 (m, 2H), 7.33 (d, J = 15.3 Hz, 1H), 7.23 (d, J = 8.4 Hz, 1H), 7.09 (t, J = 7.5 Hz, 1H), 6.60 (s, 1H), 4.78- 4.69 (m, 1H), 3.89 (s, 3H), 3.75 (s, 3H), 3.17 (s, 4H), 3.06 (s, 6H), 2.45 (s, 2H), 1.99 (s, 5H), 1.65 (s, 2H). | Rt = 2.28; 623.3 [M + H]+ |
| | (E)-7-cyclopentyl-2-((5-(4-(4-(2- | | |
| | methoxyphenyl)-4-oxobut-2- | | |
| | enoyl)piperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 151 | 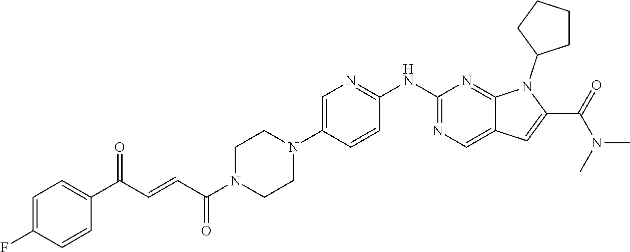 [SMILES: CN(C)C(=O)c1cc2cnc(Nc3ccc(N4CCN(C(=O)/C=C/C(=O)c5ccc(F)cc5)CC4)cn3)nc2n1C1CCCC1] | ¹H NMR (400 MHz, DMSO) δ 8.96 (s, 1H), 8.20- 8.11 (m, 2H), 7.99 (s, 1H), 7.94 (d, J = 2.8 Hz, 1H), 7.82 (d, J = 15.2 Hz, 1H), 7.72 (d, J = 9.3 Hz, 1H), 7.53 (d, J = 15.2 Hz, 1H), 7.42 (t, J = 8.8 Hz, 2H), 6.79 (s, 1H), 4.80 (p, J= 8.7 Hz, 1H), 3.80 (q, J = 6.1 Hz, 4H), 3.24 (s, 4H), 3.06 (s, 6H), 2.33 (d, J = 11.1 Hz, 2H), 1.99 (s, 4H), 1.66 (t, J = 6.1 Hz, 2H). | Rt = 2.25; 611.3 [M + H]+ |
| | (E)-7-cyclopentyl-2-((5-(4-(4-(4- | | |
| | fluorophenyl)-4-oxobut-2- | | |
| | enoyl)piperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 158 | 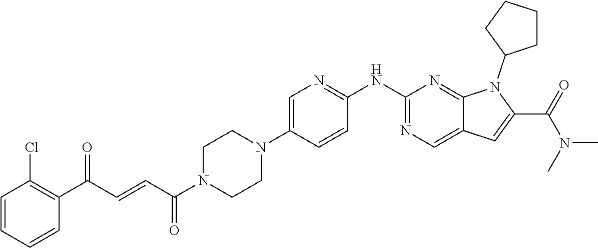 [SMILES: CN(C)C(=O)c1cc2cnc(Nc3ccc(N4CCN(C(=O)/C=C/C(=O)c5ccccc5Cl)CC4)cn3)nc2n1C1CCCC1] | ¹H NMR (400 MHz, DMSO) δ 9.36 (s, 1H), 8.77 (s, 1H), 8.18 (d, J = 9.1 Hz, 1H), 8.03 (d, J = 2.9 Hz, 1H), 7.68-7.57 (m, 3H), 7.56-7.45 (m, 2H), 7.33 (d, J = 15.6 Hz, 1H), 7.22 (d, J = 15.5 Hz, 1H), 6.61 (s, 1H), 4.78-4.69 (m, 1H), 3.76-3.68 (m, 4H), 3.17 (s, 3H), 3.06 (s, 4H), 2.44 (s, 1H), 1.99 (s, 3H), 1.66 (s, 1H) | Rt = 2.27; 627.3 [M + H]+ |
| | (E)-2-((5-(4-(4-(2-chlorophenyl)-4- | | |
| | oxobut-2-enoyl)piperazin-1-yl)pyridin- | | |
| | 2-yl)amino)-7-cyclopentyl-N,N- | | |
| | dimethyl-7H-pyrrolo[2,3-d]pyrimidine- | | |
| | 6-carboxamide | | |
| Compound 159 | 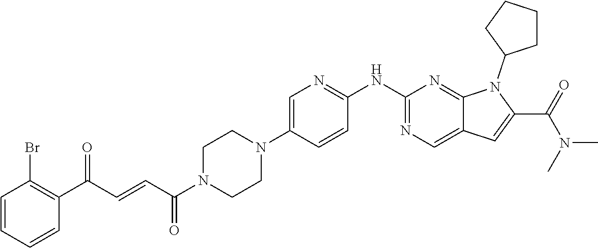 [SMILES: CN(C)C(=O)c1cc2cnc(Nc3ccc(N4CCN(C(=O)/C=C/C(=O)c5ccccc5Br)CC4)cn3)nc2n1C1CCCC1] | ¹H NMR (400 MHz, DMSO) δ 9.36 (s, 1H), 8.77 (s, 1H), 8.19 (d, J = 9.0 Hz, 1H), 8.03 (d, J = 2.9 Hz, 1H), 7.82-7.75 (m, 1H), 7.64-7.45 (m, 3H), 7.29 (d, J = 15.6 Hz, 1H), 7.18 (d, J = 15.6 Hz, 1H), 6.61 (s, 1H), 4.79-4.70 (m, 1H), 3.71 (d, J = 14.7 Hz, 3H), 3.17 (s, 3H), 3.07 (s, 4H), 2.45 (s, 1H), 1.99 (s, 2H), 1.66 (s, 1H) | Rt = 2.28; 671.3 [M + H]+ |
| | (E)-2-((5-(4-(4-(2-bromophenyl)-4- | | |
| | oxobut-2-enoyl)piperazin-1-yl)pyridin- | | |
| | 2-yl)amino)-7-cyclopentyl-N,N- | | |
| | dimethyl-7H-pyrrolo[2,3-d]pyrimidine- | | |
| | 6-carboxamide | | |
| Compound 160 | 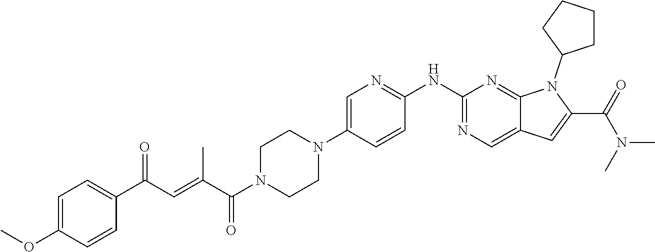 | ¹H NMR (400 MHz, DMSO) δ 9.34 (s, 1H), 8.76 (s, 1H), 8.17 (d, J = 9.1 Hz, 1H), 8.06-7.98 (m, 3H), 7.47 (dd, J = 9.1, 3.0 Hz, 1H), 7.17 (d, J = 1.6 Hz, 1H), 7.10-7.03 (m, 2H), 6.61 (s, 1H), 4.78-4.69 (m, 1H), 3.86 (s, 3H), 3.68 (s, 1H), 3.39 (s, 2H), 3.33 (s, 7H), 3.20 (s, 1H), 2.44 (s, 1H), 2.12 (d, J = 1.5 Hz, 3H), 1.98 (s, 3H), 1.65 (s, 1H). | Rt = 2.18; 637.3 [M + H]+ |
| | (E)-7-cyclopentyl-2-((5-(4-(4-(4- | | |
| | methoxyphenyl)-2-methyl-4-oxobut-2- | | |
| | enoyl)piperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 165 | 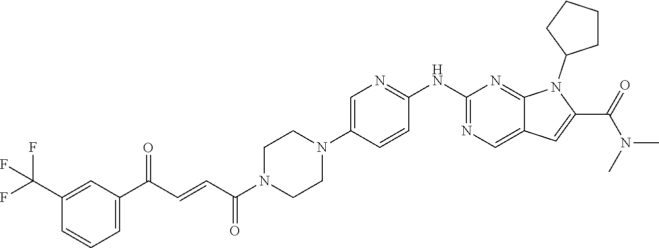 [SMILES: CN(C)C(=O)c1cc2cnc(Nc3ccc(N4CCN(C(=O)/C=C/C(=O)c5cccc(C(F)(F)F)c5)CC4)cn3)nc2n1C1CCCC1] | ¹H NMR (400 MHz, DMSO) δ 8.99 (s, 1H), 8.36 (d, J = 7.9 Hz, 1H), 8.30 (s, 1H), 8.08 (t, J = 8.0 Hz, 2H), 7.96 (d, J = 2.9 Hz, 1H), 7.90-7.81 (m, 2H), 7.72 (d, J = 9.4 Hz, 1H), 7.57 (d, J = 15.3 Hz, 1H), 6.81 (s, 1H), 4.81 (p, J = 8.7 Hz, 1H), 3.80 (q, J = 5.8 Hz, 4H), 3.26 (d, J = 5.4 Hz, 4H), 3.07 (s, 6H), 2.36- 2.29 (m, 2H), 2.01 (d, J = 13.3 Hz, 4H), 1.66 (s, 2H). | 661.3 [M + H]+ Rt = 2.38; |
| | (E)-7-cyclopentyl-N,N-dimethyl-2-((5- | | |
| | (4-(4-oxo-4-(3- | | |
| | (trifluoromethyl)phenyl)but-2- | | |
| | enoyl)piperazin-1-yl)pyridin-2- | | |
| | yl)amino)-7H-pyrrolo[2,3-d]pyrimidine- | | |
| | 6-carboxamide | | |
| Compound 200 | 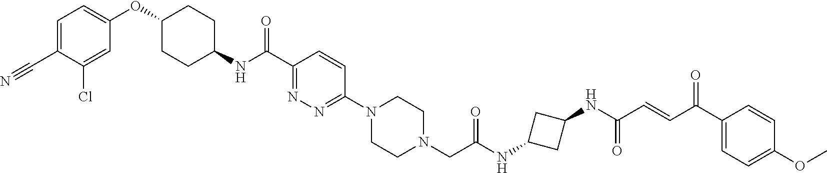 [SMILES: COc1ccc(C(=O)/C=C/C(=O)N[C@H]2C[C@H](NC(=O)CN3CCN(c4ccc(C(=O)N[C@H]5CC[C@H](Oc6ccc(C#N)c(Cl)c6)CC5)nn4)CC3)C2)cc1] | ¹H NMR (400 MHz, DMSO) δ 8.98 (d, J = 7.1 Hz, 1H), 8.64 (d, J = 8.2 Hz, 1H), 8.21 (s, 1H), 8.07- 8.00 (m, 2H), 7.86 (dd, J = 9.1, 5.2 Hz, 2H), 7.77 (d, J = 15.1 Hz, 1H), 7.42-7.33 (m, 2H), 7.18-7.07 (m, 2H), 6.95 (d, J = 15.2 Hz, 1H), 4.53 (d, J = 10.5 Hz, 1H), 4.39 (dd, J = 14.7, 7.5 | Rt = 2.93, 755.3 [M + H]+ |
| | N-((1r,4r)-4-(3-chloro-4- | Hz, 1H), 3.88 (s, 4H), 3.77 | |
| | cyanophenoxy)cyclohexyl)-6-(4-(2- | (s, 2H), 3.00 (s, 1H), 2.56 | |
| | (((1r,3r)-3-((E)-4-(4-methoxyphenyl)-4- | (d, J= 17.1 Hz, 5H), 2.35 (s, | |
| | oxobut-2-enamido)cyclobutyl)amino)-2- | 1H), 2.28-2.21 (m, 2H), | |
| | oxoethyl)piperazin-1-yl)pyridazine-3- | 2.10 (t, J = 9.7 Hz, 2H), | |
| | carboxamide | 1.90 (d, J = 12.3 Hz, 2H), | |
| | | 1.68 (d, J = 12.4 Hz, 1H), | |
| | | 1.62 (d, J = 12.0 Hz, 1H), | |
| | | 1.52 (q, J = 11.6 Hz, 2H), | |
| | | 1.40 (s, 1H), 1.31-1.15 (m, | |
| | | 1H). | |
| Compound 201 | 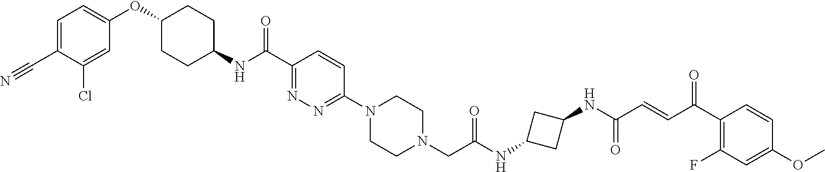 [SMILES: COc1ccc(C(=O)/C=C/C(=O)N[C@H]2C[C@H](NC(=O)CN3CCN(c4ccc(C(=O)N[C@H]5CC[C@H](Oc6ccc(C#N)c(Cl)c6)CC5)nn4)CC3)C2)c(F)c1] | ¹H NMR (400 MHz, DMSO) δ 8.99 (d, J = 7.1 Hz, 1H), 8.64 (d, J = 8.2 Hz, 1H), 8.19 (d, J = 7.6 Hz, 1H), 7.90-7.77 (m, 3H), 7.52 (dd, J = 15.2, 3.2 Hz, 1H), 7.42-7.33 (m, 2H), 7.14 (dd, J = 8.8, 2.4 Hz, 1H), 7.01 (dd, J = 13.5, 2.4 Hz, 1H), 6.98-6.88 (m, 2H), 4.54 (s, 1H), 4.37 (dd, | Rt = 2.97; 773.3 [M + H]+ |
| | N-((1r,4r)-4-(3-chloro-4- | J = 14.6, 7.2 Hz, 2H), 3.88 | |
| | cyanophenoxy)cyclohexyl)-6-(4-(2- | (s, 5H), 3.76 (s, 4H), 3.00 | |
| | (((1r,3r)-3-((E)-4-(2-fluoro-4- | (s, 2H), 2.58 (d, J = 5.3 Hz, | |
| | methoxyphenyl)-4-oxobut-2- | 4H), 2.32 (d, J = 8.0 Hz, | |
| | enamido)cyclobutyl)amino)-2- | 2H), 2.27-2.19 (m, 2H), | |
| | oxoethyl)piperazin-1-yl)pyridazine-3- | 2.11 (d, J = 12.0 Hz, 2H), | |
| | carboxamide | 1.90 (d, J = 12.2 Hz, 2H), | |
| | | 1.58 (dq, J = 52.8, 11.9 Hz, | |
| | | 4H); 19F NMR (376 MHz, | |
| | | DMSO) δ −107.2. | |
| Compound 202 | 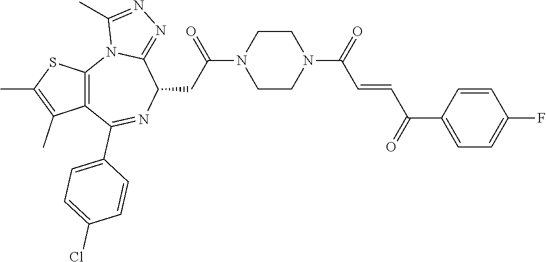 [SMILES: Cc1sc2c(c1C)C(c1ccc(Cl)cc1)=N[C@@H](CC(=O)N1CCN(C(=O)/C=C/C(=O)c3ccc(F)cc3)CC1)c1nnc(C)n1-2] | ¹H NMR (400 MHz, DMSO) δ 8.16 (ddd, J = 8.8, 5.8, 2.7 Hz, 2H), 7.81 (d, J = 15.2 Hz, 1H), 7.55- 7.37 (m, 7H), 4.60 (t, J = 6.7 Hz, 1H), 3.81-3.71 (m, 4H), 3.70-3.64 (m, 1H), 3.60 (q, J = 9.0 Hz, 4H), 3.45 (d, J = 16.6 Hz, 1H), 2.61 (s, 3H), 2.42 (s, 3H), 1.64 (s, 3H). | Rt 2.22; 643.3 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(4- | | |
| | fluorophenyl)but-2-ene-1,4-dione | | |
| Compound 203 | 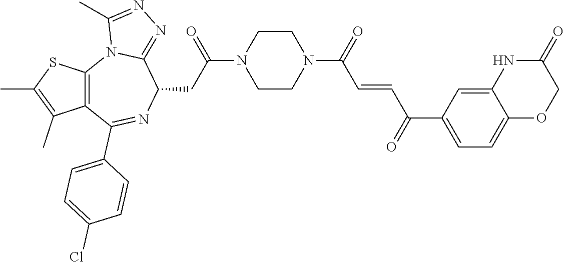 [SMILES: Cc1sc2c(c1C)C(c1ccc(Cl)cc1)=N[C@@H](CC(=O)N1CCN(C(=O)/C=C/C(=O)c3ccc4c(c3)NC(=O)CO4)CC1)c1nnc(C)n1-2] | ¹H NMR (400 MHz, DMSO) δ 10.89 (s, 1H), 7.80-7.68 (m, 2H), 7.59 (s, 1H), 7.53-7.41 (m, 5H), 7.10 (d, J = 8.5 Hz, 1H), 4.73 (s, 2H), 4.60 (t, J = 6.8 Hz, 1H), 3.75 (d, J = 7.8 Hz, 4H), 3.63 (dd, J = 30.2, 13.9 Hz, 5H), 3.44 (dt, J = 15.2, 8.1 Hz, 1H), 2.61 (s, 3H), 2.45-2.40 (m, 3H), 1.64 (s, 3H). | Rt = 2.25; 699.1 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(3-oxo-3,4- | | |
| | dihydro-2H-benzo[b][1,4]oxazin-6- | | |
| | yl)but-2-ene-1,4-dione | | |
| Compound 204 | 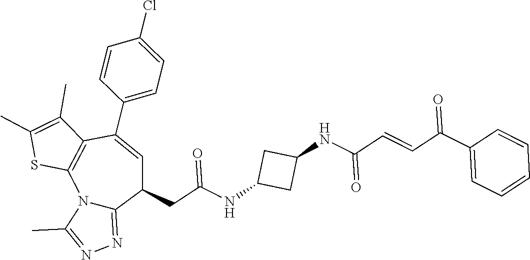 [SMILES: Cc1sc2c(c1C)C(c1ccc(Cl)cc1)=C[C@@H](CC(=O)N[C@H]1C[C@H](NC(=O)/C=C/C(=O)c3ccccc3)C1)c1nnc(C)n1-2] | ¹H NMR (400 MHz, DMSO) δ 9.07 (d, J = 7.2 Hz, 1H), 8.74 (d, J = 7.1 Hz, 1H), 8.08 (d, J = 7.7 Hz, 2H), 7.85-7.72 (m, 2H), 7.65 (t, J = 7.6 Hz, 2H), 7.57 (d, J = 8.3 Hz, 2H), 7.49 (d, J = 8.2 Hz, 2H), 7.03 (d, J = 15.4 Hz, 1H), 4.58 (t, J = 7.1 Hz, 2H), 4.54-4.47 (m, 3H), 4.46 (s, 2H), 4.38 (s, 1H), 3.30 (qd, J = 15.1, 7.2 Hz, 2H), 2.68 (s, 3H), 2.48 (s, 3H), 2.35 (dt, J = 12.6, 6.7 Hz, 4H), 1.68 (s, 3H) | Rt = 2.91; 627.2 [M + H]+ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-oxo-4- | | |
| | phenylbut-2-enamide | | |
| Compound 205 | 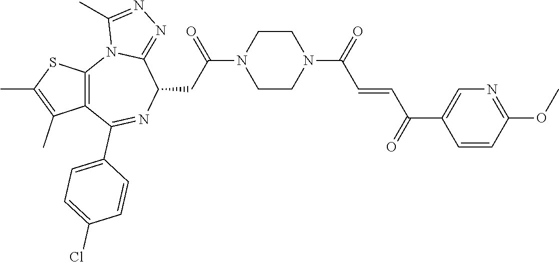 | ¹H NMR (400 MHz, DMSO) δ 8.95 (s, 1H), 8.30 (d, J = 8.8 Hz, 1H), 7.78 (d, J = 15.2 Hz, 1H), 7.55- 7.41 (m, 5H), 7.00 (d, J = 8.8 Hz, 1H), 4.60 (t, J = 6.8 Hz, 1H), 3.98 (s, 3H), 3.81- 3.72 (m, 4H), 3.66 (q, J = 7.6 Hz, 1H), 3.58 (s, 4H), 3.45 (d, J = 16.5 Hz, 1H), 2.61 (s, 3H), 2.43 (s, 3H), 1.64 (s, 3H). | Rt = 291; 659.2 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(6- | | |
| | methoxypyridin-3-yl)but-2-ene-1,4- | | |
| | dione | | |
| Compound 206 | 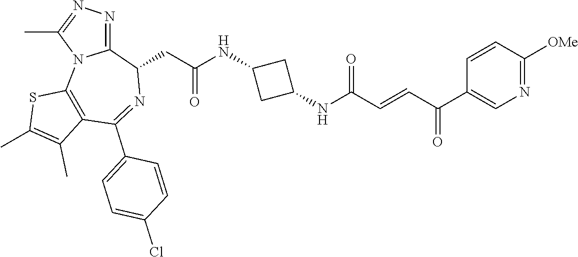 [SMILES: COc1ccc(C(=O)/C=C/C(=O)N[C@H]2C[C@@H](NC(=O)C[C@@H]3N=C(c4ccc(Cl)cc4)c4c(sc(C)c4C)-n4c(C)nnc43)C2)cn1] | ¹H NMR (400 MHz, DMSO) δ 8.92 (d, J = 2.5 Hz, 1H), 8.86 (d, J = 7.1 Hz, 1H), 8.52 (d, J = 7.4 Hz, 1H), 8.28 (dd, J = 8.8, 2.5 Hz, 1H), 7.75 (d, J = 15.3 Hz, 1H), 7.50 (d, J = 8.3 Hz, 2H), 7.42 (d, J = 8.3 Hz, 2H), 7.04-6.96 (m, 2H), 4.50 (t, J = 7.1 Hz, 1H), 4.07-3.99 (m, 1H), 3.98 (s, 3H), 3.23 (dd, J = 7.5, 2.6 Hz, 2H), 2.60 (s, 3H), 2.42 (s, 3H), 1.92 (dt, J = 21.2, 9.8 Hz, 2H), 1.63 (s, 3H). | Rt = 2.82; 658.2 [M + H]+ |
| | (E)-N-((1R,3s)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-(6- | | |
| | methoxypyridin-3-yl)-4-oxobut-2- | | |
| | enamide | | |
| Compound 207 | 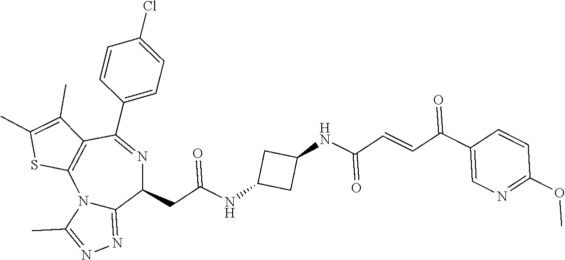 | ¹H NMR (400 MHz, DMSO) δ 8.99 (d, J = 7.2 Hz, 1H), 8.92 (d, J = 2.5 Hz, 1H), 8.67 (d, J = 7.1 Hz, 1H), 8.28 (dd, J = 8.7, 2.5 Hz, 1H), 7.75 (d, J = 15.3 Hz, 1H), 7.51 (d, J = 8.5 Hz, 2H), 7.47-7.39 (m, 2H), 6.99 (dd, J = 12.0, 3.3 Hz, 2H), 4.54-4.40 (m, 2H), 4.34 (q, J = 6.7 Hz, 1H), 3.97 (s, 3H), 3.24 (qd, J = 15.0, 7.1 Hz, 2H), 2.60 (s, 3H), 2.42 (s, 3H), 2.29 (dq, J = 12.1, 6.5 Hz, 4H), 1.63 (s, 3H). | Rt = 2.82; 658.2 [M + H]+ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-(6- | | |
| | methoxypyridin-3-yl)-4-oxobut-2- | | |
| | enamide | | |
| Compound 208 | 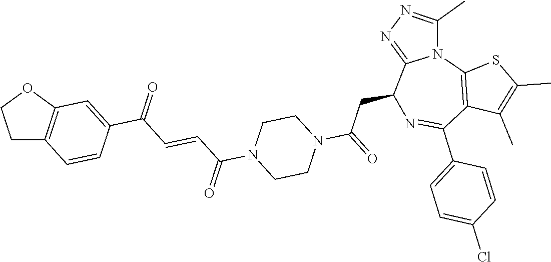 [SMILES: Cc1sc2c(c1C)C(c1ccc(Cl)cc1)=N[C@@H](CC(=O)N1CCN(C(=O)/C=C/C(=O)c3ccc4c(c3)OCC4)CC1)c1nnc(C)n1-2] | ¹H NMR (400 MHz, DMSO) δ 7.76 (d, J = 15.1 Hz, 1H), 7.60 (d, J = 7.8 Hz, 1H), 7.53-7.40 (m, 7H), 7.33 (s, 1H), 4.66-4.56 (m, 3H), 3.75 (d, J = 10.3 Hz, 3H), 3.67 (dt, J = 15.1, 7.1 Hz, 2H), 3.59 (d, J = 9.3 Hz, 4H), 3.45 (d, J = 17.5 Hz, 1H), 3.28 (t, J = 8.7 Hz, 5H), 2.61 (s, 4H), 2.55 (s, 4H), 2.42 (s, 4H), 1.64 (s, 3H). | Rt = 2.43; 669.1 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(2,3- | | |
| | dihydrobenzofuran-6-yl)but-2-ene-1,4- | | |
| | dione | | |
| Compound 209 | 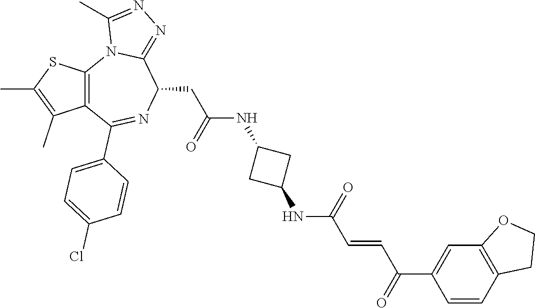 [SMILES: Cc1sc2c(c1C)C(c1ccc(Cl)cc1)=N[C@@H](CC(=O)N[C@H]1C[C@H](NC(=O)/C=C/C(=O)c3ccc4c(c3)OCC4)C1)c1nnc(C)n1-2] | ¹H NMR (400 MHz, DMSO) δ 8.71 (d, J = 7.1 Hz, 1H), 8.38 (d, J = 6.9 Hz, 1H), 7.65 (d, J = 15.4 Hz, 1H), 7.56-7.37 (m, 7H), 7.28 (d, J = 1.5 Hz, 1H), 6.91 (d, J = 15.5 Hz, 1H), 4.62 (t, J = 8.7 Hz, 2H), 4.54 (dd, J = 7.7, 6.2 Hz, 1H), 4.45 (d, J = 6.7 Hz, 1H), 4.36 (q, J = 6.8 Hz, 1H), 3.27 (dt, J = 12.3, 8.5 Hz, 4H), 2.61 (s, 3H), 2.43 (s, 3H), 2.36-2.26 (m, 4H), 1.66 (s, 3H). | Rt = 2.92; 670.2 [M + H]+ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-(2,3- | | |
| | dihydrobenzofuran-6-yl)-4-oxobut-2- | | |
| | enamide | | |
| Compound 210 | 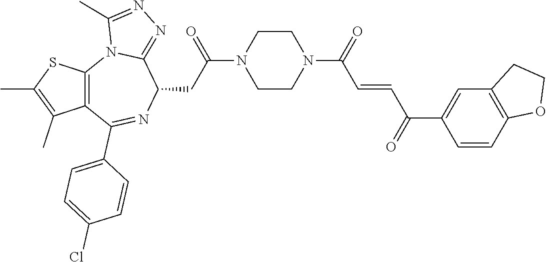 [SMILES: Cc1sc2c(c1C)C(c1ccc(Cl)cc1)=N[C@@H](CC(=O)N1CCN(C(=O)/C=C/C(=O)c3ccc4c(c3)CCO4)CC1)c1nnc(C)n1-2] | ¹H NMR (400 MHz, CDCl₃) δ 7.95-7.82 (m, 3H), 7.40 (dd, J = 14.9, 5.0 Hz, 1H), 7.34 (d, J = 8.3 Hz, 2H), 7.31-7.24 (m, 2H), 6.79 (d, J = 8.4 Hz, 1H), 4.76 (t, J = 6.7 Hz, 1H), 4.63 (t, J = 8.8 Hz, 2H), 3.90 (s, 2H), 3.82-3.63 (m, 5H), 3.60-3.41 (m, 3H), 3.21 (t, J = 8.8 Hz, 2H), 2.62 (s, 3H), 2.34 (s, 3H), 1.61 (s, 3H). | Rt = 2.67; 670.3 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(2,3- | | |
| | dihydrobenzofuran-5-yl)but-2-ene-1,4- | | |
| | dione | | |
| Compound 211 | 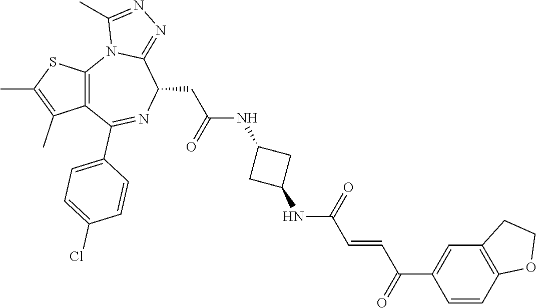 [SMILES: Cc1sc2c(c1C)C(c1ccc(Cl)cc1)=N[C@@H](CC(=O)N[C@H]1C[C@H](NC(=O)/C=C/C(=O)c3ccc4c(c3)CCO4)C1)c1nnc(C)n1-2] | ¹H NMR (400 MHz, DMSO) δ 9.01 (d, J = 7.3 Hz, 1H), 8.73 (d, J = 7.1 Hz, 1H), 8.01 (d, J = 1.8 Hz, 1H), 7.95 (dd, J = 8.5, 2.0 Hz, 1H), 7.83 (d, J = 15.2 Hz, 1H), 7.57 (d, J = 8.3 Hz, 2H), 7.49 (d, J = 8.5 Hz, 2H), 7.03-6.94 (m, 2H), 4.73 (t, J = 8.8 Hz, 2H), 4.61-4.46 (m, 2H), 4.39 (q, J = 7.1 Hz, 1H), 3.38-3.21 (m, 4H), 2.67 (s, 3H), 2.48 (s, 3H), 2.39-2.28 (m, 4H), 1.68 (s, 3H). | Rt = 2.90; 670.1 [M + H]+ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-(2,3- | | |
| | dihydrobenzofuran-5-yl)-4-oxobut-2- | | |
| | enamide | | |
| Compound 212 | 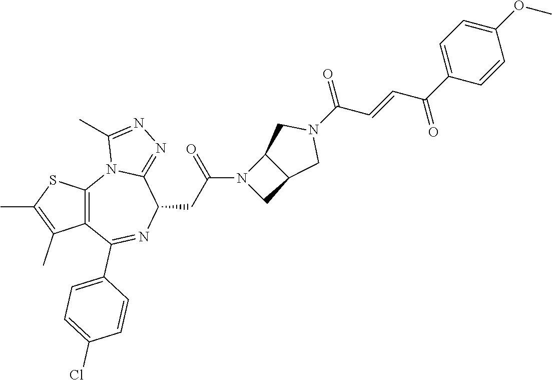 | ¹H NMR (400 MHz, DMSO) δ 7.97 (d, J = 8.5 Hz, 2H), 7.73 (d, J = 15.3 Hz, 1H), 7.44 (d, J = 9.9 Hz, 4H), 7.27 (d, J = 15.3 Hz, 1H), 7.12-7.03 (m, 2H), 4.91 (s, 1H), 4.52 (t, J = 6.8 Hz, 1H), 4.34 (s, 1H), 4.16 (d, J = 12.5 Hz, 1H), 3.89 (s, 3H), 3.49 (s, 2H), 3.28 (s, 2H), 3.21 (d, J = 6.9 Hz, 2H), 2.60 (s, 1H), 2.59 (s, 3H), 2.42 (s, 3H), 1.63 (s, 3H). | Rt = 2.49; 670.3 [M + H]+ |
| | (E)-1-((1S,5R)-6-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6-yl)acetyl)-3,6- | | |
| | diazabicyclo[3.2.0]heptan-3-yl)-4-(4- | | |
| | methoxyphenyl)but-2-ene-1,4-dione | | |
| Compound 213 | 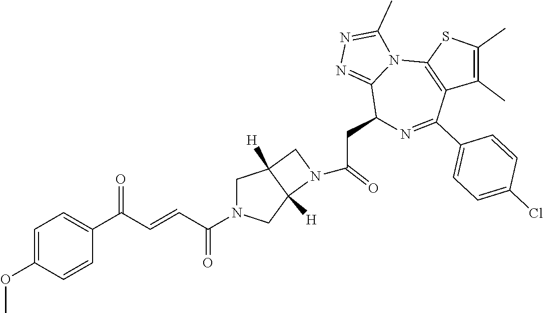 [SMILES: COc1ccc(C(=O)/C=C/C(=O)N2C[C@@H]3CN(C(=O)C[C@@H]4N=C(c5ccc(Cl)cc5)c5c(sc(C)c5C)-n5c(C)nnc54)[C@@H]3C2)cc1] | ¹H NMR (400 MHz, DMSO) δ 7.97 (d, J = 8.5 Hz, 2H), 7.68 (d, J = 15.5 Hz, 1H), 7.44 (s, 3H), 7.20 (s, OH), 7.07 (d, J = 8.8 Hz, 2H), 4.54 (t, J = 6.9 Hz, 1H), 4.26 (s, OH), 4.13 (d, J = 12.4 Hz, 1H), 3.89 (s, 3H), 3.48 (s, 1H), 3.28 (s, 1H), 3.19 (dd, J = 15.3, 7.2 Hz, 1H), 2.58 (s, 3H), 2.49 (s, 9H), 2.43 (s, 3H), 1.67 (s, 3H). | Rt = 2.60; 669.2 [M + H]+ |
| | (E)-1-((1R,5S)-6-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6-yl)acetyl)-3,6- | | |
| | diazabicyclo[3.2.0]heptan-3-yl)-4-(4- | | |
| | methoxyphenyl)but-2-ene-1,4-dione | | |
| Compound 214 | 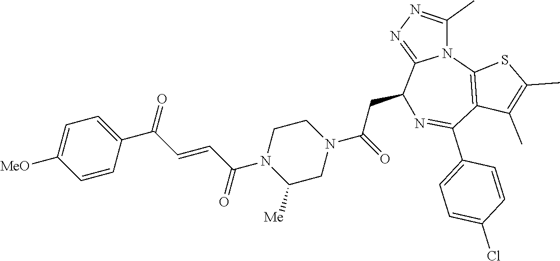 [SMILES: COc1ccc(C(=O)/C=C/C(=O)N2CCN(C(=O)C[C@@H]3N=C(c4ccc(Cl)cc4)c4c(sc(C)c4C)-n4c(C)nnc43)C[C@@H]2C)cc1] | ¹H NMR (400 MHz, DMSO) δ 8.04-7.96 (m, 2H), 7.69 (d, J = 15.4 Hz, 1H), 7.45 (s, 3H), 7.34 (d, J = 15.4 Hz, 1H), 7.14-7.05 (m, 2H), 4.66 (t, J = 6.7 Hz, 1H), 4.54 (s, 1H), 4.23 (d, J = 13.3 Hz, 1H), 4.11 (d, J= 13.7 Hz, 2H), 3.90 (s, 3H), 3.69 (dd, J = 15.8, 6.9 Hz, 1H), 3.44 (dd, J = 15.9, 6.5 Hz, 1H), 3.38 (s, 1H), 2.87 (s, 4H), 2.61 (s, 3H), 2.44 (s, 3H), 1.69 (s, 3H), 1.23 (d, J = 6.7 Hz, 3H). | Rt = 2.26, 671.4 [M + H]+ |
| | (E)-1-((S)-4-(2-((S)-4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetyl)-2-methylpiperazin-1-yl)-4-(4- | | |
| | methoxyphenyl)but-2-ene-1,4-dione | | |
| Compound 215 | 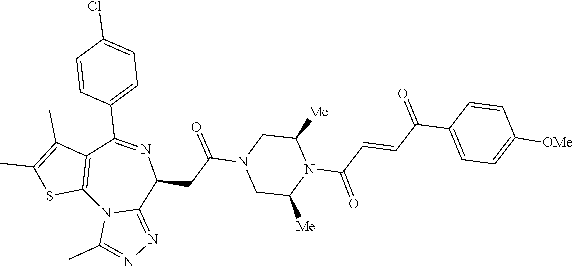 [SMILES: COc1ccc(C(=O)/C=C/C(=O)N2[C@H](C)CN(C(=O)C[C@@H]3N=C(c4ccc(Cl)cc4)c4c(sc(C)c4C)-n4c(C)nnc43)C[C@@H]2C)cc1] | ¹H NMR (400 MHz, DMSO) δ 8.11-8.04 (m, 2H), 7.84 (d, J = 15.1 Hz, 1H), 7.54-7.41 (m, 5H), 7.11 (d, J = 8.8 Hz, 2H), 4.65 (s, 1H), 4.30 (t, J = 13.1 Hz, 2H), 4.14 (t, J = 15.9 Hz, 1H), 3.88 (s, 3H), 2.88 (s, 1H), 2.61 (d, J = 1.5 Hz, 3H), 2.43 (s, 3H), 1.64 (s, 3H), 1.46 (d, J = 17.6 Hz, 2H), 1.36 (s, 1H), 1.18 (s, 2H). | Rt = 2.31; 685.4 [M + H]+ |
| | (E)-1-((2S,6R)-4-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6-yl)acetyl)-2,6- | | |
| | dimethylpiperazin-1-yl)-4-(4- | | |
| | methoxyphenyl)but-2-ene-1,4-dione | | |
| Compound 216 | 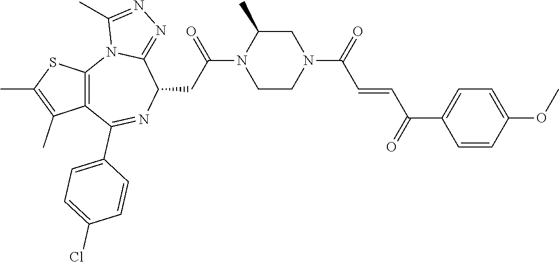 | ¹H NMR (400 MHz, DMSO) δ 8.04-7.97 (m, 2H), 7.72 (d, J = 15.3 Hz, 1H), 7.46 (s, 4H), 7.36 (d, J = 15.3 Hz, 1H), 7.14-7.05 (m, 2H), 4.66 (t, J = 6.8 Hz, 2H), 4.19 (d, J = 11.7 Hz, 2H), 4.04 (s, 1H), 3.91 (s, 3H), 3.63 (dd, J = 15.9, 6.9 Hz, 1H), 3.50 (dd, J = 16.2, 6.6 Hz, 1H), 3.30 (m, 3H), 2.62 (s, 3H), 2.44 (s, 3H), 1.69 (s, 3H), 1.25 (d, J = 6.7 Hz, 3H). | Rt = 2.45; 672.2 [M + H]+ |
| | (E)-1-((S)-4-(2-((S)-4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetyl)-3-methylpiperazin-1-yl)-4-(4- | | |
| | methoxyphenyl)but-2-ene-1,4-dione | | |
| Compound 217 | 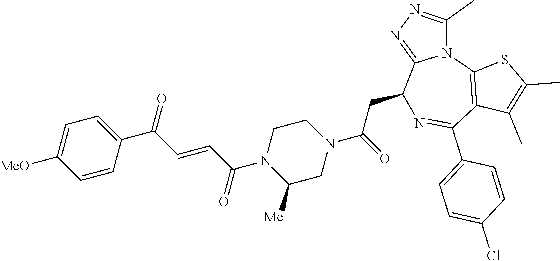 [SMILES: COc1ccc(C(=O)/C=C/C(=O)N2CCN(C(=O)C[C@@H]3N=C(c4ccc(Cl)cc4)c4c(sc(C)c4C)-n4c(C)nnc43)C[C@H]2C)cc1] | ¹H NMR (400 MHz, DMSO) δ 8.05-7.97 (m, 2H), 7.70 (d, J = 15.4 Hz, 1H), 7.46 (s, 3H), 7.35 (d, J = 15.3 Hz, 1H), 7.14-7.02 (m, 2H), 4.66 (t, J = 6.6 Hz, 1H), 4.56 (s, 1H), 4.21 (d, J = 12.9 Hz, 1H), 4.11 (d, J = 13.5 Hz, 2H), 3.91 (s, 3H), 3.65 (dd, J = 16.1, 6.7 Hz, 1H), 3.52 (dd, J = 16.1, 6.6 Hz, 1H), 3.21 (s, 0H), 2.62 (s, 3H), 2.44 (s, 3H), 1.69 (s, 3H), 1.28 (d, J = 6.7 Hz, 2H). | Rt = 2.47; 671.1 [M + H]+ |
| | (E)-1-((R)-4-(2-((S)-4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetyl)-2-methylpiperazin-1-yl)-4-(4- | | |
| | methoxyphenyl)but-2-ene-1,4-dione | | |
| Compound 218 | 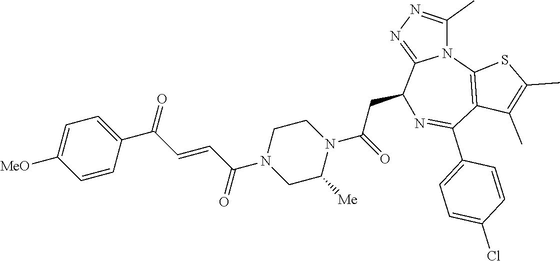 [SMILES: COc1ccc(C(=O)/C=C/C(=O)N2CCN(C(=O)C[C@@H]3N=C(c4ccc(Cl)cc4)c4c(sc(C)c4C)-n4c(C)nnc43)[C@H](C)C2)cc1] | ¹H NMR (400 MHz, DMSO) δ 8.06-7.98 (m, 2H), 7.73 (d, J = 15.5 Hz, 1H), 7.47 (s, 3H), 7.37 (d, J = 15.3 Hz, 1H), 7.16-7.09 (m, 2H), 4.67 (d, J = 6.6 Hz, 2H), 4.19 (d, J = 12.8 Hz, 2H), 3.92 (s, 3H), 3.68 (dd, J = 16.1, 6.8 Hz, 1H), 3.48 (dd, J = 16.0, 6.7 Hz, 1H), 2.86 (s, 23H), 2.63 (s, 3H), 2.51 (s, 22H), 2.46 (s, 5H), 1.71 (s, 3H), 1.23 (d, J = 6.7 Hz, 3H). | Rt = 2.44; 671.2 [M + H]+ |
| | (E)-1-((R)-4-(2-((S)-4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetyl)-3-methylpiperazin-1-yl)-4-(4- | | |
| | methoxyphenyl)but-2-ene-1,4-dione | | |
| Compound 219 | 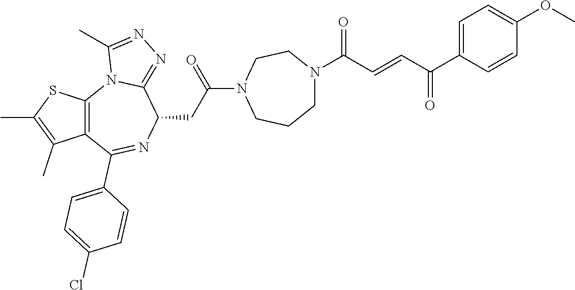 | ¹H NMR (400 MHZ, DMSO) δ 7.97 (d, J = 8.4 Hz, 2H), 7.44 (s, 4H), 7.06 (d, J = 8.4 Hz, 2H), 4.65 (t, J = 6.6 Hz, 1H), 3.89 (s, 3H), 3.80 (s, 3H), 3.71 (s, 3H), 3.64 (dd, J = 16.1, 6.6 Hz, 3H), 3.52-3.40 (m, 1H), 2.61 (d, J = 6.8 Hz, 3H), 2.44 (d, J = 5.6 Hz, 3H), 1.91 (s, 2H), 1.67 (s, 3H). | Rt = 2.17; 672.3 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)-1,4-diazepan-1-yl)-4-(4- | | |
| | methoxyphenyl)but-2-ene-1,4-dione | | |
| Compound 220 | 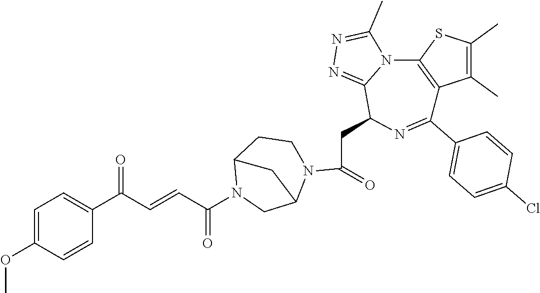 [SMILES: COc1ccc(C(=O)/C=C/C(=O)N2CC3CC2CCN3C(=O)C[C@@H]2N=C(c3ccc(Cl)cc3)c3c(sc(C)c3C)-n3c(C)nnc32)cc1] | ¹H NMR (400 MHz, DMSO) δ 8.05-7.97 (m, 1H), 7.76 (d, J = 15.1 Hz, 0H), 7.46 (d, J = 2.4 Hz, 2H), 7.14-7.06 (m, 1H), 5.13 (s, 0H), 4.68-4.60 (m, 1H), 3.91 (s, 2H), 3.61 (s, 0H), 2.62 (s, 2H), 2.46 (d, J = 17.0 Hz, 10H), 1.84 (s, 1H), 1.69 (s, 3H). | Rt = 2.11; 683.2 [M + H]+ |
| | (E)-1-(2-(2-((S)-4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetyl)-2,6-diazabicyclo[3.2.1]octan- | | |
| | 6-y1)-4-(4-methoxyphenyl)but-2-ene- | | |
| | 1,4-dione | | |
| Compound 221 | 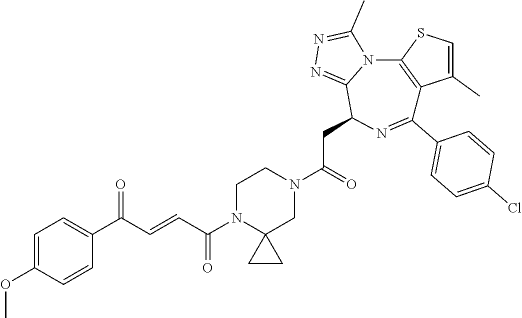 | ¹H NMR (400 MHz, DMSO) δ 8.05-7.97 (m, 2H), 7.69 (d, J = 15.4 Hz, 1H), 7.46 (s, 5H), 7.15- 7.06 (m, 2H), 4.65 (t, J = 6.6 Hz, 1H), 3.91 (s, 3H), 3.78 (d, J = 13.4 Hz, 3H), 3.62 (s, 2H), 3.46 (dd, J = 16.0, 6.7 Hz, 1H), 2.62 (s, 3H), 2.44 (s, 3H), 1.69 (s, 3H), 1.07 (s, 2H) | Rt = 3.15; 683.2 [M + H]+ |
| | (S,E)-1-(7-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)-4,7-diazaspiro[2.5]octan-4- | | |
| | yl)-4-(4-methoxyphenyl)but-2-ene-1,4- | | |
| | dione | | |
| Compound 223 | 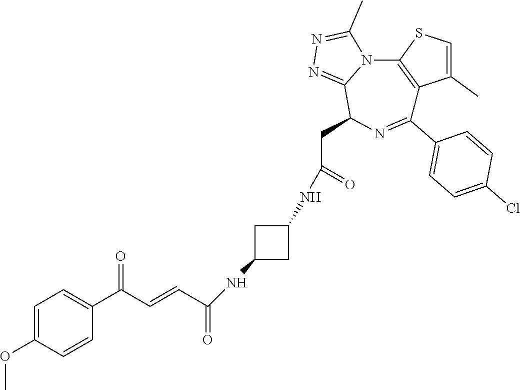 | ¹H NMR (400 MHz, CDCl₃) δ 7.97-7.89 (m, 2H), 7.84 (d, J = 15.0 Hz, 1H), 7.42 (s, 1H), 7.37- 7.21 (m, 4H), 7.04 (s, 1H), 6.96 - 6.85 (m, 3H), 4.60 (t, J = 7.0 Hz, 1H), 4.56-4.43 (m, 2H), 3.81 (s, 3H), 3.55 (dd, J = 14.7, 7.8 Hz, 1H), 3.36 (dd, J = 14.6, 6.3 Hz, 1H), 2.65 (s, 3H), 2.40 (dt, J = 15.1, 6.9 Hz, 3H), 1.58 (s, 3H). | Rt = 2.61; 657.2 [M + H]+ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-(4- | | |
| | methoxyphenyl)-4-oxobut-2-enamide | | |
| Compound 224 | 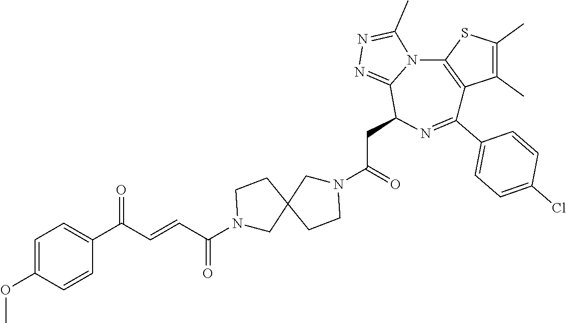 [SMILES: COc1ccc(C(=O)/C=C/C(=O)N2CCC3(CCN(C(=O)C[C@@H]4N=C(c5ccc(Cl)cc5)c5c(sc(C)c5C)-n5c(C)nnc54)C3)C2)cc1] | ¹H NMR (400 MHz, DMSO) δ 8.10-8.01 (m, 2H), 7.88-7.77 (m, 1H), 7.57-7.46 (m, 2H), 7.45 (s, 2H), 7.24 (dt, J = 15.0, 5.3 Hz, 1H), 7.14-7.07 (m, 2H), 4.56 (s, 1H), 3.87 (d, J = 1.2 Hz, 3H), 3.81 (dd, J = 14.4, 7.0 Hz, 2H), 3.73- 3.63 (m, 1H), 3.61-3.41 (m, 4H), 2.63-2.55 (m, 3H), 2.42 (s, 3H), 1.95 (dq, J = 40.5, 7.5 Hz, 4H), 1.64 (s, 3H). | Rt = 2.69; 697.2 [M + H]+ |
| | (E)-1-(7-(2-((S)-4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetyl)-2,7-diazaspiro[4.4]nonan-2- | | |
| | yl)-4-(4-methoxyphenyl)but-2-ene-1,4- | | |
| | dione | | |
| Compound 225 | 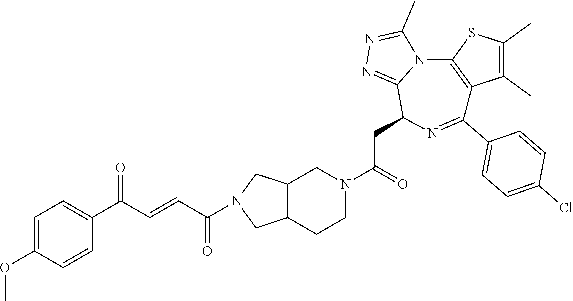 [SMILES: COc1ccc(C(=O)/C=C/C(=O)N2CC3CCN(C(=O)C[C@@H]4N=C(c5ccc(Cl)cc5)c5c(sc(C)c5C)-n5c(C)nnc54)CC3C2)cc1] | ¹H NMR (400 MHz, DMSO) δ 8.00 (dd, J = 19.0, 7.3 Hz, 3H), 7.71 (dd, J = 23.3, 15.4 Hz, 2H), 7.45 (d, J = 13.7 Hz, 6H), 7.25- 7.15 (m, 1H), 7.09 (dd, J = 12.1, 7.8 Hz, 4H), 4.64 (t, J = 6.7 Hz, 1H), 3.89 (d, J = 7.2 Hz, 6H), 3.75 (d, J = 7.7 Hz, 2H), 3.65-3.57 (m, 3H), 3.47 (d, J = 9.0 Hz, 2H), 2.60 (d, J = 11.8 Hz, 4H), 2.43 (d, J = 2.8 Hz, 8H), 1.78 (s, 1H), 1.67 (d, J = 6.9 Hz, 6H) | Rt = 2.99; 697.2 [M + H]+ |
| | (E)-1-(5-(2-((S)-4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetyl)octahydro-2H-pyrrolo[3,4- | | |
| | c]pyridin-2-yl)-4-(4- | | |
| | methoxyphenyl)but-2-ene-1,4-dione | | |
| Compound 226 | 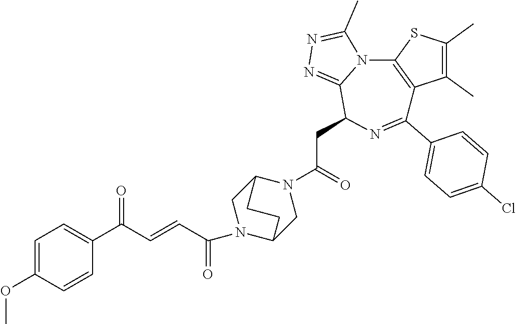 [SMILES: COc1ccc(C(=O)/C=C/C(=O)N2CC3CCC2CN3C(=O)C[C@@H]2N=C(c3ccc(Cl)cc3)c3c(sc(C)c3C)-n3c(C)nnc32)cc1] | ¹H NMR (400 MHz, DMSO) δ 8.02 (dd, J = 8.6, 4.3 Hz, 2H), 7.72 (s, 1H), 7.46 (s, 4H), 7.28 (s, 1H), 7.11 (d, J = 8.2 Hz, 2H), 4.65 (s, 3H), 4.03 (s, 1H), 3.92 (d, J = 3.2 Hz, 3H), 3.51 (d, J = 65.0 Hz, 4H), 2.63 (d, J = 3.2 Hz, 3H), 2.45 (s, 3H), 2.25 (s, 1H), 1.97 (d, J = 30.9 Hz, 4H), 1.70 (s, 3H). | Rt = 2.70; 683.2 [M + H]+ |
| | (E)-1-(5-(2-((S)-4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetyl)-2,5-diazabicyclo[2.2.2]octan- | | |
| | 2-yl)-4-(4-methoxyphenyl)but-2-ene- | | |
| | 1,4-dione | | |
| Compound 227 | 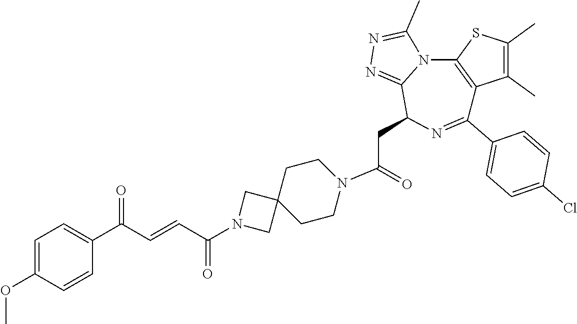 [SMILES: COc1ccc(C(=O)/C=C/C(=O)N2CC3(CCN(C(=O)C[C@@H]4N=C(c5ccc(Cl)cc5)c5c(sc(C)c5C)-n5c(C)nnc54)CC3)C2)cc1] | ¹H NMR (400 MHz, CDCl₃) δ 8.02-7.88 (m, 3H), 7.33 (d, J = 8.3 Hz, 2H), 7.26 (d, J = 8.3 Hz, 2H), 6.99 (dd, J = 15.0, 7.8 Hz, 1H), 6.91 (d, J = 8.6 Hz, 2H), 4.73 (t, J = 6.6 Hz, 1H), 4.07-3.97 (m, 2H), 3.85 (d, J = 15.7 Hz, 5H), 3.75-3.63 (m, 2H), 3.55- 3.43 (m, 2H), 2.60 (s, 3H), 2.33 (s, 3H), 1.93 (s, 1H), 1.88 (s, 1H), 1.75 (s, 2H). | Rt = 2.71; 697.2 [M + H]+ |
| | (S,E)-1-(7-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)-2,7-diazaspiro[3.5]nonan-2- | | |
| | yl)-4-(4-methoxyphenyl)but-2-ene-1,4- | | |
| | dione | | |
| Compound 228 | 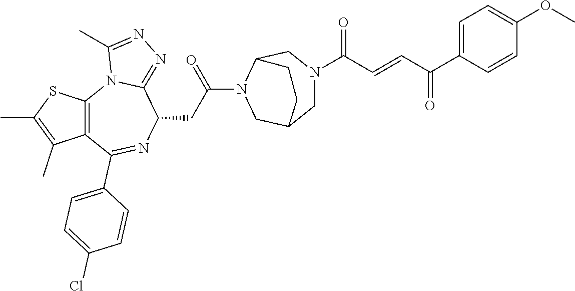 | ¹H NMR (400 MHz, DMSO) δ 7.99 (dd, J = 8.7, 2.0 Hz, 2H), 7.50-7.34 (m, 6H), 7.11-7.04 (m, 2H), 4.73 (s, 1H), 4.66 (t, J = 6.8 Hz, 1H), 4.27 (m, 2H), 3.90 (d, J = 1.4 Hz, 3H), 3.80- 3.18 (m, 6H), 2.88 (s, 1H), 2.64-2.58 (m, 3H), 2.43 (s, 3H), 1.80 (d, J = 44.7 Hz, 4H), 1.68 (d, J = 5.4 Hz, 3H). | Rt = 3.06; 698.2 [M + H]+ |
| | (E)-1-(6-(2-((S)-4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetyl)-3,6-diazabicyclo[3.2.2]nonan- | | |
| | 3-yl)-4-(4-methoxyphenyl)but-2-ene- | | |
| | 1,4-dione | | |
| Compound 229 | 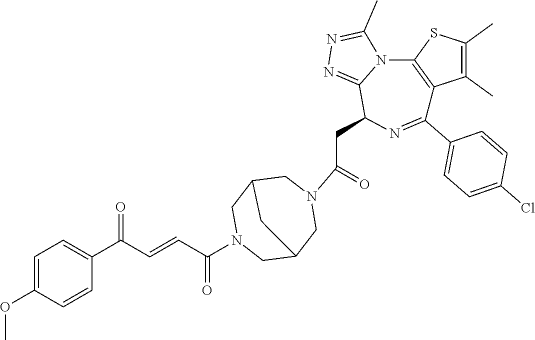 [SMILES: COc1ccc(C(=O)/C=C/C(=O)N2CC3CC(C2)CN(C(=O)C[C@@H]2N=C(c4ccc(Cl)cc4)c4c(sc(C)c4C)-n4c(C)nnc42)C3)cc1] | ¹H NMR (400 MHz, DMSO) δ 7.87 (d, J = 7.5 Hz, 1H), 7.47-7.36 (m, 4H), 7.23 (s, 1H), 6.93 (s, 1H), 4.58 (s, 1H), 4.40 (s, 1H), 3.86 (s, 2H), 3.45 (s, 1H), 3.23 (s, 1H), 2.58 (s, 3H), 2.37 (s, 2H), 2.07 (s, 1H), 1.95 (s, 3H), 1.59 (s, 3H). | Rt = 2.67; 697.2 [M + H]+ |
| | (E)-1-(7-(2-((S)-4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetyl)-3,7-diazabicyclo[3.3.1]nonan- | | |
| | 3-yl)-4-(4-methoxyphenyl)but-2-ene- | | |
| | 1,4-dione | | |
| Compound 230 | 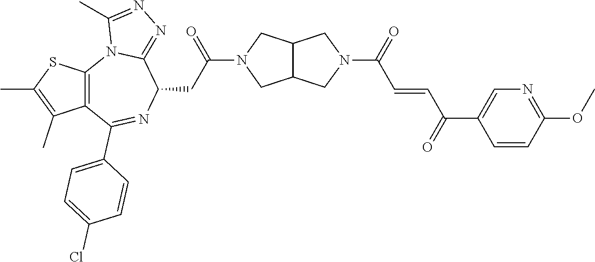 | ¹H NMR (400 MHz, DMSO) δ 8.05-7.98 (m, 2H), 7.75 (dd, J = 15.3, 7.9 Hz, 1H), 7.46 (s, 4H), 7.20 (d, J = 15.1 Hz, 1H), 7.13- 7.06 (m, 2H), 4.59 (t, J = 6.8 Hz, 1H), 3.94 (m, 2H), 3.89 (s, 3H), 3.75 (m, 2H), 3.65 (m, 2H), 3.45 (m, 2H), 3.39 (m, 2H), 3.16 (m, 1 H), 2.95 (m, 1 H), 2.61 (s, 3H), 2.43 (d, J = 0.9 Hz, 3H), 1.66 (s, 3H). | Rt = 2.95; 683.2 [M + H]+ |
| | (E)-1-(5-(2-((S)-4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetyl)hexahydropyrrolo[3,4- | | |
| | c]pyrrol-2(1H)-yl)-4-(4- | | |
| | methoxyphenyl)but-2-ene-1,4-dione | | |
| Compound 231 | 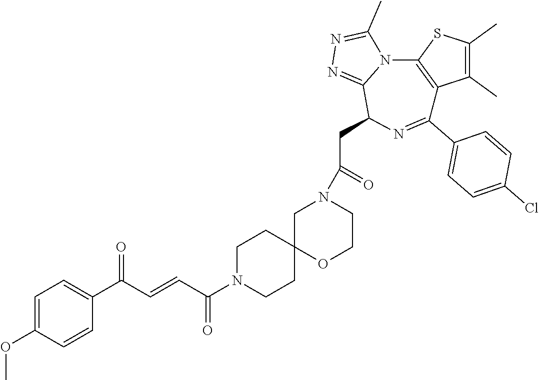 [SMILES: COc1ccc(C(=O)/C=C/C(=O)N2CCC3(CC2)CN(C(=O)C[C@@H]2N=C(c4ccc(Cl)cc4)c4c(sc(C)c4C)-n4c(C)nnc42)CCO3)cc1] | ¹H NMR (400 MHz, DMSO) broad rotomer populations in spectra | Rt = 2.64; 727.3 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)-1-oxa-4,9- | | |
| | diazaspiro[5.5]undecan-9-yl)-4-(4- | | |
| | methoxyphenyl)but-2-ene-1,4-dione | | |
| Compound 232 | 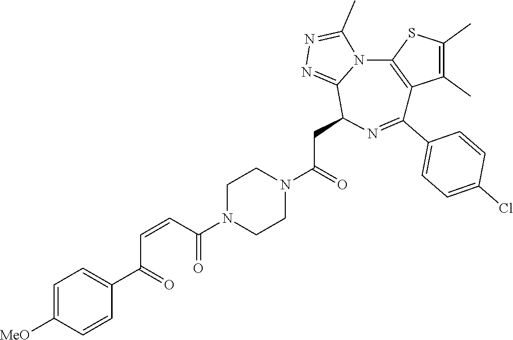 [SMILES: COc1ccc(C(=O)/C=C\C(=O)N2CCN(C(=O)C[C@@H]3N=C(c4ccc(Cl)cc4)c4c(sc(C)c4C)-n4c(C)nnc43)CC2)cc1] | ¹H NMR (400 MHz, DMSO) δ 7.98-7.90 (m, 2H), 7.51-7.42 (m, 4H), 7.16-7.03 (m, 3H), 6.77 (d, J = 12.0 Hz, 1H), 4.62 (t, J = 6.7 Hz, 1H), 3.88 (s, 3H), 3.64 (dd, J = 16.0, 6.9 Hz, 3H), 3.54 (s, 2H), 3.47 (dd, J = 16.1, 6.6 Hz, 2H), 2.61 (s, 3H), 2.43 (s, 3H), 1.67 (s, 3H) | Rt = 2.61; 657.2 [M + H]+ |
| | (S,Z)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(4- | | |
| | methoxyphenyl)but-2-ene-1,4-dione | | |
| Compound 233 | 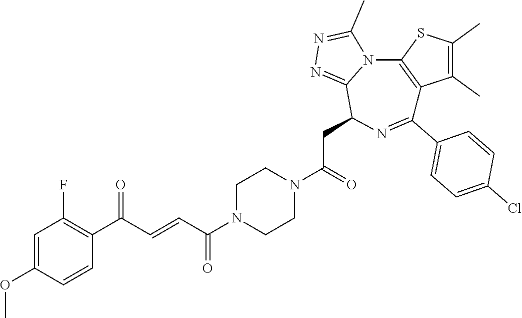 [SMILES: COc1ccc(C(=O)/C=C/C(=O)N2CCN(C(=O)C[C@@H]3N=C(c4ccc(Cl)cc4)c4c(sc(C)c4C)-n4c(C)nnc43)CC2)c(F)c1] | ¹H NMR (400 MHz, DMSO) δ 7.62-7.39 (m, 3H), 7.06-6.93 (m, 1H), 3.89 (s, 1H), 3.76 (s, 1H), 3.59 (d, J = 15.9 Hz, 2H), 2.61 (s, 1H), 2.48 (s, 1H), 2.43 (s, 1H), 1.64 (s, 1H); 19F NMR (376 MHz, DMSO) δ −106.9 | Rt = 2.61; 675.2 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(2-fluoro-4- | | |
| | methoxyphenyl)but-2-ene-1,4-dione | | |
| Compound 234 | 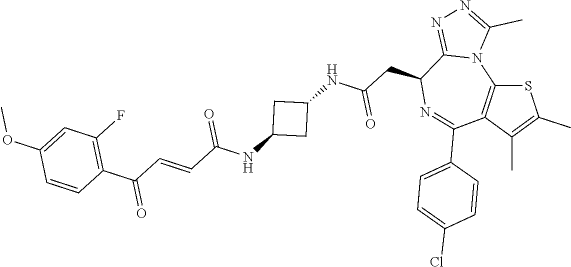 | ¹H NMR (400 MHz, DMSO) δ 9.04 (d, J = 7.2 Hz, 1H), 8.72 (d, J = 7.1 Hz, 1H), 7.88 (t, J = 8.8 Hz, 1H), 7.63-7.54 (m, 3H), 7.48 (d, J = 8.5 Hz, 2H), 7.07 (dd, J = 13.6, 2.5 Hz, 1H), 7.03-6.94 (m, 2H), 4.60-4.44 (m, 2H), 4.38 (d, J = 7.1 Hz, 1H), 3.94 (s, 3H), 3.29 (qd, J = 15.0, 7.1 Hz, 2H), 2.66 (s, 3H), 2.47 (s, 3H), 2.33 (s, 2H), 1.68 (s, 3H); 19F NMR (376 MHz, DMSO) δ −107.2 | Rt = 2.95; 675.2 [M + H]⁺ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-(2-fluoro-4- | | |
| | methoxyphenyl)-4-oxobut-2-enamide | | |
| Compound 235 | 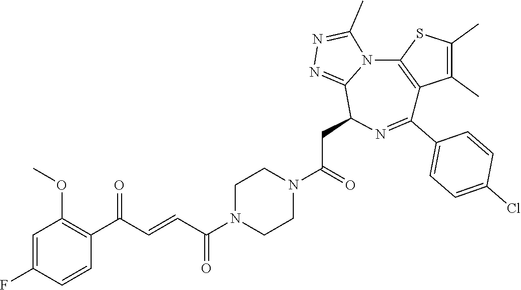 [SMILES: COc1cc(F)ccc1C(=O)/C=C/C(=O)N1CCN(C(=O)C[C@@H]2N=C(c3ccc(Cl)cc3)c3c(sc(C)c3C)-n3c(C)nnc32)CC1] | ¹H NMR (400 MHz, DMSO) δ 7.68 (t, J = 7.5 Hz, 1H), 7.55-7.42 (m, 5H), 7.33 (dd, J = 15.5, 7.1 Hz, 1H), 7.19-7.12 (m, 1H), 6.93 (t, J = 8.7 Hz, 1H), 4.60 (t, J = 6.7 Hz, 1H), 3.92 (d, J = 3.2 Hz, 3H), 3.74 (s, 2H), 3.57 (s, 2H), 3.45 (dd, J = 16.6, 6.3 Hz, 2H), 2.61 (s, 3H), 2.48 (s, 3H), 2.43 (s, 3H), 1.64 (s, 2H); 19F NMR (376 MHz, DMSO) δ −103.5 | Rt = 2.62; 675.2 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(4-fluoro-2- | | |
| | methoxyphenyl)but-2-ene-1,4-dione | | |
| Compound 236 | 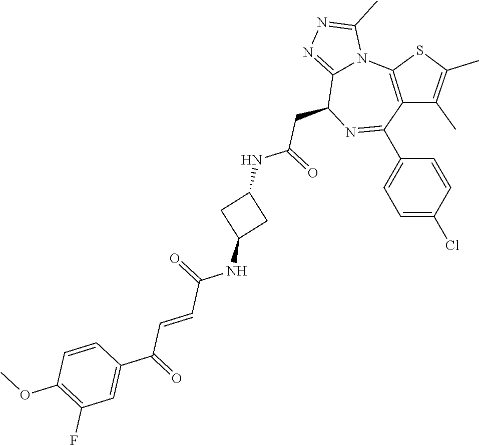 | ¹H NMR (400 MHz, DMSO) δ 9.05 (d, J = 7.2 Hz, 1H), 8.73 (d, J = 7.1 Hz, 1H), 8.01 (dd, J = 8.6, 2.2 Hz, 1H), 7.90 (dd, J = 12.1, 2.1 Hz, 1H), 7.84 (d, J = 15.2 Hz, 1H), 7.57 (d, J = 8.4 Hz, 2H), 7.54-7.45 (m, 2H), 7.40 (t, J = 8.6 Hz, 1H), 7.03 (d, J = 15.2 Hz, 1H), 4.61-4.46 (m, 2H), 4.39 (q, J = 6.8 Hz, 1H), 4.02 (s, 3H), 3.30 (qd, J = 15.0, 7.1 Hz, 2H), 2.66 (s, 3H), 2.47 (s, 3H), 2.35 (h, J = 5.9 Hz, 4H), 1.68 (s, 3H); 19F NMR (376 MHz, DMSO) δ −134.2 | Rt = 2.94; 675.2 [M + H]⁺ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-(3-fluoro-4- | | |
| | methoxyphenyl)-4-oxobut-2-enamide | | |
| Compound 237 | 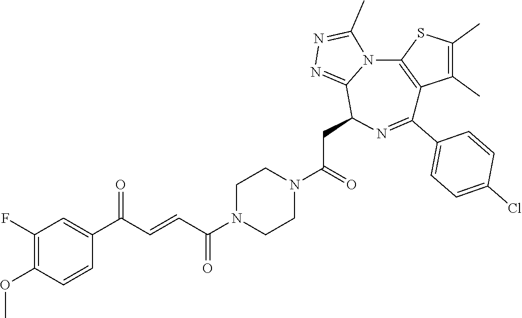 [SMILES: COc1ccc(C(=O)/C=C/C(=O)N2CCN(C(=O)C[C@@H]3N=C(c4ccc(Cl)cc4)c4c(sc(C)c4C)-n4c(C)nnc43)CC2)cc1F] | ¹H NMR (400 MHz, DMSO) δ −7.98 (d, J = 8.8 Hz, 1H), 7.85 (dd, J = 23.5, 13.7 Hz, 2H), 7.54-7.42 (m, 5H), 7.35 (t, J = 8.5 Hz, 1H), 4.60 (t, J = 6.7 Hz, 1H), 3.97 (s, 3H), 3.75 (s, 3H), 3.58 (s, 3H), 3.45 (d, J = 16.5 Hz, 2H), 3.32 (s, 14H), 2.61 (s, 3H), 2.43 (s, 3H), 1.64 (s, 3H); 19F NMR (376 MHz, DMSO) δ −134.2 | Rt = 2.71; 675.2 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(3-fluoro-4- | | |
| | methoxyphenyl)but-2-ene-1,4-dione | | |
| Compound 238 | 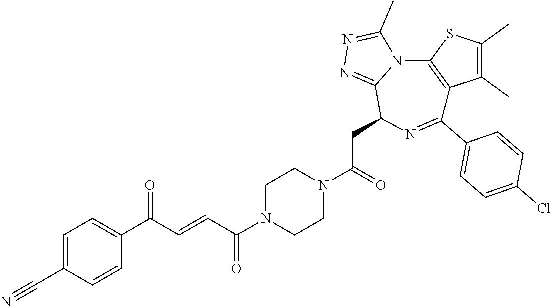 [SMILES: Cc1sc2c(c1C)C(c1ccc(Cl)cc1)=N[C@@H](CC(=O)N1CCN(C(=O)/C=C/C(=O)c3ccc(C#N)cc3)CC1)c1nnc(C)n1-2] | ¹H NMR (400 MHz, CDCl₃) δ 8.15 (d, J = 8.2 Hz, 2H), 7.96 (dd, J = 14.9, 5.8 Hz, 1H), 7.85 (d, J = 8.2 Hz, 2H), 7.58 (dd, J = 14.9, 3.6 Hz, 1H), 7.42 (d, J = 8.3 Hz, 2H), 7.36 (d, J = 8.4 Hz, 2H), 4.83 (t, J = 6.8 Hz, 1H), 4.05-3.72 (m, 7H), 3.56 (ddt, J = 38.0, 22.0, 7.4 Hz, 3H), 2.69 (s, 3H), 2.43 (s, 3H), 1.70 (s, 3H). | Rt = 2.65; 652.2 [M + H]+ |
| | (S,E)-4-(4-(4-(2-(4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetyl)piperazin-1-yl)-4-oxobut-2- | | |
| | enoyl)benzonitrile | | |
| Compound 239 | 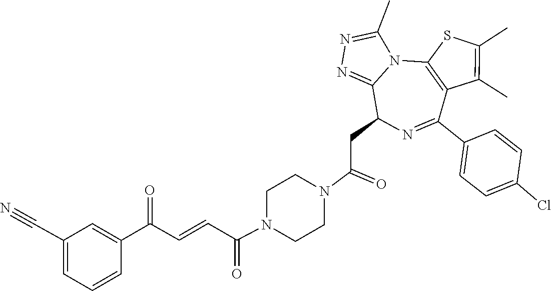 [SMILES: Cc1sc2c(c1C)C(c1ccc(Cl)cc1)=N[C@@H](CC(=O)N1CCN(C(=O)/C=C/C(=O)c3cccc(C#N)c3)CC1)c1nnc(C)n1-2] | ¹H NMR (400 MHz, CDCl₃) δ 8.36 (d, J = 2.0 Hz, 1H), 8.28 (d, J = 7.9 Hz, 1H), 8.00-7.89 (m, 2H), 7.70 (t, J = 7.8 Hz, 1H), 7.59 (dd, J = 14.8, 3.8 Hz, 1H), 7.42 (d, J = 8.4 Hz, 2H), 7.36 (d, J = 8.4 Hz, 2H), 4.83 (t, J = 6.8 Hz, 1H), 4.05-3.97 (m, 2H), 3.97-3.72 (m, 4H), 3.56 (ddt, J = 36.6, 21.8, 7.3 Hz, 3H), 2.70 (s, 3H), 2.43 (s, 3H), 1.70 (s, 3H). | Rt 2.64; 652.2 [M + H]+ |
| | (S,E)-3-(4-(4-(2-(4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetyl)piperazin-1-yl)-4-oxobut-2- | | |
| | enoyl)benzonitrile | | |
| Compound 240 | 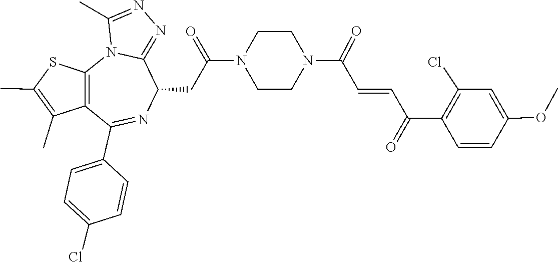 | ¹H NMR (400 MHz, DMSO) δ 7.71 (dd, J = 8.6, 2.6 Hz, 1H), 7.50 (d, J = 8.7 Hz, 2H), 7.47-7.28 (m, 4H), 7.19 (d, J = 2.4 Hz, 1H), 7.11-7.04 (m, 1H), 4.60 (t, J = 6.8 Hz, 1H), 3.88 (s, 3H), 3.76-3.67 (m, 4H), 3.56 (s, 4H), 3.45 (dd, J = 16.1, 6.2 Hz, 2H), 2.61 (s, 3H), 2.43 (s, 3H), 1.64 (s, 3H). | Rt = 2.66; 692.2 [M + H]+ |
| | (S,E)-1-(2-chloro-4-methoxyphenyl)-4- | | |
| | (4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)but-2-ene-1,4- | | |
| | dione | | |
| Compound 241 | 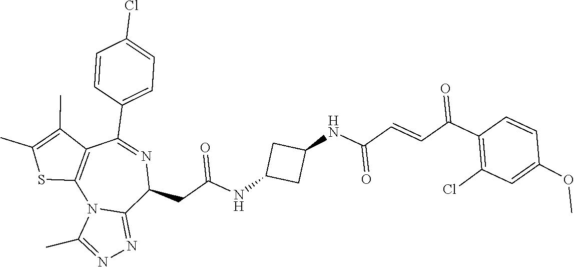 | ¹H NMR (400 MHz, DMSO) δ 9.02 (d, J = 7.3 Hz, 1H), 8.71 (d, J = 7.2 Hz, 1H), 7.69 (d, J = 8.6 Hz, 1H), 7.56 (d, J = 8.7 Hz, 2H), 7.48 (d, J = 8.5 Hz, 2H), 7.32 (d, J = 15.5 Hz, 1H), 7.24 (d, J = 2.4 Hz, 1H), 7.12 (dd, J = 8.6, 2.5 Hz, 1H), 6.80 (d, J = 15.5 Hz, 1H), 4.55 (dd, J = 8.1, 6.1 Hz, 1H), 4.47 (q, J = 6.7 Hz, 1H), 4.36 (t, J = 6.7 Hz, 1H), 3.92 (s, 3H), 3.28 (qd, J = 14.7, 6.9 Hz, 2H), 2.66 (s, 3H), 2.47 (s, 3H), 2.31 | Rt = 3.00; 691.2 [M + H]+ |
| | (E)-4-(2-chloro-4-methoxyphenyl)-N- | (q, J = 10.1 Hz, 3H), 1.70- | |
| | ((1S,3r)-3-(2-((S)-4-(4-chlorophenyl)- | 1.66 (m, 3H), 1.30 (s, 1H). | |
| | 2,3,9-trimethyl-6H-thieno[3,2- | | |
| | f][1,2,4]triazolo[4,3-a][1,4]diazepin-6- | | |
| | yl)acetamido)cyclobutyl)-4-oxobut-2- | | |
| | enamide | | |
| Compound 242 | 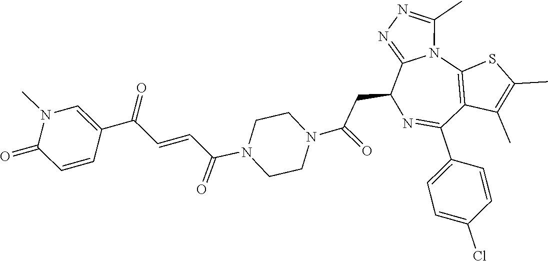 [SMILES: Cc1sc2c(c1C)C(c1ccc(Cl)cc1)=N[C@@H](CC(=O)N1CCN(C(=O)/C=C/C(=O)c3ccc(=O)n(C)c3)CC1)c1nnc(C)n1-2] | ¹H NMR (400 MHz, DMSO) δ 8.69 (d, J = 2.7 Hz, 1H), 7.92 (dd, J = 9.5, 2.7 Hz, 1H), 7.61 (d, J = 15.3 Hz, 1H), 7.45 (s, 4H), 7.37 (d, J = 15.3 Hz, 1H), 6.47 (d, J = 9.5 Hz, 1H), 4.65 (t, J = 6.7 Hz, 1H), 3.70 (s, 6H), 3.65 (d, J = 6.6 Hz, 2H), 3.57 (s, 3H), 3.47 (dd, J = 16.1, 6.4 Hz, 1H), 2.85 (s, 8H), 2.61 (d, J = 3.8 Hz, 3H), 2.43 (s, 3H), 1.68 (s, 3H). | Rt = 2.15; 658.1 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(1-methyl-6- | | |
| | oxo-1,6-dihydropyridin-3-y1)but-2-ene- | | |
| | 1,4-dione | | |
| Compound 243 | 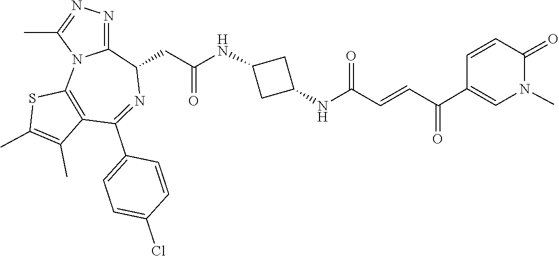 | ¹H NMR (400 MHz, DMSO) δ 9.01 (d, J = 2.7 Hz, 1H), 8.89 (d, J = 7.3 Hz, 1H), 8.57 (d, J = 7.4 Hz, 1H), 8.00 (dd, J = 9.5, 2.6 Hz, 1H), 7.81 (d, J = 15.1 Hz, 1H), 7.56 (d, J = 8.3 Hz, 2H), 7.48 (d, J = 8.4 Hz, 2H), 7.07 (d, J = 15.0 Hz, 1H), 6.55 (d, J = 9.6 Hz, 1H), 4.55 (t, J = 7.1 Hz, 1H), 4.05 (dq, J = 15.8, 7.8 Hz, 2H), 3.61 (s, 3H), 3.28 (d, J = 6.9 Hz, 2H), 2.66 (s, 4H), 2.47 (s, 3H), 2.04- 1.89 (m, 2H), 1.69 (s, 3H). | Rt = 2.59; 658.2 [M + H]+ |
| | (E)-N-((1R,3s)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-(1-methyl-6- | | |
| | oxo-1,6-dihydropyridin-3-yl)-4-oxobut- | | |
| | 2-enamide | | |
| Compound 244 | 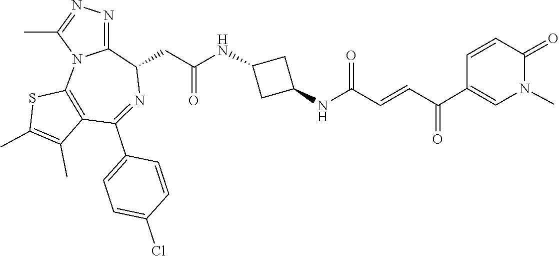 | ¹H NMR (400 MHz, DMSO) δ 9.01-8.93 (m, 2H), 8.67 (d, J = 7.1 Hz, 1H), 7.94 (dd, J = 9.6, 2.6 Hz, 1H), 7.76 (d, J = 15.0 Hz, 1H), 7.51 (d, J = 8.3 Hz, 2H), 7.43 (d, J = 8.4 Hz, 2H), 7.00 (d, J = 15.0 Hz, 1H), 6.50 (d, J = 9.6 Hz, 1H), 4.55-4.41 (m, 2H), 4.38-4.30 (m, 1H), 3.56 (s, 3H), 3.23 (td, J = 15.4, 6.6 Hz, 2H), 2.61 (s, 3H), 2.42 (s, 3H), 2.33-2.24 (m, 4H), 1.63 (s, 3H). | Rt = 2.62; 659.2 [M + H]+ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-(1-methyl-6- | | |
| | oxo-1,6-dihydropyridin-3-yl)-4-oxobut- | | |
| | 2-enamide | | |
| Compound 245 | 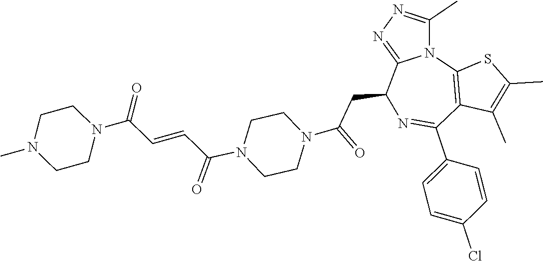 | ¹H NMR (400 MHz, DMSO) δ 7.53-7.41 (m, 4H), 7.29 (s, 2H), 4.59 (t, J = 6.7 Hz, 1H), 3.72 (d, J = 13.2 Hz,4H), 3.63 (s, 1H), 3.55 (s, 6H), 3.44 (dd, J = 16.4, 6.4 Hz, 1H), 2.60 (s, 3H), 2.42 (s, 3H), 2.32 (d, J = 9.6 Hz, 4H), 2.19 (s, 3H), 1.64 (s, 3H). | Rt = 1.72; 649.5 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(4- | | |
| | methylpiperazin-1-yl)but-2-ene-1,4- | | |
| | dione | | |
| Compound 246 | 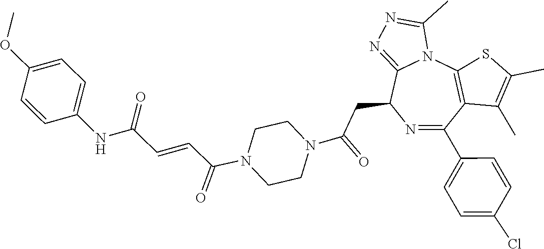 [SMILES: COc1ccc(NC(=O)/C=C/C(=O)N2CCN(C(=O)C[C@@H]3N=C(c4ccc(Cl)cc4)c4c(sc(C)c4C)-n4c(C)nnc43)CC2)cc1] | ¹H NMR (400 MHz, DMSO) δ 10.36 (s, 1H), 7.62 (d, J = 8.5 Hz, 2H), 7.53-7.38 (m, 5H), 7.08 (d, J = 14.9 Hz, 1H), 6.93 (d, J = 8.9 Hz, 2H), 4.60 (t, J = 6.7 Hz, 1H), 3.77 (s, 2H), 3.74 (s, 4H), 3.72-3.60 (m, 3H), 3.57 (s, 2H), 3.45 (dd, J = 16.4, 6.4 Hz, 1H), 2.61 (s, 3H), 2.45-2.40 (m, 3H), 1.66-1.62 (m, 3H). | Rt = 1.95; 672.4 [M + H]+ |
| | (S,E)-4-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-N-(4- | | |
| | methoxyphenyl)-4-oxobut-2-enamide | | |
| Compound 247 | 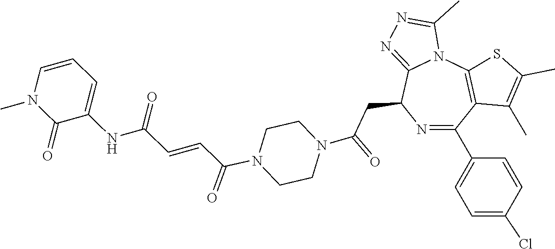 | ¹H NMR (400 MHz, DMSO) δ 9.98 (s, 1H), 8.41 (d, J = 7.4 Hz, 1H), 7.59- 7.47 (m, 5H), 7.45 (s, 2H), 6.34 (t, J = 7.1 Hz, 1H), 4.65 (t, J = 6.7 Hz, 1H), 3.80 (d, J = 12.0 Hz, 3H), 3.72 (dd, J = 16.6, 7.2 Hz, 2H), 3.61 (d, J = 18.8 Hz, 6H), 3.56-3.47 (m, 1H), 2.66 (s, 3H), 2.51-2.46 (m, 3H), 1.72-1.67 (m, 3H), 1.30 (s, 1H). | Rt = 1.88; 673.4 [M + H]+ |
| | (S,E)-4-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-N-(1-methyl-2- | | |
| | oxo-1,2-dihydropyridin-3-yl)-4-oxobut- | | |
| | 2-enamide | | |
| Compound 248 | 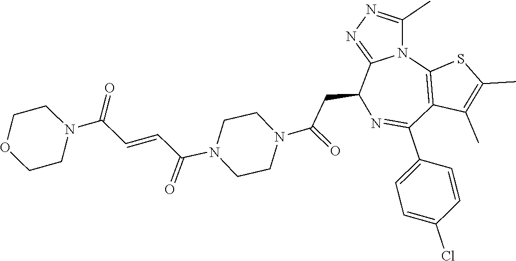 [SMILES: Cc1sc2c(c1C)C(c1ccc(Cl)cc1)=N[C@@H](CC(=O)N1CCN(C(=O)/C=C/C(=O)N3CCOCC3)CC1)c1nnc(C)n1-2] | ¹H NMR (400 MHz, DMSO) δ 7.53-7.41 (m, 4H), 7.30 (d, J = 8.2 Hz, 2H), 4.59 (t, J = 6.7 Hz, 1H), 3.73 (d, J = 13.5 Hz, 4H), 3.57 (d, J = 15.4 Hz, 10H), 2.61 (s, 3H), 2.43 (s, 3H), 1.64 (s, 3H). | Rt = 1.85; 636.4 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4- | | |
| | morpholinobut-2-ene-1,4-dione | | |
| Compound 249 | 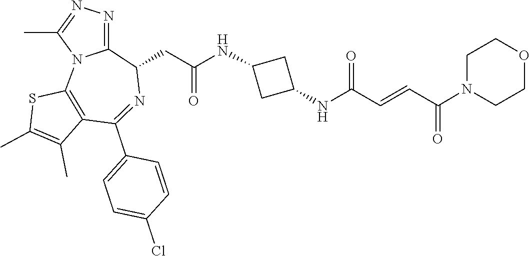 [SMILES: Cc1sc2c(c1C)C(c1ccc(Cl)cc1)=N[C@@H](CC(=O)N[C@H]1C[C@@H](NC(=O)/C=C/C(=O)N3CCOCC3)C1)c1nnc(C)n1-2] | ¹H NMR (400 MHz, DMSO) δ 8.72 (d, J = 7.2 Hz, 1H), 8.51 (d, J = 7.3 Hz, 1H), 7.53-7.38 (m, 4H), 7.24 (d, J = 15.0 Hz, 1H), 7.19 (s, 1H), 6.84 (d, J = 15.1 Hz, 1H), 4.49 (t, J = 7.1 Hz, 1H), 3.96 (p, J = 8.1 Hz, 2H), 3.61-3.51 (m, 8H), 3.27-3.15 (m, 3H), 2.60 (s, 6H), 2.42 (s, 3H), 1.89 (dq, J = 13.9, 9.6 Hz, 2H), 1.63 (s, 3H), 1.26- 1.19 (m, 1H) | Rt = 2.57; 636.2 [M + H]+ |
| | (E)-N-((1R,3s)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-morpholino- | | |
| | 4-oxobut-2-enamide | | |
| Compound 250 | 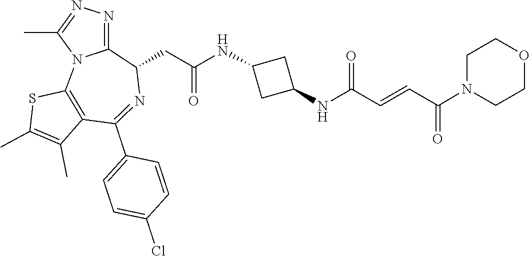 [SMILES: Cc1sc2c(c1C)C(c1ccc(Cl)cc1)=N[C@@H](CC(=O)N[C@H]1C[C@H](NC(=O)/C=C/C(=O)N3CCOCC3)C1)c1nnc(C)n1-2] | ¹H NMR (400 MHz, DMSO) δ 8.81 (d, J = 7.3 Hz, 1H), 8.65 (d, J = 7.1 Hz, 1H), 7.50 (d, J = 8.6 Hz, 2H), 7.47-7.39 (m, 2H), 7.25 (d, J = 15.1 Hz, 1H), 6.82 (d, J = 15.0 Hz, 1H), 4.54-4.35 (m, 2H), 4.31 (q, J = 6.7 Hz, 1H), 3.56 (dd, J = 16.7, 3.4 Hz, 7H), 3.33- 3.14 (m, 10H), 2.60 (s, 3H), 2.44-2.39 (m, 3H), 2.32- 2.19 (m, 4H), 1.65-1.60 (m, 3H). | Rt = 2.56; 636.2 [M + H]+ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-morpholino- | | |
| | 4-oxobut-2-enamide | | |
| Compound 251 | 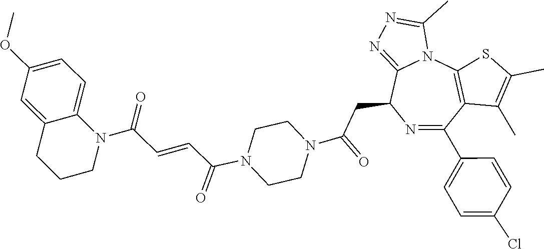 [SMILES: COc1ccc2c(c1)CCCN2C(=O)/C=C/C(=O)N1CCN(C(=O)C[C@@H]2N=C(c3ccc(Cl)cc3)c3c(sc(C)c3C)-n3c(C)nnc32)CC1] | ¹H NMR (400 MHz, MeOD) δ 7.53-7.42 (m, 4H), 7.14 (d, J = 15.0 Hz, 1H), 6.99 (d, J = 8.6 Hz, 1H), 6.88-6.78 (m, 2H), 4.73 (t, J = 6.9 Hz, 1H), 4.61 (s, 1H), 3.92-3.76 (m, 9H), 3.72 (d, J = 17.0 Hz, 2H), 3.66 (d, J = 6.9 Hz, 2H), 2.76 (s, 1H), 2.72 (s, 3H), 2.47 (s, 3H), 2.00 (p, J = 6.7 Hz, 2H), 1.72 (s, 3H). | Rt = 2.05; 712.4 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(6-methoxy- | | |
| | 3,4-dihydroquinolin-1(2H)-yl)but-2-ene- | | |
| | 1,4-dione | | |
| Compound 252 | 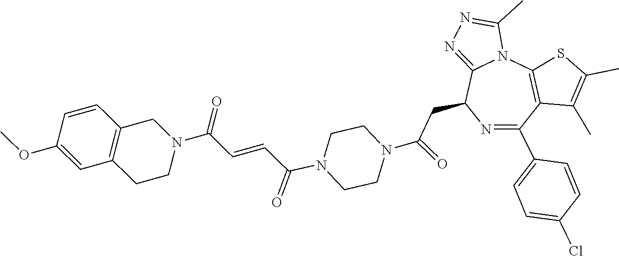 | ¹H NMR (400 MHz, DMSO) δ 7.53-7.42 (m, 4H), 7.42-7.26 (m, 2H), 6.79 (d, J = 8.3 Hz, 2H), 4.73 (s, 1H), 4.60 (dd, J = 13.9, 7.3 Hz, 2H), 3.79 (d, J = 6.0 Hz, 2H), 3.73 (s, 6H), 3.70-3.60 (m, 2H), 3.56 (s, 3H), 3.45 (dd, J = 16.5, 6.3 Hz, 1H), 2.87 (s, 1H), 2.80 (s, 1H), 2.61 (s, 3H), 2.43 (s, 3H), 1.64 (s, 3H). | Rt = 2.16; 712.4 [M + H]+ |
| | (S,E)-1-(4-(2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)acetyl)piperazin-1-yl)-4-(6-methoxy- | | |
| | 3,4-dihydroisoquinolin-2(1H)-y1)but-2- | | |
| | ene-1,4-dione | | |
| Compound 253 | 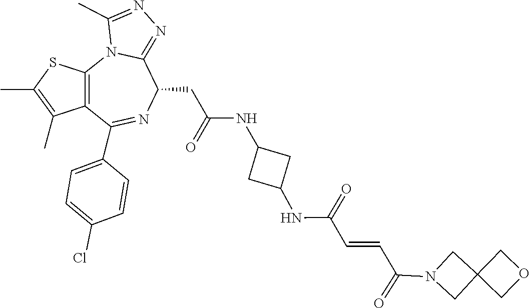 [SMILES: Cc1sc2c(c1C)C(c1ccc(Cl)cc1)=N[C@@H](CC(=O)NC1CC(NC(=O)/C=C/C(=O)N3CC4(COC4)C3)C1)c1nnc(C)n1-2] | ¹H NMR (400 MHz, DMSO) δ 8.71 (d, J = 7.3 Hz, 1H), 8.50 (d, J = 7.3 Hz, 1H), 7.50 (d, J = 8.4 Hz, 2H), 7.47-7.38 (m, 2H), 6.82 (d, J = 15.2 Hz, 1H), 6.73 (d, J = 15.2 Hz, 1H), 4.67 (s, 4H), 4.49 (t, J = 7.1 Hz, 1H), 4.45 (s, 2H), 4.12 (s, 2H), 3.95 (q, J = 8.0 Hz, 2H), 3.25-3.19 (m, 2H), 2.60 (s, 4H), 2.42 (s, 3H), 1.95-1.80 (m, 2H), 1.62 (s, 3H). | Rt = 2.52; 649.2 [M + H]+ |
| | (S,E)-N-(3-(2-(4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetamido)cyclobutyl)-4-oxo-4-(2- | | |
| | oxa-6-azaspiro[3.3]heptan-6-yl)but-2- | | |
| | enamide | | |
| Compound 254 | 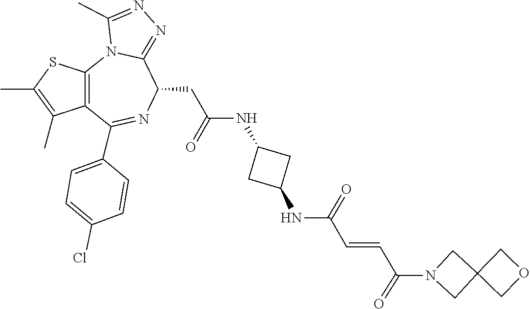 [SMILES: Cc1sc2c(c1C)C(c1ccc(Cl)cc1)=N[C@@H](CC(=O)N[C@H]1C[C@H](NC(=O)/C=C/C(=O)N3CC4(COC4)C3)C1)c1nnc(C)n1-2] | ¹H NMR (400 MHz, DMSO) δ 8.86 (d, J = 7.2 Hz, 1H), 8.66 (d, J = 7.0 Hz, 1H), 7.51 (d, J = 8.4 Hz, 2H), 7.42 (d, J = 8.6 Hz, 2H), 6.81 (d, J = 15.2 Hz, 1H), 6.74 (d, J = 15.2 Hz, 1H), 4.67 (s, 4H), 4.50 (dd, J = 8.1, 6.2 Hz, 1H), 4.47- 4.36 (m, 3H), 4.33-4.25 (m, 1H), 4.12 (s, 2H), 3.23 (qd, J = 15.0, 7.1 Hz, 3H), 2.60 (s, 3H), 2.42 (s, 3H), 2.25 (s, 2H), 1.63 (s, 3H). | Rt = 2.50; 649.2 [M + H]+ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-oxo-4-(2- | | |
| | oxa-6-azaspiro[3.3 ]heptan-6-yl)but-2- | | |
| | enamide | | |
| Compound 255 | 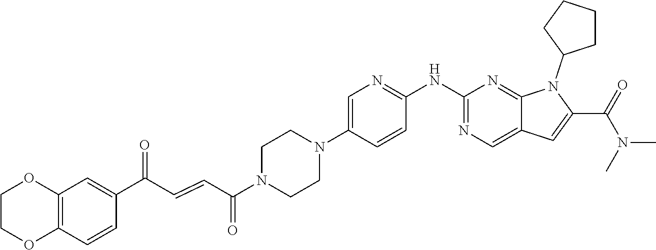 [SMILES: CN(C)C(=O)c1cc2cnc(Nc3ccc(N4CCN(C(=O)/C=C/C(=O)c5ccc6c(c5)OCCO6)CC4)cn3)nc2n1C1CCCC1] | ¹H NMR (400 MHz, DMSO) δ 9.35 (s, 1H), 8.77 (s, 1H), 8.19 (d, J = 9.1 Hz, 1H), 8.04 (d, J = 2.9 Hz, 1H), 7.76 (d, J = 15.2 Hz, 1H), 7.62 (dd, J = 8.5, 2.2 Hz, 1H), 7.56-7.42 (m, 2H), 7.03 (d, J = 8.5 Hz, 1H), 6.61 (s, 1H), 4.73 (q, J = 8.8 Hz, 1H), 4.40-4.28 (m, 3H), 3.76 (t, J = 5.1 Hz, 3H), 3.33 (s, 3H), 3.18 (q, J = 5.1 Hz, 3H), 2.44 (q, J = 8.9 Hz, 2H), 2.05-1.96 (m, 3H), 1.66 (s, 1H). | Rt = 2.30; 651.3 [M + H]+ |
| | (E)-7-cyclopentyl-2-((5-(4-(4-(2,3- | | |
| | dihydrobenzo[b][1,4]dioxin-6-yl)-4- | | |
| | oxobut-2-enoyl)piperazin-1-yl)pyridin- | | |
| | 2-yl)amino)-N,N-dimethyl-7H- | | |
| | pyrrolo[2,3-d]pyrimidine-6- | | |
| | carboxamide | | |
| Compound 256 | 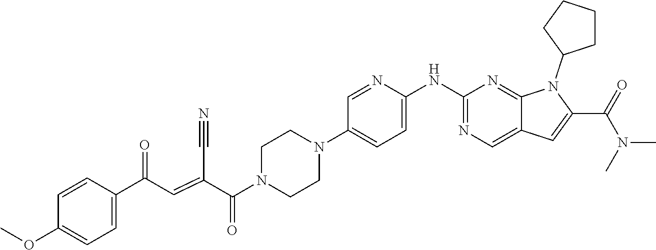 | ¹H NMR (400 MHz, DMSO) δ 11.59 (s, 1H), 9.02 (s, 1H), 8.33 (d, J = 17.1 Hz, 1H), 8.19-8.03 (m, 3H), 7.96-7.86 (m, 1H), 7.58 (dd, J = 9.5, 5.9 Hz, 1H), 7.19-7.07 (m, 2H), 6.85 (s, 1H), 4.82 (p, J = 8.7 Hz, 1H), 3.89 (d, J = 3.4 Hz, 3H), 3.84 - 3.72 (m, 4H), 3.31 (s, 2H), 3.24 (s, 2H), 3.07 (s, 6H), 2.31 (s, 2H), 2.10-1.94 (m, 4H), 1.66 (q, J = 6.0 Hz, 2H). | 649.2 [M + H]+ Rt = 2.08; |
| | (E)-2-((5-(4-(2-cyano-4-(4- | | |
| | methoxyphenyl)-4-oxobut-2- | | |
| | enoyl)piperazin-1-yl)pyridin-2- | | |
| | yl)amino)-7-cyclopentyl-N,N-dimethyl- | | |
| | 7H-pyrrolo[2,3-d]pyrimidine-6- | | |
| | carboxamide | | |
| Compound 257 | 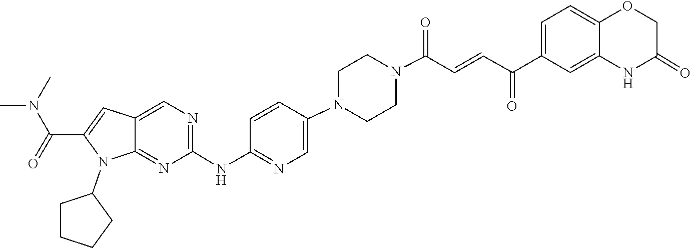 [SMILES: CN(C)C(=O)c1cc2cnc(Nc3ccc(N4CCN(C(=O)/C=C/C(=O)c5ccc6c(c5)NC(=O)CO6)CC4)cn3)nc2n1C1CCCC1] | ¹H NMR (400 MHz, DMSO) δ 10.90 (s, 1H), 9.33 (s, 1H), 8.76 (s, 1H), 8.18 (d, J = 9.1 Hz, 1H), 8.03 (d, J = 2.9 Hz, 1H), 7.79-7.68 (m, 2H), 7.59 (d, J = 2.1 Hz, 1H), 7.54-7.45 (m, 2H), 7.10 (d, J = 8.4 Hz, 1H), 6.60 (s, 1H), 4.73 (s, 3H), 3.77 (s, 4H), 3.18 (s, 5H), 3.06 (s, 6H), 2.44 (s, 2H), 1.99 (s, 4H), 1.65 (s, 2H). | Rt = 2.18; 664.2 [M + H]+ |
| | (E)-7-cyclopentyl-N,N-dimethyl-2-((5- | | |
| | (4-(4-oxo-4-(3-oxo-3,4-dihydro-2H- | | |
| | benzo[b][1,4]oxazin-6-yl)but-2- | | |
| | enoyl)piperazin-1-yl)pyridin-2- | | |
| | yl)amino)-7H-pyrrolo[2,3-d]pyrimidine- | | |
| | 6-carboxamide | | |
| Compound 258 | 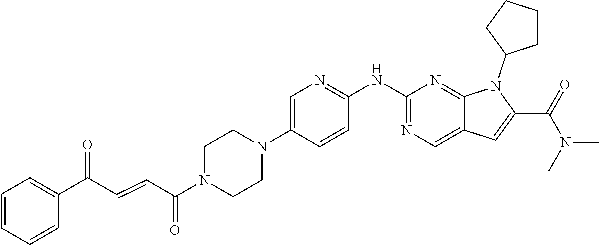 [SMILES: CN(C)C(=O)c1cc2cnc(Nc3ccc(N4CCN(C(=O)/C=C/C(=O)c5ccccc5)CC4)cn3)nc2n1C1CCCC1] | ¹H NMR (400 MHz, DMSO) δ 9.35 (s, 1H), 8.77 (s, 1H), 8.19 (d, J = 9.1 Hz, 1H), 8.09-8.01 (m, 3H), 7.81 (d, J = 15.3 Hz, 1H), 7.76-7.67 (m, 1H), 7.60 (t, J = 7.7 Hz, 2H), 7.56-7.45 (m, 2H), 6.60 (s, 1H), 4.73 (q, J = 8.8 Hz, 1H), 3.78 (d, J = 6.3 Hz, 3H), 3.32 (s, 6H), 3.18 (d, J = 5.3 Hz, 3H), 2.44 (s, 1H), 2.02- 1.96 (m, 3H), 1.65 (s, 1H). | Rt = 2.30; 593.3 [M + H]+ |
| | (E)-7-cyclopentyl-N,N-dimethyl-2-((5- | | |
| | (4-(4-oxo-4-phenylbut-2- | | |
| | enoyl)piperazin-1-yl)pyridin-2- | | |
| | yl)amino)-7H-pyrrolo[2,3-d]pyrimidine- | | |
| | 6-carboxamide | | |
| Compound 259 | 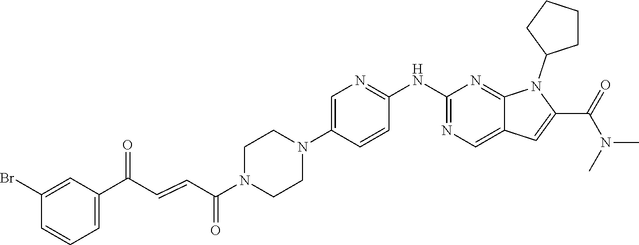 [SMILES: CN(C)C(=O)c1cc2cnc(Nc3ccc(N4CCN(C(=O)/C=C/C(=O)c5cccc(Br)c5)CC4)cn3)nc2n1C1CCCC1] | ¹H NMR (400 MHz, DMSO) δ 9.37 (s, 1H), 8.77 (s, 1H), 8.23-8.15 (m, 2H), 8.06 (dd, J = 11.2, 5.3 Hz, 2H), 7.96-7.89 (m, 1H), 7.78 (d, J = 15.3 Hz, 1H), 7.61-7.46 (m, 3H), 6.62 (s, 1H), 4.79-4.70 (m, 1H), 3.77 (s, 4H), 3.19 (s, 4H), 3.07 (s, 6H), 2.45 (s, 2H), 1.99 (s, 4H), 1.66 (s, 2H). | Rt = 2.35; 671.2 [M + H]+ |
| | (E)-2-((5-(4-(4-(3-bromophenyl)-4- | | |
| | oxobut-2-enoyl)piperazin-1-yl)pyridin- | | |
| | 2-yl)amino)-7-cyclopentyl-N,N- | | |
| | dimethyl-7H-pyrrolo[2,3-d]pyrimidine- | | |
| | 6-carboxamide | | |
| Compound 260 | 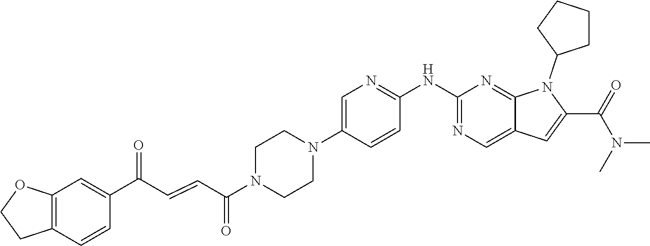 [SMILES: CN(C)C(=O)c1cc2cnc(Nc3ccc(N4CCN(C(=O)/C=C/C(=O)c5ccc6c(c5)OCC6)CC4)cn3)nc2n1C1CCCC1] | ¹H NMR (400 MHz, DMSO) δ 11.42 (s, 1H), 9.00 (s, 1H), 8.12-8.05 (m, 1H), 7.96 (d, J = 2.9 Hz, 1H), 7.81-7.70 (m, 2H), 7.59 (dd, J = 7.7, 1.6 Hz, 1H), 7.53-7.40 (m, 2H), 7.33 (d, J = 1.5 Hz, 1H), 6.82 (s, 1H), 4.81 (p, J = 8.8 Hz, 1H), 4.61 (t, J = 8.7 Hz, 2H), 3.78 (d, J = 9.6 Hz, 3H), 3.63 (s, 1H), 3.33- 3.21 (m, 6H), 2.32 (d, J = 10.7 Hz, 1H), 2.07-1.97 (m, 3H), 1.70-1.62 (m, 2H). | Rt = 2.32; 635.3 [M + H]+ |
| | (E)-7-cyclopentyl-2-((5-(4-(4-(2,3- | | |
| | dihydrobenzofuran-6-yl)-4-oxobut-2- | | |
| | enoyl)piperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 261 | 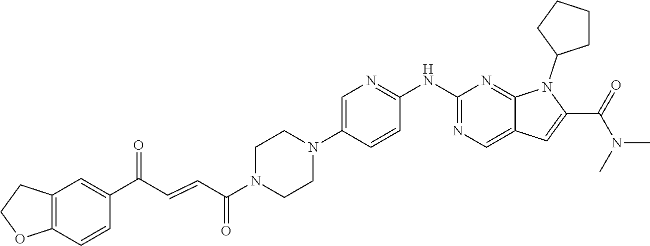 [SMILES: CN(C)C(=O)c1cc2cnc(Nc3ccc(N4CCN(C(=O)/C=C/C(=O)c5ccc6c(c5)CCO6)CC4)cn3)nc2n1C1CCCC1] | ¹H NMR (400 MHz, DMSO) δ 9.33 (s, 1H), 8.76 (s, 1H), 8.18 (d, J = 9.1 Hz, 1H), 8.01 (dd, J = 20.1, 2.4 Hz, 2H), 7.92 (dd, J = 8.4, 2.0 Hz, 1H), 7.81 (d, J = 15.2 Hz, 1H), 7.53-7.43 (m, 2H), 6.92 (d, J = 8.4 Hz, 1H), 6.60 (s, 1H), 4.71 (dt, J = 25.1, 8.8 Hz, 3H), 3.77 (s, 3H), 3.27 (t, J = 8.7 Hz, 3H), 3.18 (d, J = 5.2 Hz, 4H), 3.06 (s, 5H), 2.44 (s, 2H), 1.99 (s, 3H), 1.65 (s, 1H | Rt = 2.32; 635.3 [M + H]+ |
| | (E)-7-cyclopentyl-2-((5-(4-(4-(2,3- | | |
| | dihydrobenzofuran-5-yl)-4-oxobut-2- | | |
| | enoyl)piperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 262 | 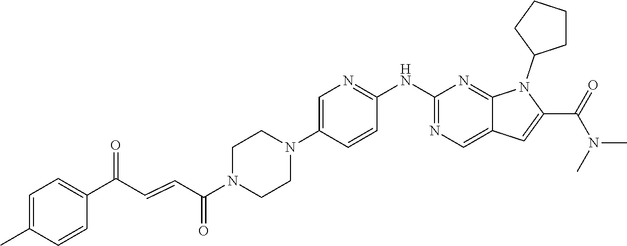 | ¹H NMR (400 MHz, DMSO) δ 8.84 (s, 1H), 8.02- 7.92 (m, 4H), 7.81 (d, J = 15.2 Hz, 1H), 7.50 (d, J = 15.2 Hz, 1H), 7.40 (d, J = 8.0 Hz, 2H), 6.68 (s, 1H), 4.77 (p, J = 8.7 Hz, 1H), 3.78 (d, J = 5.2 Hz, 4H), 3.62 (s, 1H), 3.19 (d, J = 4.9 Hz, 4H), 2.44 (s, 1H), 2.42 (s, 4H), 1.99 (s, 3H), 1.66 (s, 2H). | Rt = 2.38; 607.3 [M + H]+ |
| | (E)-7-cyclopentyl-N,N-dimethyl-2-((5- | | |
| | (4-(4-oxo-4-(p-tolyl)but-2- | | |
| | enoyl)piperazin-1-yl)pyridin-2- | | |
| | yl)amino)-7H-pyrrolo[2,3-d]pyrimidine- | | |
| | 6-carboxamide | | |
| Compound 263 | 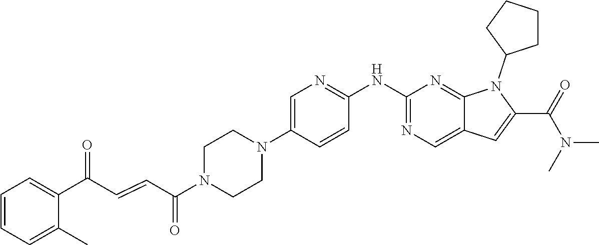 [SMILES: Cc1ccccc1C(=O)/C=C/C(=O)N1CCN(c2ccc(Nc3ncc4cc(C(=O)N(C)C)n(C5CCCC5)c4n3)nc2)CC1] | ¹H NMR (400 MHz, DMSO) δ 9.35 (s, 1H), 8.77 (s, 1H), 8.18 (d, J = 9.1 Hz, 1H), 8.03 (d, J = 3.0 Hz, 1H), 7.72-7.65 (m, 1H), 7.54-7.44 (m, 2H), 7.43- 7.27 (m, 4H), 6.60 (s, 1H), 4.74 (t, J = 8.8 Hz, 1H), 3.74 (d, J = 9.0 Hz, 4H), 3.17 (q, J = 4.8 Hz, 4H), 3.07 (d, J = 9.0 Hz, 6H), 2.43 (s, 4H), 1.98 (s, 3H), 1.68-1.62 (m, 2H), 1.11 (s, 2H). | Rt = 2.29; 607.3 [M + H]+ |
| | (E)-7-cyclopentyl-N,N-dimethyl-2-((5- | | |
| | (4-(4-oxo-4-(o-tolyl)but-2- | | |
| | enoyl)piperazin-1-yl)pyridin-2- | | |
| | yl)amino)-7H-pyrrolo[2,3-d]pyrimidine- | | |
| | 6-carboxamide | | |
| Compound 264 | 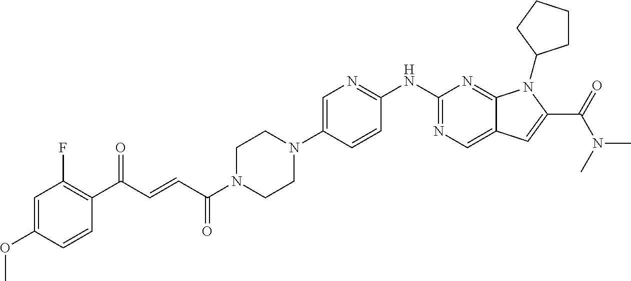 [SMILES: COc1ccc(C(=O)/C=C/C(=O)N2CCN(c3ccc(Nc4ncc5cc(C(=O)N(C)C)n(C6CCCC6)c5n4)nc3)CC2)c(F)c1] | ¹H NMR (400 MHz. DMSO) δ 8.94 (s, 1H), 7.98- 7.93 (m, 1H), 7.85 (t, J = 8.8 Hz, 1H), 7.77 (s, 1H), 7.58 (dd, J = 15.1, 3.4 Hz, 1H), 7.48 (d, J = 15.1 Hz, 1H), 7.06-6.93 (m, 2H), 6.78 (s, 1H), 4.80 (t, J = 8.7 Hz, 1H), 3.89 (s, 3H), 3.79 (d, J = 13.4 Hz, 4H), 3.23 (s, 4H), 3.07 (s, 6H), 2.34 (s, 1H), 2.03-1.96 (m, 3H), 1.66 (d, J = 7.6 Hz, 2H); 19F NMR (376 MHz, DMSO) δ −106.9 | Rt = 2.21; 641.3 [M + H]+ |
| | (E)-7-cyclopentyl-2-((5-(4-(4-(2-fluoro- | | |
| | 4-methoxyphenyl)-4-oxobut-2- | | |
| | enoyl)piperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 265 | 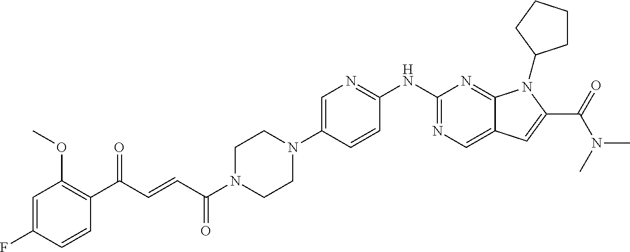 [SMILES: COc1cc(F)ccc1C(=O)/C=C/C(=O)N1CCN(c2ccc(Nc3ncc4cc(C(=O)N(C)C)n(C5CCCC5)c4n3)nc2)CC1] | ¹H NMR (400 MHz, DMSO) δ 9.34 (s, 1H), 8.76 (s, 1H), 8.18 (d, J = 9.0 Hz, 1H), 8.03 (d, J = 3.0 Hz, 1H), 7.72-7.63 (m, 1H), 7.53-7.45 (m, 2H), 7.34 (d, J = 15.3 Hz, 1H), 7.15 (dd, J = 11.4, 2.3 Hz, 1H), 6.93 (td, J = 8.5, 2.5 Hz, 1H), 6.61 (s, 1H), 4.79-4.70 (m, 1H), 3.91 (s, 3H), 3.75 (d, J = 5.8 Hz, 4H), 3.17 (s, 3H), 3.06 (s, 5H), 1.99 (s, 3H), 1.65 (s, 1H); 19F NMR (376 MHz, DMSO) δ −103.5 | Rt = 2.21; 641.3 [M + H]+ |
| | (E)-7-cyclopentyl-2-((5-(4-(4-(4-fluoro- | | |
| | 2-methoxyphenyl)-4-oxobut-2- | | |
| | enoyl)piperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 266 | 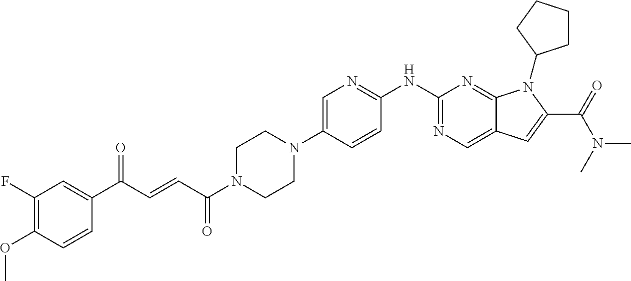 [SMILES: COc1ccc(C(=O)/C=C/C(=O)N2CCN(c3ccc(Nc4ncc5cc(C(=O)N(C)C)n(C6CCCC6)c5n4)nc3)CC2)cc1F] | ¹H NMR (400 MHz, DMSO) δ 9.36 (s, 1H), 8.77 (s, 1H), 8.19 (d, J = 9.1 Hz, 1H), 8.04 (d, J = 3.0 Hz, 1H), 8.01-7.94 (m, 1H), 7.87 (dd, J = 12.2, 2.1 Hz, 1H), 7.81 (d, J = 15.2 Hz, 1H), 7.54-7.45 (m, 2H), 7.35 (t, J = 8.6 Hz, 1H), 6.61 (s, 1H), 4.75 (q, J = 8.8 Hz, 1H), 3.97 (s, 3H), 3.77 (t, J = 5.0 Hz, 3H), 3.22 3.15 (m, 3H), 3.06 (s, 4H), 2.44 (s, 1H), 1.99 (s, 3H), 1.65 (s, 1H); 19F NMR (376 MHz, DMSO) δ −134.2 | Rt = 2.35; 641.3 [M + H]+ |
| | (E)-7-cyclopentyl-2-((5-(4-(4-(3-fluoro- | | |
| | 4-methoxyphenyl)-4-oxobut-2- | | |
| | enoyl)piperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 267 | 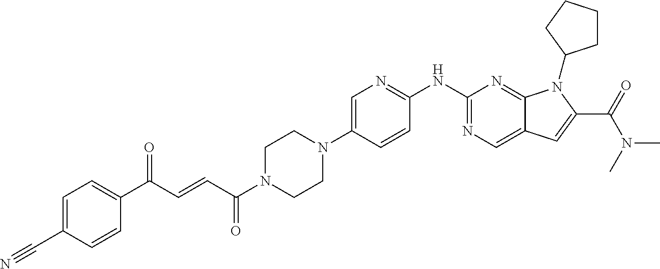 [SMILES: CN(C)C(=O)c1cc2cnc(Nc3ccc(N4CCN(C(=O)/C=C/C(=O)c5ccc(C#N)cc5)CC4)cn3)nc2n1C1CCCC1] | ¹H NMR (400 MHz, CDCl₃) δ 8.72 (s, 1H), 8.44 (d, J = 9.2 Hz, 1H), 8.16 (d, J = 8.2 Hz, 2H), 8.04-7.93 (m, 2H), 7.85 (d, J = 8.2 Hz, 2H), 7.62 (d, J = 14.9 Hz, 1H), 7.42-7.35 (m, 1H), 7.28 (s, 4H), 6.47 (s, 1H), 4.83 (p, J = 8.8 Hz, 1H), 3.97 (t, J = 5.1 Hz, 2H), 3.85 (d, J = 5.2 Hz, 2H), 3.20 (d, J = 13.9 Hz, 9H), 2.58 (d, J = 10.3 Hz, 1H), 2.10 (d, J = 15.4 Hz, 3H), 1.70 (m, 1H) | Rt = 2.23; 618.3 [M + H]+ |
| | (E)-2-((5-(4-(4-(4-cyanophenyl)-4- | | |
| | oxobut-2-enoyl)piperazin-1-yl)pyridin- | | |
| | 2-yl)amino)-7-cyclopentyl-N,N- | | |
| | dimethyl-7H-pyrrolo[2,3-d]pyrimidine- | | |
| | 6-carboxamide | | |
| Compound 268 | 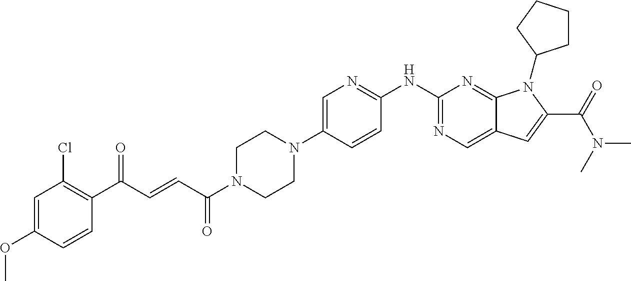 [SMILES: COc1ccc(C(=O)/C=C/C(=O)N2CCN(c3ccc(Nc4ncc5cc(C(=O)N(C)C)n(C6CCCC6)c5n4)nc3)CC2)c(Cl)c1] | ¹H NMR (400 MHz, DMSO) δ 9.33 (s, 1H), 8.77 (s, 1H), 8.18 (d, J = 9.1 Hz, 1H), 8.03 (d, J = 3.0 Hz, 1H), 7.70 (d, J = 8.6 Hz, 1H), 7.48 (dd, J = 9.1, 3.0 Hz, 1H), 7.35 (s, 2H), 7.19 (d, J = 2.5 Hz, 1H), 7.07 (dd, J = 8.7, 2.5 Hz, 1H), 6.61 (s, 1H), 4.75 (q, J = 8.7 Hz, 1H), 3.88 (s, 3H), 3.73 (d, J = 5.5 Hz, 4H), 3.32 (s, 5H), 3.17 (d, J = 5.3 Hz, 4H), 2.44 (s, 1H), 1.99 (s, 3H), 1.66 (s, 2H). | Rt = 2.39; 657.2 [M + H]+ |
| | (E)-2-((5-(4-(4-(2-chloro-4- | | |
| | methoxyphenyl)-4-oxobut-2- | | |
| | enoyl)piperazin-1-yl)pyridin-2- | | |
| | yl)amino)-7-cyclopentyl-N,N-dimethyl- | | |
| | 7H-pyrrolo[2,3-d]pyrimidine-6- | | |
| | carboxamide | | |
| Compound 269 | 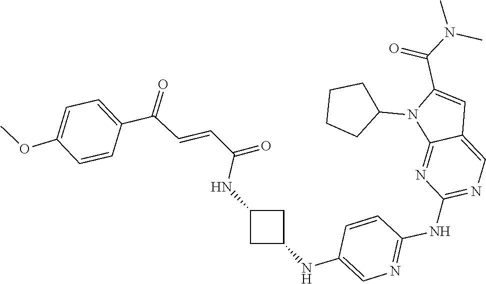 [SMILES: COc1ccc(C(=O)/C=C/C(=O)N[C@H]2C[C@@H](Nc3ccc(Nc4ncc5cc(C(=O)N(C)C)n(C6CCCC6)c5n4)nc3)C2)cc1] | ¹H NMR (400 MHz, DMSO) δ 9.04 (s, 1H), 8.85 (d, J = 7.4 Hz, 1H), 8.72 (d, J = 1.3 Hz, 1H), 8.07-8.01 (m, 2H), 7.97 (d, J = 8.9 Hz, 1H), 7.79 (dd, J = 15.2, 1.2 Hz, 1H), 7.67 (d, J = 2.8 Hz, 1H), 7.14-7.07 (m, 2H), 7.04-6.91 (m, 2H), 6.58 (d, J = 1.2 Hz, 1H), 5.82 (d, J = 6.7 Hz, 1H), 4.71 (q, J = 8.7 Hz, 1H), 4.09 (p, J = 7.7 Hz, 1H), 3.88 (d, J = 1.1 Hz, 3H), 3.61 (q, J = 7.5 Hz, 1H), 3.06 (s, 4H), 2.78 (d, J = 8.2 Hz, 2H), 2.42 (s, 1H), 1.96 (s, 2H), 1.79 (q, J = 9.3 Hz, 2H), 1.64 (d, J = 6.6 Hz, | Rt = 1.89; 623.5 [M + H]+ |
| | 7-cyclopentyl-2-((5-(((1s,3s)-3-((E)-4- | 1H). | |
| | (4-methoxyphenyl)-4-oxobut-2- | | |
| | enamido)cyclobutyl)amino)pyridin-2- | | |
| | yl)amino)-N,N-dimethyl-7H- | | |
| | pyrrolo[2,3-d]pyrimidine-6- | | |
| | carboxamide | | |
| Compound 270 | 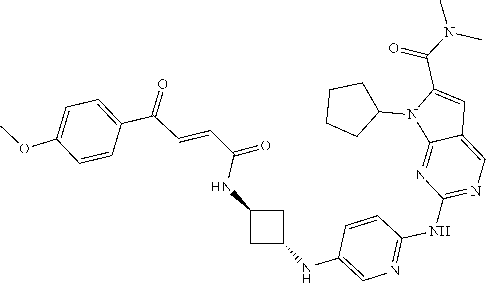 [SMILES: COc1ccc(C(=O)/C=C/C(=O)N[C@H]2C[C@H](Nc3ccc(Nc4ncc5cc(C(=O)N(C)C)n(C6CCCC6)c5n4)nc3)C2)cc1] | ¹H NMR (400 MHz, DMSO) δ 9.04 (s, 1H), 8.97 (d, J = 7.2 Hz, 1H), 8.72 (d, J = 1.2 Hz, 1H), 8.05 (d, J = 8.4 Hz, 2H), 7.98 (d, J = 8.9 Hz, 1H), 7.79 (d, J = 15.2 Hz, 1H), 7.63 (d, J = 2.8 Hz, 1H), 7.14-7.07 (m, 2H), 6.96 (dd, J = 13.3, 5.5 Hz, 2H), 6.57 (d, J = 1.2 Hz, 1H), 5.95 (d, J = 6.2 Hz, 1H), 4.76-4.67 (m, 1H), 4.47 (q, J = 7.1 Hz, 1H), 3.95 (s, 1H), 3.88 (d, J = 1.2 Hz, 3H), 3.05 (s, 4H), 2.44- 2.29 (m, 4H), 1.95 (s, 2H), 1.63 (s, 1H). | Rt = 1.90; 623.5 [M + H]+ |
| | 7-cyclopentyl-2-((5-(((1r,3r)-3-((E)-4- | | |
| | (4-methoxyphenyl)-4-oxobut-2- | | |
| | enamido)cyclobutyl)amino)pyridin-2- | | |
| | yl)amino)-N,N-dimethyl-7H- | | |
| | pyrrolo[2,3-d]pyrimidine-6- | | |
| | carboxamide | | |
| Compound 271 | 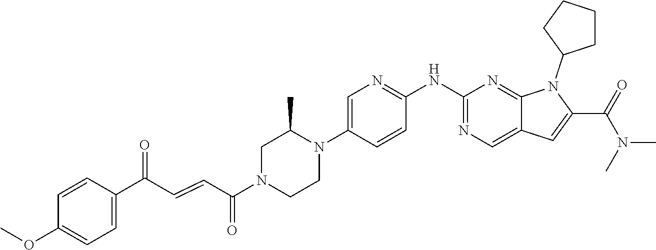 | ¹H NMR (400 MHz, DMSO) δ 9.34 (s, 1H), 8.77 (s, 1H), 8.18 (d, J = 9.0 Hz, 1H), 8.07 (dd, J = 8.7, 6.4 Hz, 2H), 8.00 (s, 1H), 7.84 (dd, J = 15.2, 11.4 Hz, 1H), 7.56-7.42 (m, 2H), 7.14- 7.07 (m, 2H), 6.61 (s, 1H), 4.74 (t, J = 8.8 Hz, 1H), 4.17 (d, J = 12.9 Hz, 1H), 3.95 (d, J = 12.4 Hz, 1H), 3.88 (s, 4H), 3.75 (s, 1H), 3.25 (d, J = 12.5 Hz, 1H), 3.06 (s, 5H), 2.44 (s, 1H), 1.98 (s, 3H), 1.65 (s, 1H), 0.91 (dd, J = 6.3, 4.5 Hz, | Rt = 2.34; 637.3 [M + H]+ |
| | (R,E)-7-cyclopentyl-2-((5-(4-(4-(4- | 3H). | |
| | methoxyphenyl)-4-oxobut-2-enoyl)-2- | | |
| | methylpiperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 272 | 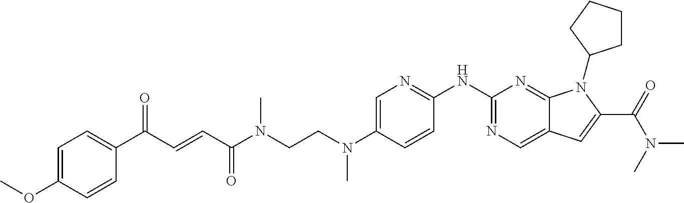 | ¹H NMR (400 MHz, DMSO) δ 8.76 (s, 1H), 7.94 (d, J = 8.7 Hz, 1H), 7.90 (s, 1H), 7.89-7.81 (m, 1H), 7.58 (dd, J = 20.0, 15.1 Hz, 1H), 7.30-7.19 (m, 1H), 7.02-6.94 (m, 1H), 6.91 (s, 1H), 6.64 (d, J = 15.3 Hz, 1H), 4.76-4.67 (m, 1H), 3.82 (s, 1H), 3.74 (s, 1H), 3.66 (d, J = 5.5 Hz, 1H), 3.58 (s, 2H), 3.12 (s, 1H), 3.05 (d, J = 12.2 Hz, 5H), | Rt = 2.18; 625.3 [M + H]+ |
| | (E)-7-cyclopentyl-2-((5-((2-(4-(4- | 2.96 (s, 1H), 2.87 (s, 1H), | |
| | methoxyphenyl)-N-methyl-4-oxobut-2- | 2.37 (s, 1H), 1.96 (s, 2H), | |
| | enamido)ethyl)(methyl)amino)pyridin- | 1.62 (s, 1H). | |
| | 2-yl)amino)-N,N-dimethyl-7H- | | |
| | pyrrolo[2,3-d]pyrimidine-6- | | |
| | carboxamide | | |
| Compound 273 | 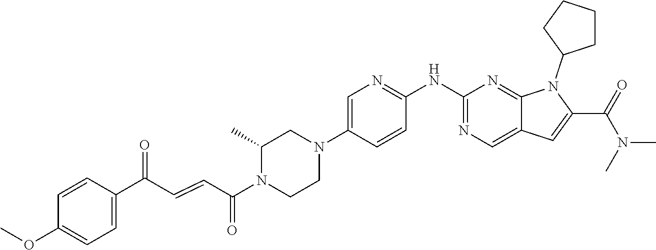 | ¹H NMR (400 MHz, DMSO) δ 8.76 (d, J = 13.3 Hz, 2H), 8.14 (d, J = 9.1 Hz, 1H), 8.08-8.00 (m, 3H), 7.74 (d, J = 15.3 Hz, 1H), 7.47-7.37 (m, 2H), 7.15- 7.07 (m, 2H), 6.57 (s, 1H), 4.78 (p, J = 8.6 Hz, 1H), 4.60 (s, 0H), 4.18 (s, 0H), 3.90 (s, 3H), 3.62 (d, J = 11.9 Hz, 1H), 3.54-3.46 (m, 1H), 3.07 (d, J = 2.3 Hz, 8H), 2.96 (dd, J = 12.2, 3.8 Hz, 1H), 2.84-2.73 (m, 1H), 2.43 (d, J = 10.2 Hz, 1H), 2.01 (d, J = 12.1 Hz, | Rt = 2.37; 637.3 [M + H]+ |
| | (R,E)-7-cyclopentyl-2-((5-(4-(4-(4- | 3H), 1.67 (s, 1H), 1.38 (d, J = | |
| | methoxyphenyl)-4-oxobut-2-enoyl)-3- | 6.7 Hz, 3H). | |
| | methylpiperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 274 | 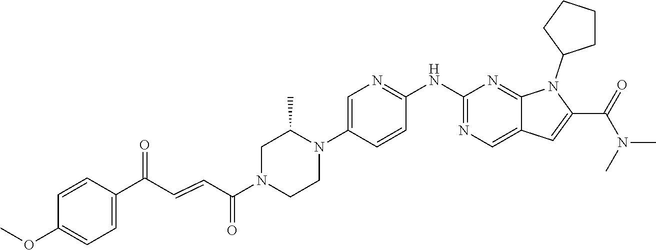 | ¹H NMR (400 MHz, MeOD) δ 8.75 (s, 1H), 8.26 (d, J = 9.0 Hz, 1H), 8.14- 8.07 (m, 2H), 8.04-7.92 (m, 2H), 7.66-7.52 (m, 2H), 7.14-7.06 (m, 2H), 6.64 (s, 1H), 4.78 (p, J = 8.8 Hz, 1H), 4.23 (d, J = 13.0 Hz, 1H), 4.09-3.95 (m, 1H), 3.92 (s, 5H), 3.81- 3.70 (m, 1H), 3.31-3.20 (m, 2H), 3.17 (s, 6H), 2.08 (d, J = 6.4 Hz, 3H), 1.74 (s, 1H), 1.04 (dd, J = 6.3, 1.8 Hz, 3H). | Rt = 2.92; 637.3 [M + H]+ |
| | (S,E)-7-cyclopenty1-2-((5-(4-(4-(4- | | |
| | methoxyphenyl)-4-oxobut-2-enoyl)-2- | | |
| | methylpiperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 275 | 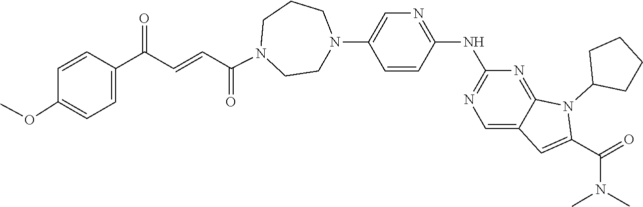 [SMILES: COc1ccc(C(=O)/C=C/C(=O)N2CCCN(c3ccc(Nc4ncc5cc(C(=O)N(C)C)n(C6CCCC6)c5n4)nc3)CC2)cc1] | ¹H NMR (400 MHz, DMSO) δ 9.28 (s, 1H), 8.73 (d, J = 16.3 Hz, 1H), 8.02 (dd, J = 18.3, 9.0 Hz, 1H), 7.96-7.80 (m, 2H), 7.42- 7.17 (m, 2H), 7.09-7.00 (m, 1H), 6.89-6.82 (m, 1H), 6.58 (d, J = 4.3 Hz, 1H), 4.71 (q, J = 9.3 Hz, 1H), 3.86-3.73 (m, 4H), 3.71-3.49 (m, 4H), 3.06 (s, 3H), 2.45-2.18 (m, 2H), 1.95 (s, 2H), 1.61 (d, J = 4.0 | Rt = 2.17; 637.3 [M + H]+ |
| | (E)-7-cyclopentyl-2-((5-(4-(4-(4- | Hz, 2H). | |
| | methoxyphenyl)-4-oxobut-2-enoyl)-1,4- | | |
| | diazepan-1-yl)pyridin-2-yl)amino)-N,N- | | |
| | dimethy1-7H-pyrrolo[2,3-d]pyrimidine- | | |
| | 6-carboxamide | | |
| Compound 276 | 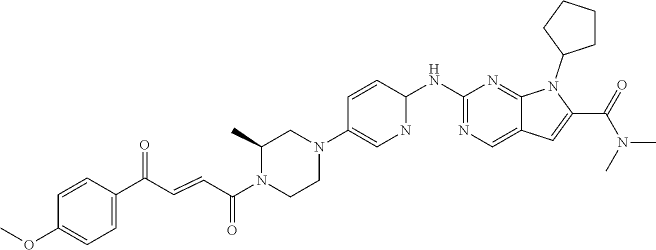 | ¹H NMR (400 MHz, DMSO) δ 8.76 (d, J = 12.1 Hz, 2H), 8.14 (d, J = 9.0 Hz, 1H), 8.07-8.00 (m, 3H), 7.74 (d, J = 15.3 Hz, 1H), 7.47-7.37 (m, 2H), 7.11 (d, J = 8.8 Hz, 2H), 6.57 (s, 1H), 4.78 (p, J = 8.8 Hz, 1H), 4.60 (s, 1H), 4.19 (s, 1H), 3.90 (s, 3H), 3.62 (d, J = 11.8 Hz, 1H), 3.50 (d, J = 12.1 Hz, 1H), 3.42 (s, 1H), 3.07 (s, 6H), 2.97 (dd, J = 12.2, 3.8 Hz, 1H), 2.78 (t, J = 11.3 Hz, 1H), 2.43 (d, J = 10.8 Hz, 2H), 2.00 (s, 4H), | Rt = 2.37; 637.3 [M + H]+ |
| | (S,E)-7-cyclopentyl-2-((5-(4-(4-(4- | 1.67 (s, 2H), 1.38 (d, J = 6.7 | |
| | methoxyphenyl)-4-oxobut-2-enoyl)-3- | Hz, 3H). | |
| | methylpiperazin-1-yl)pyridin-2- | | |
| | yl)amino)-N,N-dimethyl-7H- | | |
| | pyrrolo[2,3-d]pyrimidine-6- | | |
| | carboxamide | | |
| Compound 277 | 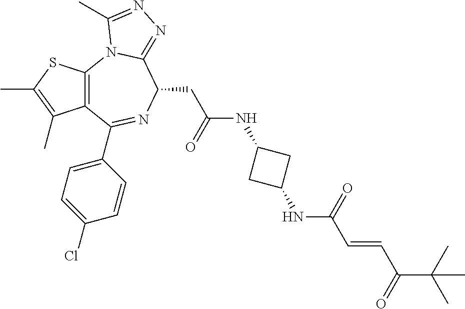 [SMILES: Cc1sc2c(c1C)C(c1ccc(Cl)cc1)=N[C@@H](CC(=O)N[C@H]1C[C@@H](NC(=O)/C=C/C(=O)C(C)(C)C)C1)c1nnc(C)n1-2] | ¹H NMR (400 MHz, DMSO) δ 8.84 (d, J = 7.2 Hz, 1H), 8.55 (d, J = 7.3 Hz, 1H), 7.55 (d, J = 8.4 Hz, 2H), 7.53-7.38 (m, 3H), 6.92 (d, J = 15.2 Hz, 1H), 4.55 (t, J = 7.1 Hz, 1H), 4.02 (hept, J = 8.3 Hz, 2H), 3.33-3.22 (m, 2H), 2.68 (s, 1H), 2.66 (s, 3H), 2.60 (s, 1H), 2.47 (s, 3H), 2.02- 1.88 (m, 2H), 1.68 (s, 3H), 1.19 (s, 9H). | Rt = 2.89; 608.3 [M + H]+ |
| | (E)-N-((1R,3s)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-5,5-dimethyl- | | |
| | 4-oxohex-2-enamide | | |
| Compound 278 | 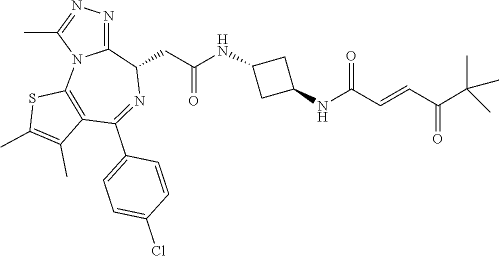 [SMILES: Cc1sc2c(c1C)C(c1ccc(Cl)cc1)=N[C@@H](CC(=O)N[C@H]1C[C@H](NC(=O)/C=C/C(=O)C(C)(C)C)C1)c1nnc(C)n1-2] | ¹H NMR (400 MHz, DMSO) δ 8.97 (d, J = 7.2 Hz, 1H), 8.71 (d, J = 7.1 Hz, 1H), 7.56 (d, J = 8.4 Hz, 2H), 7.51-7.39 (m, 3H), 6.91 (d, J = 15.3 Hz, 1H), 4.56 (dd, J = 8.0, 6.2 Hz, 1H), 4.48 (q, J = 6.9 Hz, 1H), 4.37 (q, J = 6.7 Hz, 1H), 3.29 (qd, J = 15.0, 7.2 Hz, 2H), 2.66 (s, 3H), 2.47 (s, 3H), 2.37-2.25 (m, 3H), 1.68 (s, 3H), 1.19 (s, 9H). | Rt = 2.90; 607.2 [M + H]+ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-5,5-dimethyl- | | |
| | 4-oxohex-2-enamide | | |
| Compound 279 | 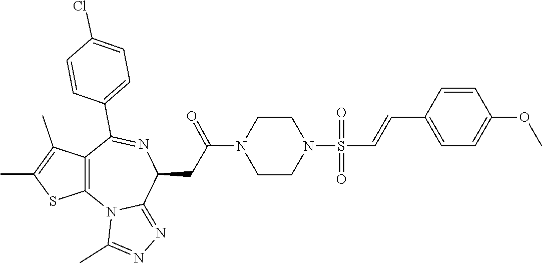 | ¹H NMR (400 MHz, DMSO) δ 7.78-7.70 (m, 2H), 7.53-7.43 (m, 2H), 7.43-7.35 (m, 3H), 7.15 (d, J = 15.5 Hz, 1H), 7.04- 6.96 (m, 2H), 4.55 (t, J = 6.7 Hz, 1H), 3.81 (s, 4H), 3.61 (dd, J = 24.3, 7.7 Hz, 3H), 3.44 (dd, J = 16.3, 6.6 Hz, 1H), 3.23 (d, J = 12.0 Hz, 1H), 3.14 (dd, J = 15.3, 8.2 Hz, 2H), 3.08 (s, 1H), 2.59 (s, 3H), 2.41 (s, 3H), 1.61 (s, 3H), 1.32-1.22 (m, 1H). | Rt 3.11; 665.2 [M + H]+ |
| | (S,E)-2-(4-(4-chlorophenyl)-2,3,9- | | |
| | trimethyl-6H-thieno[3,2- | | |
| | f][1,2,4]triazolo[4,3-a][1,4]diazepin-6- | | |
| | yl)-1-(4-((4- | | |
| | methoxystyryl)sulfonyl)piperazin-1- | | |
| | yl)ethan-1-one | | |
| Compound 280 | 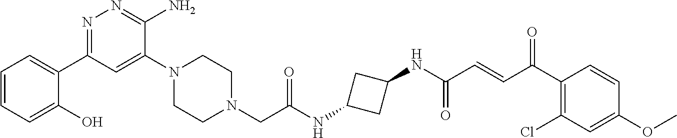 [SMILES: COc1ccc(C(=O)/C=C/C(=O)N[C@H]2C[C@H](NC(=O)CN3CCN(c4cc(-c5ccccc5O)nnc4N)CC3)C2)c(Cl)c1] | ¹H NMR (400 MHz, DMSO) δ 14.23 (s, 1H), 8.97 (d, J = 7.1 Hz, 1H), 8.16 (d, J = 7.6 Hz, 1H), 7.94 (dd, J = 8.4, 1.6 Hz, 1H), 7.52 (s, 1H), 7.29- 7.21 (m, 2H), 7.07 (dd, J = 8.6, 2.4 Hz, 1H), 6.90 (td, J = 5.6, 2.7 Hz, 2H), 6.75 (d, | Rt = 1.79; 620.1 [M + H]+ |
| | (E)-N-((1r,3r)-3-(2-(4-(3-amino-6-(2- | J = 15.5 Hz, 1H), 6.26 (s, | |
| | hydroxyphenyl)pyridazin-4- | 2H), 4.36 (dq, J = 19.6, 7.1 | |
| | yl)piperazin-1- | Hz, 2H), 3.87 (s, 3H), 3.17 | |
| | yl)acetamido)cyclobutyl)-4-(2-chloro-4- | (s, 3H), 3.01 (s, 2H), 2.68 | |
| | methoxyphenyl)-4-oxobut-2-enamide | (s, 3H), 2.39-2.28 (m, 2H), | |
| | | 2.25-2.14 (m, 2H). | |
| Compound 281 | 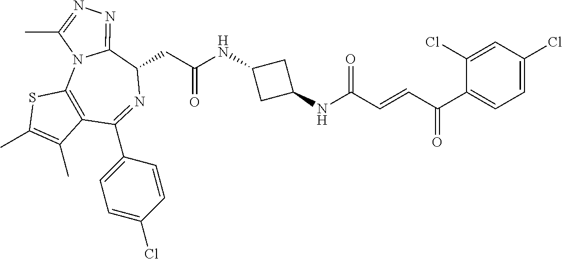 [SMILES: Cc1sc2c(c1C)C(c1ccc(Cl)cc1)=N[C@@H](CC(=O)N[C@H]1C[C@H](NC(=O)/C=C/C(=O)c3ccc(Cl)cc3Cl)C1)c1nnc(C)n1-2] | ¹H NMR (400 MHz, DMSO) δ 8.96 (d, J = 7.3 Hz, 1H), 8.65 (d, J = 7.1 Hz, 1H), 7.83 (d, J = 1.8 Hz, 1H), 7.68-7.58 (m, 2H), 7.51 (d, J = 8.5 Hz, 2H), 7.42 (d, J = 8.4 Hz, 2H), 7.12 (d, J = 15.7 Hz, 1H), 6.69 (d, J = 15.7 Hz, 1H), 4.53-4.45 (m, 1H), 4.40 (q, J = 6.7 Hz, 1H), 4.30 (d, J = 6.9 Hz, 1H), 3.22 (qd, J = 15.0, 7.2 Hz, 3H), 2.60 (s, 3H), 2.42 (s, 3H), 2.27 (d, J = 10.9 Hz, 2H), 1.62 (s, 3H) | Rt = 2.57; 695.0 [M + H]+ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-(2,4- | | |
| | dichlorophenyl)-4-oxobut-2-enamide | | |
| Compound 282 | 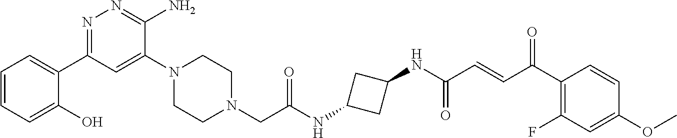 [SMILES: COc1ccc(C(=O)/C=C/C(=O)N[C@H]2C[C@H](NC(=O)CN3CCN(c4cc(-c5ccccc5O)nnc4N)CC3)C2)c(F)c1] | ¹H NMR (400 MHz, DMSO) δ 14.23 (s, 1H), 8.99 (d, J = 7.1 Hz, 1H), 8.17 (d, J = 7.6 Hz, 1H), 7.94 (dd, J = 8.3, 1.7 Hz, 1H), 7.82 (t, J = 8.8 Hz, 1H), 7.56-7.47 (m, 2H), 7.25 (ddd, J = 8.5, 7.2, 1.5 Hz, 1H), 7.01 (dd, J = 13.5, | Rt = 1.74; 604.2 [M + H]+ |
| | (E)-N-((1r,3r)-3-(2-(4-(3-amino-6-(2- | 2.4 Hz, 1H), 6.98-6.86 (m, | |
| | hydroxyphenyl)pyridazin-4- | 4H), 6.26 (s, 2H), 4.45- | |
| | yl)piperazin-1- | 4.31 (m, 2H), 3.88 (s, 3H), | |
| | yl)acetamido)cyclobutyl)-4-(2-fluoro-4- | 3.17 (s, 3H), 3.01 (s, 2H), | |
| | methoxyphenyl)-4-oxobut-2-enamide | 2.69 (d, J = 5.2 Hz, 4H), | |
| | | 2.35 (s, 1H), 2.28-2.17 (m, | |
| | | 2H). | |
| Compound 283 | 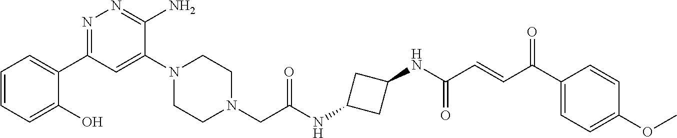 [SMILES: COc1ccc(C(=O)/C=C/C(=O)N[C@H]2C[C@H](NC(=O)CN3CCN(c4cc(-c5ccccc5O)nnc4N)CC3)C2)cc1] | ¹H NMR (400 MHz, DMSO) δ 14.24 (s, 1H), 8.98 (d, J = 7.1 Hz, 1H), 8.18 (d, J = 7.6 Hz, 1H), 8.08-7.99 (m, 2H), 7.95 (dd, J = 8.3, 1.7 Hz, 1H), 7.77 (d, J = 15.2 Hz, 1H), 7.53 (s, 1H), 7.30-7.21 (m, 1H), 7.11 (d, J = 8.7 Hz, | Rt = 1.70; 586.2 [M + H]+ |
| | (E)-N-((1r,3r)-3-(2-(4-(3-amino-6-(2- | 2H), 7.00-6.87 (m, 3H), | |
| | hydroxyphenyl)pyridazin-4- | 6.26 (s, 2H), 4.39 (dt, J = | |
| | yl)piperazin-1- | 19.5, 6.4 Hz, 2H), 3.88 (s, | |
| | yl)acetamido)cyclobutyl)-4-(4- | 3H), 3.18 (s, 3H), 3.02 (s, | |
| | methoxyphenyl)-4-oxobut-2-enamide | 2H), 2.69 (d, J = 6.0 Hz, | |
| | | 3H), 2.36 (dt, J = 13.8, 7.6 | |
| | | Hz, 2H), 2.29-2.19 (m, | |
| | | 2H). | |
| Compound 284 | 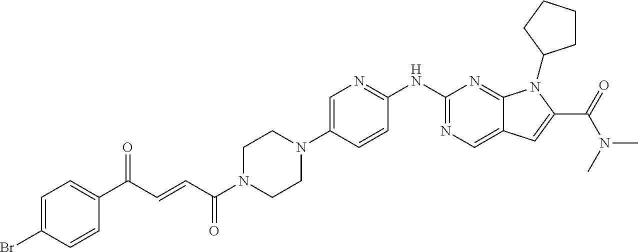 [SMILES: CN(C)C(=O)c1cc2cnc(Nc3ccc(N4CCN(C(=O)/C=C/C(=O)c5ccc(Br)cc5)CC4)cn3)nc2n1C1CCCC1] | ¹H NMR (400 MHz, DMSO) δ 9.33 (s, 1H), 8.76 (s, 1H), 8.18 (d, J = 9.1 Hz, 1H), 8.06-7.95 (m, 2H), 7.84-7.72 (m, 3H), 7.56- 7.45 (m, 2H), 6.61 (s, 1H), 4.78-4.70 (m, 1H), 3.77 (s, 3H), 3.18 (s, 3H), 3.06 (s, 4H), 2.44 (s, 1H), 2.08 (s, 2H), 1.98 (s, 3H), 1.65 (s, 1H). | Rt = 2.36; 671.3 [M + H]+ |
| | (E)-2-((5-(4-(4-(4-bromophenyl)-4- | | |
| | oxobut-2-enoyl)piperazin-1-yl)pyridin- | | |
| | 2-yl)amino)-7-cyclopentyl-N,N- | | |
| | dimethyl-7H-pyrrolo[2,3-d]pyrimidine- | | |
| | 6-carboxamide | | |
| Compound 285 | 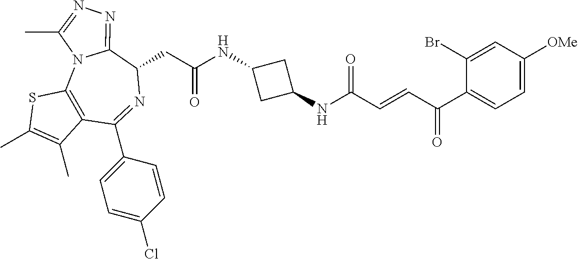 [SMILES: COc1ccc(C(=O)/C=C/C(=O)N[C@H]2C[C@H](NC(=O)C[C@@H]3N=C(c4ccc(Cl)cc4)c4c(sc(C)c4C)-n4c(C)nnc43)C2)c(Br)c1] | ¹H NMR (400 MHz, DMSO) δ 8.96 (d, J = 7.3 Hz, 1H), 8.65 (d, J = 7.1 Hz, 1H), 7.59 (d, J = 8.6 Hz, 1H), 7.51 (d, J = 8.3 Hz, 2H), 7.42 (d, J = 8.2 Hz, 2H), 7.34 (d, J = 2.5 Hz, 1H), 7.22 (d, J = 15.6 Hz, 1H), 7.10 (dd, J = 8.6, 2.4 Hz, 1H), 6.71 (d, J = 15.6 Hz, 1H), 4.49 (t, J = 7.1 Hz, 1H), 4.44-4.36 (m, 1H), 4.31 (d, J = 7.1 Hz, 1H), 3.86 (s, 3H), 3.29-3.14 (m, 2H), 2.60 (s, 3H), 2.42 (s, 3H), 2.27 (s, 2H), 2.22 (s, | Rt = 2.94; 735.2 [M + H]+ |
| | (E)-4-(2-bromo-4-methoxyphenyl)-N- | 1H), 1.62 (s, 3H). | |
| | ((1S,3r)-3-(2-((S)-4-(4-chlorophenyl)- | | |
| | 2,3,9-trimethyl-6H-thieno[3,2- | | |
| | f][1,2,4]triazolo[4,3-a][1,4]diazepin-6- | | |
| | yl)acetamido)cyclobutyl)-4-oxobut-2- | | |
| | enamide | | |
| Compound 286 | 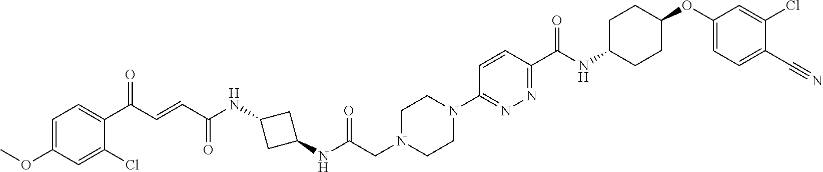 [SMILES: COc1ccc(C(=O)/C=C/C(=O)N[C@H]2C[C@H](NC(=O)CN3CCN(c4ccc(C(=O)N[C@H]5CC[C@H](Oc6ccc(C#N)c(Cl)c6)CC5)nn4)CC3)C2)c(Cl)c1] | ¹H NMR (400 MHz, DMSO) δ 8.97 (d, J = 7.2 Hz, 1H), 8.63 (d, J = 8.2 Hz, 1H), 8.18 (d, J = 7.5 Hz, 1H), 7.85 (dd, J = 9.1, 6.4 Hz, 2H), 7.63 (d, J = 8.6 Hz, 1H), 7.42-7.32 (m, 2H), 7.25 (d, J = 15.5 Hz, 1H), 7.21-7.10 (m, 2H), 7.07 (dd, J = 8.6, 2.5 Hz, 1H), 6.75 (d, J = 15.5 Hz, 1H), | Rt = 3.00; 790.3 [M + H]+ |
| | N-((1r,4r)-4-(3-chloro-4- | 4.53 (dd, J = 9.7, 4.9 Hz, | |
| | cyanophenoxy)cyclohexyl)-6-(4-(2- | 1H), 4.42-4.28 (m, 2H), | |
| | (((1r,3r)-3-((E)-4-(2-chloro-4- | 3.87 (s, 3H), 3.76 (t, J = 5.2 | |
| | methoxyphenyl)-4-oxobut-2- | Hz, 4H), 2.99 (s, 2H), 2.57 | |
| | enamido)cyclobutyl)amino)-2- | (t, J = 5.1 Hz, 4H), 2.39- | |
| | oxoethyl)piperazin-1-yl)pyridazine-3- | 2.28 (m, 2H), 2.21 (dd, J = | |
| | carboxamide | 8.1, 4.7 Hz, 2H), 2.11 (d, J = | |
| | | 11.7 Hz, 2H), 1.90 (d, J = | |
| | | 12.1 Hz, 2H), 1.65 (q, J = | |
| | | 12.2 Hz, 2H), 1.52 (q, J = | |
| | | 11.7 Hz, 2H). | |
| Compound 287 | 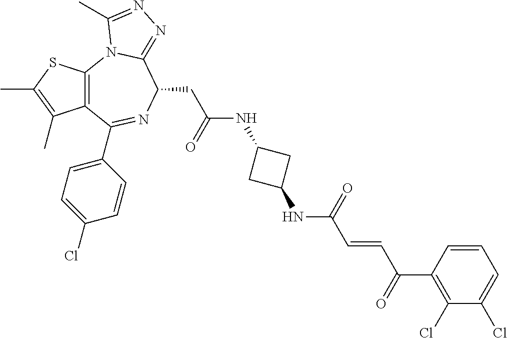 [SMILES: Cc1sc2c(c1C)C(c1ccc(Cl)cc1)=N[C@@H](CC(=O)N[C@H]1C[C@H](NC(=O)/C=C/C(=O)c3cccc(Cl)c3Cl)C1)c1nnc(C)n1-2] | ¹H NMR (400 MHz, DMSO) δ 8.96 (d, J = 7.2 Hz, 1H), 8.64 (d, J = 7.1 Hz, 1H), 7.90-7.81 (m, 1H), 7.58-7.46 (m, 4H), 7.42 (d, J = 8.6 Hz, 2H), 7.08 (d, J = 15.8 Hz, 1H), 6.67 (d, J = 15.8 Hz, 1H), 4.49 (dd, J = 8.1, 6.2 Hz, 1H), 4.40 (q, J = 6.9 Hz, 1H), 4.29 (q, J = 6.9 Hz, 1H), 3.24 (ddd, J = 29.8, 14.6, 7.4 Hz, 2H), 2.60 (s, 3H), 2.41 (s, 3H), 2.24 (td, J = 14.8, 6.6 Hz, 4H), 1.62 (s, 3H). | Rt = 3.08; 697.1 [M + H]+ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-(2,3- | | |
| | dichlorophenyl)-4-oxobut-2-enamide | | |
| Compound 288 | 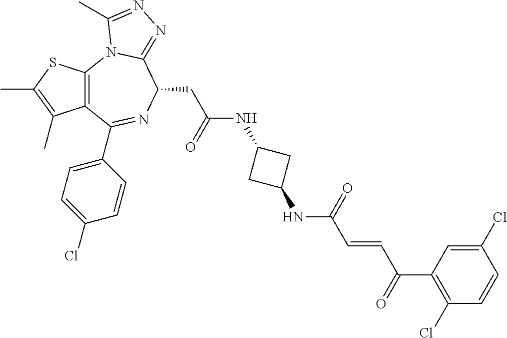 [SMILES: Cc1sc2c(c1C)C(c1ccc(Cl)cc1)=N[C@@H](CC(=O)N[C@H]1C[C@H](NC(=O)/C=C/C(=O)c3cc(Cl)ccc3Cl)C1)c1nnc(C)n1-2] | ¹H NMR (400 MHz, DMSO) δ 8.98 (d, J = 7.2 Hz, 1H), 8.65 (d, J = 7.1 Hz, 1H), 7.75-7.62 (m, 3H), 7.50 (d, J = 8.3 Hz, 2H), 7.42 (d, J = 8.3 Hz, 2H), 7.09 (d, J = 15.8 Hz, 1H), 6.69 (d, J = 15.8 Hz, 1H), 4.49 (t, J = 7.1 Hz, 1H), 4.40 (d, J = 7.2 Hz, 1H), 4.30 (d, J = 6.8 Hz, 1H), 3.20 (td, J = 15.2, 7.2 Hz, 3H), 2.60 (s, 3H), 2.41 (s, 3H), 2.23 (d, J = 17.4 Hz, 4H), 1.62 (s, 3H). | Rt = 2.26; 697.2 [M + H]+ |
| | (E)-N-((1S,3r)-3-(2-((S)-4-(4- | | |
| | chlorophenyl)-2,3,9-trimethyl-6H- | | |
| | thieno[3,2-f][1,2,4]triazolo[4,3- | | |
| | a][1,4]diazepin-6- | | |
| | yl)acetamido)cyclobutyl)-4-(2,5- | | |
| | dichlorophenyl)-4-oxobut-2-enamide | | |

### Example 21: Cell Culture and Preparation of Cell Lysates

### Cell Culture

C33A cells were purchased from American Type Culture Collection (ATCC) and were cultured in Dulbecco's Modified Eagle Medium (DMEM) containing 10% (v/v) fetal bovine serum (FBS) and maintained at 37° C. with 5% CO₂. 22RV1 cells were purchased from the ATCC and were cultured in RPMI-1640 Medium containing 10% (v/v) FBS and maintained at 37° C. with 5% CO₂. HEK293T cells were obtained from the UC Berkeley Cell Culture Facility and were cultured in DMEM containing 10% (v/v) FBS and maintained at 37° C. with 5% CO₂. K562 cells were obtained from the UC Berkeley Cell Culture Facility and were cultured in Iscove's Modified Dulbecco's Medium (IMDM) containing 10% (v/v) FBS and maintained at 37° C. with 5% CO₂. MV-4-11 cells were obtained from the ATCC and were cultured in IMDM containing 10% (v/v) FBS and maintained at 37° C. with 5% CO₂. A549 cells were obtained from the ATCC and were cultured in F-12K Medium containing 10% (v/v) FBS and maintained at 37° C. with 5% CO₂. Mino cells were obtained from the ATCC and were cultured in RPMI-1640 Medium containing 10% (v/v) FBS and maintained at 37° C. with 5% CO₂. LNCaP cells were obtained from the ATCC and were cultured in RPMI-1640 Medium containing 10% (v/v) FBS and maintained at 37° C. with 5% CO₂. Unless otherwise specified, all cell culture materials were purchased from Gibco. It is not known whether HEK293T cells are from male or female origin.

### Preparation of Cell Lysates

Cells were washed twice with cold PBS, scraped, and pelleted by centrifugation (700 g, 5 min, 4° C.). Pellets were resuspended in PBS, sonicated, clarified by centrifugation (12,000 g, 10 min, 4° C.), and lysate was transferred to new low-adhesion microcentrifuge tubes. Proteome concentrations were determined using BCA assay and lysate was diluted to appropriate working concentrations.

### Example 22: Western Blotting, Degradation Assays

Proteins were resolved by SDS/PAGE and transferred to nitrocellulose membranes using the Trans-Blot Turbo transfer system (Bio-Rad). Membranes were blocked with 5% BSA in Tris-buffered saline containing Tween 20 (TBS-T) solution for 30 min at RT, washed in TBS-T, and probed with primary antibody diluted in recommended diluent per manufacturer overnight at 4° C. After 3 washes with TBS-T, the membranes were incubated in the dark with IR680- or IR800-conjugated secondary antibodies at 1:10,000 dilution in 5% BSA in TBS-T at RT for 1 h. After 3 additional washes with TBST, blots were visualized using an Odyssey Li-Cor fluorescent scanner. The membranes were stripped using ReBlot Plus Strong Antibody Stripping Solution (EMD Millipore) when additional primary antibody incubations were performed. Antibodies used in this study were CDK4 (Abcam ab108357), Vinculin (Abcam ab129002), GAPDH (Cell Signaling Technology 14C10), RNF126 (Santa Cruz Biotechnology sc-376005), BRD4 (Abcam ab128874), Beta Actin (Cell Signaling Technology 13E5), PDE5 (Abcam ab259945), AR-V7 (Abcam ab273500), c-Abl (Santa Cruz Biotechnology sc-23), SMARCA2 (Abcam ab240648), LRRK2 (Abcam ab133474), BTK (Cell Signaling Technology D3H5), Androgen Receptor (Abcam ab133273).

TABLE 6
BRD9 (long and short) Degradation Assay data
for Exemplary Compounds

| Compound | DC50 (BRD4 |
|---|---|
| No. | long/BRD4 short) |
| 139 | >1 μM/>1 μM |
| 140 | <100 nM/<100 nM |
| 141 | >1 μM/>1 μM |
| 142 | <100 nM/<100 nM |
| 143 | <100 nM/<100 nM |
| 144 | >1 μM/>1 μM |
| 202 | <100 nM/<100 nM |
| 203 | 10 nM-100 nM/10 nM-100 nM |
| 204 | 10 nM-100 nM/10 nM-100 nM |
| 205 | 10 nM-100 nM/10 nM-100 nM |
| 206 | 10 nM-100 nM/10 nM-100 nM |
| 207 | 10 nM-100 nM/10 nM-100 nM |
| 208 | <10 nM/<10 nM |
| 209 | 100 nM-1 μM/100 nM-1 μM |
| 210 | <100 nM/<100 nM |
| 211 | 10 nM-100 nM/10 nM-100 nM |
| 212 | 100 nM-1 μM/10 nM-100 nM |
| 213 | 100 nM-1 μM/100 nM-1 μM |
| 214 | 10 nM-100 nM/10 nM-100 nM |
| 215 | <10 nM/10 nM-100 nM |
| 216 | <10 nM/<10 nM |
| 217 | <10 nM/<10 nM |
| 218 | <10 nM/<10 nM |
| 219 | 100 nM-1 μM/100 nM-1 μM |
| 221 | 10 nM-100 nM/10 nM-100 nM |
| 222 | 1 nM-10 nM/1 nM-10 nM |
| 223 | 8 nM/12 nM |
| 224 | 1 nM-10 nM/1 nM-10 nM |
| 225 | <10 nM/100 nM-1 μM |
| 226 | 10 nM-100 nM/10 nM-100 nM |
| 227 | 10 nM-100 nM/10 nM-100 nM |
| 228 | 10 nM-100 nM/10 nM-100 nM |
| 229 | 100 nM-1 μM/100 nM-1 μM |
| 230 | 10 nM-100 nM/10 nM-100 nM |
| 231 | 10 nM-100 nM/1 nM-10 nM |
| 232 | 10 nM-100 nM/10 nM-100 nM |
| 233 | <100 nM/<100 nM |
| 234 | 50 nM/150 nM |
| 235 | <100 nM/<100 nM |
| 236 | 90 nM/190 nM |
| 237 | <100 nM/<100 nM |
| 238 | 100 nM-1 μM/<100 nM |
| 239 | 100 nM-1 μM/100 nM-1 μM |
| 240 | <10 nM/10 nM-100 nM |
| 241 | 90 nM/180 nM |
| 242 | 80 nM/40 nM |
| 243 | 100 nM-1 μM/100 nM-1 μM |
| 244 | 100 nM-1 μM/1 μM |
| 245 | >5 μM/>5 μM |
| 246 | >5 μM/100 nM-1 μM |
| 247 | 100 nM-1 μM/100 nM-1 μM |
| 248 | >5 μM/>5 μM |
| 249 | >5 μM/>5 μM |
| 250 | >5 μM/>5 μM |
| 251 | >5 μM/10 nM-100 nM |
| 252 | >5 μM/>5 μM |
| 253 | >5 μM/>5 μM |
| 254 | >5M/>5 μM |
| 277 | 10 nM-100 nM/10 nM-100 nM |
| 278 | 10 nM-100 nM/10 nM-100 nM |
| 279 | >5 μM/>5 μM |
| 281 | 100 nM-1 μM/100 nM-1 μM |
| 287 | 1 μM-5 μM/1 μM-5 μM |
| 288 | 100 nM-1 μM/100 nM-1 μM |

TABLE 7
CDK4 Degradation Assay data for Exemplary Compounds

| Compound | |
|---|---|
| No. | DC50 (CDK4) |
| 146 | >10 μM |
| 148 | >10 μM |
| 151 | >10 μM |
| 158 | >10 μM |
| 159 | >10 μM |
| 160 | >10 μM |
| 165 | >10 μM |
| 255 | >10 μM |
| 256 | >10 μM |
| 257 | >10 μM |
| 258 | >10 μM |
| 259 | >10 μM |
| 260 | >10 μM |
| 261 | >10 μM |
| 262 | 1 μM-10 μM |
| 263 | >10 μM |
| 264 | >10 μM |
| 265 | >10 μM |
| 266 | >10 μM |
| 267 | >10 μM |
| 268 | >10 μM |
| 269 | >10 μM |
| 270 | >10 μM |
| 271 | 1 μM-10 μM |
| 272 | >10 μM |
| 273 | 1 μM-10 μM |
| 274 | 1 μM-10 μM |
| 275 | 1 μM-10 μM |
| 276 | 1 μM-10 μM |
| 284 | >10 μM |

TABLE 8
Androgen Receptor Degradation assay data for
Exemplary Compounds

| Compound | DC50 (Androgen |
|---|---|
| No. | Receptor) |
| 200 | >10 μM |
| 201 | >10 μM |
| 286 | >10 μM |

TABLE 9
SMARCA2 Degradation assay data for Exemplary Compounds

| Compound | |
|---|---|
| No. | DC50 (SMARCA2) |
| 282 | >5 μM |
| 283 | >5 μM |

TABLE 10
RNF126 Binding Assay data for Exemplary Compounds

| Compound | IC50 (RNF126 |
|---|---|
| No. | binding) |
| 145 | >50 μM |
| 147 | 1 μM-10 μM |
| 149 | 1 μM-10 μM |
| 150 | 1 μM-10 μM |
| 152 | 100 nM-1 μM |
| 153 | 100 nM-1 μM |
| 154 | 100 nM-1 μM |
| 155 | 100 nM-1 μM |
| 156 | <100 nM |
| 157 | <100 nM |
| 161 | 100 nM-1 μM |
| 162 | <100 nM |
| 163 | <100 nM |
| 164 | <100 nM |
| 166 | 10 μM-50 μM |
| 400 | 100 nM-1 μM |
| 401 | <100 nM |
| 402 | 100 nM-1 μM |
| 403 | 100 nM-1 μM |
| 404 | <100 nM |
| 405 | 100 nM-1 μM |
| 406 | 100 nM-1 μM |
| 407 | <100 nM |
| 408 | 100 nM-1 μM |
| 409 | 100 nM-1 μM |
| 411 | 10 μM-50 μM |
| 412 | 100 nM-1 μM |
| 413 | 100 nM-1 μM |
| 414 | <100 nM |
| 415 | <100 nM |
| 416 | <100 nM |
| 417 | 100 nM-1 μM |
| 418 | 100 nM-1 μM |
| 419 | 100 nM-1 μM |

### Example 23: Expression and Purification of Recombinant RNF126 Protein

RNF126 mammalian expression plasmid with a C-terminal FLAG tag was purchased from Origene (Origene Technologies Inc., RC204986). The plasmid was transformed into NEB 5-alpha Competent E. coli (DH5) cells (NEB product no. C2987H). The following day, a single transformed colony was used to inoculate 50 ml of nutrient rich LB medium containing kanamycin (50 μg/ml) and was incubated at 37° C. overnight, with agitation (250 rpm). A Miniprep (Qiagen) kit was used to isolate the plasmid before sequence verification with appropriate primers.

HEK293T cells were grown to 30-50% confluency in DMEM supplemented with 10% FBS (Corning) and maintained at 37° C. with 5% CO₂. Immediately before transfection, media was replaced with DMEM containing 5% FBS. Each plate was transfected with 24 μg of overexpression plasmid with 24 μL Lipofectamine 3000 (Invitrogen) in Opti-MEM. After 48 h cells were collected in PBS, lysed by sonication, and batch bound with anti-DYKDDDDK resin (GenScript, L00432) for 2 hours. Lysate and resin were washed with PBS and eluted with 133.33 μg/ml 3×FLAG peptide (ApexBio, A6001) in PBS. Five elutions were performed for 15 minutes each. Elutions were concentrated and the protein was stored in PBS. Concentration and purity was determined using the BCA assay and western blotting.

### Example 24: IsoTOP-ABPP Chemoproteomic Experiments

IsoTOP-ABPP studies were done as previously reported (Weerapana, E. et al. Nature 468, 790-795 (2010); Spradlin, J. N. et al. Nat. Chem. Biol. 15, 747-755 (2019); Grossman, E. A. et al. Cell Chem. Biol. 24, 1368-1376.e4 (2017)). All of the isoTOP-ABPP datasets were prepared as described below using the IA-alkyne probe. Cells were lysed by probe sonication in PBS and protein concentrations were measured by BCA assay. Cells were treated for 2 h with either DMSO vehicle or compound before cell collection and lysis. Proteomes were subsequently labeled with IA-alkyne labeling (200 μM) for 1 h at room temperature. CuAAC was used by sequential addition of tris(2-carboxyethyl)phosphine (1 mM, Strem, 15-7400), tris[(1-benzyl-1H-1,2,3-triazol-4-yl)methyl]amine (34 μM, Sigma, 678937), copper(II) sulfate (1 mM, Sigma, 451657) and biotin-linker-azide—the linker functionalized with a tobacco etch virus (TEV) protease recognition sequence as well as an isotopically light or heavy valine for treatment of control or treated proteome, respectively. After CuAAC, proteomes were precipitated by centrifugation at 6,500 g, washed in ice-cold methanol, combined in a 1:1 control:treated ratio, washed again, then denatured and resolubilized by heating in 1.2% SDS-PBS to 90° C. for 5 min. Insoluble components were precipitated by centrifugation at 6,500 g and soluble proteome was diluted in 5 ml 0.2% SDS-PBS. Labeled proteins were bound to streptavidin-agarose beads (170 μl resuspended beads per sample, Thermo Fisher, 20349) while rotating overnight at 4° C. Bead-linked proteins were enriched by washing three times each in PBS and water, then resuspended in 6 M urea/PBS, reduced in DTT (9.26 mM, ThermoFisher, R0861), and alkylated with iodoacetamide (18 mM, Sigma, I6125), before being washed and resuspended in 2 M urea/PBS and trypsinized overnight with 0.5 μg/μL sequencing grade trypsin (Promega, V5111). Tryptic peptides were eluted off Beads were washed three times each in PBS and water, washed in TEV buffer solution (water, TEV buffer, 100 μM dithiothreitol) and resuspended in buffer with Ac-TEV protease (Invitrogen, 12575-015) and incubated overnight. Peptides were diluted in water and acidified with formic acid (1.2 M, Fisher, A117-50) and prepared for analysis.

### Example 25: IsoTOP-ABPP Mass Spectrometry Analysis

Peptides from all chemoproteomic experiments were pressure-loaded onto a 250 μm inner diameter fused silica capillary tubing packed with 4 cm of Aqua C18 reverse-phase resin (Phenomenex, 04A-4299), which was previously equilibrated on an Agilent 600 series high-performance liquid chromatograph using the gradient from 100% buffer A to 100% buffer B over 10 min, followed by a 5 min wash with 100% buffer B and a 5 min wash with 100% buffer A. The samples were then attached using a MicroTee PEEK 360 m fitting (Thermo Fisher Scientific p-888) to a 13 cm laser pulled column packed with 10 cm Aqua C18 reverse-phase resin and 3 cm of strong-cation exchange resin for isoTOP-ABPP studies. Samples were analyzed using an Q Exactive Plus mass spectrometer (Thermo Fisher Scientific) using a five-step Multidimensional Protein Identification Technology (MudPIT) program, using 0, 25, 50, 80 and 100% salt bumps of 500 mM aqueous ammonium acetate and using a gradient of 5-55% buffer B in buffer A (buffer A: 95:5 water:acetonitrile, 0.1% formic acid; buffer B 80:20 acetonitrile:water, 0.1% formic acid). Data were collected in data-dependent acquisition mode with dynamic exclusion enabled (60 s). One full mass spectrometry (MS1) scan (400-1,800 mass-to-charge ratio (m/z)) was followed by MS2 scans of the nth most abundant ions. Heated capillary temperature was set to 200° C. and the nanospray voltage was set to 2.75 kV.

Data were extracted in the form of MS1 and MS2 files using Raw Extractor v.1.9.9.2 (Scripps Research Institute) and searched against the Uniprot human database using ProLuCID search methodology in IP2 v.3-v.5 (Integrated Proteomics Applications, Inc.) (Xu, T. et al. ProLuCID: An improved SEQUEST-like algorithm with enhanced sensitivity and specificity. J. Proteomics 129, 16-24 (2015)). Cysteine residues were searched with a static modification for carboxyaminomethylation (+57.02146) and up to two differential modifications for methionine oxidation and either the light or heavy TEV tags (+464.28596 or +470.29977, respectively). Peptides were required to be fully tryptic peptides and to contain the TEV modification. ProLUCID data were filtered through DTASelect to achieve a peptide false-positive rate below 5%. Only those probe-modified peptides that were evident across two out of three biological replicates were interpreted for their isotopic light to heavy ratios. For those probe-modified peptides that showed ratios greater than two, we only interpreted those targets that were present across all three biological replicates, were statistically significant and showed good quality MS1 peak shapes across all biological replicates. Light versus heavy isotopic probe-modified peptide ratios are calculated by taking the mean of the ratios of each replicate paired light versus heavy precursor abundance for all peptide-spectral matches associated with a peptide. The paired abundances were also used to calculate a paired sample t-test P value in an effort to estimate constancy in paired abundances and significance in change between treatment and control. P values were corrected using the Benjamini-Hochberg method.

### Example 26: Gel-Based ABPP

Recombinant RNF126 (0.1 g/sample) was pre-treated with either DMSO vehicle or covalent ligand at 37° C. for 30 min in 25 μL of PBS, and subsequently treated with of IA-Rhodamine (concentrations designated in figure legends) (Setareh Biotech) at room temperature for 1 h in the dark. The reaction was stopped by addition of 4×reducing Laemmli SDS sample loading buffer (Alfa Aesar). After boiling at 95° C. for 5 min, the samples were separated on precast 4-20% Criterion TGX gels (Bio-Rad). Probe-labeled proteins were analyzed by in-gel fluorescence using a ChemiDoc MP (Bio-Rad).

### Example 27: Compound 122-Alkyne Pulldown Quantitative Proteomics

Cells were treated with either DMSO vehicle or compound (Compound 122-alkyne 10 μM) for 6 h. Cells were harvested and lysed by probe sonication in PBS and protein concentrations were measured by BCA assay. CuAAC was used by sequential addition of tris(2-carboxyethyl)phosphine (893 μM, Strem, 15-7400), tris[(1-benzyl-1H-1,2,3-triazol-4-yl)methyl]amine (91 μM, Sigma, 678937), copper(II) sulfate (893 M, Sigma, 451657) and biotin picolyl azide (179 μM, Sigma, 900912). After 1 h, proteomes were precipitated by centrifugation at 6,500 g, washed in ice-cold methanol, combined to attain 10 mg per sample, washed again, then denatured and resolubilized by heating in 1.2% SDS-PBS to 90° C. for 5 min. The soluble proteome was diluted with 4 mL of PBS and labeled proteins were bound to streptavidin-agarose beads (170 μl resuspended beads per sample, Thermo Fisher, 20349) while rotating overnight at 4° C. Bead-linked proteins were enriched by washing three times each in PBS and water, then resuspended in 6 M urea/PBS, reduced in DTT (9.26 mM, ThermoFisher, R0861), and alkylated with iodoacetamide (18 mM, Sigma, I6125), before being washed and resuspended in 50 mM Triethylammonium bicarbonate (TEAB) and trypsinized overnight with 0.5 μg/μL sequencing grade trypsin (Promega, V5111). Tryptic peptides were eluted off. Individual samples were then labeled with isobaric tags using commercially available TMTsixplex (Thermo Fisher Scientific, P/N 90061) kits, in accordance with the manufacturer's protocols. Tagged samples (20 g per sample) were combined, dried with SpeedVac, resuspended with 300 μL 0.1% TFA in H2O, and fractionated using high pH reversed-phase peptide fractionation kits (Thermo Scientific, P/N 84868) according to manufacturer's protocol. Fractions were dried with SpeedVac, resuspended with 50 μL 0.1% FA in H2O, and analyzed by LC-MS/MS as described below.

Quantitative TMT-based proteomic analysis was performed as previously described using a Thermo Eclipse with FAIMS LC-MS/MS (King, E. A. et al. Chemoproteomics-Enabled Discovery of a Covalent Molecular Glue Degrader Targeting NF-κB. 2022.05.18.492542 Preprint at https://doi.org/10.1101/2022.05.18.492542 (2022)). Acquired MS data was processed using ProLuCID search methodology in IP2 v.3-v.5 (Integrated Proteomics Applications, Inc.) (Xu, T. et al. J. Proteomics 129, 16-24 (2015)). Trypsin cleavage specificity (cleavage at K, R except if followed by P) allowed for up to 2 missed cleavages. Carbamidomethylation of cysteine was set as a fixed modification, methionine oxidation, and TMT-modification of N-termini and lysine residues were set as variable modifications. Reporter ion ratio calculations were performed using summed abundances with most confident centroid selected from 20 ppm window. Only peptide-to-spectrum matches that are unique assignments to a given identified protein within the total dataset are considered for protein quantitation. High confidence protein identifications were reported with a <1% false discovery rate (FDR) cut-off. Differential abundance significance was estimated using ANOVA with Benjamini-Hochberg correction to determine p-values.

### Example 28: Quantitative TMT Proteomics Analysis

Cells were treated with either DMSO vehicle or compound (Compound 130, 1 μM) for 24 h and lysate was prepared as described above. Briefly, 25-100 μg protein from each sample was reduced, alkylated and tryptically digested overnight. Individual samples were then labeled with isobaric tags using commercially available TMTsixplex (Thermo Fisher Scientific, P/N 90061) kits, in accordance with the manufacturer's protocols. Tagged samples (20 μg per sample) were combined, dried with SpeedVac, resuspended with 300 μL 0.1% TFA in H2O, and fractionated using high pH reversed-phase peptide fractionation kits (Thermo Scientific, P/N 84868) according to manufacturer's protocol. Fractions were dried with SpeedVac, resuspended with 50 μL 0.1% FA in H2O, and analyzed by LC-MS/MS as described below.

Quantitative TMT-based proteomic analysis was performed as previously described using a Thermo Eclipse with FAIMS LC-MS/MS (King, E. A. et al. Chemoproteomics-Enabled Discovery of a Covalent Molecular Glue Degrader Targeting NF-κB. 2022.05.18.492542 Preprint at https://doi.org/10.1101/2022.05.18.492542 (2022)). Acquired MS data was processed using ProLuCID search methodology in IP2 v.3-v.5 (Integrated Proteomics Applications, Inc.) (Xu, T. et al. ProLuCID: An improved SEQUEST-like algorithm with enhanced sensitivity and specificity. J. Proteomics 129, 16-24 (2015)). Trypsin cleavage specificity (cleavage at K, R except if followed by P) allowed for up to 2 missed cleavages. Carbamidomethylation of cysteine was set as a fixed modification, methionine oxidation, and TMT-modification of N-termini and lysine residues were set as variable modifications. Reporter ion ratio calculations were performed using summed abundances with most confident centroid selected from 20 ppm window. Only peptide-to-spectrum matches that are unique assignments to a given identified protein within the total dataset are considered for protein quantitation. High confidence protein identifications were reported with a <1% false discovery rate (FDR) cut-off. Differential abundance significance was estimated using ANOVA with Benjamini-Hochberg correction to determine p-values.

### Example 29: Knockdown Studies

Short-hairpin oligonucleotides were used to knock down the expression of RNF126 in C33A cells. For lentivirus production, lentiviral plasmids and packaging plasmids (pMD2.5G, Addgene catalog no. 12259 and psPAX2, Addgene catalog no. 12260) were transfected into HEK293T cells using Lipofectamine 2000 (Invitrogen). Lentivirus was collected from filtered cultured medium and used to infect the target cell line with 1:1000 dilution of polybrene. Target cells were selected over 3 d with 1 g/ml of puromycin for C33A cells and 7.5 g ml-1 for HEK293T cells. The short-hairpin sequences which were used for generation of the knockdown lines were:
RNF126: TGCCATCATCACACAGCTCCT (SEQ ID NO: 1) (Sigma RNF126 MISSION shRNA Bacterial Glycerol Stock, TRCN0000368954).
MISSION TRC1.5 pLKO.1- or TRC2 pLKO.5-puro Non-Mammalian shRNA Control (Sigma) was used as a control shRNA.

### EQUIVALENTS AND SCOPE

This application refers to various issued patents, published patent applications, journal articles, and other publications, all of which are incorporated herein by reference in their entirety. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from.

1. A molecular glue degrader compound of Formula (I): 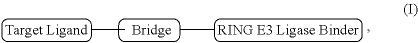
or a pharmaceutically acceptable salt, hydrate, solvate, prodrug, stereoisomer, or tautomer thereof, wherein:
(i) the Target Ligand is capable of binding to a target protein;
(ii) Bridge is absent or a linker; and
(iii) the RING E3 Ligase Binder is capable of binding to a RING E3 ubiquitin ligase (e.g., RNF126 or BCA2).

2. (canceled)

3. The molecular glue degrader compound of claim 1, wherein RING E3 Ligase Binder binds to a RING E3 Zinc ubiquitin ligase.

4. The molecular glue degrader compound of claim 1, wherein the RING E3 Ligase Binder binds to RNF126 E3 ubiquitin ligase or BCA2 E3 ubiquitin ligase.

5. (canceled)

6. The molecular glue degrader compound of claim 1, wherein the RING E3 Ligase Binder binds to a cysteine residue within a RING E3 ubiquitin ligase.

7-11. (canceled)

12. The molecular glue degrader compound of claim 1, wherein the target protein selected from a tyrosine kinase, a serine/threonine kinase, a bromodomain-containing protein, an epigenetic protein, and a misfolded protein.

13. The molecular glue degrader compound of claim 1, wherein the target protein is selected from AR, BCL-2/BCL, BCL-XL, BCR-ABL, BRD2, BRD3, BRD4, BRD9, BRDT, BTK, BUB1, BUB1B, c-ABL, CDK1, CDK2, CDK3, CDK4, CDK5, CDK6, CDK7, CDK8, CDK9, CDK10, CDK11B, CDK12, CDK13, CDK14, CDK15, CDK16, CDK17, CDK18, CDK19, CDK20, CHEK1, CKS1B, CKS2, CSNK1A1, CSNK1E, CTNNB1, DSTYK, EEF2K, estrogen receptor (ER), ETNK1, FASTKD5, HRAS, ITPKB, KRAS, LRKK2, MAPKAPK2 (MK2), MARK2, MAP3K2, MELK, MYC, MYCN, NEK6, NRAS, PANK2, PANK3, PDE5, PHKA1, PHKA2, PKN2, PLK1, PTK6, RIOK2, SKP2, SMARCA2, SMARCA4, STAT1, STAT2, STAT3, STAT4, STAT5A, STAT5B, STAT6, TK1, TTK, and UCK2.

14-16. (canceled)

17. The molecular glue degrader compound of claim 1, having a structure of Formula (II): 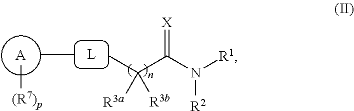
or a pharmaceutically acceptable salt, hydrate, solvate, stereoisomer, or tautomer thereof, wherein:
X is O or S;
Ring A is selected from the group consisting of an aryl, a heteroaryl, a cycloalkyl, and a heterocycloalkyl;
L is absent or a linker;
R¹ is 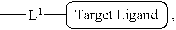
wherein L¹ is absent or a linker and the Target Ligand comprises a moiety capable of binding to a target protein; or
—NR¹ is a Target Ligand;
R² is selected from the group consisting of hydrogen and C₁₋₆ alkyl; or
R¹ and R² together with the atoms to which they are attached form a 5- to 10-membered heteroaryl or a 3- to 12-membered heterocycloalkyl, wherein the heteroaryl or heterocycloalkyl are each substituted with one R⁸, wherein R⁸ is 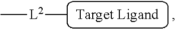
wherein L² is a linker, and the heteroaryl or heterocycloalkyl are each substituted with 0-4 occurrences of R⁹;
each R⁹ is independently selected from the group consisting of —OH, halogen, C₁₋₆ alkyl, C₁₋₆ alkoxyl, C₁₋₆ haloalkyl, alkynyl, —CN, C₁₋₆ haloalkoxyl, —O-aryl, and heteroaryl; or
two R⁹ on the same carbon atom together with the same carbon atom to which they are attached form a C₃₋₈ cycloalkyl or an oxo; or
two R⁹ on adjacent carbon atoms together with the adjacent carbon atoms to which they are attached form a C₃₋₈ cycloalkyl; or
two R⁹ on non-adjacent carbon atoms together with the non-adjacent carbon atoms to which they are attached form a bridging ring;
R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl;
R⁴ and R⁵ are each independently selected from the group consisting of hydrogen, —OH, halogen, —CN, C₁₋₆ alkyl, and C₁₋₆ haloalkyl;
R^{4′} and R^{5′} are each independently selected from the group consisting of hydrogen, —OH, halogen, —CN, C₁₋₆ alkyl, and C₁₋₆ haloalkyl;
R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or
R^{6a} and R^{6b}, together with the atoms to which they are attached, form an oxo group;
R^{6c} is independently selected from the group consisting of hydrogen, C₁₋₆ alkyl, C₁₋₆ hydroxyalkyl, C₁₋₆ haloalkyl, and C₃₋₈ cycloalkyl;
R⁷ are each independently selected from the group consisting of —OH, halogen, C₁₋₆ alkyl, C₁₋₆ alkoxyl, C₁₋₆ haloalkyl, alkynyl, —CN, C₁₋₆ haloalkoxyl, —O-aryl, and heteroaryl; or
two R⁷ on the same carbon atom together with the atoms which they are attached, form an oxo group; or
two R⁷ on adjacent atoms together with the atoms to which they are attached form an optionally substituted 5- to 10-membered aryl or a 3- to 12-membered heterocycloalkyl;
n is 0, 1, or 2; and
p is 0, 1, 2, 3, or 4.

18. (canceled)

19. The molecular glue degrader compound of claim 17, having a structure of Formula (IU-f): 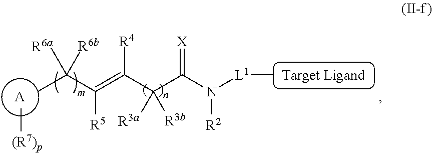
or a pharmaceutically acceptable salt, hydrate, solvate, stereoisomer, or tautomer thereof,
wherein:
X is O or S;
Ring A is selected from the group consisting of a 6- to 10-membered aryl, a 5- to 10-membered heteroaryl, a 3- to 12-membered cycloalkyl, and a 3- to 12-membered heterocycloalkyl;
L¹ is absent or a linker;
Target Ligand comprises a moiety capable of binding to a target protein;
R² is selected from the group consisting of hydrogen and C₁₋₆ alkyl;
R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or
R^{3a} and R^{3b}, together with the atoms to which they are attached, form an oxo group;
R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or
R^{6a} and R^{6b}, together with the atoms to which they are attached, form an oxo group;
R⁷ are each independently selected from the group consisting of —OH, halogen, C₁₋₆ alkyl, C₁₋₆ alkoxyl, C₁₋₆ haloalkyl, and C₁₋₆ haloalkoxyl;
m is 0, 1, or 2;
n is 0, 1, or 2; and
p is 0, 1, 2, 3, or 4

20. The molecular glue degrader compound of claim 17, having a structure of Formula (II-g): 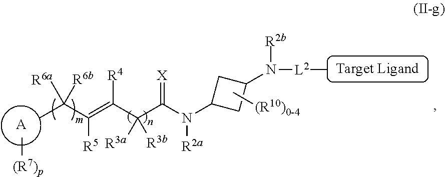
or a pharmaceutically acceptable salt, hydrate, solvate, stereoisomer, or tautomer thereof, wherein:
X is O or S;
Ring A is selected from the group consisting of a 6- to 10-membered aryl, a 5- to 10-membered heteroaryl, a 3- to 12-membered cycloalkyl, and a 3- to 12-membered heterocycloalkyl;
L² is a linker substituted with 0-4 occurrences of R⁹;
Target Ligand comprises a moiety capable of binding to a target protein;
R^{2a} and R^{2b} are each independently hydrogen, C₁₋₆ alkyl, and C₃₋₈ cycloalkyl;
R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or
R^{3a} and R^{3b}, together with the atoms to which they are attached, form an oxo group; R⁴ and R⁵ are each independently selected from the group consisting of hydrogen, —OH, halogen, —CN, C₁₋₆ alkyl, and C₁₋₆ haloalkyl;
R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or
R^{6a} and R^{6b}, together with the atoms to which they are attached, form an oxo group; each
R⁷ is independently selected from the group consisting of —OH, halogen, C₁₋₆ alkyl, C₁₋₆ alkoxyl, C₁₋₆ haloalkyl, and C₁₋₆ haloalkoxyl;
each R⁹ is independently selected from the group consisting of —OH, halogen, C₁₋₆ alkyl, C₁₋₆ alkoxyl, C₁₋₆ haloalkyl, and C₁₋₆ haloalkoxyl; or
two R⁹ on the same carbon atom together with the same carbon atom to which they are attached form a C₃₋₈ cycloalkyl or an oxo; or
two R⁹ on adjacent carbon atoms together with the adjacent carbon atoms to which they are attached form a C₃₋₈ cycloalkyl; or
two R⁹ on non-adjacent carbon atoms together with the non-adjacent carbon atoms to which they are attached form a bridging ring;
each R¹⁰ is independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl;
m is 0, 1, or 2;
n is 0, 1, or 2; and
p is 0, 1, 2, 3, or 4.

21. The molecular glue degrader compound of claim 17, having a structure of Formula (II-h): 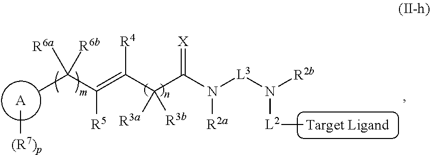
or a pharmaceutically acceptable salt, hydrate, solvate, stereoisomer, or tautomer thereof, wherein:
X is O or S;
Ring A is selected from the group consisting of a 6- to 10-membered aryl, a 5- to 10-membered heteroaryl, a 3- to 12-membered cycloalkyl, and a 3- to 12-membered heterocycloalkyl;
L² is a linker substituted with 0-4 occurrences of R⁹;
L³ is trans-cyclobutane-1,3-diyl or cis-cyclobutane-1,3-diyl, wherein the cyclobutane ring is optionally substituted with 1, 2, 3, or 4 occurrences of R¹⁰;
Target Ligand comprises a moiety capable of binding to a target protein;
R^{2a} and R^{2b} are each independently selected from the group consisting of hydrogen, C₁₋₆ alkyl, and C₃₋₈ cycloalkyl;
R^{3a} and R^{3b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl;
R⁴ and R⁵ are each independently selected from the group consisting of hydrogen, —OH, halogen, —CN, C₁₋₆ alkyl, and C₁₋₆ haloalkyl;
R^{6a} and R^{6b} are each independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl; or
R^{6a} and R^{6b}, together with the atoms to which they are attached, form an oxo group; each R⁷ is independently selected from the group consisting of —OH, halogen, C₁₋₆ alkyl, C₁₋₆ alkoxyl, C₁₋₆ haloalkyl, and C₁₋₆ haloalkoxyl;
each R⁹ is independently selected from the group consisting of —OH, halogen, C₁₋₆ alkyl, C₁₋₆ alkoxyl, C₁₋₆ haloalkyl, and C₁₋₆ haloalkoxyl; or
two R⁹ on the same carbon atom together with the same carbon atom to which they are attached form a C₃₋₈ cycloalkyl or an oxo; or
two R⁹ on adjacent carbon atoms together with the adjacent carbon atoms to which they are attached form a C₃₋₈ cycloalkyl; or
two R⁹ on non-adjacent carbon atoms together with the non-adjacent carbon atoms to which they are attached form a bridging ring;
each R¹⁰ is independently selected from the group consisting of hydrogen, —OH, halogen, C₁₋₆ alkyl, C₁₋₆ alkoxyl, C₁₋₆ hydroxyalkyl, C₁₋₆ haloalkyl, C₁₋₆ haloalkoxyl, and C₃₋₈ cycloalkyl;
m is 0, 1, or 2;
n is 0, 1, or 2; and
p is 0, 1, 2, 3, or 4.

22. The molecular glue degrader compound of claim 17, wherein L¹ and/or L² is absent or selected from the group consisting of —C(O)—, —S(O)₂—, C₁₋₈ alkylene, C₂₋₈ alkenylene, C₂₋₈ alkynylene, C₁₋₈ heteroalkylene, C₂₋₈ heteroalkenylene, C₂₋₈ heteroalkynylene, *C(O)—C₁₋₈ alkylene, *C(O)—C₁₋₈ heteroalkylene, *C(O)—C₁₋₈ alkylene-O, *C₁₋₈ alkylene-C(O)—, *C₁₋₈ heteroalkylene-C(O)—, *C₁₋₈ alkylene-C(O)—, *C₁₋₈ heteroalkylene-C(O)—, wherein * denotes the point of attachment to the N atom in Formula (II).

23. (canceled)

24. The molecular glue degrader compound of claim 17, having a structure of Formula (III): 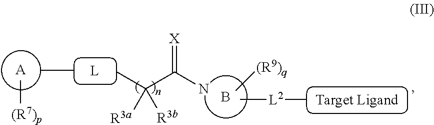
or a pharmaceutically acceptable salt, hydrate, solvate, stereoisomer, or tautomer thereof, wherein A, B, L, L², X, R², R^{3a}, R^{3b}, R⁷, R⁹, n, p, q, and the Target Ligand and variables therein, are defined in claim 17.

25. The molecular glue degrader compound of claim 17, having a structure of Formula (III-a): 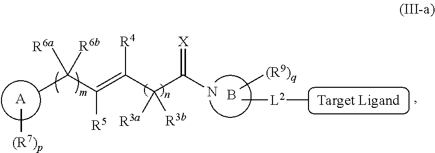
or a pharmaceutically acceptable salt, hydrate, solvate, stereoisomer, or tautomer thereof, wherein A, B, L², X, R⁴, R⁵, R^{3a}, R^{3b}, R^{6a}, R^{6b}, R⁷, R⁹, m, n, p, q, Target Ligand, and variables therein are as defined in claim 17.

26-27. (canceled)

28. The molecular glue degrader compound of claim 17, wherein L² is absent or selected from the group consisting of —C(O)—, —S(O)₂—, C₁₋₈ alkylene, C₂₋₈ alkenylene, C₂₋₈ alkynylene, C₁₋₈ heteroalkylene, C₂₋₈ heteroalkenylene, C₂₋₈ heteroalkynylene, *C(O)—C₁₋₈ alkylene, *C(O)—C₁₋₈ heteroalkylene, *C(O)—C₁₋₈ alkylene-O, *C₁₋₈ alkylene-C(O)—, *C₁₋₈ heteroalkylene-C(O)—, *C₁₋₈ alkylene-C(O)—, *C₁₋₈ heteroalkylene-C(O)—, wherein * denotes the point of attachment to the N atom in Formula (II).

29. The molecular glue degrader compound of claim 17, wherein L² is absent.

30. The molecular glue degrader compound of claim 17, having a structure of Formula (V): 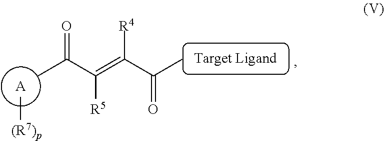
or a pharmaceutically acceptable salt, hydrate, solvate, stereoisomer, or tautomer thereof, wherein A, R⁴, R⁵, R⁷, p, Target Ligand, and variables therein are as defined in claim 17.

31. The molecular glue degrader compound of claim 17, having a structure of Formula (VI): 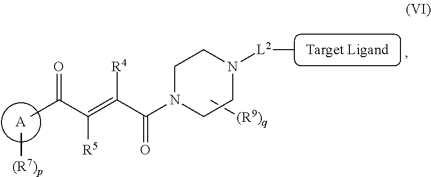
or a pharmaceutically acceptable salt, hydrate, solvate, stereoisomer, or tautomer thereof, wherein A, L², R⁴, R⁵, R⁷, R⁹, p, q, Target Ligand, and variables therein are as defined in claim 17.

32. The molecular glue degrader compound of claim 17, having a structure of Formula (VIII): 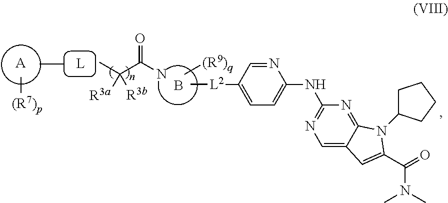
or a pharmaceutically acceptable salt, hydrate, solvate, stereoisomer, or tautomer thereof, wherein A, B, L, L², R^{3a}, R^{3b}, R⁷, R⁹, n, p, q, and variables therein are as defined in claim 17.

33. The molecular glue degrader compound of claim 17, having a structure of Formula (IX-b): 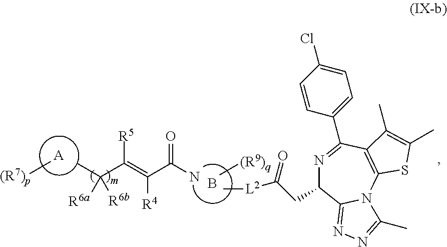
or a pharmaceutically acceptable salt, hydrate, solvate, stereoisomer, or tautomer thereof, wherein A, B, L², R⁴, R⁵, R^{6a}, R^{6b}, R⁷, R⁹, m, p, q, and variables therein are as defined in claim 17.

34. The molecular glue degrader compound of claim 17, having a structure of Formula (IX-e): 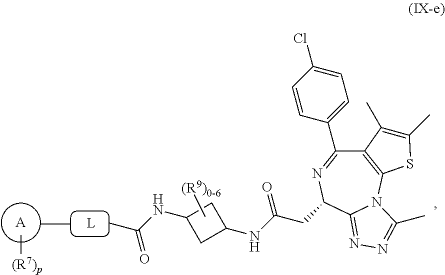
or a pharmaceutically acceptable salt, hydrate, solvate, stereoisomer, or tautomer thereof, wherein A, L, R⁷, R⁹, p, and variables therein are as defined in claim 17.

35. The molecular glue degrader compound of claim 1, wherein the molecular glue degrader compound or an intermediate thereof is selected from a compound in Table 1.

36-37. (canceled)

38. The molecular glue degrader compound of claim 1, wherein the molecular glue degrader compound is capable of modulating one or more of:
(i) reducing the stability of the target protein;
(ii) reducing the half-life of the target protein;
(iii) facilitating trafficking of the target protein to the proteasome;
(iv) increasing the level of ubiquitination of the target protein; and
(v) increasing the rate of degradation (e.g., proteasomal degradation) of the target protein.

39-46. (canceled)

47. The molecular glue degrader compound of claim 1, wherein the RING E3 Ligase Binder binds to an allosteric site within the RING E3 ubiquitin ligase.

48-53. (canceled)

54. A pharmaceutical composition comprising a molecular glue degrader compound of claim 1, and one or more pharmaceutically acceptable carriers.

55. A method of providing a molecular glue degrader compound to a subject, wherein the molecular glue degrader compound is as defined in claim 1.

56. A method of treating a disease, disorder, or condition in a subject, wherein the method comprises administering to the subject a molecular glue degrader compound as defined in claim 1.

57-60. (canceled)

61. A method of ubiquitinating a target protein comprising contacting a cell or sample with a molecular glue compound as defined in claim 1.

62. A method of degrading a target protein comprising contacting a cell or sample with a molecular glue compound as defined in claim 1.

63-74. (canceled)
